(19) United States
(12) Patent Application Publication
(10) Pub. No.: US 2023/0181528 A1
(43) Pub. Date: Jun. 15, 2023

(54) COMPOSITIONS AND METHODS FOR TREATING NEOPLASIA

(71) Applicants: The Broad Institute, Inc., Cambridge, MA (US); Dana-Farber Cancer Institute, Inc., Boston, MA (US)

(72) Inventors: Andrew AGUIRRE, Boston, MA (US); Brenton PAOLELLA, Cambridge, MA (US); Francisca VAZQUEZ, Cambridge, MA (US); Jasper Edgar NEGGERS, Boston, MA (US)

(73) Assignees: The Broad Institute, Inc., Cambridge, MA (US); Dana-Farber Cancer Institute, Inc., Boston, MA (US)

(21) Appl. No.: 18/061,369

(22) Filed: Dec. 2, 2022

Related U.S. Application Data
(63) Continuation of application No. PCT/US2021/035976, filed on Jun. 4, 2021.
(60) Provisional application No. 63/035,454, filed on Jun. 5, 2020.
(60) Provisional application No. 63/041,229, filed on Jun. 19, 2020.

(51) Int. Cl. A61K 31/403; A61K 31/404; A61P 35/00; A61K 38/46; A61K 38/21; C12N 15/113; A61K 31/7105; C12N 15/90; C12N 15/11; C12N 9/22

(52) U.S. Cl. CPC A61K 31/403; A61K 31/404; A61P 35/00; A61K 38/465; A61K 38/21; C12N 15/1137; A61K 31/7105; C12N 15/907; C12N 15/11; C12N 9/22; C12N 2310/14; C12N 2310/531; C12N 2320/30; C12N 2310/20

(57) ABSTRACT

The disclosure is directed to compositions and methods that are useful for the treatment of a neoplasia. Specifically, methods for inducing cell death or reducing cell survival of a neoplastic cell (e.g., rhabomyosarcoma) and methods of treating a subject having a neoplasia characterized by a loss of VPS4 expression are disclosed.

### CROSS-REFERENCE TO RELATED APPLICATION

This application is a continuation of PCT International Application No. PCT/US2021/035976, filed Jun. 4, 2021, which claims priority to and the benefit of U.S. Provisional Application No. 63/035,454, filed Jun. 5, 2020, and U.S. Provisional Application No. 63/041,229, filed Jun. 19, 2020, the entire contents of each of which are incorporated herein by reference.

### STATEMENT OF RIGHTS TO INVENTIONS MADE UNDER FEDERALLY SPONSORED RESEARCH

This invention was made with government support under Grant No. K08 CA218420-02 awarded by the National Institutes of Health. The government has certain rights in the invention.

### SEQUENCE LISTING

The present application contains a Sequence Listing which has been submitted electronically in XML format following conversion from the originally filed TXT format.

The content of the electronic XML Sequence Listing, (Date of creation: Dec. 2, 2022; Size: 88,222 bytes; Name: 167741-023503US_SL.xml), and the original TXT format, is herein incorporated by reference in its entirety.

### BACKGROUND OF THE INVENTION

Discovery of new biomarker-linked cancer therapeutic targets may enable drug development and ultimately lead to advances in clinical care. Somatic copy number alterations (CNAs) leading to loss of tumor suppressor gene (TSG) function constitute important driver events in tumorigenesis. Unfortunately, there are few existing therapeutic options to target the oncogenic processes evoked by tumor suppressor inactivation. Accordingly, there exists a need for drugs that target tractable synthetic lethal interactions with common somatic CNAs.

### SUMMARY OF THE INVENTION

As described below, the present invention features compositions and methods for treating neoplasia characterized by a reduction in or the loss of VPS4A and/or VPS4B (i.e., VPS4A or VPS4B, or both VPS4A and VPS4B). In some embodiments, the neoplasia is further characterized by a reduction in or loss of SMAD4, or CDH1.

In one aspect, the invention features a method for inducing cell death or reducing cell survival of a rhabdomyosarcoma cell characterized by a loss of VPS4B expression. The method involves contacting the cell with an agent that inhibits the expression or activity of VPS4A, thereby inducing cell death or reducing cell survival of the rhabdomyosarcoma cell.

In another aspect, the invention features a method for inducing cell death or reducing cell survival of a rhabdomyosarcoma cell characterized by a loss of VPS4A expression. The method involves contacting the cell with an agent that inhibits the expression or activity of VPS4B, thereby inducing cell death or reducing cell survival of the rhabdomyosarcoma cell.

In any of the above aspects, the method further involves contacting the cell with an agent that inhibits the expression or activity of ULK3, CHMP1A, CHMP1B, VTA1, and/or IST1.

In one aspect, the invention features a method for inducing cell death or reducing cell survival of a neoplastic cell characterized by a loss of VPS4A expression. The method involves contacting the cell with an agent that inhibits the expression or activity of ULK3, CHMP1A, CHMP1B, VTA1, and/or IST1, thereby inducing or promoting cell death or reducing cell survival of the neoplastic cell.

In another aspect, the invention features a method for inducing cell death or reducing cell survival of a neoplastic cell characterized by a loss of VPS4B expression. The method involves contacting the cell with an agent that inhibits the expression or activity of ULK3, CHMP1A, CHMP1B, VTA1, and/or IST1, thereby inducing or promoting cell death or reducing cell survival of the neoplastic cell.

In any of the above aspects, the method further involves contacting the cell with an agent that inhibits the expression or activity of VPS4B. In any of the above aspects, the method further involves contacting the cell with an agent that inhibits the expression or activity of VPS4A.

In any of the above aspects, the neoplastic cell is a brain, bladder, bile, blood, breast, duct, colon, colorectal, esophageal, gastric, germ cell, liver, ovarian, pancreatic, uterine, or lung cancer cell. In any of the above aspects, the neoplastic cell is a pancreatic cancer cell. In any of the above aspects, the neoplastic cell is a renal cell carcinoma or a pancreatic ductal adrenocarcinoma. In any of the above aspects, the neoplastic cell is a sarcoma cell. In embodiments, the sarcoma is an osteosarcoma cell or a rhabdomyosarcoma cell. In embodiments, the sarcoma is a pediatric rhabdomyosarcoma cell.

In any of the above aspects, the rhabdomyosarcoma or neoplastic cell is further characterized by a loss of SMAD4 or CDH1. In any of the above aspects, the neoplastic cell lacks detectable levels of SMAD4 or CDH1 polypeptide or polynucleotide expression.

In any of the above aspects, the method further involves contacting the cell with an interferon. In embodiments, the interferon is interferon-β.

In any of the above aspects, the rhabdomyosarcoma cell or neoplastic cell is a mammalian cell. In embodiments, the mammalian cell is a human cell.

In one aspect, the invention features a method for treating a subject having a neoplasia characterized by a loss of VPS4A expression. The method involves administering to the subject an agent that inhibits the expression or activity of ULK3, CHMP1A, CHMP1B, VTA1, or IST1, thereby inducing or promoting cell death or reducing cell survival of the neoplasia

In any of the above aspects, the method involves administering an agent that inhibits the expression or activity of VPS4B.

In one aspect, the invention features a method for treating a subject having a neoplasia characterized by a loss of VPS4B expression. The method involves contacting the cell with an agent that inhibits the expression or activity of ULK3, CHMP1A, CHMP1B, VTA1, and IST1, thereby inducing or promoting cell death or reducing cell survival of the neoplasia.

In any of the above aspects, the method further involves administering an agent that inhibits the expression or activity of VPS4A.

In any of the above aspects, the neoplasia is a brain, bladder, bile, blood, breast, duct, colon, colorectal, esophageal, gastric, germ cell, liver, ovarian, pancreatic, uterine, or lung cancer. In embodiments, the cancer is a pancreatic cancer. In any of the above aspects, the neoplasia is a renal carcinoma or a pancreatic ductal adrenocarcinoma. In any of the above aspects, the neoplasia is a sarcoma. In embodiments, the sarcoma is an osteosarcoma or a rhabdomyosarcoma. In embodiments, the sarcoma is a pediatric rhabdomyosarcoma.

In any of the above aspects, the neoplasia is further characterized by a loss of SMAD4 or CDH1. In any of the above aspects, the neoplasia lacks detectable levels of SMAD4 or CDH1 polypeptide or polynucleotide expression.

In one aspect, the invention features a method for treating a selected subject having cancer characterized by a loss of VPS4A expression. The method involves administering an agent that inhibits the expression of VPS4B, ULK3, CHMP1A, CHMP1B, VTA1, and/or IST1, thereby treating the subject. The subject is selected if the cancer is determined to have VPS4A dependency. Dependency is determined using a multivariate model, where levels of a VPS4B marker and levels of at least one of a CHMP4B, ITCH, and ISG15 marker are used as inputs to the model. In embodiments, the method involves administering an agent that inhibits the expression or activity of VPS4A.

In any of the above aspects, the VPS4B, CHMP4B, and ITCH marker levels are used as inputs to the model. In any of the above aspects, the VPS4B, CHMP4B, ITCH, and ISG15 marker levels are used as inputs to the model.

In any of the above aspects, the markers are polypeptides and/or polynucleotides. In embodiments, the polynucleotides are mRNA molecules.

In any of the above aspects, the method further involves detecting levels of the markers in a biological sample derived from the subject.

In any of the above aspects, the biological sample is a fluid or tissue sample. In embodiments, the fluid sample is a blood, cerebrospinal fluid, phlegm, saliva, fecal, or urine sample. In embodiments, the tissue sample is a biopsy sample.

In any of the above aspects, the multivariate model is a linear model. In any of the above aspects, the multivariate model has an improved capacity to predict VPS4A dependency of a cancer, as compared to a univariate model using any one of the VPS4B, CHMP4B, ITCH, and ISG15 markers as input.

In any of the above aspects, the cancer is a brain, bladder, bile, blood, breast, duct, colon, colorectal, esophageal, gastric, germ cell, liver, ovarian, pancreatic, uterine, or lung cancer. In any of the above aspects, the cancer is a pancreatic cancer. In any of the above aspects, the cancer is a renal carcinoma or a pancreatic ductal adrenocarcinoma.

In any of the above aspects, the cancer is a sarcoma. In embodiments, the sarcoma is an osteosarcoma or a rhabdomyosarcoma. In embodiments, the sarcoma is a pediatric rhabdomyosarcoma.

In any of the above aspects, the cancer is further characterized by a loss of SMAD4 or CDH1. In any of the above aspects, the cancer lacks detectable levels of SMAD4 or CDH1 polypeptide or polynucleotide expression.

In any of the above aspects, the method further involves administering an interferon. In embodiments, the interferon is interferon-β.

In any of the above aspects, the agent contains a small molecule compound, polypeptide, or polynucleotide. In any of the above aspects, the agent contains SU6668 and/or MSC1094308. In embodiments, the polynucleotide is an inhibitory nucleic acid molecule. In embodiments, the inhibitory nucleic acid molecule is an siRNA, shRNA, miRNA, ribozyme, or antisense RNA. In embodiments, the inhibitory nucleic acid molecule is shRNA and containing a sequence, from 5′ to 3′, selected from the three sequences GCAAGAAGCCAGUCAAAGAGA (SEQ ID NO: 1), CGAGAAGCUGAAGGAUUAUUU (SEQ ID NO: 2), and GCCGAGAAGCUGAAGGAUUAU (SEQ ID NO: 3); any of the three sequences truncated by 1, 2, 3, 4, or 5 nucleotides at the 5′ and/or 3′ end; and variants of any of the three sequences containing 1, 2, 3, 4, or 5 nucleobase substitutions.

In any of the above aspects, the agent contains a genome editing system or a CRISPR interference system. In embodiments, the genome editing system is a CRISPR-spCas9 system containing a single-guide RNA (sgRNA). In embodiments, the sgRNA targets VPS4A and contains a sequence, from 5′ to 3′, selected from the four sequences ACUCACACUUGAUAGCGUGG (SEQ ID NO: 4), GGGCCGCACGAAGUACCUGG (SEQ ID NO: 5), AUUGUUAUUCCCCACCCCUG (SEQ ID NO: 6), and CCACUUAGAAACAAGAUCAG (SEQ ID NO: 7); any of the four sequences truncated by 1, 2, 3, 4, or 5 nucleotides at the 5′ and/or 3′ end; and variants of any of the four sequences containing 1, 2, 3, 4, or 5 nucleobase substitutions.

In any of the above aspects, the rhabdomyosarcoma cell or neoplastic cell is in a subject. In any of the above aspects, the subject is an animal. Any of the above aspects, the animal is a mammal. In any of the above aspects, the mammal is a human.

The invention provides compositions and methods for treating neoplasia characterized by a reduction in or the loss of VPS4A or VPS4B. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “cadherin 1 (CDH1) polypeptide” is meant a polypeptide or fragment thereof having activity associated with cell adhesion and having at least about 85% identity to NCBI Reference Sequence Accession No. NP_004351.1. The sequence of an exemplary CDH1 polypeptide is provided below:

(SEQ ID NO: 8)
MGPWSRSLSALLLLLQVSSWLCQEPEPCHPGFDAESYTFTVPRRHLERG
RVLGRVNFEDCTGRQRTAYFSLDTRFKVGTDGVITVKRPLRFHNPQIHF
LVYAWDSTYRKFSTKVTLNTVGHHHRPPPHQASVSGIQAELLTFPNSSP
GLRRQKRDWVIPPISCPENEKGPFPKNLVQIKSNKDKEGKVFYSITGQG
ADTPPVGVFIIERETGWLKVTEPLDRERIATYTLFSHAVSSNGNAVEDP
MEILITVTDQNDNKPEFTQEVFKGSVMEGALPGTSVMEVTATDADDDVN
TYNAAIAYTILSQDPELPDKNMFTINRNTGVISVVTTGLDRESFPTYTL
VVQAADLQGEGLSTTATAVITVTDTNDNPPIFNPTTYKGQVPENEANVV
ITTLKVTDADAPNTPAWEAVYTILNDDGGQFVVTTNPVNNDGILKTAKG
LDFEAKQQYILHVAVTNVVPFEVSLTTSTATVTVDVLDVNEAPIFVPPE
KRVEVSEDFGVGQEITSYTAQEPDTFMEQKITYRIWRDTANWLEINPDT
GAISTRAELDREDFEHVKNSTYTALIIATDNGSPVATGTGTLLLILSDV
NDNAPIPEPRTIFFCERNPKPQVINIIDADLPPNTSPFTAELTHGASAN
WTIQYNDPTQESIILKPKMALEVGDYKINLKLMDNQNKDQVTTLEVSVC
DCEGAAGVCRKAQPVEAGLQIPAILGILGGILALLILILLLLLFLRRRA
VVKEPLLPPEDDTRDNVYYYDEEGGGEEDQDFDLSQLHRGLDARPEVTR
NDVAPTLMSVPRYLPRPANPDEIGNFIDENLKAADTDPTAPPYDSLLVF
DYEGSGSEAASLSSLNSSESDKDQDYDYLNEWGNRFKKLADMYGGGED
D.

By “CDH1 polynucleotide” is meant a polynucleotide encoding a CDH1 polypeptide. A nucleic acid sequence encoding an exemplary CDH1 corresponds to NCBI Reference Sequence Accession No. NM_004360.5, which is provided below:

(SEQ ID NO: 9)
AGTGGCGTCGGAACTGCAAAGCACCTGTGAGCTTGCGGAAGTCAGTTCAGACTCCAGCCCG
CTCCAGCCCGGCCCGACCCGACCGCACCCGGCGCCTGCCCTCGCTCGGCGTCCCCGGCCAGC
CATGGGCCCTTGGAGCCGCAGCCTCTCGGCGCTGCTGCTGCTGCTGCAGGTCTCCTCTTGGCT
CTGCCAGGAGCCGGAGCCCTGCCACCCTGGCTTTGACGCCGAGAGCTACACGTTCACGGTGC
CCCGGCGCCACCTGGAGAGAGGCCGCGTCCTGGGCAGAGTGAATTTTGAAGATTGCACCGG
TCGACAAAGGACAGCCTATTTTTCCCTCGACACCCGATTCAAAGTGGGCACAGATGGTGTGA
TTACAGTCAAAAGGCCTCTACGGTTTCATAACCCACAGATCCATTTCTTGGTCTACGCCTGGG
ACTCCACCTACAGAAAGTTTTCCACCAAAGTCACGCTGAATACAGTGGGGCACCACCACCGC
CCCCCGCCCCATCAGGCCTCCGTTTCTGGAATCCAAGCAGAATTGCTCACATTTCCCAACTCC
TCTCCTGGCCTCAGAAGACAGAAGAGAGACTGGGTTATTCCTCCCATCAGCTGCCCAGAAAA
TGAAAAAGGCCCATTTCCTAAAAACCTGGTTCAGATCAAATCCAACAAAGACAAAGAAGGC
AAGGTTTTCTACAGCATCACTGGCCAAGGAGCTGACACACCCCCTGTTGGTGTCTTTATTATT
GAAAGAGAAACAGGATGGCTGAAGGTGACAGAGCCTCTGGATAGAGAACGCATTGCCACAT
ACACTCTCTTCTCTCACGCTGTGTCATCCAACGGGAATGCAGTTGAGGATCCAATGGAGATT
TTGATCACGGTAACCGATCAGAATGACAACAAGCCCGAATTCACCCAGGAGGTCTTTAAGG
GGTCTGTCATGGAAGGTGCTCTTCCAGGAACCTCTGTGATGGAGGTCACAGCCACAGACGCG
GACGATGATGTGAACACCTACAATGCCGCCATCGCTTACACCATCCTCAGCCAAGATCCTGA
GCTCCCTGACAAAAATATGTTCACCATTAACAGGAACACAGGAGTCATCAGTGTGGTCACCA
CTGGGCTGGACCGAGAGAGTTTCCCTACGTATACCCTGGTGGTTCAAGCTGCTGACCTTCAA
GGTGAGGGGTTAAGCACAACAGCAACAGCTGTGATCACAGTCACTGACACCAACGATAATC
CTCCGATCTTCAATCCCACCACGTACAAGGGTCAGGTGCCTGAGAACGAGGCTAACGTCGTA
ATCACCACACTGAAAGTGACTGATGCTGATGCCCCCAATACCCCAGCGTGGGAGGCTGTATA
CACCATATTGAATGATGATGGTGGACAATTTGTCGTCACCACAAATCCAGTGAACAACGATG
GCATTTTGAAAACAGCAAAGGGCTTGGATTTTGAGGCCAAGCAGCAGTACATTCTACACGTA
GCAGTGACGAATGTGGTACCTTTTGAGGTCTCTCTCACCACCTCCACAGCCACCGTCACCGT
GGATGTGCTGGATGTGAATGAAGCCCCCATCTTTGTGCCTCCTGAAAAGAGAGTGGAAGTGT
CCGAGGACTTTGGCGTGGGCCAGGAAATCACATCCTACACTGCCCAGGAGCCAGACACATTT
ATGGAACAGAAAATAACATATCGGATTTGGAGAGACACTGCCAACTGGCTGGAGATTAATC
CGGACACTGGTGCCATTTCCACTCGGGCTGAGCTGGACAGGGAGGATTTTGAGCACGTGAA
GAACAGCACGTACACAGCCCTAATCATAGCTACAGACAATGGTTCTCCAGTTGCTACTGGAA
CAGGGACACTTCTGCTGATCCTGTCTGATGTGAATGACAACGCCCCCATACCAGAACCTCGA
ACTATATTCTTCTGTGAGAGGAATCCAAAGCCTCAGGTCATAAACATCATTGATGCAGACCT
TCCTCCCAATACATCTCCCTTCACAGCAGAACTAACACACGGGGCGAGTGCCAACTGGACCA
TTCAGTACAACGACCCAACCCAAGAATCTATCATTTTGAAGCCAAAGATGGCCTTAGAGGTG
GGTGACTACAAAATCAATCTCAAGCTCATGGATAACCAGAATAAAGACCAAGTGACCACCT
TAGAGGTCAGCGTGTGTGACTGTGAAGGGGCCGCTGGCGTCTGTAGGAAGGCACAGCCTGT
CGAAGCAGGATTGCAAATTCCTGCCATTCTGGGGATTCTTGGAGGAATTCTTGCTTTGCTAAT
TCTGATTCTGCTGCTCTTGCTGTTTCTTCGGAGGAGAGCGGTGGTCAAAGAGCCCTTACTGCC
CCCAGAGGATGACACCCGGGACAACGTTTATTACTATGATGAAGAAGGAGGCGGAGAAGAG
GACCAGGACTTTGACTTGAGCCAGCTGCACAGGGGCCTGGACGCTCGGCCTGAAGTGACTC
GTAACGACGTTGCACCAACCCTCATGAGTGTCCCCCGGTATCTTCCCCGCCCTGCCAATCCC
GATGAAATTGGAAATTTTATTGATGAAAATCTGAAAGCGGCTGATACTGACCCCACAGCCCC
GCCTTATGATTCTCTGCTCGTGTTTGACTATGAAGGAAGCGGTTCCGAAGCTGCTAGTCTGA
GCTCCCTGAACTCCTCAGAGTCAGACAAAGACCAGGACTATGACTACTTGAACGAATGGGG
CAATCGCTTCAAGAAGCTGGCTGACATGTACGGAGGCGGCGAGGACGACTAGGGGACTCGA
GAGAGGCGGGCCCCAGACCCATGTGCTGGGAAATGCAGAAATCACGTTGCTGGTGGTTTTTC
AGCTCCCTTCCCTTGAGATGAGTTTCTGGGGAAAAAAAAGAGACTGGTTAGTGATGCAGTTA
GTATAGCTTTATACTCTCTCCACTTTATAGCTCTAATAAGTTTGTGTTAGAAAAGTTTCGACT
TATTTCTTAAAGCTTTTTTTTTTTTCCCATCACTCTTTACATGGTGGTGATGTCCAAAAGATAC
CCAAATTTTAATATTCCAGAAGAACAACTTTAGCATCAGAAGGTTCACCCAGCACCTTGCAG
ATTTTCTTAAGGAATTTTGTCTCACTTTTAAAAAGAAGGGGAGAAGTCAGCTACTCTAGTTCT
GTTGTTTTGTGTATATAATTTTTTAAAAAAAATTTGTGTGCTTCTGCTCATTACTACACTGGTG
TGTCCCTCTGCCTTTTTTTTTTTTTTAAGACAGGGTCTCATTCTATCGGCCAGGCTGGAGTGCA
GTGGTGCAATCACAGCTCACTGCAGCCTTGTCCTCCCAGGCTCAAGCTATCCTTGCACCTCA
GCCTCCCAAGTAGCTGGGACCACAGGCATGCACCACTACGCATGACTAATTTTTTAAATATT
TGAGACGGGGTCTCCCTGTGTTACCCAGGCTGGTCTCAAACTCCTGGGCTCAAGTGATCCTC
CCATCTTGGCCTCCCAGAGTATTGGGATTACAGACATGAGCCACTGCACCTGCCCAGCTCCC
CAACTCCCTGCCATTTTTTAAGAGACAGTTTCGCTCCATCGCCCAGGCCTGGGATGCAGTGA
TGTGATCATAGCTCACTGTAACCTCAAACTCTGGGGCTCAAGCAGTTCTCCCACCAGCCTCCT
TTTTATTTTTTTGTACAGATGGGGTCTTGCTATGTTGCCCAAGCTGGTCTTAAACTCCTGGCCT
CAAGCAATCCTTCTGCCTTGGCCCCCCAAAGTGCTGGGATTGTGGGCATGAGCTGCTGTGCC
CAGCCTCCATGTTTTAATATCAACTCTCACTCCTGAATTCAGTTGCTTTGCCCAAGATAGGAG
TTCTCTGATGCAGAAATTATTGGGCTCTTTTAGGGTAAGAAGTTTGTGTCTTTGTCTGGCCAC
ATCTTGACTAGGTATTGTCTACTCTGAAGACCTTTAATGGCTTCCCTCTTTCATCTCCTGAGT
ATGTAACTTGCAATGGGCAGCTATCCAGTGACTTGTTCTGAGTAAGTGTGTTCATTAATGTTT
ATTTAGCTCTGAAGCAAGAGTGATATACTCCAGGACTTAGAATAGTGCCTAAAGTGCTGCAG
CCAAAGACAGAGCGGAACTATGAAAAGTGGGCTTGGAGATGGCAGGAGAGCTTGTCATTGA
GCCTGGCAATTTAGCAAACTGATGCTGAGGATGATTGAGGTGGGTCTACCTCATCTCTGAAA
ATTCTGGAAGGAATGGAGGAGTCTCAACATGTGTTTCTGACACAAGATCCGTGGTTTGTACT
CAAAGCCCAGAATCCCCAAGTGCCTGCTTTTGATGATGTCTACAGAAAATGCTGGCTGAGCT
GAACACATTTGCCCAATTCCAGGTGTGCACAGAAAACCGAGAATATTCAAAATTCCAAATTT
TTTTCTTAGGAGCAAGAAGAAAATGTGGCCCTAAAGGGGGTTAGTTGAGGGGTAGGGGGTA
GTGAGGATCTTGATTTGGATCTCTTTTTATTTAAATGTGAATTTCAACTTTTGACAATCAAAG
AAAAGACTTTTGTTGAAATAGCTTTACTGTTTCTCAAGTGTTTTGGAGAAAAAAATCAACCC
TGCAATCACTTTTTGGAATTGTCTTGATTTTTCGGCAGTTCAAGCTATATCGAATATAGTTCT
GTGTAGAGAATGTCACTGTAGTTTTGAGTGTATACATGTGTGGGTGCTGATAATTGTGTATTT
TCTTTGGGGGTGGAAAAGGAAAACAATTCAAGCTGAGAAAAGTATTCTCAAAGATGCATTTT
TATAAATTTTATTAAACAATTTTGTTAAA.

By “charged multivesicular body protein 1A (CHMP1A) polypeptide” is meant a polypeptide or fragment thereof having activity associated with multivesicular body sorting of proteins to the interiors of lysosomes and having at least about 85% identity to NCBI Reference Sequence Accession No. NP_001076783.1. The sequence of an exemplary CHMP1A polypeptide is provided below:

(SEQ ID NO: 10)
MDVHGEAAGEAGQEGGEGLQGGAGQSEEGPSAEKCRVCPCVCRERHPQE
ERRCELASDGVPRRRSGLQGADSCDYEGGDQEYGPGDQSPGQGPEHHGP
AEGLLSDGQVRAAGAEPGRPYIGDGGLHELGHHPDHAAGAGGQPHHADR
RGEWPGGAGPAQPAARGRLCRGRELCAQPGGPAVTEVGRLEELAVPRRC
APPLPRDVLEGSCPLPTASCLCADPAGLRPAATLRLSPARPAWP.

By “CHMP1A polynucleotide” is meant a polynucleotide encoding a CHMP1A polypeptide. A nucleic acid sequence encoding an exemplary CHMP1A corresponds to NCBI Reference Sequence Accession No. NM_001083314.4, which is provided below:

(SEQ ID NO: 11)
GGCGACCCCGGAAGTCCCCGCCGGGTGCAGCTTGGTCGGTTCGATCGCCGCCGGGACCTGAC
ACCGCCCGGAGTTGGCGTCCCTTCTCCCTCTCCGAGTGCTGCTCCTGTCATTGTGGCCATGGA
CGTTCACGGCGAAGCAGCTGGAGAAGCTGGCCAAGAAGGCGGAGAAGGACTCCAAGGCGG
AGCAGGCCAAAGTGAAGAAGGCCCTTCTGCAGAAAAATGTAGAGTGTGCCCGTGTGTATGC
CGAGAACGCCATCCGCAAGAAGAACGAAGGTGTGAACTGGCTTCGGATGGCGTCCCGCGTA
GACGCAGTGGCCTCCAAGGTGCAGACAGCTGTGACTATGAAGGGGGTGACCAAGAATATGG
CCCAGGTGACCAAAGCCCTGGACAAGGCCCTGAGCACCATGGACCTGCAGAAGGTCTCCTC
AGTGATGGACAGGTTCGAGCAGCAGGTGCAGAACCTGGACGTCCATACATCGGTGATGGAG
GACTCCATGAGCTCGGCCACCACCCTGACCACGCCGCAGGAGCAGGTGGACAGCCTCATCA
TGCAGATCGCCGAGGAGAATGGCCTGGAGGTGCTGGACCAGCTCAGCCAGCTGCCCGAGGG
CGCCTCTGCCGTGGGCGAGAGCTCTGTGCGCAGCCAGGAGGACCAGCTGTCACGGAGGTTG
GCCGCCTTGAGGAACTAGCCGTGCCCCGCCGGTGTGCACCGCCTCTGCCCCGTGATGTGCTG
GAAGGCTCCTGTCCTCTCCCCACCGCGTCTTGCCTTTGTGCTGACCCCGCGGGGCTGCGGCCG
GCAGCCACTCTGCGTCTCTCACCTGCCAGGCCTGCGTGGCCTTAGGGTTGTTCCTGTTCTTTT
AGGTTGGGCGGTGGGTCTGTGTCCTGGTGTTGAGTTTCTGCAAATTTCTGGGGGTGATTTCTG
TGACTCTGGGCCCACAGCGGGGAGGCCAAGAGGGGCCCTGTGGACTTTCACCCAGCACTGT
GGGGGCCTTCAGACTCTGGGGCAGCAGACATGCTGCTTCCCATCAGCCAGAGGGGGTCAGG
GCTGCCCTGTTGCCAAACAACTCCCTGAGGCCTCTCCGCACCACCTCAGCGGGCAGGAGGTC
CCACCATGTGGACAGACATAGCCCAAGGAGGCACCACAGGTCTATGTGTGCTGGGGGATGT
CAGGTGCCACCCAACGCTGTCCTGGTGGTATTTACAATGACATCCTCCTCCTCCATCACTCCA
GGGGTGGTGTCTCGGCCGCCCCTACCAGCTGGCTGAGCCCCCTGGCCTCCTGCGCTCCCTCA
CTTCCCTCAGTTCCCAAAGCTGCCCAGTCCATGGGGACAGAACCGTCACTCAGATCCACATT
CAAGTGTGCCCACCCTGCAGTCTTCATCCTCACTCAGCTGCTGCCTCTGGAGGTGCCTTTGGC
CACATGTGCTGTGCTGTTTGTCTCCTCGACAGGGAGCCTGTCCACCAGCAGGCTGCGGTCCC
AGCGGGTGCGTCTGCAGCTCCTCCCCTTGGGCAGCCTGGTTCTCCCGGAGGACCTTTCCTTGG
GGCCCTGCTTCATGACGATGCTGCCTGTGTCACCCTCTACCATCTGTAAACAACTGGGTGCCT
TCCCCGACCACACCCCAATGCCTTCCCAGCTTGGAAGCCAAGGCAGCTGATGAAGGGAGCTC
AGGAGAGCCGTCTTCAGCTGGGCTGGGGTTGGGGCTGCTGTGAGGAAAACCTGCCATTGTG
GCCCTGGAGAGTCACCAGCAGCTCTTGGGAAGGACTTGCTGGGAGGCTGAGAGAGGCTTTG
GGCACAGCCTGCTGTCTTTTCCATTTCCTAAAGTTTACTTCATTGTCTTGAGGCTTCCAGGTTT
TGTTTTTGTTTTTGCCAAAGTAGAAAAGGCAGGTGGTGGGCGGCTGGCAGGGAGTGCGGGTC
CCCGCCCCTCTTCAGTCCTGCCCTCCCCTCCTCAGTCCTGCCCACCCCGTGCAGCCCATGCTG
AGGCTGCAGTGGTGTCGTGGGTGTTACGTGCAGGAACGTGGAGACCCTGACGTGGGCTCACT
GCGTTTGGTTTTCTTTTCAGAACTTGGGAGCCCCCAGGGAGGGGCTAGTGTTGGTAGGTCCT
AGACGTGGTTCCCTCCAGCCTCCCCAAAATCAACCCTGGTGTTGAGAGAACGTCCTTCTGTC
CATCGTGGGTAACAGCCTTGGGGAGGGTGCAGAGCTCTGCAGAGCCATGGGCCAGGTGGGG
CTGCCTCAGTCCTGTCCCCTTGGGCACTGAGGAGAGGGGCCCATTCACCTTTCTCCTAGAAT
GCTGTTGTAAATAAACAAATGGATCCCTGGAAA.

By “charged multivesicular body protein 1B (CHMP1B) polypeptide” is meant a polypeptide or fragment thereof having activity associated with degradation of surface receptor proteins and formation of endocytic multivesicular bodies and having at least about 85% identity to GenBank Accession No. AAH12733.3. The sequence of an exemplary CHMP1B polypeptide is provided below:

(SEQ ID NO: 12)
MSNMEKHLFNLKFAAKELSRSAKKCDKEEKAEKAKIKKAIQKGNMEVAR
IHAENAIRQKNQAVNFLRMSARVDAVAARVQTAVTMGKVTKSMAGVVKS
MDATLKTMNLEKISALMDKFEHQFETLDVQTQQMEDTMSSTTTLTTPQN
QVDMLLQEMADEAGLDLNMELPQGQTGSVGTSVASAEQDELSQRLARLR
DQV.

By “charged multivesicular body protein 1B (CHMP1B) polynucleotide” is meant a polynucleotide encoding a CHMP1B polypeptide. A nucleic acid sequence encoding an exemplary CHMP1B corresponds to GenBank Accession No. BC012733.2, which is provided below:

(SEQ ID NO: 13)
CTGCCTTCGGCGCGCTTCTCAGCAGGGCCGCCGACCCAAAGGAGCCGTC
TGACTATGTCTAACATGGAGAAACACCTGTTCAACCTGAAGTTCGCGGC
CAAAGAACTGAGTAGGAGTGCCAAAAAATGCGATAAGGAGGAAAAGGCC
GAAAAGGCCAAAATTAAAAAGGCCATTCAGAAGGGCAACATGGAAGTTG
CGAGGATACACGCCGAAAATGCCATCCGCCAGAAGAACCAGGCGGTGAA
TTTCTTGAGAATGAGTGCGCGAGTCGATGCAGTGGCTGCCAGGGTCCAG
ACGGCGGTGACGATGGGCAAGGTGACCAAGTCGATGGCTGGTGTGGTTA
AGTCGATGGATGCGACATTGAAGACCATGAATCTGGAGAAGATTTCTGC
TTTGATGGACAAATTCGAGCACCAGTTTGAGACTCTGGACGTCCAGACG
CAGCAAATGGAAGACACGATGAGCAGCACGACGACGCTCACCACTCCCC
AGAACCAAGTGGATATGCTGCTCCAGGAAATGGCAGATGAGGCGGGCCT
CGACCTCAACATGGAGCTGCCGCAGGGCCAGACCGGCTCCGTGGGCACG
AGCGTGGCTTCGGCGGAGCAGGATGAACTGTCTCAGAGACTGGCCCGCC
TTCGGGATCAAGTGTGACGGCAGAACCCGCTCTGAGGTTTCCTGGCCAT
AGCCACCCTTTGAAATGCTCTCTGTGTGTTAGAGAGATACTATACCCTA
GAAACTCTGAACACGCCAGAATGCTGAAATGCCCTTCTACCTTTGGGTT
TACAGCCCCCTCCACATAAATTAAGAAATTCAGTATTTCTGCACTCTTA
GCTGGATTCTAAAGTTCTGTATAGCTCGTAATGATGGTATTTTTATAGC
AGCCTTTTAACAGAACTAGTTAATTTCGTGTATATGAATCTTTCTCGAA
GATCTGGTCAAAACTGTATTCAGTTTCCTGCCCAGAATGATCAGATTGA
AGGTGGTTGGTTTTTATTATTATTTAGTGTGATTGATAGTATCTAGAAT
GGCAGGTGGTGCATAAAAGTTAAAGAGAGGGGAAAGATTACTTAGTTTG
GTTATACAGTTATAAACACCATGCAGTGTATTCGGTGGACTGTGCTATT
TCTGTTTATCCTTTGGGTTTTGGTTTTTGTTTTTTTTTTTTGCCTTCAC
AGTGAGACTGCAAATGATTGTTCTCATAACGTATATTATTAATAAATGT
GGTCCTATAATTTATACTGAAAAAAAAAAAAAAAAAAAAAAAAAAAAAA
AAAAAAAAAAAAAAAAAAAAAAAAAAAAAAAAAAAAAAAA.

By “charged multivesicular body protein 4B (CHMP4B) polypeptide” is meant a polypeptide or fragment thereof having activity associated with degradation of surface receptor proteins and formation of endocytic multivesicular bodies and having at least about 85% identity to GenBank Accession No. AAH33859.1. The sequence of an exemplary CHMP4B polypeptide is provided below:

(SEQ ID NO: 14)
MSVFGKLFGAGGGKAGKGGPTPQEAIQRLRDTEEMLSKKQEFLEKKIEQ
ELTAAKKHGTKNKRAALQALKRKKRYEKQLAQIDGTLSTIEFQREALEN
ANTNTEVLKNMGYAAKAMKAAHDNMDIDKVDELMQDIADQQELAEEIST
AISKPVGFGEEFDEDELMAELEELEQEELDKNLLEISGPETVPLPNVPS
IALPSKPAKKKEEEDDDMKELENWAGSM.

By “CHMP4B polynucleotide” is meant a polynucleotide encoding a CHMP4B polypeptide. A nucleic acid sequence encoding an exemplary CHMP4B corresponds to GenBank Accession No. BC033859.1, which is provided below:

(SEQ ID NO: 15)
GGCGGCAGGGAGCGGCGGGACTGGGAGCGGGCGCGGGAGCCGACCCGAGCCGAGCCGAGC
CGAGCCGAGCCGGAGCGGGCGGCGAAGGCCGGCGCGGCGAGCAGCAACCATGTCGGTGTTC
GGGAAGCTGTTCGGGGCTGGAGGGGGTAAGGCCGGCAAGGGCGGCCCGACCCCCCAGGAG
GCCATCCAGCGGCTGCGGGACACGGAAGAGATGTTAAGCAAGAAACAGGAGTTCCTGGAGA
AGAAAATCGAGCAGGAGCTGACGGCCGCCAAGAAGCACGGCACCAAAAACAAGCGCGCGG
CCCTCCAGGCACTGAAGCGTAAGAAGAGGTATGAGAAGCAGCTGGCGCAGATCGACGGCAC
ATTATCAACCATCGAGTTCCAGCGGGAGGCCCTGGAGAATGCCAACACCAACACCGAGGTG
CTCAAGAACATGGGCTATGCCGCCAAGGCCATGAAGGCGGCCCATGACAACATGGACATCG
ATAAAGTTGATGAGTTAATGCAGGACATTGCTGACCAGCAAGAACTTGCAGAGGAGATTTC
AACAGCAATTTCGAAACCTGTAGGGTTTGGAGAAGAGTTTGACGAGGATGAGCTCATGGCG
GAATTAGAAGAACTAGAACAGGAGGAACTAGACAAGAATTTGCTGGAAATCAGTGGACCCG
AAACAGTCCCTCTACCAAATGTTCCCTCTATAGCCCTACCATCAAAACCCGCCAAGAAGAAA
GAAGAGGAGGACGACGACATGAAGGAATTGGAGAACTGGGCTGGATCCATGTAATGGGGTC
CAGCGCTGGCTGGGCCCAGACAGACTGTGGTGGCCTGCGCAGCGAGCAGGCGTGTGCGTGT
GTGGGGCAGGCAGGATGTGGTGCAGGCAGGTTCCATCGCTTTCGACTCTCACTCCAAAGCAG
TAGGGCCGCGTTGCTGCTCACTCTCTGCATAGCATGGTCTGCACCTGGGAGATGGGCGGGGG
GAGGGGGGCGGGCGGGGTGGGAAGTGCCTGCTGTTTATAATGTTGAATTTCTGTAAAATAA
ACTGTATTTGCAAATCCAACATTGAGCTTCTGGACTACGCTGACTCCACTGCTGAATCCTCAA
TGGAAAGGGTCGACTGGTTGCAGTTGAAATGACCTGAAATGTAGCCTCTGTCCTTGTAAGTC
AGTTGACTTGCCGCACATCTCTTTGTGTACTTGTACGGTACTGGCAGAAAAGTCATTTTTCAA
AAGCCATAGGCTTTTCCTTGCCCTTAGCTGTAATAATGCATCTGATTTTGATTTCCTCCAGAG
CTGTGTTTCTGTCCATCACCTGTGTATTGGCCCTGTGTTTACCACTCTGGCCCACTCCTCACCC
CCTTGCTCCCCTGGTCTTCTGGAGTTTGTGACATTGATTTGAAATGGATGGTGTTCTCTTGAG
AGCAAGTGAGATTGTTAGAATTAAGTTCCAACTATACAGTTTTCTAACATAGCTATAAGGTC
CTTGTTGCTGTTTGTGATAACTGATAGATAACTCATTGGAAACGTGCATACATTTATATTCAG
ATGAAATTATGGTTTGCACTGTCTATTAAATATCTCGATTAATTTTCAAAAAAAAAAAAAAA
AAAAAAAAAAAAAAAAAAAAAAAAAAAAAAAAAAAA.

By “Ubiquitin-Like Modifier ISG15 (ISG15) polypeptide” is meant a polypeptide or fragment thereof having activity associated with natural killer (NK)-cell proliferation and having at least about 85% identity to GenBank Accession No. AAA36128.1. The sequence of an exemplary ISG15 polypeptide is provided below:

(SEQ ID NO: 16)
MGWDLTVKMLAGNEFQVSLSSSMSVSELKAQITQNIGVHAFQQRLAVHP
SGVALQDRVPLASQGLGPGSTVLLVVDKCDEPLSILVRNNKGRSSTYEV
RLTQTVAHLKQQVSGLEGVQDDLFWLTFEGKPLEDQLPLGEYGLKPLST
VFMNLRLRGGGTEPGGRS.

By “ISG15 polynucleotide” is meant a polynucleotide encoding a ISG15 polypeptide. A nucleic acid sequence encoding an exemplary ISG15 corresponds to GenBank Accession No. M13755.1, which is provided below:

(SEQ ID NO: 17)
CGGCTGAGAGGCAGCGAACTCATCTTTGCCAGTACAGGAGCTTGTGCCG
TGGCCCACAGCCCACAGCCCACAGCCATGGGCTGGGACCTGACGGTGAA
GATGCTGGCGGGCAACGAATTCCAGGTGTCCCTGAGCAGCTCCATGTCG
GTGTCAGAGCTGAAGGCGCAGATCACCCAGAAGATTGGCGTGCACGCCT
TCCAGCAGCGTCTGGCTGTCCACCCGAGCGGTGTGGCGCTGCAGGACAG
GGTCCCCCTTGCCAGCCAGGGCCTGGGCCCTGGCAGCACGGTCCTGCTG
GTGGTGGACAAATGCGACGAACCTCTGAGCATCCTGGTGAGGAATAACA
AGGGCCGCAGCAGCACCTACGAGGTCCGGCTGACGCAGACCGTGGCCCA
CCTGAAGCAGCAAGTGAGCGGGCTGGAGGGTGTGCAGGACGACCTGTTC
TGGCTGACCTTCGAGGGGAAGCCCCTGGAGGACCAGCTCCCGCTGGGGG
AGTACGGCCTCAAGCCCCTGAGCACCGTGTTCATGAATCTGCGCCTGCG
GGGAGGCGGCACAGAGCCTGGCGGGCGGAGCTAAGGGCCTCCACCAGCA
TCCGAGCAGGATCAAGGGCCGGAAATAAAGGCTGTTGTAAGAGAAT.

By “ITCHY E3 UBIQUITIN PROTEIN LIGASE (ITCH) polypeptide” is meant a polypeptide or fragment thereof having ubiquitination activity and having at least about 85% identity to GenBank Accession No. AAC04845.1. The sequence of an exemplary ITCH polypeptide is provided below:

(SEQ ID NO: 18)
GDKEPTETIGDLSICLDGLQLESEVVTNGETTCSESASQNDDGSRSKD
ETRVSTNGSDDPEDAGAGENRRVSGNNSPSLSNGGFKPSRPPRPSRPP
PPTPRRPASVNGSPSATSESDGSSTGSLPPTNTNTNTSEGATSGLIIP
LTISGGSGPRPLNPVTQAPLPPGWEQRVDQHGRVYYVDHVEKRTTWDR
PEPLPPGWERRVDNMGRIYYVDHFTRTTTWQRPTLESVRNYEQWQLQR
SQLQGAMQQFNQRFIYGNQDLFATSQSKEFDPLGPLPPGWEKRTDSNG
RVYFVNHNTRITQWEDPRSQGQLNEKPLPEGWEMRFTVDGIPYFVDHN
RRTTTYIDPRTGKSALDNGPQIAYVRDFKAKVQYFRFWCQQLAMPQHI
KITVTRKTLFEDSFQQIMSFSPQDLRRRLWVIFPGEEGLDYGGVAREW
FFLLSHEVLNPMYCLFEYAGKDNYCLQINPASYINPDHLKYFRFIGRF
IAMALFHGKFIDTGFSLPFYKRILNKPVGLKDLESIDPEFYNSLIWVK
ENNIEECDLEMYFSVDKEILGEIKSHDLKPNGGNILVTEENKEEYIRM
VAEWRLSRGVEEQTQAFFEGFNEILPQQYLQYFDAKELEVLLCGMQEI
DLNDWQRHAIYRHYARTSKQIMWFWQFVKEIDNEKRMRLLQFVTGTCR
LPVGGFADLMGSNGPQKFCIEKVGKENWLPRSHTCFNRLDLPPYKSYE
QLKEKLLFAIEETEGFGQE.

By “ITCH polynucleotide” is meant a polynucleotide encoding an ITCH polypeptide. A nucleic acid sequence encoding an exemplary ITCH corresponds to GenBank Accession No. AF038564.1, which is provided below:

(SEQ ID NO: 19)
GGTGACAAAGAGCCAACAGAGACAATAGGAGACTTGTCAATTTGTCTTGA
TGGGCTACAGTTAGAGTCTGAAGTTGTTACCAATGGTGAAACTACATGTT
CAGAAAGTGCTTCTCAGAATGATGATGGCTCCAGATCCAAGGATGAAACA
AGAGTGAGCACAAATGGATCAGATGACCCTGAAGATGCAGGAGCTGGTGA
AAATAGGAGAGTCAGTGGGAATAATTCTCCATCACTCTCAAATGGTGGTT
TTAAACCTTCTAGACCTCCAAGACCTTCACGACCACCACCACCCACCCCA
CGTAGACCAGCATCTGTCAATGGTTCACCATCTGCCACTTCTGAAAGTGA
TGGGTCTAGTACAGGCTCTCTGCCGCCGACAAATACAAATACAAATACAT
CTGAAGGAGCAACATCTGGATTAATAATTCCTCTTACTATATCTGGAGGC
TCAGGCCCTAGGCCATTAAATCCTGTAACTCAAGCTCCCTTGCCACCTGG
TTGGGAGCAGAGAGTGGACCAGCACGGGCGAGTTTACTATGTAGATCATG
TTGAGAAAAGAACAACATGGGATAGACCAGAACCTCTACCTCCTGGCTGG
GAACGGCGGGTTGACAACATGGGACGTATTTATTATGTTGACCATTTCAC
AAGAACAACAACGTGGCAGAGGCCAACACTGGAATCCGTCCGGAACTATG
AACAATGGCAGCTACAGCGTAGTCAGCTTCAAGGAGCAATGCAGCAGTTT
AACCAGAGATTCATTTATGGGAATCAAGATTTATTTGCTACATCACAAAG
TAAAGAATTTGATCCTCTTGGTCCATTGCCACCTGGATGGGAGAAGAGAA
CAGACAGCAATGGCAGAGTATATTTCGTCAACCACAACACACGAATTACA
CAATGGGAAGACCCCAGAAGTCAAGGTCAATTAAATGAAAAGCCCTTACC
TGAAGGTTGGGAAATGAGATTCACAGTGGATGGAATTCCATATTTTGTGG
ACCACAATAGAAGAACTACCACCTATATAGATCCCCGCACAGGAAAATCT
GCCCTAGACAATGGACCTCAGATAGCCTATGTTCGGGACTTCAAAGCAAA
GGTTCAGTATTTCCGGTTCTGGTGTCAGCAACTGGCCATGCCACAGCACA
TAAAGATTACAGTGACAAGAAAAACATTGTTTGAGGATTCCTTTCAACAG
ATAATGAGCTTCAGTCCCCAAGATCTGCGAAGACGTTTGTGGGTGATTTT
TCCAGGAGAAGAAGGTTTAGATTATGGAGGTGTAGCAAGAGAATGGTTCT
TTCTTTTGTCACATGAAGTGTTGAACCCAATGTATTGCCTGTTTGAATAT
GCAGGGAAGGATAACTACTGCTTGCAGATAAACCCCGCTTCTTACATCAA
TCCAGATCACCTGAAATATTTTCGTTTTATTGGCAGATTTATTGCCATGG
CTCTGTTCCATGGGAAATTCATAGACACGGGTTTTTCTTTACCATTCTAT
AAGCGTATCTTGAACAAACCAGTTGGACTCAAGGATTTAGAATCTATTGA
TCCAGAATTTTACAATTCTCTCATCTGGGTTAAGGAAAACAATATTGAGG
AATGTGATTTGGAAATGTACTTCTCCGTTGACAAAGAAATTCTAGGTGAA
ATTAAGAGTCATGATCTGAAACCTAATGGTGGCAATATTCTTGTAACAGA
AGAAAATAAAGAGGAATACATCAGAATGGTAGCTGAGTGGAGGTTGTCTC
GAGGTGTTGAAGAACAGACACAAGCTTTCTTTGAAGGCTTTAATGAAATT
CTTCCCCAGCAATATTTGCAATACTTTGATGCAAAGGAATTAGAGGTCCT
TTTATGTGGAATGCAAGAGATTGATTTGAATGACTGGCAAAGACATGCCA
TCTACCGTCATTATGCAAGGACCAGCAAACAAATCATGTGGTTTTGGCAG
TTTGTTAAAGAAATTGATAATGAGAAGAGAATGAGACTTCTGCAGTTTGT
TACTGGAACCTGCCGATTGCCAGTAGGAGGATTTGCTGATCTCATGGGGA
GCAATGGACCACAGAAATTCTGCATTGAAAAAGTTGGGAAAGAAAATTGG
CTACCCAGAAGTCATACCTGTTTTAATCGCCTGGACCTGCCACCATACAA
GAGCTATGAGCAACTGAAGGAAAAGCTGTTGTTTGCCATAGAAGAAACAG
AAGGATTTGGACAAGAGTAACTTCTGAGAACTTGCACCATGAATGGGCAA
GAACTTATTTGCAATGTTTGTCCTTCTCTGCCTGTTGCACATCTTGTAAA
ATTGGACAATGGCTCTTTAGAGAGTTATCTGAGTGTAAGTAAATTAATGT
TCTCATTTAAAAAAAAAAAAAAAAAAA.

By “IST1 Factor Associated with ESCRT-III (IST1) polypeptide” is meant a polypeptide or fragment thereof capable of binding microtubule-interacting and transport (MIT) domain-containing proteins, such as VPS4, and having at least about 85% identity to NCBI Reference Sequence Accession No. NP_001257908.1. The sequence of an exemplary IST1 polypeptide is provided below:

(SEQ ID NO: 20)
MHKLSVEAPPKILVERYLIEIAKNYNVPYEPDSVVMAEAPPGVETDLIDV
GFTDDVKKGGPGRGGSGGFTAPVGGPDGTVPMPMPMPMPMPSANTPFSYP
LPKGPSDFNGLPMGTYQAFPNIHPPQIPATPPSYESVDDINADKNISSAQ
IVGPGPKPEASAKLPSRPADNYDNFVLPELPSVPDTLPTASAGASTSASE
DIDFDDLSRRFEELKKKT.

By “IST1 polynucleotide” is meant a polynucleotide encoding a IST1 polypeptide. A nucleic acid sequence encoding an exemplary IST1 corresponds to NCBI Reference Sequence Accession No. NM_001270979.1, which is provided below:

(SEQ ID NO: 21)
CCGCCCGCGCTTGTTGTGCTGAGGCCGAGGGAGTCGCCATTTTGGATGGT
GAACCCTGAAGTCGGTGTCTGCTGCGTTCACGGCAGGATTCGGTTAGCTA
ATGCACAAGCTGAGTGTGGAAGCCCCACCCAAAATCCTGGTGGAGAGATA
CCTGATTGAAATTGCAAAGAATTACAACGTACCCTATGAACCTGACTCTG
TGGTCATGGCAGAAGCTCCTCCTGGGGTAGAGACAGATCTTATTGATGTT
GGATTCACAGATGATGTGAAGAAAGGAGGCCCTGGAAGAGGAGGGAGTGG
TGGCTTCACAGCACCAGTTGGTGGACCTGATGGAACGGTGCCAATGCCCA
TGCCCATGCCCATGCCTATGCCATCTGCAAATACGCCTTTCTCATATCCA
CTGCCAAAGGGACCATCAGATTTCAATGGACTGCCAATGGGGACTTATCA
GGCCTTTCCCAATATTCATCCACCTCAGATACCAGCAACTCCCCCATCGT
ATGAATCTGTAGATGACATTAATGCTGATAAGAATATCTCTTCTGCACAG
ATTGTTGGTCCTGGACCCAAGCCAGAAGCCTCTGCAAAGCTTCCTTCCAG
ACCTGCAGATAACTATGACAACTTTGTCCTACCAGAGTTGCCATCTGTGC
CAGACACACTACCAACTGCATCTGCTGGTGCCAGCACCTCAGCATCTGAA
GACATTGACTTTGATGATCTTTCCCGGAGGTTTGAAGAGCTGAAAAAGAA
AACATAGGTCTCTTAAACCAGGCAACTTTCACGTTTTGGGAGTTGAGACT
GAGCAATTTCTCCTTGTAACAAAGAATCTCCATGAAATTCTGTTTCATCT
GTTAACCGTCACTCAGCACAACACTCCCTCTGGGCTCTCTTCCTGCTCCT
CCAGATTCTGCTGCTTTCCAGTTCTCTGTTGATCCTGAGACTAACAATTG
GAGACTGAGGCCAGAGCAACTGGCTCCTGGCAGCTGTGCTTGTCCGTTTC
CTGTCAGAGTGATCCCAGGTTTCCTCCTGGCCCGTCCCATGGTCCCTCCA
CAGGAGTGTGAGAGGATGGGGGAAGCACTGTGGGAAGACCACCAAAGATG
GCTGGACAGTGGGAGAGAGCACGTTGTGAAGCATCCCAGCCTCGTGTTGA
GGTTCCAGACTTAGAAACAGACCCCTCTGTACAGGGGGATTGTGGTGAGT
GAGAATCAAGGCCACCTTGTGTGTTTTCTCACTCTCGAATGCAAGTGGGA
GAGGGAAAATGACTCGGGACGCCATTGTAACGGTTCCTGGAAGCTGGGCC
CTCTCATTGGCATATACAGTACTCCTCGCTGCAGGGCACTGTCCCACCGG
GATCCAGTTGCAAAGTTTGTCTTGACAGTTGAAGGCCTCGCTTAGTTGTA
CTGGATTCTCAGGGAGCCCTCTGTGGCCTTTTGCTTTGCGTGCTGTTTCC
CTTGTACCAGAGGGCGGCACCGTGGAAATTCTGTTTTCCCTGTAGCATAT
TGTGTTGGATTGCATTACTGGCAGAGAAAGGACAAGGTGCCATTCAAGTC
CTAGGGTGGGCTTCCAGCTGCCTTAATAGAAGTACTCAAGTCTTTTGGGT
AGTGAGCTGGAAAGCCTACAGGAAAAGAGGGGTACCTGTTTTCATTTGAA
AACTTTGATTCATGGAACCTTTAAAACTAATCTCAGAAAAATTTTTGGTG
CCCATGCAGCTGTAGTTGTTCACTGCTTTCCTGGATGGATGGGACTCTTA
TGTCATAACTTCTGTTACTCCTTTGGCCCATAGCTAAGGTCATCCTTCCC
CACAGGGGTGGCTTTGGGATTGGATGATACAGCTTTTGCTTCTGTGTAGT
ATACCTGTACATACTTGTTTCAGGCAGCCTTTCTTTAATGTTTTCAGTTG
GTTTGTATTCTGTAGCTCAGTAGCTGCTAATAAAGTTAAAGATCCTGTGT
CCTGCTTTCTCTACTGTTTGGTCAGATGATGAAGTATAAATCTGGATTTT
AGTGGAGAATATTTTAACCAAGTGATCCATGTAAACCAGCCCTTAGTTTC
AGGATTCCTGGAGCAGTACAGAGCTCATTTTGATCTCTGTATTTAGATAG
CCCAGATAGCATCTGTGAATTTCCTTGGTCTTCCTCAGTGGCAGGGCTCC
GCATCTGCCATGCCTCATCCTTGGATGTTTATTTTTAGTAAGTTGCAGTG
AGCTCACTTCTGACTGCTTCATTTCCCGCAGCCATCCTGGGCCATGGGTG
GTGAGTTCTGTTACTTGCTGCTTGGCAGCAGAGCTAGTTTGTCTCGTAGT
ATTCTTGCATTGTTAATCCTATCTTGACAGTGCCAAGATCCATAAGAACT
TGGGACCAAGGGGATTTTGATTCCTAACTTACAGAATTAAAAACAAAGTA
TATTATAATTTTAAAGCTATGCCATGCAAAGATAAGTAGTAATACGCTAA
TAAATACTAAGCCAAGTAGGAGATAACAGGATTAAGGTAGTTCATCTAAA
ACTTCAGTGTCACTGCCCACTGCTGTCGTGGCTGCTTGCTCAGATGAGGT
TTTTTGGGGCCAACTGATTCCTAACAAATTTGAGAGCTTCTGTAGCAGTG
TATCTATTAGTGCAGCTTCTTTCTGAGTATTGAAGACAAAAAGAGAAAAG
TGAGAAAATTGAAATTACTGCTAATAGTGGAGTAAAAAAAGTACCAAAGA
TTTTTAAAAAATTAGTAAATGTAAAGATACTATTTCTTTAATAATTTGCA
GTCAGGCATTGGAGTGTTTCCACTAATGGTTGCGAGCCAACTATTTATAG
GACAATGGCTATAGAATATTATGTAGTCTTATAAATTGGGTTTCCTAGGA
AGATAGCTGGCAGAGCTTTTGAAACTAATAAAGGGAATATGATACTGTGC
ATTATAAATTATGTCAGCCCGTCATCTTAGGGGCAGTTACAGTGGAGCTT
TCCCAGTGATATAACAGCATGCTAGTTTATCTTTTAGTTACCTACCTTAA
GCGACTTTCTTTCTTTTCCAAAGGCCATGAGAATGGCCGAAACGAAAAGA
TTAATTACCACAAGTACCATCAAGATGACACTGGTGAGGATCAGAAAGGT
GCCCAGGACTGGGTCTAAAGCGAAAACCTGGGAAAACAATAAGAACACTT
GCAGTGACTGACAATTTAGTTTATACTTTACAAACATAAGCTATATGCTA
GTGTTTGGGATCTTATCAGAAGAAAAGCTTATCCGAAGGAAACTTAGGGA
GAGAGTCAAAAGATAATAAGAAGGAAAATACTTTTAAATGGACAGAAGAG
CAGGAATGACTCATTTTAAGGAGGTTAAGATAATTGTGACTGCAGGGCTT
TCACAAGAAAACAGGTGAGTTGCAGTGGAATTGGAAATGATTCAAATGTC
ACATGAGTTTTGGCAAAAAATACATCTAATCATGGAAGCTAAAAGTGGGA
GGGGGGTAAGCAAACACTGAAAGATTAAATGCATACCAGAAATGAGAATG
CACACTGTCTCTGCCATATCAAGAAACTACAACCCTAAACACAGGAGCAT
TCCAATAAAAGTTAACAGGTTGGCCACAGAAAA.

By “SMAD family member 4 (SMAD4) polypeptide” is meant a polypeptide or fragment thereof having signal transduction activity associated with transcriptional activation of target genes, such as the TGF-beta receptor, and having at least about 85% identity to GenBank Accession No. AHA34186.1. The sequence of an exemplary SMAD4 polypeptide is provided below:

(SEQ ID NO: 22)
PEYWCSIAYFEMDVQVGETFKVPSSCPIVTVDGYVDPSGGDRFCLSQLSN
VHRTEAIERA.

By “SMAD4 polynucleotide” is meant a polynucleotide encoding a SMAD4 polypeptide. A nucleic acid sequence encoding an exemplary SMAD4 corresponds to GenBank Accession No. KF572433.1, which is provided below:

(SEQ ID NO: 23)
ATTTATTTCCTATAGCTCCTGAGTATTGGTGTTCCATTGCTTACTTTGAA
ATGGATGTTCAGGTAGGAGAGACATTTAAGGTTCCTTCAAGCTGCCCTAT
TGTTACTGTTGATGGATACGTGGACCCTTCTGGAGGAGATCGCTTTTGTT
TGAGTCAACTCTCCAATGTCCACAGGACAGAAGCCATTGAGAGAGCAAGG
TATTGATTGTATAGTCAGATAGTT.

By “UNC51-like Kinase 3 (ULK3) polypeptide” is meant a polypeptide or fragment thereof having kinase activity and having at least about 85% identity to GenBank Accession No. BAG57541.1. The sequence of an exemplary ULK3 polypeptide is provided below:

(SEQ ID NO: 24)
MQRNGSASRGLEKTRLRLCREARIPESAFLTGLTRESWEARCWCAKDTRE
VVAIKCVAKKSLNKASVENLLTEIEILKGIRHPHIVQLKDFQWDSDNIYL
IMEFCAGGDLSRFIHTRRILPEKVARVFMQQLASALQFLHERNISHLDLK
PQNILLSSLEKPHLKLADFGFAQHMSPWDEKHVLRGSPLYMAPEMVCQRQ
YDARVDLWSMGVILYEALFGQPPFASRSFSELEEKIRSNRVIELPLRPLL
SRDCRDLLQRLLERDPSRRISFQDFFAHPWVDLEHMPSGESLGRATALVV
QAVKKDQEGDSAAALSLYCKALDFFVPALHYEVDAQRKEAIKAKVGQYVS
RAEELKAIVSSSNQALLRQGTSARDLLREMARDKPRLLAALEVASAAMAK
EEAAGGEQDALDLYQHSLGELLLLLAAEPPGRRRELLHTEVQNLMARAEY
LKEQVKMRESRWEADTLDKEGLSESVRSSCTLQ.

By “ULK3 polynucleotide” is meant a polynucleotide encoding a ULK3 polypeptide. A nucleic acid sequence encoding an exemplary ULK3 corresponds to GenBank Accession No. AK294245.1, which is provided below:

(SEQ ID NO: 25)
AGAATGCAAAGAAATGGATCAGCTAGCCGAGGGTTGGAGAAGACCAGGCT
GAGGCTGTGTCGGGAAGCCAGGATTCCAGAATCAGCATTCCTCACTGGCC
TCACAAGGGAAAGCTGGGAAGCCCGGTGCTGGTGTGCAAAGGACACTCGT
GAAGTGGTAGCCATAAAGTGTGTAGCCAAGAAAAGTCTGAACAAGGCATC
GGTGGAGAACCTCCTCACGGAGATTGAGATCCTCAAGGGCATTCGACATC
CCCACATTGTGCAGCTGAAAGACTTTCAGTGGGACAGTGACAATATCTAC
CTCATCATGGAGTTTTGCGCAGGGGGCGACCTGTCTCGCTTCATCCATAC
CCGCAGGATTCTGCCTGAGAAGGTGGCGCGTGTCTTCATGCAGCAATTAG
CTAGCGCCCTGCAATTCCTGCATGAACGGAATATCTCTCACCTGGATCTG
AAGCCACAGAACATTCTACTGAGCTCCTTGGAGAAGCCCCACCTAAAACT
GGCAGACTTTGGTTTCGCACAACACATGTCCCCGTGGGATGAGAAGCACG
TGCTCCGTGGCTCCCCCCTCTACATGGCCCCCGAGATGGTGTGCCAGCGG
CAGTATGACGCCCGCGTGGACCTCTGGTCCATGGGGGTCATCCTGTATGA
AGCCCTCTTCGGGCAGCCCCCCTTTGCCTCCAGGTCGTTCTCGGAGCTGG
AAGAGAAGATCCGTAGCAACCGGGTCATCGAGCTCCCCTTGCGGCCCCTG
CTCTCCCGAGACTGCCGGGACCTACTGCAGCGGCTCCTGGAGCGGGACCC
CAGCCGTCGCATCTCCTTCCAGGACTTTTTTGCGCACCCCTGGGTGGACC
TGGAGCACATGCCCAGTGGGGAGAGTCTGGGGCGAGCAACCGCCCTGGTG
GTGCAGGCTGTGAAGAAAGACCAGGAGGGGGATTCAGCAGCTGCCTTATC
ACTCTACTGCAAGGCTCTGGACTTCTTTGTACCTGCCCTGCACTATGAAG
TGGATGCCCAGCGGAAGGAGGCAATTAAGGCAAAGGTGGGGCAGTACGTG
TCCCGGGCTGAGGAGCTCAAGGCCATCGTCTCCTCTTCCAATCAGGCCCT
GCTGAGGCAGGGGACCTCTGCCCGAGACCTGCTCAGAGAGATGGCCCGGG
ACAAGCCACGCCTCCTAGCTGCCCTGGAAGTGGCTTCAGCTGCCATGGCC
AAGGAGGAGGCCGCCGGCGGGGAGCAGGATGCCCTGGACCTGTACCAGCA
CAGCCTGGGGGAGCTACTGCTGTTGCTGGCAGCGGAGCCCCCGGGCCGGA
GGCGGGAGCTGCTTCACACTGAGGTTCAGAACCTCATGGCCCGAGCTGAA
TACTTGAAGGAGCAGGTCAAGATGAGGGAATCTCGCTGGGAAGCTGACAC
CCTGGACAAAGAGGGACTGTCGGAATCTGTTCGTAGCTCTTGCACCCTTC
AGTGACCCTAGAAGAATGATTGGACAGTGCTGAGACCCCCATATCCCAGA
GTCCCCAGCCTCCCTCAGGTTACTCTGCACCCCACAGATGGTTTGATGGC
TGTGCTGTATACTGGAGGGGAGGGCAGGACTCTGGGAGAACAGCACTTCT
TTCATGAGACCTTTGTTACTCGGTGGTTACT.

By “VPS4A polypeptide” is meant a polypeptide or fragment thereof having ATPase activity and having at least about 85% identity to NCBI Accession No. NP_037377.1. The sequence of an exemplary VPS4A polypeptide is provided below:

(SEQ ID NO: 26)
MTTSTLQKAIDLVTKATEEDKAKNYEEALRLYQHAVEYFLHAIKYEAHSD
KAKESIRAKCVQYLDRAEKLKDYLRSKEKHGKKPVKENQSEGKGSDSDSE
GDNPEKKKLQEQLMGAVVMEKPNIRWNDVAGLEGAKEALKEAVILPIKFP
HLFTGKRTPWRGILLFGPPGTGKSYLAKAVATEANNSTFFSVSSSDLMSK
WLGESEKLVKNLFELARQHKPSIIFIDEVDSLCGSRNENESEAARRIKTE
FLVQMQGVGNNNDGTLVLGATNIPWVLDSAIRRRFEKRIYIPLPEEAARA
QMFRLHLGSTPHNLTDANIHELARKTEGYSGADISIIVRDSLMQPVRKVQ
SATHFKKVCGPSRTNPSMMIDDLLTPCSPGDPGAMEMTWMDVPGDKLLEP
VVCMSDMLRSLATTRPTVNADDLLKVKKFSEDFGQES.

By “VPS4A polynucleotide” is meant a polynucleotide encoding a VPS4A polypeptide. A nucleic acid sequence encoding an exemplary VPS4A is provided below:

(SEQ ID NO: 27)
GCCCTCGGACTCGGCTCCCGCTGCGAGCGGCCGCCCTGCCCGCGCACCGC
GCTCAGCGCCCACCGCCGGGCTTCCCGCGCCGGACCCAGTACCTCGGCTC
CCCGGGGCCGGACCGAGGCCGCAAGCAGCGCCGCGGGGTGTGGGGCGGAC
CCAGGAGATGAAATGACAACGTCAACCCTCCAGAAAGCCATTGATCTGGT
GACGAAAGCCACAGAGGAGGACAAAGCCAAGAACTACGAGGAGGCGCTGC
GGCTGTACCAGCATGCGGTGGAGTACTTCCTCCACGCTATCAAGTATGAG
GCCCACAGCGACAAGGCCAAGGAGAGCATTCGAGCCAAGTGCGTGCAGTA
CCTAGACCGGGCCGAGAAGCTGAAGGATTATTTACGAAGCAAAGAGAAAC
ACGGCAAGAAGCCAGTCAAAGAGAACCAGAGTGAGGGCAAGGGCAGTGAC
AGTGACAGTGAAGGGGATAATCCGGAGAAAAAGAAACTGCAAGAACAGCT
GATGGGTGCCGTCGTGATGGAGAAGCCCAACATACGGTGGAACGACGTGG
CCGGGCTGGAGGGGGCCAAGGAGGCCCTCAAAGAAGCTGTCATTTTGCCA
ATCAAATTCCCACACTTGTTCACAGGCAAGCGCACCCCCTGGCGGGGGAT
TCTGCTGTTCGGACCCCCTGGCACAGGGAAATCCTACCTGGCCAAAGCCG
TGGCAACAGAGGCCAACAACTCCACCTTCTTCTCTGTGTCCTCCTCAGAT
CTGATGTCCAAGTGGCTGGGGGAGAGTGAAAAGCTGGTCAAGAACCTGTT
TGAGCTGGCCAGGCAGCACAAGCCCTCCATCATCTTCATCGATGAGGTGG
ATTCCCTCTGCGGGTCCCGAAATGAAAATGAGAGTGAGGCCGCCCGGAGG
ATCAAAACGGAGTTCTTGGTCCAGATGCAGGGGGTGGGGAATAACAATGA
TGGGACTCTGGTTCTTGGAGCCACAAACATCCCATGGGTGTTGGATTCGG
CCATCAGGAGGAGGTTTGAAAAACGAATTTATATCCCCTTGCCGGAGGAA
GCTGCCCGCGCCCAGATGTTCCGGTTGCATCTCGGGAGCACTCCCCACAA
CCTCACGGATGCAAACATCCACGAGCTGGCCCGGAAGACGGAAGGCTACT
CGGGCGCGGACATCAGCATCATCGTGCGGGACTCTCTCATGCAGCCCGTG
AGGAAGGTGCAGTCGGCCACACACTTCAAAAAGGTCTGTGGCCCCTCTCG
CACCAACCCCAGCATGATGATTGATGACCTCCTGACTCCATGCTCACCAG
GGGACCCAGGAGCCATGGAGATGACTTGGATGGATGTCCCTGGGGACAAA
CTCTTAGAGCCTGTGGTTTGCATGTCGGACATGCTGCGGTCTCTGGCCAC
CACCCGGCCCACGGTGAATGCAGACGACCTCCTGAAAGTGAAGAAATTCT
CAGAGGACTTTGGGCAAGAGAGTTAAAAGCTGCTTCACTTGGGCAATGGT
GAAGGTGGGAGGTTGATTGGGGCAAATCCAGGCACTCCCCATGTCAACAG
CCAGACAGGGCTCCAGGGCTTGTCCCAGTCAATACAGAGTTCCCTCTGCT
GTCTGGCCGTCTGCCAGGGAGCCAGAAGGAAGGGCCTTGCAGCCACAGAG
ACACTCCACTGCCCTGGGGCACACAGTGGACACTGCTCTTCCTACTTCCT
CCTCTCCTGGATGCTCATCAGCTCCTTCTGCCTCCCCCCCTTTTTTTTCC
ATCTTTTGTTCCCCTAAATTAATGCTGCTTGGATTTTCATCTTATTTATA
AAGATAAAATCACCTGGAAGTGTCAAGGAGTGGGGCGGGGTGGCGGGGGA
GAAGCAGCCGTGCTGCCAGGTCACCCAGACCTCCAGACAGCCGGCTAGCC
CCACTGCCCGTTCCTTTTACGCCCAAGTTTTGCTCCTTGAGAGCAGATTG
GCTGATGCCCCTGCAACCCCAGCCCAAGCTCTGCCTCAAAGACCGAGTGA
CATAAGCCATTCCCACCCTCCTAGGTTCACATCCAGGGCTGTGTCTTCCT
TGGGGGAGGAGATGGTGTCGTTTAGATCAGGGTAAGGCAGTCAGGCGGGT
GTTCACCACTGCCTTTTCTTCCTCTGAGCGTGAGAACACTGAACCCAGCC
ACTGCCCCTGGGTCCCTGTCCTGGAAATGGTCTAATAAATCCTTTTCCCT
TCTTGAGCTACCCAAGTGATCTCATCTTTCCTTGACTCTTAACTGAGACT
CTGAAGGGCCACCCTTGCTTCCAATTAAAAGACCCTCTGGTTTTCTGTCT
CCTCTCTTTTCTAAAGCAGAGCAGGAAACAGAACACTGCAGTTCTTAGGC
CTGTTCTCCAGTCAGGTGTGCAAGGCCTCCTCCCCTTAAGGCCTCAGAAG
TTTGGCTGGGGATGTTTGTGGGATCCAGACAGTTCTTGCCGTTGTTCGGC
CTACAGATCAGAGACTGCAAAGTGGGAGCCCTGCGTACCAGACCCGTCCT
GCAGCTGTGTTTGTTTGGTTTGGTCTGCCGCTAACATTTAAAAGTCGAGA
GTTGCTGGGCGCGGTGGCTCACGCCTGTAATCCCAGCACTTTGGGCCGCT
AACATTTAAAAGTCGAGAGTTGCTGGGCGCGGTGGCTCACGCCTGTAATC
CCAGCACTTTGGGAGGCCGACGCGGGCGGATCACAAGGTCAGGAAATCGA
GAACATCCTGGCTAACACAGTGAAACCCCGTCTCACTAAAAATAAAAAAA
TTAGCCGGGCGTGGTGGTGGGCGCCTGCAGTCCCAGCTACTTGGGAGGCT
GAGGCAGGAGAATGGCGTGAACCCGGGGGACGGAGCTTGCAGTGAGCCGA
GATCGCGCCACTGCACTCCAGCCTGGGCGACAGAGCAAGACTCTGTCTCA
AAAAAAAAAAAAAAGTCGAGAGTTGACATAAAAATTCAGAATTCTGACTT
CTAGAAGATTAAATTAGGTAATGTTGGGCCTGCGTCCTCATGGGGCAGCC
TGTCAGCGGCCACTAAGTAGAGGCTGCTCTGTACAGAAGGGGTCCCCCAT
CCCCACTCATCTGTGTTGCCTGCCCAGATAGCCCCATGGCCATTTCAGTT
TGCGACCCCCAACAGACCAAGGACCCAGAGCTGGCAGCCCTGGACCCACG
TCCGGCAGGCAGTGAAGCTGCAGAGGCTGTCCCACGTTTACCCCATGCAT
ATAAACAGACCAAAGTCAAAGCACATTCACACAGAAAGACCTGGCCTAGT
AAATGAAGCTTATAGCCTGCAAAGGGCTTTGTTCCAACCTTCCCTCTCCA
AAACCCAGTACTGCTCCTCTACCTTTCAAACCTGGTTGGGCCAGAACTGA
TTACATGGCCAACTCTTGGCTCACACTGGGCTTGCACTGGGCCATTTCTT
TGCTCTGGTTCAGAGCCTCTGCAGAAGTGAGCTAACTTTGGATCAGTAGC
TCCAACACTTGGCTCCAGTGCTGGCAGGTTACCCCTCAACCATAGCAGCT
GGGATCTTTGTGGCTTGGGGGTGCTTGTGTAAGTTCTTGGTTATGTGCCC
TCAGCCACCAGGATATGAACCGTGTCTGCAGAGCTTCACTTTAGTGAGCT
TACAAGTTTTTAAATTTTTACTTCTAGTTACATTTACTTGAATCAGAACG
TTGTTTCCTCATTCCTACTGCTTAAACACCTTGACAAGTCCTAGGGGTTA
ACAAAGGTTAGCATGGCTATGGTCCATCCCTGTGCTCTAGTTAGAGCGTG
AAGACACCTGACTTTCCAGTTGTCTCTCTCCATGACCAGCAACAGGAACC
ACTGACGCTGAACTTTGGACAGTGGCCTCAGACTCTGGCTGCCAGCACAC
AACCTGCCATCATCGATGTTAAACATGCTGACATGTGCAGAGGAGTTTCC
TCCCTGAAATGCTCTGAAATTCACTTCTCTGCCTGGGGATTCTGTTATAA
ACCTTCTGCCTACATTGGCTTTCACTGTGGAAGTTGATTTCTAAAACTCT
GATGAGCTCACAACGTTGACATGTATATGCGTTTTTTGTGAGTGCTTCCT
GCTCAAAGTGGGAGAGATTTTACTGGAAATGCAATAAAGTTTGCATTTTA
TTGCTA.

By “VPS4B polypeptide” is meant a polypeptide or fragment thereof having ATPase activity and having at least about 85% identity to NCBI Accession NP_004860.2. The sequence of an exemplary VPS4B polypeptide is provided below:

(SEQ ID NO: 28)
MSSTSPNLQKAIDLASKAAQEDKAGNYEEALQLYQHAVQYFLHVVKYEAQ
GDKAKQSIRAKCTEYLDRAEKLKEYLKNKEKKAQKPVKEGQPSPADEKGN
DSDGEGESDDPEKKKLQNQLQGAIVIERPNVKWSDVAGLEGAKEALKEAV
ILPIKFPHLFTGKRTPWRGILLFGPPGTGKSYLAKAVATEANNSTFFSIS
SSDLVSKWLGESEKLVKNLFQLARENKPSIIFIDEIDSLCGSRSENESEA
ARRIKTEFLVQMQGVGVDNDGILVLGATNIPWVLDSAIRRRFEKRIYIPL
PEPHARAAMFKLHLGTTQNSLTEADFRELGRKTDGYSGADISIIVRDALM
QPVRKVQSATHFKKVRGPSRADPNHLVDDLLTPCSPGDPGAIEMTWMDVP
GDKLLEPVVSMSDMLRSLSNTKPTVNEHDLLKLKKFTEDFGQEG.

By “VPS4B polynucleotide” is meant a polynucleotide encoding a VPS4B polypeptide. A nucleic acid sequence encoding an exemplary VPS4B is provided below:

(SEQ ID NO: 29)
AGAGGGCCTCGGGATTGCGGAAGTTTGGTGGGGAGGGTCGGAGCTCTGGT
GGAGAGAGTGTTGTCTAAAACAAGTTCCGGAAGGGAGGCTGCCCTTCGCG
GTCCGAGAACCACCGGCCTCCCCAGTTTGAGGGCTGTTACCCCGTGCGCG
CTTCGACGTTGCTGCTGTTGGCTCTCCTCGCCCCTCGTTCCCTTGGGAAC
CGCCTGGGAACTCCGCCATGTCATCCACTTCGCCCAACCTCCAGAAAGCG
ATAGATCTGGCTAGCAAAGCAGCGCAAGAAGACAAGGCTGGGAACTACGA
AGAAGCCCTTCAGCTCTATCAGCATGCTGTGCAGTATTTTCTTCATGTCG
TTAAATATGAAGCACAGGGTGATAAAGCCAAGCAAAGTATCAGGGCAAAG
TGTACAGAATATCTTGATAGAGCAGAAAAACTAAAGGAGTACCTGAAAAA
TAAAGAGAAAAAAGCACAGAAGCCAGTGAAAGAAGGACAGCCGAGTCCAG
CAGATGAGAAGGGGAATGACAGTGATGGGGAAGGAGAATCTGATGATCCT
GAAAAAAAGAAACTACAGAATCAACTTCAAGGTGCCATTGTTATAGAACG
ACCAAATGTGAAATGGAGTGACGTTGCTGGACTTGAAGGAGCCAAAGAAG
CACTGAAAGAGGCTGTGATACTGCCTATTAAATTTCCTCATCTTTTTACA
GGCAAGAGAACACCTTGGAGGGGAATCCTATTATTTGGGCCGCCTGGAAC
AGGAAAGTCCTACTTAGCCAAAGCTGTAGCAACAGAAGCCAACAACTCAA
CATTTTTTTCAATATCTTCCTCTGATCTTGTTTCTAAGTGGCTAGGTGAA
AGTGAAAAACTGGTTAAGAATTTATTCCAACTTGCCAGAGAGAACAAGCC
CTCCATTATCTTCATTGATGAAATTGATTCTCTCTGTGGTTCAAGAAGTG
AAAATGAAAGTGAAGCCGCACGTAGAATTAAGACGGAGTTCCTAGTGCAA
ATGCAAGGGGTTGGTGTAGACAATGATGGAATTTTGGTTCTGGGAGCTAC
AAATATACCCTGGGTTCTGGATTCTGCCATTAGGCGAAGATTTGAGAAAC
GAATTTATATTCCCTTGCCGGAACCCCATGCCCGAGCAGCAATGTTTAAA
CTGCACCTAGGGACCACTCAGAACAGTCTCACGGAAGCAGACTTTCGGGA
ACTTGGGAGGAAAACAGATGGTTATTCAGGGGCAGATATAAGTATCATTG
TACGTGATGCCCTTATGCAGCCTGTTAGGAAAGTACAGTCAGCTACTCAT
TTTAAAAAGGTTCGCGGACCTTCCCGAGCTGATCCTAACCATCTTGTAGA
TGATCTGCTAACACCTTGCTCTCCAGGTGACCCTGGTGCCATTGAAATGA
CATGGATGGATGTCCCTGGAGATAAACTTTTGGAGCCAGTTGTTTCCATG
TCGGATATGTTGCGGTCACTATCTAACACAAAACCTACAGTCAATGAACA
TGACTTGTTGAAATTAAAGAAGTTTACAGAAGATTTTGGTCAAGAAGGCT
AAGCCAAAGACAAGGAAGATGCTTACCATATGTATTCTTTCTTTCATAGA
TATTTTTGTCTATTTGGATCGCATTAATTGTTTCCAGTAAAACTCTTTTA
CCACAGGGAAATACACATCTCACTTCAGAGTTCCATTAGGTTTTATATTG
TACTTTTCCTCCATTACTTATTAAATACTCCTATTAACAAAAGGTACAAA
ATAACAGGTTATGAGGAAATGAGCGATATATGAACGGCATAAAAACAGAA
ATTACCCAGTAAAAAGGATGTCAGAAATTGACATACAAATATTTACAATT
TTTATGAATGGTGGTCTTTGCAAAGAGCATTTATATTTTCTTTTTTTTTT
ACTAAAATGATATAGGGTTTATTTTATATTTTCAAAAAAATTGTTAAACA
TCATTCTTATCAATGTAAAATTTACGTTATTAAAAAATTACAAATGATAG
AATCTTTACTCAGGGATGGGCAATAAAACAGCAAAGAGCTTTGTGATTGG
GTTTGAAAGATTTTGAATTATAGACAGTGCTCTTAATAGTTTTTAATAAG
TTGATATTTTTTTCTGTGTAGATTTAAATAATTTCTTTAAAAGCGTAAGC
TTTGGCTGGGCATGGTGGCTTATGCCTGTAATCCCAGCACTTTGGGAGGC
TACGATGGGTGGATCACCTGAGGTCAGGAGTTCAAGACCAGTCTGGCCAA
CATGGTGAAATGCCGTCTCTACTAAAAATACAAAAATTAGCTGGGCATGA
TGGCGAGTGTCTGTAATCCCAGCTACTCAGGAGGCTGAGGCGGGAGAATC
ACTTGAACCTGGGAGGCAGAGGTTGCAGTGAGCTGAGATCGCGCTACTGC
ATTCCAGCCTGGGCGCCGAGATAGCGCCATTGCACTCCAGCCTGGGCGAC
TGAATAAAACTCTGTCTCAAAAAAAAACAAAAACGGTAAGCTTTTATATT
TAATTAACTGATACCTTTCACTGTGCAATCTCAAGAGGAAGATTTTCTAA
ATTAGACATTGTCTTTACCTTAAAGAAGAAAATTGTCTATTTCAGTGTCT
TTTTTAGCAAGATTGACCAAAACAGGTGGTAGGTGTGTAATTTTTAACTG
TCATGAAATACTGTAATGCGCACCTTTGCTAATAAGGAAATGGTCTCCCT
TTGAATTATGGGAAGTTTCTTGTCTGTGATATGGGGTGTCTGTGTACATA
TATGTATATTTCCTTTTTTAATACAGGATAATAATATGGGGTCTTGTCCT
TCACCTTTTAAGTTCAGGGAACAGTTTGTCCTGACCACACATATGTTATC
AGTGTAATGCTTTATGAAGCTTTAGAAATGAGCTCATGACTCATTAAAAA
ATAATAAACGATGCTATTTTAATATTTTCAAATAAATAATCTGAGGATGT
GTTTCATACTTTAGCATCAACGCTGAGTTGTGCCTTGTAGAATTGCATTG
TCTATACATGCAAAAGACTATTAAGTTAGGATGAACTAGATTCCCTAATT
ACAGTGAAATTCTATCTTGCAAATGCTATAGAATAACTTGCATTTTAAAG
TGTATTTGCACATTTTACATATGCTATGTGGTTGCCTTTGGGTTTTCTGT
ACAGATTGTTTTTGTTATTAAATGGAAAAGGCCTGAATTATGCTCTTATG
TGAAGTAAATTGATATAACCTATTGACTGTATTCTGTGTATTATTCTTTA
TTTTGGGACCTTCTGTGTAAAAGAAAATATTACCTAAATTGGTTAGATAC
AAAGAAATCATTAAAATTATTAATGTATATATGAAAA.

By “Vesicle Trafficking 1 (VTA1) polypeptide” is meant a polypeptide or fragment thereof having activity associated with trafficking of the multivescicular body and having at least about 85% identity to GenBank Accession No. AAH06989.1. The sequence of an exemplary VTA1 polypeptide is provided below:

(SEQ ID NO: 30)
MAALAPLPPLPAQFKSIQHHLRTAQEHDKRDPVVAYYCRLYAMQTGMKID
SKTPECRKFLSKLMDQLEALKKQLGDNEAITQEIVGCAHLENYALKMFLY
ADNEDRAGRFHKNMIKSFYTASLLIDVITVFGELTDENVKHRKYARWKAT
YIHNCLKNGETPQAGPVGIEEDNDIEENEDAGAASLPTQPTQPSSSSTYD
PSNMPSGNYTGIQIPPGAHAPANTPAEVPHSTGVASNTIQPTPQTIPAID
PALFNTISQGDVRLTPEDFARAQKYCKYAGSALQYEDVSTAVQNLQKALK
LLTTGRE.

By “VTA1 polynucleotide” is meant a polynucleotide encoding a VTA1 polypeptide. A nucleic acid sequence encoding an exemplary VTA1 corresponds to GenBank Accession No. BC006989.1, which is provided below:

(SEQ ID NO: 31)
AGGAAGTGGTGAGTTCGGAGTAGAGATGGCCGCGCTTGCACCGCTGCCCC
CGCTCCCCGCACAGTTCAAGAGCATACAGCATCATCTGAGGACGGCTCAG
GAGCATGACAAGCGAGACCCTGTGGTGGCTTATTACTGTCGTTTATACGC
AATGCAGACTGGAATGAAGATCGATAGTAAAACTCCTGAATGTCGCAAAT
TTTTATCAAAGTTAATGGATCAGTTAGAAGCTCTAAAGAAGCAGTTGGGT
GATAATGAAGCTATTACTCAAGAAATAGTGGGCTGTGCCCATTTGGAGAA
TTATGCTTTGAAAATGTTTTTGTATGCAGACAATGAAGATCGTGCTGGAC
GATTTCACAAAAACATGATCAAGTCCTTCTATACTGCAAGTCTTTTGATA
GATGTCATAACAGTATTTGGAGAACTCACTGATGAAAATGTGAAACACAG
GAAGTATGCCAGATGGAAGGCAACATACATCCATAATTGTTTAAAGAATG
GGGAGACTCCTCAAGCAGGCCCTGTTGGAATTGAAGAAGATAATGATATT
GAAGAAAATGAAGATGCTGGAGCAGCCTCTCTGCCCACTCAGCCAACTCA
GCCATCATCATCTTCAACTTATGACCCAAGCAACATGCCATCAGGCAACT
ATACTGGAATACAGATTCCTCCGGGTGCACACGCTCCAGCTAATACACCA
GCAGAAGTGCCTCACAGCACAGGTGTAGCAAGTAATACTATCCAACCTAC
TCCACAGACTATACCTGCCATTGATCCCGCACTTTTCAATACAATTTCCC
AGGGGGATGTTCGTCTAACCCCAGAAGACTTTGCTAGAGCTCAGAAGTAC
TGCAAATATGCTGGCAGTGCTTTGCAGTATGAAGATGTAAGCACTGCTGT
CCAGAATCTACAAAAGGCTCTCAAGTTACTGACGACAGGCAGAGAATGAA
GCCTTTGTATGACAGACCCATGTATTTTTGGCATGAGGAACTAACAGTCC
ATTACTCTATCTTCAGCCTATCAGGATCACAGTTTTAAGGAAGACTTGGT
TTTGTTGAATATGACAATGAAATCTGTGTGTATCAGATTTTTATTGAAGC
ATTCATCAGCAGCCTCAACCAGTTTTCATTGTCCATTTACTAGATTCAAT
CGTCTCTGAGTATATAGGGCTGATGTTAGCAAGACCCTAAAAATGTCCAT
TGAACCCTGCTTCAAAAAATGAAAACACACCTCTATAAAATGTGTACTGG
GAATAAGCTTTGTATTTACATACATTAGGGGAATTTTTTAAAATCTGTAA
TGTTTGGACAAACAGATGATATTACTTTGCTATAAAATTATAAATGTAAC
TTTTAATAAAGATAGCCAGAATATTCTAAATTAGAAATTACGTTTTTGTT
TCCCTCAAGACATAAAACAAATATAAACATTCTAAACTGCTGGATGAATC
TGAAAAGACATTAAGTTCAAATTTTAATTTATTCTCATATTAAATATAAC
TCCATTAAAAGTTTAAAATTTCATGGGAGAAAATATAATAAGGTAAAGAG
GTAGAATCACTTTCAGACTTAAGAATAATGTTGATTTCCCAAGTGCTTTA
CCTTATCTGTTAAAGCGTAAGATGAATTGGTATTTGCTTCATAGGCAGTT
TGACTGCATGTATTAGAGAATGAAAAGAAGATATTTGTAGTAATGCCTGG
AAACTTGGTGCTTTAAATTAAGGTACTCCTCTGCTGCTGTAGAATGGATT
CCACACAGTGGATAGCTATGGGTGATTCAGAATATTATGTTTAGATTCCC
ATTTGTTAAGTTTATAAGTTTTGTGGGGAATTATGAACTTACTGTGTACT
ACCTGCATTTGTGCTGTGTGAAAAATAAATACAAGGATTCGTTTAGCTAA
TTCAACAAAAAAAAAAAAAAAAAAAAAAAAAAAAAA.

By “agent” is meant any small molecule chemical compound, antibody, nucleic acid molecule, or polypeptide, or fragments thereof. An agent for use in the disclosure may include, but is not limited to, an anti-VPS4A antibody, a VPS4A siRNA, a VPS4A shRNA, a VPS4A miRNA, a VPS4A ribozyme, a VPS4A antisense RNA, a nucleic acid that decreases VPS4A expression, a vector expressing at least one nucleic acid that decreases VPS4A nucleic acid expression; an anti-VPS4B antibody, a VPS4B siRNA, a VPS4B shRNA, a VPS4B miRNA, a VPS4B ribozyme, a VPS4B antisense RNA, a nucleic acid that decreases VPS4B expression, a vector expressing at least one nucleic acid that decreases VPS4B nucleic acid expression; an anti-ULK3 antibody, an ULK3 siRNA, an ULK3 shRNA, an ULK3 miRNA, an ULK3 ribozyme, an ULK3 antisense RNA, a nucleic acid that decreases ULK3 expression, a vector expressing at least one nucleic acid that decreases ULK3 nucleic acid expression; an anti-CHMP1A antibody, a CHMP1A siRNA, a CHMP1A shRNA, a CHMP1A miRNA, a CHMP1A ribozyme, a CHMP1A antisense RNA, a nucleic acid that decreases CHMP1A expression, a vector expressing at least one nucleic acid that decreases CHMP1A nucleic acid expression; an anti-CHMP1B antibody, a CHMP1B siRNA, a CHMP1B shRNA, a CHMP1B miRNA, a CHMP1B ribozyme, a CHMP1B antisense RNA, a nucleic acid that decreases CHMP1B expression, a vector expressing at least one nucleic acid that decreases CHMP1B nucleic acid expression; an anti-IST1 antibody, a IST1 siRNA, a IST1 shRNA, a IST1 miRNA, a IST1 ribozyme, a IST1 antisense RNA, a nucleic acid that decreases IST1 expression, a vector expressing at least one nucleic acid that decreases IST1 nucleic acid expression; an anti-VTA1 antibody, a VTA1 siRNA, a VTA1 shRNA, a VTA1 miRNA, a VTA1 ribozyme, a VTA1 antisense RNA, a nucleic acid that decreases VTA1 expression, a vector expressing at least one nucleic acid that decreases VTA1 nucleic acid expression; an anti-IST1 antibody, a IST1 siRNA, a IST1 shRNA, a IST1 miRNA, a IST1 ribozyme, a IST1 antisense RNA, a nucleic acid that decreases IST1 expression, a vector expressing at least one nucleic acid that decreases IST1 nucleic acid expression and any combinations thereof. Further non-limiting examples of agents include a guide RNA targeting VPS4A, a guide RNA targeting VPS4B, a guide RNA targeting ULK3, a guide RNA targeting CHMP1A, a guide RNA targeting CHMP1B, a guide RNA targeting VTA1, a guide RNA targeting IST1, polypeptides or polynucleotides encoding polypeptides for targeted gene editing or for CRISPR interference, and various combinations thereof. The agent can also be a small molecule inhibitor of VPS4A, VPS4B (e.g., MSC1094308), ULK3 (e.g., SU6668), CHMP1A, CHMP1B, VTA1, or IST1.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antisense nucleic acid”, it is meant a non-enzymatic nucleic acid molecule that binds to target RNA by means of RNA-RNA or RNA-DNA interactions and alters the activity of the target RNA (for a review, see Stein and Cheng. Science 261:1004-1012, 1993; Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of antisense strategies, see Schmajuk N A et al. J Biol Chem, 274(31):21783-21789, 1999; Delihas N et al., Nat Biotechnol. 15(8):751-753, 1997; Aboul-Fadl T, Curr Medicinal Chem 12:763-771, 2005.) In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include cancers, such as brain, bladder, bile, blood, breast, duct (e.g., bile duct or pancreatic duct), colon, colorectal, esophageal, gastric, germ cell, liver, ovarian, pancreatic, uterine, and lung cancer, renal cell carcinoma, pancreatic ductal adrenocarcinoma, and sarcomas, such as, osteosarcoma and rhabdomyosarcoma (e.g., pediatric rhabdomyosarcoma (RMS)). In embodiments, the cancer is a pediatric cancer. In embodiments, the cancer occurs in an adult subject.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, an effective amount induces apoptosis in a neoplastic cell, reduces cell survival, reduces proliferation, or otherwise reduces or stabilizes cancer progression.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Non-limiting examples of markers include a VPS4A polypeptide or polynucleotide, a VPS4B polypeptide or polynucleotide, a CDH1 polypeptide or polynucleotide, a ULK3 polypeptide or polynucleotide, a SMAD4 polypeptide or polynucleotide, a CHMP4B polypeptide or polynucleotide, an ISG15 polypeptide or polynucleotide, and an ITCH polypeptide or polynucleotide.

By “MSC1094308” is meant a compound corresponding to CAS Number 2219320-08-6 and having the structure

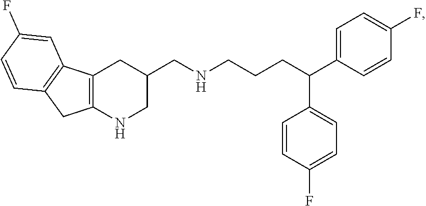 [SMILES: Fc1ccc(C(CCCNCC2CNC3=C(C2)c2cc(F)ccc2C3)c2ccc(F)cc2)cc1]

or a pharmaceutically acceptable salt or solvate thereof.

As used herein, “obtaining” as in “obtaining an agent” includes synthesizing, purchasing, or otherwise acquiring the agent.

By “neoplasia” is meant any disease that is caused by or results in inappropriately high levels of cell division, inappropriately low levels of apoptosis, or both.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

By “obtaining” as in “obtaining the inhibitory nucleic acid molecule” is meant synthesizing, purchasing, or otherwise acquiring the inhibitory nucleic acid molecule.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant protein of the invention, or an RNA molecule).

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “SU6668” is meant a compound corresponding to CAS Number 252916-29-3 and having the structure

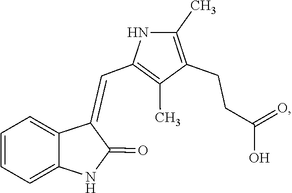 [SMILES: Cc1[nH]c(/C=C2\C(=O)Nc3ccccc32)c(C)c1CCC(=O)O]

or a pharmaceutically acceptable salt or solvate thereof.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. Non-limiting examples of animals include a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1A is a schematic detailing the concept behind the synthetic lethality analysis workflow. For each of 50 selected tumor suppressor genes (see also Table 5), its copy number was correlated across cancer cell lines with gene dependency scores from CRISPR-SpCas9 or RNAi interference loss-of-function screens. Correlations with dependent genes localizing to the same chromosomal arm as the tumor suppressor genes are classified as cis interactions while interactions with genes localized to other chromosomal arms are classified as trans interactions.

FIG. 1B is a q-q plot showing significant positive Pearson's correlations between gene dependency scores and tumor suppressor gene deletion (i.e. lower copy number correlates with lower/more negative dependency scores=synthetic lethal interactions). For each pair, the dependency gene is listed first, followed by the associated tumor suppressor gene. Log-normalized q-values are shown for CRISPR-SpCas9 (y-axis) and RNAi (x-axis) gene dependency scores. Two-sided p-values were calculated using a t-distribution and adjusted using a Benjamini-Hochberg false-discovery rate (FDR) of 10% (q-value<0.1, above grey area). Highly significant correlations are highlighted (unboxed) with emphasis on the VPS4A-SMAD4 and VPS4B-CDH1 synthetic lethal interactions (boxed).

FIG. 1C is a bar graph showing the number of significant synthetic lethal interactions for each tumor suppressor is shown, which only includes positive dependency-copy number correlations in both RNAi and CRISPR-SpCas9 datasets at the q-value <0.1 FDR threshold.

FIG. 1D is a plot showing Log-normalized q-values for all significant and positive correlating gene dependencies for the 10 most correlated tumor suppressor genes. CRISPR dots indicate gene dependency q-values as scored by CRISPR-SpCas9, while RNAi dots indicate RNAi scores. CRISPR dots: VPS4A; GRHL2; TUBB4B; KRAS; WDR77; PRMT5; CDK6; EGFR; AIFM1; WDR77; CCND1; ARF1; TRA2B (upper). RNAi dots: TTC24; JUNB; PRMT5 (upper); WDR77 (upper); FUBP1; GRK2; RPL36A; RPM1; VCP; TRA2B (lower).

FIG. 1E shows a schematic of the genomic location of SMAD4 and VPS4B on human chromosome 18.

FIG. 1F is a scatter plot showing Pearson's correlation coefficient between SMAD4 (x-axis) and VPS4B (y-axis) log₂-normalized relative copy numbers in 1,657 cancer cell lines (p-value <0.0001; F-test). r: 0.784. n:1.657.

FIG. 1G is an illustration showing ESCRT- and VPS4-mediated reverse topology membrane remodeling.

FIG. 1H is a volcano plot showing Pearson's correlation coefficients between CRISPR-SpCas9 gene dependency scores and SMAD4 copy number (x-axis) and the log₁₀-normalized q-value for each of these correlations (y-axis) across 622 cancer cell lines from the DepMap (19Q3). Each dot represents a different gene dependency. Highly significant dependencies are shown above the grey-shaded area. Horizontal dashed line: 10% false discovery rate threshold (q-value <0.1, Benjamini-Hochberg).

FIG. 1I is a smoothed histogram showing the distribution of CHMP4B dependency scores from the CRISPR-SpCas9 DepMap dataset (19Q3) across cancer cell lines. The amount of dependent cell lines (dependency score <−0.5, center dashed line) over the total amount of probed cell lines is shown in the top left corner. The left dashed line at −1 indicates the CRISPR score for a set of highly essential genes, while the right black dashed line at 0 indicates the CRISPR score of negative control targeting guides.

FIG. 1J presents boxplots showing the distribution of CRISPR-SpCas9 CHMP4B gene dependency scores across cancer cell lines with neutral or reduced VPS4B or VPS4A copy number (Log₂ relative copy number <0.66). Boxes indicate 25^{th} and 75^{th} percentiles with median. Whiskers indicate 10^{th} and 90^{th} percentiles, outliers are shown as circles. **** two-tailed p-value <0.0001; ANOVA Kruskal-Wallis non-parametric test with Dunn's correction.

FIG. 1K is a bar graph showing the frequency of VPS4A-dependent cancer cell lines by tumor lineage. Two lines (upper and lower) for each tumor lineage were classified as dependent when they show a score <−0.5 for CRISPR-SpCas9 (upper bar) or RNAi (lower bar). For each tumor lineage, the number of dependent cell lines over the total amount of lines is shown.

FIG. 1L presents a plot that provides a summary of VPS4B copy number alterations in TCGA Pan-Cancer Atlas patient samples categorized by tumor type. Values show log₂-normalized VPS4B copy number relative to the mean sample ploidy. Each dot represents a patient sample and darker dots denote samples with deep VPS4B loss (score <−0.75). Grey bars indicate mean VPS4B copy number+standard deviation.

FIGS. 1.1A-1.1G presents a histogram that show the discovery of synthetic lethal interactions with genomic loss of established tumor suppressors

FIG. 1.1A provides a smoothened histogram showing the density distribution of Pearson's correlation coefficients of cis correlations from the synthetic lethal interaction analysis. Coefficients are shown for CRISPR-SpCas9 (tall peak) and RNA interference (short peak).

FIG. 1.1B provides a volcano plot showing the Pearson's correlation coefficient (x-axis) and −log 10-normalized false discovery-corrected significance q-value of the correlations (y-axis) for all cis correlations between CRISPR-SpCas9 gene dependencies and tumor suppressor gene copy number. Horizontal dashed line represents 10% false discovery rate threshold (q-value <0.1, Benjamini-Hochberg). Each dot represents an interaction between a gene's dependency score and copy number of a tumor suppressor gene. The 20 most significant interactions for both positive and negative correlations are labeled.

FIG. 1.1C is like FIG. 1.1B, except with RNAi instead of CRISPR-SpCas9 gene dependency scores.

FIG. 1.1D provides a smoothened histogram showing the density distribution of Pearson's correlation coefficients of trans correlations from the synthetic lethal interaction analysis. Coefficients are shown for CRISPR-SpCas9 (tall peak) and RNA interference (short peak).

FIG. 1.1E provides a volcano plot showing the Pearson's correlation coefficient (x-axis) and −log 10-normalized false discovery-corrected significance q-value of the correlations (y-axis) for all trans correlations between CRISPR-SpCas9 gene dependencies and tumor suppressor gene copy number. Horizontal dashed line represents 10% false discovery rate threshold (q-value <0.1, Benjamini-Hochberg). Each dot represents an interaction between a gene's dependency score and copy number of a tumor suppressor gene. The 20 most significant interactions for both positive and negative correlations are labeled.

FIG. 1.1F is like FIG. 1.1E, except with RNAi instead of CRISPR-SpCas9 gene dependency scores.

FIG. 1.1G provides a heatmap showing all overlapping, significant, positive correlations (synthetic lethal interactions) between gene dependency scores (x-axis) and tumor suppressor gene copy number (y-axis). The number of significant interactions is shown next to each tumor suppressor. The heatmap shading indicates in which dataset the interaction was most significant; dark grey (CRISPR-SpCas9), intermediate shades of grey (equal), light grey (RNAi). For example, dark grey boxes (CRISPR-SpCas9): SMAD4/VPS4A; SMAD4/GRHI2; light grey boxes (RNAi): CDKN2B/PRMT5; CDKN2A/PRMT5. In order from left to right, the top of FIG. 1.1G lists the following: VPS4A, GRHL2, TUBB4B, KRAS, KLF5, GRK2, EGFR, ITGA3, SCAP, UPF1, RAPGEF1, RAB6A, TTC24, JUNB, COPS4, SREBF1, METAPI, GMNN, AKAP9, LIMS1, UBE2Z, CSNK1D, COPS7A, PSMD6, WDR77, PRMT5, KIF2C, CCND1, NFE2L2, CDK6, WWTR1, GYPA, MSI2, RBL1, PRSS42, IP09, LMO2, AIFM1, EDF1, CIAPIN1, FUBP1, ADAR, ESRI, PHF2, PCM1, FBXO11, EAF1, ARF1, ERBB3, UBE2H, GAB1, ERBB2, RPL36A, MTA2, MTOR, HSP9AB1, SH2D2A, AHCYL1, EXOC2, TUBB, USP5, RRM1, VCP, DCPS, PSMA1, PCDHB13, TRA2B, ZC3H18, H2AFZ, SRRT, ACTG1, POLR2J, MED1, MYBL2, MED12, RUNX1, MED16, MYT1, TEAD1, OR5M1, RPL22, ZFC3H1, ATR, METTLI, MED1, CHD4, IGF1R, ZFP36, PLXNA1, AMOTL2, DDX39B, SMARCA2, CDC4, CUL4B, PPP2R1A, ITGAV, CSTF3, MDM2, CD33, SAMD4B, MEIS3, WDR92, SDHAF3, NAA15, NONO, FBXW11, PIK3CB, BRAF, CMA1, SNRPB, ARF4, VPS4B, MNT, MAGOH, YME1L1, CDK2, SKP2, RAB1A, SNRPB2, SPDEF, DDX5, ELMSAN1, SF3B4, GRPEL1, ATP6AP1, ARID1B, and ACTB.

FIGS. 2A-2J show the validation of VPS4A as a dependency in cancer cells with copy loss of VPS4B.

FIG. 2A provides a plot that shows seven-day viability assays from 8 VPS4B^{neutral} and 10 VPS4B^{loss} cell lines stably transduced with CRISPR-SpCas9. Each dot represents the mean viability effect (y-axis) of cells infected with the indicated sgRNA of at least three wells of a 96-well plate as measured by CellTiter-Glo® luminescence. Black bars indicate the mean cell viability effect across all three VPS4A sgRNAs. Viability scores are normalized on a scale from 0 (negative controls: average effect of 3 sgRNA cutting controls) to −1 (positive controls: average effect of 3 sgRNAs against pan-essential genes; see methods). Single representative results from at least two independent experiments are shown. **** two-tailed p-value <0.0001; unpaired t-test on the grand mean of all sgRNAs in the VPS4B^{neutral} and VPS4B^{loss} groups. For example, a VPS4A dendency score corresponding to YAPC at 0-1 (sgVPS4A1); 0.25-0.35 (sgVPS4A3); and 0.3-0.4 (sgVPS4A2).

FIG. 2B plots seven-day viability assays from 2 VPS4B^{neutral} and 5 VPS4B^{loss} cell lines stably transduced with the C9-11 mutated shSeed2 control (left) (Marine et al. J Biomol Screen. 17(3):370-378, 2012) and shVPS4A-2 (right) tetracycline-inducible RNAi reagents after treatment with 0.5 μM doxycycline (0.222 μg/mL). Each dot represents a technical replicate and shows relative cell viability (y-axis) compared to untreated cells as measured by CellTiter-Glo® luminescence. Boxplots indicate 25^{th}-75^{th} percentiles with median and whiskers indicate maximal outlier values. Plot represents a representative experiment that was repeated at least once. Ns: not significant, ****p-value <0.0001 (Two-way repeated measures ANOVA with Sidak's multiple comparisons test).

FIG. 2C is a graph that plots ten-day proliferation curve of VPS4B^{loss} SNU213 pancreatic cancer cells stably transduced with the tetracycline-inducible RNAi system for the C9-11 mutated shSeed2 control (diamonds and squares) or shVPS4A-2 (triangles). Cells were either grown in control or 1 μM doxycycline (dox; 0.444 μg/mL) containing cell culture medium, which was refreshed every 2-4 days. The y-axis indicates the fold increase in total viable cells compared to the starting amount as measured by trypan blue exclusion. Icons represent mean±standard deviation of 2 independent experiments.

FIG. 2D is a graph that plots in vivo tumor growth of VPS4B^{loss} SMSCTR rhabdomyosarcoma cells stably transduced with either the shSeed2 control (diamond; (+Dox) beige square) or the shVPS4A-2 (triangle; (+Dox) indigo inverted triangle) tetracycline-inducible RNAi systems after injection into the flank of immune-compromised NOG mice. Once tumors reached ˜300 mm³, mice were randomized to either a vehicle or doxycycline (dox; 625 ppm) diet. Each dot represents a single measurement and each line follows tumor volume (y-axis) of an individual mouse tumor over time (x-axis). **** p-value <0.0001; Bonferroni-corrected log-rank Mantel-Cox analysis.

FIG. 2E provides a Kaplan-Meier survival plot of NOG mice bearing subcutaneous SMSCTR xenografts described in FIG. 2D. Survival (y-axis) is plotted against time (x-axis). Dox: doxycycline. Crosses indicate censored mice. **** p-value <0.0001; Bonferroni-corrected log-rank Mantel-Cox analysis.

FIG. 2F provides a digitized immunoblot for VPS4A and Vinculin from SMSCTR xenograft tumors in NOG mice 7 days post randomization (dox: doxycycline) as visualized by Protein Simple capillary-based luminescence.

FIG. 2G provides a graph showing in vivo tumor growth of VPS4B^{loss} SNU213 pancreatic cancer cells stably transduced with either the shSeed2 control (diamonds, squares) or the shVPS4A-2 (triangles) tetracycline-inducible RNAi systems after injection into the flank of immune-compromised NOG mice. Once tumors reached ˜300 mm³, mice were randomized to either a vehicle or doxycycline (dox; 625 ppm) diet. Each dot represents a single measurement and each line follows tumor volume (y-axis) of an individual mouse tumor over time (x-axis). **** p-value <0.0001; Bonferroni-corrected log-rank Mantel-Cox analysis.

FIG. 2H provides a Kaplan-Meier survival plot of NOG mice bearing subcutaneous SNU213 xenografts described in FIG. 2G. Survival (y-axis) is plotted against time (x-axis). Dox: doxycycline. Crosses indicate censored mice. **** p-value <0.0001; Bonferroni-corrected log-rank Mantel-Cox analysis.

FIG. 2I provides four plots showing Caspase 3/7 apoptosis activity (y-axis) over time (x-axis) in four cancer cell lines as measured by IncuCyte® fluorescence live cell imaging. Cells stably expressing SpCas9 were lentivirally transduced with an sgRNA targeting VPS4A. Caspase 3/7 signal was normalized relative to time matched uninfected cells; see Methods. Dots and errors bars represent means±standard error of a single experiment using the average of 4 images per well from 3 different wells. **** p-value<0.0001; repeated measures two-way ANOVA.

FIG. 2J presents plots showing cell cycle distribution of VPS4B^{loss} cell lines JR, SMSCTR and 59M using DAPI staining and Edu incorporation analyzed by flow cytometry four days after VPS4A ablation by CRISPR-SpCas9. Each dot represents an individual technical replicate of control sgRNA treated (sgChr2: first grouping of black dots, from Left to Right in each phase) or VPS4A targeting sgRNAs (grey dots: sgVPS4A-1 (second); sgVPS4A-2 (third); sgVPS4A-3 (fourth) groupings). Horizontal black bars represent the mean for each sgRNA. Significance determined by two tailed t-test between sgChr2 and the combination of all 3 VPS4A sgRNAs. For all panels *p<0.05, **p<0.01, ***p<0.001 and ****p<0.0001.

FIG. 2.2A is an amino acid sequence alignment of the main protein isoforms of human VPS4A (SEQ ID NO: 26) and VPS4B (SEQ ID NO: 28). Similar amino acids are highlighted in grey, while differences are highlighted in darkest shade of grey. VPS4A/B show 80.5% homology. Alignment was performed with Blosum62 of the Geneious Prime 2019 software package.

FIG. 2.2B provides a plot of the log 2-normalized relative copy number of genetic probes (y-axis) across chromosome 18 (x-axis) highlighting SMAD4 (JR: dot at top of left dotted line; RD: dot at bottom of center dotted line) and VPS4B copy number (JR: dot at bottom of right dotted line; RD: bottom of right dotted line) in the JR (top panel) and RD (bottom panel) rhabdomyosarcoma cancer cell lines. JR harbors loss of VPS4B but not SMAD4 and is sensitive to CRISPR-SpCas9-mediated knockout of VPS4A, while RD shows loss of SMAD4 but not of VPS4B and is not sensitive to knockout of VPS4A. Mb=megabase.

FIG. 2.2C provides a karyomap showing the location of VPS4A and CDH1 on chromosome 16q22.1 (human); Mb=megabase. VPS4A and CDH1 copy numbers correlate strongly across cancer cell lines. Bottom panel provides a plot showing Pearson's correlation coefficient between CDH1 (x-axis) and VPS4A (y-axis) log 2-normalized relative copy numbers in 1,657 cancer cell lines (p-value <0.0001; F-test).

FIG. 2.2D provides scatter plots showing Pearson's correlation coefficients between VPS4A (left panels) or VPS4B (right panels) CRISPR-SpCas9 (first column) or RNAi (second column) dependency scores (x-axis) and VPS4B or VPS4A (top row) or SMAD4 or CDH1 (bottom row) relative copy numbers (y-axis) across cancer cell lines.

FIG. 2.2E provide a barplot showing log 2-normalized VPS4B and A copy number relative to overall ploidy across 805 cancer cell lines that were also screened for VPS4A dependency by either CRISPR-SpCas9 or RNAi. The plot is sorted from low (left) to high (right) copy number. The shading of the bars indicates whether that cell line is sensitive to VPS4A depletion as determined by CRISPR-SpCas9 (e.g., lines at rank 500-625), RNAi (e.g., second line to the right of rank 700) or both (magenta; e.g., first line to the right of rank 450), while insensitive cell lines are greyed. The threshold for partial VPS4B copy loss (log 2-normalized relative copy number of 0.8) used in some of the analysis is shown.

FIG. 2.2F provides a Barplot showing log 2-normalized VPS4B copy number relative to overall ploidy across 805 cancer cell lines that were also screened for VPS4A dependency by either CRISPR-SpCas9 or RNAi. The plot is sorted from low (left) to high (right) copy number. The shading of the bars indicates whether that cell line is sensitive to VPS4A depletion as determined by CRISPR-SpCas9 (e.g., line at rank 300), RNAi (e.g., lines at rank 350-400) or both (magenta; 2 lines to the right of rank 500), while insensitive cell lines are greyed. The threshold for partial VPS4B copy loss (log 2-normalized relative copy number of 0.8) used in some of the analysis is shown.

FIG. 3A provides a scatter plot between VPS4B RNAseq expression (y-axis) and VPS4B relative copy number (x-axis) from 1,171 cancer cell lines in the CCLE; Pearson's correlation: 0.593, p-value: 4.589e⁻¹¹² (F-test).

FIG. 3B provides a histogram of Pearson's correlations derived from correlating each gene's RNAseq gene expression with its respective copy number from CCLE (same gene, density 2.5 right histogram). Positions of VPS4A and VPS4B in the distribution are noted by lines. Different gene with density of 7 left histogram shows null distribution of Pearson's correlation coefficients generated by permuting random pairs of gene expression and copy number profiles.

FIG. 3C provides a correlation scatter plot between VPS4B quantitative mass-spectrometry protein expression (y-axis) and VPS4B relative copy number (x-axis) from 375 cancer cell lines in the CCLE; Pearson's correlation: 0.498, p-value=8.19e⁻²⁵ (F-test). Y-axis represents log 2 protein expression of a cell line normalized to expression of the protein in a set of 10 reference cancer cell lines from various lineages (with zero as the mean reference value). X-axis represents log 2normalized relative copy number (TPM).

FIG. 3D provides a digitized VPS4B immunoblot by Protein Simple capillary-based luminescence from 23 cancer cell lines based on VPS4B copy number (n: 11 VPS4B^{neutral} and 12 VPS4B^{loss}). Total protein stains were used for loading. Relative copy number is indicated below based on CCLE copy number calls.

FIG. 3E provides a scatter plot between VPS4B protein quantitation normalized from total protein in FIG. 3D (y-axis) and VPS4B relative copy number (x-axis). Pearson's correlation: 0.652, p-value=7e−4 (F-test).

FIG. 3F provides a VPS4B immunoblot from the parental RD-SpCas9 cancer cell line (VPS4B^{neutral}) and a mixture of 2 pools of 4 monoclonal RD-SpCas9 VPS4B^{-/-} CRISPR-SpCas9 knockout cell lines.

FIG. 3G shows cell viability measured by CellTiter-Glo® luminescence of VPS4B^{neutral} RD-SpCas9 cells (left panel) and two pools of 4 monoclonal RD-SpCas9 VPS4B^{-/-} cell lines (from FIG. 3F). Each dot represents normalized cell viability from an individual assay-well treated with the indicated sgRNAs (see Methods). Horizontal black bars indicate the mean of each group. Statistics: see end of this section.

FIG. 3H provides a VPS4B immunoblot from the VPS4B^{loss} JR-SpCas9 cancer cell line and a JR-SpCas9 cancer cell line overexpressing VPS4B′ cDNA.

FIG. 3I shows cell viability measured by CellTiter-Glo® luminescence of VPS4B^{loss} JR-SpCas9 cancer cells (left) and JR-SpCas9 cells overexpressing VPS4B (right). Statistics: see end of this section.

FIG. 3J shows cell viability measured by CellTiter-Glo® luminescence of VPS4B^{loss} JR-SpCas9 cancer cells (left) and JR-SpCas9 cells overexpressing VPS4A^{WT} (center) or VPS4A^{L64A} cDNAs (right). For FIGS. 3G, 3I, and 3J, each dot represents the normalized cell viability from an individual well (see Methods). Horizontal black bars indicate the mean of each group. For all panels ns: not significant, *p<0.05, **p<0.01, ***p<0.001 and ****p<0.0001 (Unpaired t-test, comparing the mean viability effect of the negative control sgRNAs to the indicated sgRNA treatment).

FIGS. 3.3A-3D show cancer cell lines that depend on the VPS4 paralogs are also sensitive to genetic manipulation of other ESCRT members

FIG. 3.3A provides volcano plot showing the Pearson's correlation coefficient (x-axis) and −log 10-normalized false discovery-corrected significance q-value of the correlations (y-axis) for correlations between CRISPRSpCas9 gene dependency scores and CDH1 relative copy number across 624 cancer cell lines. Horizontal dashed line represents 10% false discovery rate threshold (q-value <0.1, Benjamini-Hochberg). Each dot represents an interaction between a different gene's dependency score and CDH1 copy number. The most significant interactions for both positive (right of dotted line at 0.0 and above the grey-shaded area) and negative (left of dotted line and above the grey-shaded area) correlations are labeled.

FIG. 3.3B provides volcano plot showing the Pearson's correlation coefficient (x-axis) and −log 10-normalized false discovery-corrected significance q-value of the correlations (y-axis) for correlations between CRISPRSpCas9 gene dependencies and VPS4A CRISPR dependency score. Horizontal dashed line represents 10% false discovery rate threshold (q-value <0.1, Benjamini-Hochberg). Each dot represents an interaction between a different gene's dependency score and VPS4A dependency score. The most significant interactions for both positive (generally to the right of dotted line at 0.0 and above the grey-shaded area; e.g., TIPARP, PIK3C3, NARS, WDR7, ZNF-407) and negative (between −0.15 and −0.2) correlations are labeled in addition to all correlations with ESCRT-related genes (generally near lowest points of the curve).

FIG. 3.3C shows results like FIG. 3.3B; except with VPS4B CRISPR dependency score.

FIG. 3.3D provides violin plots showing the distribution of CRISPR gene dependency scores (y-axis) for the indicated ESCRT-related gene (x-axis) categorized by VPS4A and VPS4B relative copy number status (see legend). The ESCRT-related genes are grouped by annotated function. Plots indicate the full range of observed values with black horizontal bars indicating median score and dashed lines indicating the 25-75% quartile range. The line at −1 indicates the CRISPR score for a set of highly essential genes, while the top line at 0 indicates the CRISPR score of negative control targeting guides. *p-value <0.05, **p-value <0.01, **** p-value <0.0001 (unpaired Welch's t-tests (ANOVA) with Bonferroni correction (C: 90)).

FIGS. 4A-4H show that VPS4A suppression leads to ESCRT-III filament accumulation, deformed nuclei and abscission defects in VPS4B^{loss} cancer cells.

FIG. 4A provides a schematic showing known cellular functions of the ESCRT machinery in membrane biology.

FIG. 4B provides a digitized immunoblot showing VPS4A and Vinculin protein levels in a VPS4B^{neutral} cancer cell line (KP4), and three VPS4B^{loss} cancer cell lines (PANC0403, SNU213, 59M) stably transduced with the doxycycline-inducible shVPS4A-2 RNAi system. Cells were treated for 5 days with control media or media containing 1 μM doxycycline (dox), refreshed once. Loading control: Vinculin.

FIG. 4C provides confocal immunofluorescence imaging of CHMP4B in 4 different cancer cell lines stably transduced with the doxycycline-inducible shVPS4A-2 RNAi system. Cells were imaged after 6-day incubation in control media (upper row) or media supplemented with 1 μM doxycycline (bottom row). CHMP4B was detected by immunofluorescence and images show grayscale values (white scale bars: 50 μm). Representative images from a single experiment are shown.

FIG. 4D provides a quantification of CHMP4B speckle formation in untreated (left grouping for each cell type) and doxycycline treated (right grouping for each cell type) cells from FIG. 4C on multiple confocal images (n: 3-9) from a single experiment using CellProfiler v3.1.9. KP4 untreated (n: 100 cells), KP4+dox (n: 67 cells), PANC0403 untreated (n: 311 cells), PANC0403+dox (n: 395 cells), SNU213 untreated (n: 113 cells), SNU213+dox (n: 85 cells), 59M untreated (n: 81 cells), 59M+dox (n: 48 cells). ns: not significant, **** q-value <0.0001, ** q-value <0.01 (two-tailed Brown-Forsythe ANOVA with corrected Benjamini-Yekutieli false discovery rate).

FIG. 4E presents images and a violin plot showing results from confocal fluorescence imaging of DNA using DAPI (bottom image and right of plot) of parental RD-SpCas9 cancer cells euploid for VPS4B copy and clone B2 RD-SpCas9 cancer cells with knockout of VPS4B (FIGS. 3F-3G, 6.6C). White scale bars: 50 μm. Nuclear size was quantified using CellProfiler (****p-value <0.0001, two-tailed unpaired t-test with Welch's correction).

FIG. 4F provides confocal immunofluorescence images of the inner nuclear membrane protein Emerin (Alexa Fluor 561, fluorescing perimeters) and DNA (DAPI, shaded centers) in 4 different cancer cell lines. Cells were grown for 6 days in control media (upper) or treated with 1 μM doxycycline (bottom). Arrows: micronuclei positive for both Emerin and DNA. Representative images from a single experiment are shown. White scale bars: 50 μm.

FIG. 4G presents immunofluorescence images of cytokinetic bridges and midbodies using tubulin (Alexa Fluor 488, outer region of fluorescent signal) and DNA (DAPI, centers) in 3 different cancer cell lines. Cells were grown for 4 days after induction of CRISPR-SpCas9-mediated genetic disruption of an intergenic region (sgChr2-2) or VPS4A (sgVPS4A-1). Arrows indicate cytokinetic bridges. Representative images from a single experiment are shown.

FIG. 4H presents bar graphs showing quantification of cancer cells connected to neighboring cells by cytokinetic bridges. Multiple images (n: 9-17) from FIG. 4F were quantified manually using ImageJ. Ns: not significant, **** p-value <0.0001, ** p-value <0.01 (two-tailed Fisher's Exact test with Bonferroni correction (C:2 for SMSCTR/JR)). FIG. 5A is a scatter plot between VPS4B RNAseq expression (y-axis) and VPS4B relative copy number (x-axis) from 1,171 cancer cell lines in the CCLE; Pearson's correlation: 0.593, p-value: 4.589e−112 (F-test).

FIGS. 4.4A-4.4H show that VPS4A and VPS4B undergo frequent copy loss across both adult and pediatric cancer types.

FIG. 4.4A provides a histogram showing the density distribution of VPS4A and VPS4B dependency scores in CRISPRSpCas9 (lighter shading) and RNAi (darker shading) DepMap 19Q3 datasets. The number of dependent cell lines, determined as having a dependency score <−0.5 (middle dashed line), are indicated in the upper left inset of each plot. The left-most dashed line at −1 indicates the CRISPR score for a set of highly essential genes, while the dashed line at 0 indicates the CRISPR score of negative control targeting guides. Upper panel (CRISPR—left peak; RNAi—right peak). Lower panel (CRISPR—right peak; RNAi—left peak).

FIG. 4.4B provides a bar graph showing the frequency of VPS4B-dependent cell lines by tumor lineage. Lines were classified as dependent when they show a score <−0.5 for CRISPR-SpCas9 (upper bar for each cell type) or RNAi (lower for each cell type). For each tumor type, the number of dependent cell lines over the total amount of cell lines is shown.

FIG. 4.4C provides a plot showing a summary of VPS4A copy number alterations across TCGA Pan-Cancer Atlas samples categorized by tumor type. Values show log 2-normalized VPS4A copy number relative to the mean sample ploidy. Each dot represents a patient sample and darker dots denote samples with strong VPS4A loss (score <−0.75). Grey bars indicate mean VPS4A copy number+standard deviation.

FIG. 4.4D provides plots showing Pearson's correlation coefficients between gene copy numbers for 10,712 samples from the TCGA Pan-Cancer Atlas. Left panel: scatter plot showing VPS4A relative copy number (yaxis) and CDH1 relative copy number (x-axis). Right panel: scatter plot showing VPS4B relative copy number (y-axis) and SMAD4 relative copy number (x-axis).

FIG. 4.4E is a schematic combined with a plot that provides positive (PPV; darker line) and negative predictive values (NPV; lighter line) (y-axis) of indicated gene markers on chromosome 18 (x-axis) for prediction of VPS4B copy number in TCGA Pan-Cancer Atlas samples. BCL2 copy number, located ˜70 kb upstream of VPS4B, had the highest predictive value.

FIG. 4.4F is a bar graph presenting observed instances of VPS4B copy loss in pediatric cancer samples from the Dana-Farber Cancer Institute PROFILE database. VPS4B copy number was inferred from observed BCL2 copy loss.

FIG. 4.4G presents a matrix that provides rates of copy loss of indicated genes from rhabdomyosarcoma (RMS) patient samples in the DFCI PROFILE database. Each column represents an individual patient sample. BCL2 and CDH1 copy numbers are shown to infer VPS4B and VPS4A copy numbers respectively due to their close chromosomal proximities.

FIG. 4.4H presents a heat map that provides copy number heatmap for chromosome 18 from RMS patient samples (Chen et al., Cancer Cell 24(6):710-724, 2013). Each vertical column represents an individual patient sample. The positions of SMAD4 and VPS4B are indicated by lines, histologic subtypes are listed above samples as shaded bars.

FIGS. 5A-5F show that CRISPR-SpCas9 screening reveals that ESCRT proteins and the ULK3 kinase modify sensitivity to VPS4A suppression

FIG. 5A is a schematic providing a workflow of the CRISPR-SpCas9 loss-of-function screen to identify modifiers of VPS4A dependency. SNU213 pancreatic cancer cells stably transduced with the CRISPR-SpCas9 endonuclease and the shVPS4A-2 inducible RNAi system were infected with the Brunello genome-scale lentiviral sgRNA library. The experiment was performed once in duplicate.

FIG. 5B provides a volcano plot highlighting genes for which knockout altered cell viability of VPS4A suppressed SNU213-SpCas9 cells. Each dot represents a gene. Difference in log₂-normalized mean sgRNA abundance between untreated and doxycycline treated cells (x-axis) and the STARS significance q-value of this difference (y-axis) are shown. Significant genes that sensitized cells to VPS4A suppression are shown above the shaded area to the left of the central dotted line (sensitization; top and middle left panels), while genes promoting cell proliferation and viability during VPS4A suppression are shown above the shaded area to the right of the central dotted line (resistance; top and middle right panels). STARS 5% false discovery rate threshold is shown (q-value<0.05).

FIG. 5C provides a violin plot showing log₂-normalized mean fold changes for individual sgRNA abundance after the screen over sgRNA abundance in the pDNA for genes scoring as top hits in the differential analysis (FIG. 5B) of untreated (left grouping for each gene) and doxycycline (right grouping for each gene) treated samples. Each dot represents a sgRNA targeting the indicated gene (mean of both replicates). Violin plots indicate median (horizontal black bar) with 25-75% percentiles (stripped black bars).

FIG. 5D provides a volcano plot, like FIG. 5B, showing only genes related to the ESCRT-machinery. Genes are shaded to indicate their functional annotation within the ESCRT-machinery shown in the inset. For example, ESCRT-0/Bro1: STAM2; ESCRT-1: VPS37B, VPS28; ESCRT-II: SNF8, VPS36; ESCRT-III: CHMP1A, CHMP1B, CHMP4B, CHMP5, CHMP7, IST1; VPS4/VTA1: MPS4B, VPS4A; Auxiliary: UK3, SPAST, ZFYVE19; MITD1.

FIG. 5E presents a stylized and manually annotated protein network of the top 50 scoring genes from the screen (FIG. 5B). Grey connections indicate the strength of interaction between proteins as defined by functional associations predicted using STRING (https://string-db.org). Clusters were obtained with STRING and then grouped into functional groups by manual inspection. Lighter globes (e.g., ULK3, VTA1, CHMP1A, STAM, RUNX1, TIAL1) indicate sensitizing genes while darker globes indicate resistance genes in the context of VPS4A suppression.

FIG. 5F presents a bar graph that shows a gene set enrichment plot showing statistical significance (x-axis) of Metascape (https://metascape.org) summary genesets mapping to the top 50 scoring genes from the screen using GO Biological Processes, Reactome, KEGG and CORUM-based genesets. Numbers behind geneset names indicate the number of top 50 genes in that set divided by the total amount of genes in the geneset.

FIGS. 5.5A-5.5F show validation of VPS4A as a dependency in cancer cells with copy loss of VPS4B.

FIG. 5.5A provides a VPS4A and GAPDH immunoblot in five different CRISPR-SpCas9 stable cell lines after lentiviral transduction with 3 different VPS4A targeting sgRNAs or a negative “cutting control” sgRNA targeting a gene desert on chromosome 2. For all cell lines, protein lysates were collected 4 days after sgRNA infection. GAPDH served as an internal control. The experiment was performed once.

FIG. 5.5B shows a VPS4A and GAPDH immunoblot testing the effect of 3 different tetracycline-inducible VPS4A targeting shRNAs (shVPS4A-1 to shVPS4A-3) and corresponding sequence matched C9-11 seed controls (shSeed-1-shSeed-3). Based on the ratio of VPS4A suppression between the on-target and matched C9-11 shRNAs, shVPS4A-2 and shVPS4A-3 were selected for use in subsequent experiments. GAPDH served as loading control. The experiment was performed once.

FIG. 5.5C presents well images and a bar graph that show long-term colony formation assays after sustained RNAi-mediated VPS4A suppression. Colonies were stained with crystal violet after the indicated time points. Left panel: Six cell lines stably transduced with the indicated shRNA systems (n=2 VPS4Bneutral; n=4 VPS4Bloss) were plated in triplicate in 24-well plates and treated for 8-29 days with control or doxycycline containing culture medium. Right panel: spectrophotometric quantification using absorbance of crystal violet staining after acetic acid dye extraction. Bars indicate means±standard deviation of 4 technical replicates of 3 different extracted wells. Repeated optimization experiments were initially carried out with different cell densities and harvested at different time points, in which the general trend was always similar.

FIG. 5.5D presents plots that show apoptosis induction by Annexin V flow cytometry. VPS4Bloss cell lines JR and SNU213 stably expressing CRISPR-SpCas9 were lentivirally transduced with the indicated sgRNAs and assayed 5 days post infection by flow cytometry using Annexin V coupled to FITC. Inactivation of SF3B1 was used as a positive control for apoptosis induction. Two tailed t-test; p<0.05, **p<0.01, ***p<0.001.

FIG. 5.5E provides a plot showing Caspase 3/7 apoptosis activity (y-axis) over time (x-axis) in the CRISPR-SpCas9+, VPS4A^{neutral} ES2 cancer cell line as measured by IncuCyte® fluorescence live cell imaging. Cells were infected with an sgRNA targeting VPS4A. Caspase 3/7 signal was normalized relative to time matched uninfected cells. Dots and errors bars represent means±standard error of a single experiment using the average of 4 images per well from 3 different wells. Ns: not significant; repeated measures two-way ANOVA.

FIG. 5.5F provides a plot that shows in vitro viability quality control of SNU213, where the number of live cells (y-axis) are compared over time in hours (x-axis) comparing Seed2 controls in the presence or absence of doxocyclin (+dox) to VPS4A-2 in the present or absence of doxocyclin (+dox).

FIGS. 6A-6D show Interferon signaling and CHMP4B expression modulate VPS4A dependency.

FIG. 6A provides a volcano plot showing Pearson's correlation coefficients between gene mRNA expression and CRISPR-SpCas9 VPS4A dependency scores (x-axis) and the log-normalized statistical significance (q-value) of these interactions (y-axis) across 619 CCLE cancer cell lines. Top negatively correlated genes occur to the left of −0.1, while genes localizing to Chr. 18q are generally occur right of −0.1. Grey area: values that fall below the 5% false-discovery rate cut-off (−Log(q-value)<1.31, Benjamini-Hochberg).

FIG. 6B provides a gene set enrichment plot showing statistical significance (x-axis) of Metascape (https://metascape.org) summary genesets mapping to the top 250 genes whose mRNA expression significantly anticorrelated with VPS4A CRISPR dependency score (FIG. 6A) using GO Biological Processes, Reactome and KEGG genesets. Numbers behind geneset names indicate the number of anticorrelated genes that are part of that set divided by the total amount of genes in the geneset. Genesets that are associated with interferon signaling are highlighted in bold (i.e., Response to virus; Interleukin-10 secretion; Regulation of response to biotic stimulus).

FIG. 6C provides dose-response curves showing 6-day cell viability measured by CellTiter-Glo luminescence of the KP4, PANC0403 and SNU213 pancreatic cancer cell lines stably expressing the doxycycline inducible shVPS4A-2 RNAi system. Cells were untreated (no dox) or treated with 1 μM doxycycline (dox) for 3 days. Medium and doxycycline were then refreshed and a titration of purified interferon-β or interferon-γ (ng/mL) was added on top. Cells were incubated for another 3 days before viability was measured. Each dot represents the mean of 2 experiments performed in triplicate, with error bars indicating standard deviation. Dose-response curves were fitted using a 4-parameter log-based model.

FIG. 6D provides a scatter plot showing the Pearson's correlation coefficient between prediction values from a 10-fold cross-validated multiple linear regression model (y-axis) and observed VPS4A CRISPR dependency scores (x-axis). The linear model utilizes normalized VPS4B, CHMP4B, ISG15 and ITCH mRNA expression values across 621 cancer cell lines to predict VPS4A dependency. Each of the 4 terms added significant value to the model.

FIG. 6.6A-6.6H show altered VPS4B expression modulates VPS4A dependency in cancer cells

FIG. 6.6A provides a scatter plot between VPS4B RNAseq expression (y-axis) and VPS4B relative copy number (x-axis). Correlation from 10,712 patient samples in the TCGA Pan-Cancer Atlas.

FIG. 6.6B provides a scatter plot between VPS4A RNAseq expression (y-axis) and VPS4A relative copy number (x-axis). Correlation from 1,171 cell lines in the CCLE; R: =0.51 p=1.892e−78.

FIG. 6.6C provides a VPS4B immunoblot in 16 different monoclonal cell lines derived from VPS4B^{neutral} RD+SpCas9 cell line after infection with a VPS4B targeting sgRNA. Corresponding VPS4B insertion/deletion rates from Sanger sequencing analyzed by TIDEseq are listed below each gel lane.

FIG. 6.6D provides a bar graph that shows the nsertion/deletion rate in VPS4B determined by TIDEseq in polyclonal RD-SpCas9 cells infected with the sgRNA against VPS4B. Number indicates the percentage of intact VPS4B sequencing reads.

FIG. 6.6E provides a barplot showing relative cell viability (y-axis) quantified by CellTiter-Glo® viability assay using the polyclonal RD-SpCas9 cell line that was lentivirally transduced with the VPS4B targeting sgRNA (see FIG. 6.6D; parental population from which monoclonal cultures in FIG. 6.6C were derived). Each dot represents the viability effect of an individual well transduced with the indicated sgRNA (x-axis). Relative viability was calculated by normalizing CellTiter-Glo luminescent values to the average effect of negative control sgRNAs (sgLacZ and sgChr2). Bars indicate the mean viability effect of each of the indicated sgRNAs. Error bars represent standard deviation. Significance was determined by two-tailed t-test on the mean of the negative control guides and sgVPS4A-1. The percent reduction in cell viability closely matched the estimated VPS4B indel rate from FIG. 6.6D.

FIG. 6.6F provides a VPS4A immunoblot in VPS4Bloss 59M-SpCas9 cells expressing no VPS4A open reading frame (ORF) (−) or the VPS4A^{WT} ORF after infection with negative control sgRNA, sgRNA targeting Chr. 2, or sgVPS4A-2 targeting an intron-exon junction that can inactivate the endogenous VPS4A allele, but not the ORF alleles. Immunoblots with short or long exposure are provided to illustrate VPS4A expression levels between cells only expressing endogenous VPS4A and cells expressing the ORF.

FIG. 6.6G provides a cellular proliferation curve showing the number of viable cells (y-axis) over time (x-axis) of 59MSpCas9 cells after infection and antibiotic selection of the indicated VPS4A wild-type and mutant cDNA open reading frames. Quantification of single cell counts of viable cells was performed using trypan blue exclusion during post infection cellular outgrowth.

FIG. 6.6H provides dot plots showing relative cell viability (y-axis) as measured by the CellTiter-Glo viability assay in VPS4B^{loss} 59M-SpCas9 cells (left) and VPS4B^{loss} 59M-SpCas9 cells overexpressing VPS4A^{WT} (center) or VPS4A^{L64A} ORFs (right). Each dot represents the normalized cell viability from an individual well of an assay plate treated with the indicated sgRNA (see Methods). Horizontal black bars indicate the mean of each group. For all panels *p<0.05, **p<0.01, ***p<0.001 and ****p<0.0001 (Unpaired t-test, comparing the mean viability effect of the negative control sgRNAs to the indicated sgRNA treatment).

FIGS. 7A-7D show VPS4A suppression impacts membrane biology in VPS4B^{loss} cells.

FIG. 7A provides confocal immunofluorescence images of CHMP4B, Emerin and DNA (DAPI) in 3 different rhabdomyosarcoma cell lines stably infected with the doxycycline-inducible shVPS4A-2 RNAi system. Cells were imaged after 5 days in control media (upper row) or media supplemented with 1 μM doxycycline for VPS4A suppression (bottom row). CHMP4B and Emerin were detected by immunofluorescence, while DNA was detected by staining with DAPI (white scale bars: 50 m).

FIG. 7B presents a violin plot that provides a quantification of CHMP4B speckle formation in untreated (left for each cell type) and doxycycline (dox) treated (right for each cell type) cells from FIG. 7A. Multiple confocal images (n: 5-14 images) from a single experiment were analyzed with CellProfiler v3.1.9. RD untreated (n: 46 cells), RD+dox (n: 50 cells), JR untreated (n: 243 cells), JR+dox (n: 193 cells), SMSCTR untreated (n: 172 cells), SMSCTR+dox (n: 174 cells). Ns: not significant, *q-value <0.05, **q-value <0.01 (two-tailed Brown-Forsythe ANOVA with corrected Benjamini-Yekutieli false discovery rate).

FIG. 7C provides confocal immunofluorescence images of RAB7, LC3B and SEC61B after VPS4A suppression in SNU213 pancreatic cancer cells stably transduced with the doxycycline-inducible shVPS4A-2 RNAi system. Cells were imaged after 6-day incubation in control media (upper row) or media supplemented with 1 μM doxycycline (bottom row). This experiment was performed once. Images show grayscale values (white scale bars: 50 m).

FIG. 7D presents a violin plot that provides quantification of RAB7, LC3B and SEC61B speckle formation in untreated (left in each box) and doxycycline (dox) treated (right in each box) SNU213 cells described in FIG. 7C. Multiple confocal images (n: 2-4 images) from a single experiment were analyzed with CellProfiler v3.1.9. RAB7: 50 (untreated) vs. 52 (treated) cells, LC3B: 73 (untreated) vs. 49 cells, SEC61B: 85 (untreated) vs. 30 (treated) cells. Upper panel: the amount of absolute bright speckles per cell, background corrected and thresholded, is shown for each of the 3 stains. Lower panel: the speckle size of thresholded speckles identified in cells after background correction for each of the 3 stains. Ns: not significant, * q-value <0.05, ** q-value <0.01 (two-tailed Kruskal-Wallis ANOVA corrected by two-stage Benjamini, Krieger and Yekutieli false discovery rate).

FIGS. 8A-8D illustrate screening for modifiers of VPS4A dependency reveals an important role for ESCRT proteins and the ULK3 kinase.

FIG. 8A provides a plot showing sgRNA guide-level log 2-normalized fold changes over pDNA from the two untreated replicates of the CRISPR-SpCas9 genome-scale modifier screen. Each dot represents a different sgRNA and abundance was determined using reads per million (RPM+1) normalization of Illuminabased next-generation sequencing read counts. Linear regression shows Pearson's correlation.

FIG. 8B as in FIG. 8A, but then for the two doxycycline treated replicates of the modifier screen.

FIG. 8C provides a plot showing gene-level log 2-normalized fold changes over pDNA after collapsing sgRNA guidelevel fold-changes to gene-level averages of the two untreated replicates of the CRISPR-SpCas9 genome-scale modifier screen. Each dot represents a gene and sgRNA abundance was determined using reads per million (RPM+1) normalization of Illumina-based next-generation sequencing read counts. Linear regression shows the Pearson's correlation coefficient.

FIG. 8D as in FIG. 8C, but then for the two doxycycline treated replicates of the modifier screen.

FIGS. 9A-9I show interferon signaling and CHMP4B expression modulate VPS4A dependency.

FIG. 9A provides a volcano plot showing Pearson's correlation coefficients between normalized relative quantitative protein abundance and CRISPR-SpCas9 VPS4A dependency score (x-axis) and the log-normalized statistical significance (q-value) of these interactions (y-axis) across CCLE cancer cell lines. Top negatively correlated proteins generally are shown on the top left, while top positively correlated proteins are shaded in dark grey. The plot incorporates only correlations between proteins that were detected across at least 150 cancer cell lines. Protein abundance was determined using quantitative mass-spectrometry followed by normalization to reference protein abundance values from quantitative mass-spectrometry of 10 reference cancer cell lines. Grey area: values that fall below the 5% false-discovery rate cut-off (−Log(q-value)<1.31, Benjamini-Hochberg).

FIG. 9B provides a bar graph that shows a gene set enrichment plot showing statistical significance (x-axis) of Metascape (https://metascape.org) summary genesets mapping to the proteins that significantly anticorrelated with VPS4A CRISPR dependency score (FIG. 9A) using GO Biological Processes, Reactome and KEGG genesets. Numbers behind geneset names indicate the number of anticorrelated genes that are part of that set divided by the total amount of genes in the geneset.

FIG. 9C provides a volcano plot showing Pearson's correlation coefficients between gene mRNA expression and CRISPR-SpCas9 VPS4A dependency scores (x-axis) and the log-normalized statistical significance (qvalue) of these interactions (y-axis) across 210 cancer cell lines that show VPS4B copy loss (cell lines with only 1 or 2 copies for VPS4B and a relative VPS4B copy number below <0.8). Top negatively correlated genes occur above the grey-shaded area and to the left of the vertical dotted line, while top correlated genes occur above the grey-shaded area and to the right of the vertical dotted line. Grey area: values that fall below the 5% false-discovery rate cut-off (−Log(q-value)<1.31, Benjamini-Hochberg).

FIG. 9D provides a gene set enrichment plot showing statistical significance (x-axis) of Metascape (https://metascape.org) summary genesets mapping to the genes whose mRNA expression significantly anticorrelated with VPS4A CRISPR dependency score across VPS4B-deficient cancer cell lines (FIG. 9C) using GO Biological Processes, Reactome and KEGG genesets. Numbers behind geneset names indicate the number of anticorrelated genes that are part of that set divided by the total amount of genes in the geneset.

FIG. 9E provides a boxplot showing the relative cell viability (y-axis) across 3 pancreatic cancer cell lines expressing the shVPS4A-2 inducible RNAi system (x-axis). Cells were treated for 6 days with or without doxycycline and cell viability was measured using an ATP-based luminescence readout with CellTiter-Glo and normalized to mean viability of untreated samples. Each dot indicates a separate measurement from two experiments performed in sextuplicate. Boxes indicate 10-90% percentiles with mean.

FIG. 9F provides a bar graph showing normalized mRNA expression values (y-axis) for 5 interferon receptors (x-axis) categorized across 3 pancreatic cancer cell lines (legend).

FIG. 9G provides scatter plots showing the Pearson's correlation coefficients between the prediction scores from 10-fold cross-validated linear regression models (y-axis) and observed VPS4A CRISPR dependency scores (x-axis). Linear models utilize either normalized CHMP4B mRNA expression (top left), VPS4B mRNA expression (bottom left), ISG15 mRNA expression (top right) or ITCH mRNA expression (bottom right) values across 621 CCLE cancer cell lines.

FIG. 9H provides scatter plots showing the Pearson's correlation coefficients between the prediction scores from 10-fold cross-validated linear regression models (y-axis) and observed VPS4B CRISPR dependency scores (x-axis). Linear models utilize either normalized CHMP4B mRNA expression (top left), VPS4A mRNA expression (bottom left), ISG15 mRNA expression (top right) or ITCH mRNA expression values (bottom right) across 621 CCLE cancer cell lines.

FIG. 9I provides a scatter plot showing the Pearson's correlation coefficient between prediction values from a 10-fold cross-validated multiple linear regression model (y-axis) and observed VPS4B CRISPR dependency scores (x-axis). The linear model utilizes normalized VPS4B, CHMP4B, ISG15 and ITCH mRNA expression values across 621 CCLE cancer cell lines for prediction. In this model, addition of ISG15 and ITCH expression to the VPS4A+CHMP4B linear model did not contribute significantly.

FIG. 10A provides a schematic comprising the CRISPR/RNAi gene dependency scores based on 625 cell lines (CRISPR) and 711 cells lines (RNAi), showing the absence of SMAD4 (SMAD4−) in +20% of the cell lines and the presence of SMAD4 (SMAD4+) in +80% of the cell lines.

FIG. 10B provides a scatter plot that illustrates Log-normalized q-values shown for CRISPR-SpCas9 (x-axis) and RNAi (y-axis) gene dependency scores. Two-sided p-values were calculated using a t-distribution and adjusted using a Benjamini-Hochberg false-discovery rate (FDR) of 10% (q-value<0.1, above light grey area). Essential genes, such as, HAUS1, SKA1, CHMP4B (intermediate shade of grey) and selective genes, such as VPS4A and KRAS (darker grey) are shown.

FIG. 10C presents a violin plot that shows cancer cell lines in the presence or absence of SMAD4 (x-axis) in relation to the VPS4A (y-axis) CRISPR Score.

FIG. 11 provides immunofluorescence images of cytokinetic bridges and midbodies using tubulin (Alexa Fluor 488), DNA (DAPI), Emerin, and CHMP4B in SNU213 pancreatic cancer and JR rhabdomyosarcoma cell lines. VPS4A suppression (bottom pannels) demonstrated nuclear deformation, ESCRT-III accumulation, abscission defects, G2/M arrest, and apoptosis. Arrows indicate either nuclear aberrations in the form micronuclei (SNU213) or a unresolved cytokinetic bridge (JR). Representative images from a single experiment are shown. This experiment was repeated once.

FIG. 12A provides a plot of the log 2-normalized relative copy number of genetic probes (y-axis) across chromosome 18 (x-axis) highlighting SMAD4 and VPS4B copy number (dots) in the COV413A (top panel) ovarian cancer and JR (bottom panel) rhabdomyosarcoma cancer cell lines.

FIG. 12B provides plots and a VPS4B immunoblot in rhabdomyosarcoma cell lines derived from VPS4B−/− RD and VPS4B+JR cell lines. Corresponding VPS4B relative viabilities after VPS4A knockout are shown in the plots below each gel lane.

FIG. 12C presents a violin plot that provides a quantification of VPS4B protein level as normalized to total protein (y-axis) in cancer cell lines harboring either VPS4B copy loss (n: 12 cancer cell lines) or neutral VPS4B copy number (n: 11 cancer cell lines. *** p-value <0.0005, two-tailed unpaired T-test

FIG. 12D presents a plot that provides a summary of VPS4B copy number alterations in TCGA Pan-Cancer Atlas samples categorized by tumor type. Cell abbreviations of the patient samples are identified in The Cancer Genome Atlas (https://gdc.cancer.gov/resources-tcga-users/tcga-code-tables/tcga-study-abbreviations). Copy loss of VPS4B was common across the TCGA samples. Values show log₂-normalized VPS4B copy number relative to the mean sample ploidy. Each dot represents a sample with VPS4B copy number loss. Grey bars indicate mean VPS4B copy number+standard deviation.

FIG. 13A shows a digitalized immunoprecipitation (IP) and a digitalized immunoblot (blot) for VPS4B in various cancer cell lines, where in the presence of VPS4A, the IP shows VPS4B in samples of both neutral or loss of VPS4B copy number; whereas for the blot, only for the VPS4B neutral samples is VPS4B present.

FIG. 13B shows a digitalized glycerol gradient fractionation and immunoblot for VPS4B, suggesting that VPS4A and VPS4B are predominantly in low molecular weight complexes.

FIG. 13C presents images that illustrate that VPS4A and VPS4B have a higher affinity for homomeric complexes versus heteromeric complexes.

FIG. 14A presents a VPS4B-Hcp1 progress curve comprising the time in minutes (x-axis) to the relative fluorescence units (RFU) of VPS4B at varying concentrations.

FIG. 14B presents a plot that shows the ATPas activity of different VPS4A constructs.

### DETAILED DESCRIPTION OF THE INVENTION

The invention features compositions and methods that are useful for the treatment of cancer.

The invention is based, at least in part, on the discovery that the ESCRT ATPases VPS4A and VPS4B scored as strong synthetic lethal dependencies. In the Examples provided herein below, VPS4A was found to be selectively essential in cancers harboring loss of VPS4B, which can be lost in combination with SMAD4 in some cancers on account of the proximity of VPS4B to SMAD4. It was also found that VPS4B is also selectively essential in cancers harboring loss of VPS4A, which may be lost in combination with the proximal CDH1 encoding E-cadherin. Cells with higher expression levels of CHMP4B were found to more strongly require VPS4 to be viable. Also, VPS4A/B were found to be co-essential with CHMP1A, VTA1, and IST1.

The invention is based, at least in part, also upon the discovery that expression levels of the markers VPS4B, CHMP4B, ITCH, and/or ISG15 can be used as inputs to a multivariate model to successfully predict VPS4A dependency. Methods for creating a multivariate model are known in the art (see, e.g., H. Joe, Multivariate Models and Dependence Concepts, Chapman & Hall, 1997; F. Harrell, Regression Modeling Strategies, Springer, 2001; and Stephan, et al., “PSA and new biomarkers within multivariate models to improve early detection of prostate cancer”, Cancer Letters, 249:18-29 (2007)).

As reported in detail below, CRISPR-SpCas9 and RNA-interference loss-of-function screens were conducted to identify new cancer therapeutic targets associated with genomic loss of common tumor suppressor genes. The ESCRT ATPases VPS4A and VPS4B scored as strong synthetic lethal dependencies. VPS4A suppression in VPS4B-deficient cells selectively led to ESCRT-III filament accumulation, cytokinesis defects, nuclear deformation, G2/M arrest, apoptosis and potent tumor regression. CRISPR-SpCas9 screening and integrative genomic analysis revealed other ESCRT members, regulators of abscission and interferon signaling as modifiers of VPS4A dependency. A compendium of synthetic lethal vulnerabilities are described herein and VPS4A and VPS4B were identified as promising therapeutic targets for cancer.

### Synthetic Lethality

Synthetic lethality refers to the observation that for certain gene pairs, inactivation of either gene is tolerated but combined loss-of-function of both genes results in decreased cell viability (Dobzhansky, T. Genetics 31:269-290, 1946; Hartwell et al., Science 278:1064-1068, 1997; Huang et al. Nature Reviews Drug Discovery. 11:1-16, 2019; Kaelin, The Journal of clinical investigation, 104(11):1503-1506, 1999). Synthetic lethal relationships in cancer have been defined in several different contexts. For example, BRCA1/2 mutant cancers harbor defects in homologous recombination DNA repair and are particularly sensitive to inhibition of the poly (ADP-ribose) polymerase (PARP) DNA repair enzyme. This synthetic lethal interaction has been validated in multiple human therapeutic trials, leading to clinical approval of PARP inhibitor therapy in several cancer types (Sonnenblick et al. Nature reviews Clinical oncology, 12(1):27-41, 2015).

Synthetic lethal relationships have also been observed among paralog genes for which dependency on one paralog is conferred by loss of a second functionally redundant paralog gene, as demonstrated between SMARCA2-SMARCA4, ARID1A-ARID1B, UBB-UBC and MAGOH-MAGOHB (Helming et al. Nat. Med., 20(3):251-254, 2014; Hoffman et al. Proc Natl Acad Sci USA, 111(8):3128-3133, 2014; Tsherniak, A. et al. Cell 170, 564-576 e516 (2017); Viswanathan et al. Nat Genet, 50(7):937-943, 2018). Such paralog synthetic lethality may arise when there is a concomitant loss of a driver tumor suppressor gene (TSG) and a paralog passenger gene nearby, a phenomenon that has been termed “collateral lethality”. Examples include ENO2 dependency with loss of ENO1 on chromosome lp36 or ME3 dependency with ME2 deletion at the SMAD4/18q locus (Dey et al. Nature, 542(7639:119-123, 2017; Muller et al. Nature, 488(7411):337-42, 2012). Alternatively, collateral lethality may also occur when dependency in one gene arises as a result of loss of a second functionally related non-paralog gene that is adjacent to a tumor suppressor gene, such as PRMT5 essentiality when MTAP is deleted at the CDKN2A/9p21 locus (Kryukov et al. Journal of Experimental Med, 214(10):2933-2946, 2016; Mavrakis et al. Science, 351(6278):1208-13, 2016). Besides synthetic lethality, selective vulnerabilities on genes that have themselves undergone copy number loss in cancer (Nijhawan et al. Cell, 150(4):842-854, 2012; Paolella et al. Elife, 6:e23268, 2017) have also been described. For example, heterozygous deletion of TP53 often results in bystander loss of the essential genes POLR2A, MED11, and AURKB, which sensitizes cancer cells to knockdown of these genes (Liu et al. Nature, 520(7549):697-701, 2015; McDonald, E. R., 3rd et al. Cell 170, 577-592 e510 (2017)). Since targeting synthetic lethal relationships in cancer may yield a wide therapeutic window of efficacy between tumor and normal cells, identification of pharmacologically tractable synthetic lethal targets remains a priority for oncology drug development programs.

To systematically define synthetic lethal vulnerabilities associated with genomic loss of established TSGs, genome-scale CRISPR-SpCas9 and RNA interference loss-of-function screening data was analyzed from over 600 cancer cell lines. Synthetic lethal interactions (193) were identified and prioritized with one or more of 50 common TSGs (Table 5). In particular, it was found that the paralog genes encoding vacuolar protein sorting 4 homolog A and B (VPS4A and VPS4B) were selective genetic vulnerabilities for tumors harboring genomic copy loss of SMAD4 or CDH1 due to co-deletion of VPS4B or VPS4A respectively with these genes. VPS4B is located on the long arm (q) of chromosome 18, 12.3 Mb away from SMAD4, while VPS4A is located 0.476 Mb downstream of CDH1 (encoding E-cadherin) on chromosome 16q. Co-deletion of SMAD4 and VPS4B is commonly observed in pancreatic, colorectal, stomach and renal cell carcinomas and to a lesser extent in cancers of the bile duct, lung, prostate, esophagus, uterus, cervix and ovary (Kojima, K. et al. Cancer Res 67, 8121-8130 (2007); Thiagalingam, S. et al. Nat Genet 13, 343-346 (1996); Zack, T. I. et al. Nat Genet 45, 1134-1140 (2013)). Meanwhile, loss of CDH1 and VPS4A occurs in cancers of the stomach, breast, skin, colon and prostate (Berx et al. Genomics, 26(2):281-289, 1995; Graff et al. Cancer Res, 55(22):5195-5199, 1995; Yoshiura et al. Proc. Natl. Acad. Sci. U.S.A., 92:7416-7419, 1995; Zack, T. I. et al. Nat Genet 45, 1134-1140 (2013)).

VPS4A and VPS4B function as AAA ATPases, which are critical for the regulation of endosomal sorting complex required for transport (ESCRT), a multimeric protein complex essential for inverse membrane remodeling. The ESCRT machinery is involved in a range of cellular processes, including cytokinesis, membrane repair, autophagy and endosomal processing (Schoneberg, J. et al. Nat Rev Mol Cell Biol 18, 5-17 (2017); Vietri et al. Nature Review Molecular Cell Biology, 21:25-42, 2020). Here, suppression of VPS4A in tumors with reduced copy number of VPS4B leads to accumulation of ESCRT-III filaments, cytokinesis defects, nuclear membrane abnormalities and micronucleation, ultimately resulting in G2/M cell cycle arrest and apoptosis. Furthermore, using a CRISPR-SpCas9 genome-scale modifier screen, multiple genes that promote or suppress VPS4A dependency were identified. There is a critical role for the ESCRT pathway in cancer cell survival and the VPS4 enzymes may be used as synthetic lethal targets specific for tumors harboring loss of VPS4B on chromosome 18q or loss of VPS4A on chromosome 16q.

### RNA Interference

RNA interference (RNAi) is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of shRNAs using a plasmid-based expression system is currently being used to create loss-of-function phenotypes in mammalian cells. As described herein, siRNAs that target VPS4A, VPS4B, CHMP1A, CHMP1B, ULK3, VTA1, or IST1 decrease expression of the target genes in vivo or in vitro.

### Inhibitory Nucleic Acid Molecules

VPS4A, VPS4B, CHMP1A, CHMP1B, ULK3, VTA1, or IST1 inhibitory nucleic acid molecules are essentially nucleobase oligomers that may be employed as single-stranded or double-stranded nucleic acid molecule to decrease VPS4A, VPS4B, CHMP1A, CHMP1B, ULK3, VTA1, or IST1 expression. In one approach, the VPS4A, VPS4B, CHMP1A, CHMP1B, ULK3, VTA1, or IST1 inhibitory nucleic acid molecule is a double-stranded RNA used for RNA interference (RNAi)-mediated knock-down of VPS4A or VPS4B gene expression. In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases of a nucleobase oligomer of the invention. The dsRNA can be two complementary strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference. An inhibitory nucleic acid molecule that “corresponds” to an VPS4A, VPS4B, CHMP1A, CHMP1B, ULK3, VTA1, or IST1 gene comprises at least a fragment of the double-stranded gene, such that each strand of the double-stranded inhibitory nucleic acid molecule is capable of binding to the complementary strand of the target VPS4A, VPS4B, CHMP1A, CHMP1B, ULK3, VTA1, or IST1 gene. The inhibitory nucleic acid molecule need not have perfect correspondence to the reference VPS4A, VPS4B, CHMP1A, CHMP1B, ULK3, VTA1, or IST1 sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

The inhibitory nucleic acid molecules provided by the invention are not limited to siRNAs, but include any nucleic acid molecule sufficient to decrease the expression of a VPS4A, VPS4B, CHMP1A, CHMP1B, ULK3, VTA1, or IST1 nucleic acid molecule or polypeptide. Each of the DNA sequences provided herein may be used, for example, in the discovery and development of therapeutic antisense nucleic acid molecule to decrease the expression of VPS4A, VPS4B, CHMP1A, CHMP1B, ULK3, VTA1, or IST1. The invention further provides catalytic RNA molecules or ribozymes. Such catalytic RNA molecules can be used to inhibit expression of a VPS4A, VPS4B, CHMP1A, CHMP1B, ULK3, VTA1, or IST1 nucleic acid molecule in vivo. The inclusion of ribozyme sequences within an antisense RNA confers RNA-cleaving activity upon the molecule, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference. In various embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

After a subject is diagnosed as having a neoplasia (e.g., brain, bladder, bile, breast, duct, colon, esophageal, gastric, germ cell, liver, or head and neck cancer, lung cancer, osteosarcoma, ovarian cancer, pancreatic cancer, rhabdomyosarcoma (e.g., pediatric rhabdomyosarcoma)), a method of treatment is selected by characterizing the neoplastic cell for the presence or absence of VPS4A, VPS4B, SMAD4, CDH1, CHMP4B, ITCH, and/or ISG15. Cells lacking VPS4A (e.g., VPS4A-deficient copy number) are selected for treatment with an agent that inhibits the expression or activity of VPS4B. Cells lacking VPS4B (e.g., VPS4B-deficient copy number) are selected for treatment with an agent that inhibits the expression or activity of VPS4A. Accordingly, cells deficient in one of a synthetic lethal pair, e.g., VPS4A of the VPS4A/VPS4B synthetic lethal pair, may be treated with an agent that inhibits the expression or activity of the other pair, e.g., VPS4B.

In one embodiment, the inhibitory nucleic acid molecules of the invention are administered systemically in dosages between about 1 and 100 mg/kg (e.g., 1, 5, 10, 20, 25, 50, 75, and 100 mg/kg). In other embodiments, the dosage ranges from between about 25 and 500 mg/m²/day.

### Modified Inhibitory Nucleic Acid Molecules

A desirable inhibitory nucleic acid molecule is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed to enhance the stability or efficacy of an inhibitory nucleic acid molecule.

Inhibitory nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. The nucleobase units are maintained for hybridization with a VPS4A or VPS4B nucleic acid molecule. Methods for making and using these nucleobase oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

### Polynucleotides

In general, the invention includes any nucleic acid sequence encoding an VPS4A, VPS4B, CHMP1A, CHMP1B, ULK3, VTA1, or IST1 polypeptide. Also included in the methods of the invention are any nucleic acid molecule containing at least one strand that hybridizes with such a VPS4A, VPS4B, CHMP1A, CHMP1B, ULK3, VTA1, or IST1 nucleic acid sequence (e.g., an inhibitory nucleic acid molecule, such as a dsRNA, siRNA, shRNA, or antisense molecule). The inhibitory nucleic acid molecules of the invention encoding a VPS4A, VPS4B, CHMP1A, CHMP1B, ULK3, VTA1, or IST1 polypeptide can be 19-21 nucleotides in length. In some embodiments, the inhibitory nucleic acid molecules of the invention comprise 20 or fewer (e.g., 19, 18, 17, 16, 15, 14, 13, 12, 11, 10, 9, 8, or 7) identical nucleotide residues. In yet other embodiments, the single or double stranded antisense molecules are 60%, 65%, 70%, 75%, 80%, 85%, 90%, 95%, 96%, 97%, 98%, 99% complementary to the VPS4A, VPS4B, CHMP1A, CHMP1B, ULK3, VTA1, or IST1 target sequence. An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified but need not be. For example, a nucleic acid molecule that is isolated within a cloning or expression vector may comprise only a tiny percentage of the material in the cell in which it resides. Such a nucleic acid is isolated, however, as the term is used herein, because it can be manipulated using standard techniques known to those of ordinary skill in the art.

Further embodiments can include any of the above inhibitory polynucleotides, directed to a VPS4A, VPS4B, CHMP1A, CHMP1B, ULK3, VTA1, or IST1 gene, or portions thereof.

Delivery of Polynucleotides and/or Oligonucleotides

Naked oligonucleotides or polynucleotides are capable of entering tumor cells and inhibiting the expression of VPS4A or VPS4B. Nonetheless, it may be desirable to utilize a formulation that aids in the delivery of an inhibitory nucleic acid molecule or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Polynucleotide Therapy

Polynucleotide therapy featuring a polynucleotide encoding a VPS4A, VPS4B, CHMP1A, CHMP1B, ULK3, VTA1, or IST1 inhibitory nucleic acid molecule or analog thereof is another therapeutic approach for treating a neoplasia or treating multidrug resistance in a subject. Expression vectors encoding inhibitory nucleic acid molecules can be delivered to cells of a subject having a neoplasia. The nucleic acid molecules must be delivered to the cells of a subject in a form in which they can be taken up and are advantageously expressed so that therapeutically effective levels can be achieved.

Methods for delivery of the polynucleotides to the cell according to the invention include using a delivery system such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a VPS4A or VPS4B inhibitory nucleic acid molecule, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of a VPS4A, VPS4B, CHMP1A, CHMP1B, ULK3, VTA1, or IST1 inhibitory nucleic acid molecule therapeutic to a cell of a patient diagnosed as having a neoplasia. For example, a VPS4A, VPS4B, CHMP1A, CHMP1B, ULK3, VTA1, or IST1 inhibitory nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the VPS4A or VPS4B inhibitory nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell.

VPS4A, VPS4B, CHMP1A, CHMP1B, ULK3, VTA1, or IST1 inhibitory nucleic acid molecule expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers.

For any particular subject, the specific dosage regimes should be adjusted over time according to the individual need and the professional judgment of the person administering or supervising the administration of the compositions.

### Antibodies

In other aspects, the invention provides a method of treating a disease by selectively interfering with the function of a polypeptide (e.g., VPS4A, VPS4B, CHMP1A, CHMP1B, ULK3, VTA1, or IST1). In some embodiments, the interference with the polypeptide function is achieved using an antibody binding to polypeptide.

Antibodies can be made by any of the methods known in the art utilizing a polypeptide of the invention (e.g., VPS4A, VPS4B, CHMP1A, CHMP1B, ULK3, VTA1, or IST1 polypeptide), or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding a polypeptide of the invention or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from the host. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

### Genome Editing

Therapeutic gene editing is a major focus of biomedical research, embracing the interface between basic and clinical science. A degenerating or injured neuron may be treated according to the methods of the present invention by knocking out (e.g., by deletion) or inhibiting expression of a target gene(s) (e.g., VPS4A, VPS4B, CHMP1A, CHMP1B, ULK3, VTA1, or IST1). The development of novel “gene editing” tools provides the ability to manipulate the DNA sequence of a cell (e.g., to delete a target gene) at a specific chromosomal locus, without introducing mutations at other sites of the genome. This technology effectively enables the researcher to manipulate the genome of a subject's cells in vitro or in vivo.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule may be introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. In some embodiments, no donor DNA molecule is introduced and the double-stranded break is repaired by the error-prone non-homologous end joining NHEJ pathway leading to knock-out or deletion of the target gene (e.g., through the introduction of indels or nonsense mutations). In some embodiments, an endonuclease(s) can be targeted to at least two distinct chosen sites located within a gene sequence so that chromosomal double strand breaks at the distinct sites leads to excision and deletion of a nucleotide sequence flanked by the two distinct sites.

In some embodiments, the chosen site is associated with or disposed within a nucleotide sequence encoding a gene selected from one or more of VPS4A, VPS4B, CHMP1A, CHMP1B, ULK3, VTA1, or IST1. In some embodiments, more than one chosen site is selected. In some embodiments the chosen sites are associated with at least 1, 2, 3, 4, 5, 6, or all of the foregoing genes.

Current genome editing tools use the induction of double strand breaks (DSBs) to enhance gene manipulation of cells, including the deletion or knockout of genes. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). In some embodiments a CRISPR/Cas12 system can be used for gene editing. In some embodiments, the Cas12 polypeptide is Cas12b. In some embodiments any Cas polypeptide can be used for gene editing (e.g., CasX). In various embodiments, the Cas polypeptide is selected so that a nucleotide encoding the Cas poypeptide can fit within an adeno-associated virus (AAV) capsid. For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for ˜18 bp sequences in the genome. CRISPR/Cas9, TALENs, and ZFNs have all been used in clinical trials (see, e.g., Li., H, et al., “Applications of genome editing technology in the targeted therapy of human diseases: mechanisms, advances and prospects”, Signal Transduct Target Ther., 5:1 (2020), DOI: 10.1038/s41392-019-0089-y).

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121):823-6). Genetic manipulation using engineered nucleases has been demonstrated in tissue culture cells and rodent models of diseases.

CRISPR has been used in a wide range of organisms including baker's yeast (S. cerevisiae), zebra fish, nematodes (C. elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing Cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, CasI appears to be ubiquitous among different CRISPR/Cas systems. Particular combinations of Cas genes and repeat structures have been used to define 8 CRISPR subtypes (E. coli, Y. pest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcusfuriosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

Cas9 variants have been developed or discovered that can fit into an adeno-associated virus (AAV) capsid with sgRNA. Non-limiting examples of such variants (e.g., Cas9 orthologs) suitable for use in embodiments of the invention of the disclosure include saCas9 (Staphylococcus aureus Cas9), cjCas9 (Camphylobacter jejuni Cas9), NmeCas9 (Neisseria meningitidis Cas9), and spCas9 (Streptococcus pyrogenes Cas 9). An example of a saCas9 suitable for delivery by an AAV vector is provided in Ann Ran, F. et al. “In vivo genome editing using Staphylococcus aureus Cas9”, Nature, 9:186-91, DOI: 10.1038/nature14299.

gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

### CRISPR Interference

In some embodiments, a target gene can be inhibited using CRISPR interference (CRISPRi). CRISPRi is a technique where expression of a target gene is inhibited by the binding of a nuclease-inactive CRISPR system (a CRISPRi system), optionally comprising transcriptional repressors. In some embodiments, the method of CRISPRi involves designing an sgRNA complementary to a promoter or exonic sequence of a target gene. In some embodiments, CRISPRi involves guiding a transcriptional repressor to a transcription start site of a target gene. CRISPRi has been successfully used for the repression of gene expression in mice and an exemplary method for using CRISPRi to repress a gene is provided in MacLeod, et al., “Effective CRISPR interference of an endogenous gene via a single transgene in mice”, Scientific Reports, 9:17312 (2019).

### Pharmaceutical Compositions

As reported herein, loss of VPS4A or VPS4B expression is associated with neoplasia in a variety of cell types. Accordingly, the invention provides therapeutic compositions that decrease the expression of VPS4A, VPS4B, CHMP1A, CHMP1B, ULK3, VTA1, or IST1 to treat or prevent a neoplasm. In one embodiment, the present invention provides a pharmaceutical composition comprising a VPS4A, VPS4B, CHMP1A, CHMP1B, ULK3, VTA1, or IST1 inhibitory nucleic acid molecule (e.g., an antisense, siRNA, or shRNA polynucleotide) that decreases the expression of a VPS4A, VPS4B, CHMP1A, CHMP1B, ULK3, VTA1, or IST1 nucleic acid molecule or polypeptide. If desired, the VPS4A, VPS4B, CHMP1A, CHMP1B, ULK3, VTA1, or IST1 inhibitory nucleic acid molecule is administered in combination with a chemotherapeutic agent. In various embodiments, the VPS4A, VPS4B, CHMP1A, CHMP1B, ULK3, VTA1, or IST1 inhibitory nucleic acid molecule is administered prior to, concurrently with, or following administration of a chemotherapeutic. Without wishing to be bound by theory, administration of a VPS4A, VPS4B, CHMP1A, CHMP1B, ULK3, VTA1, or IST1 inhibitory nucleic acid molecule likely enhances the accumulation or efficacy of a chemotherapeutic agent. Polynucleotides of the invention may be administered as part of a pharmaceutical composition. The compositions should be sterile and contain a therapeutically effective amount of the polypeptides or nucleic acid molecules in a unit of weight or volume suitable for administration to a subject.

An inhibitory nucleic acid molecule of the invention, other negative regulator of VPS4A, VPS4B, CHMP1A, CHMP1B, ULK3, VTA1, or IST1, or any other agent of the present invention may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for VPS4A, VPS4B, CHMP1A, CHMP1B, ULK3, VTA1, or IST1 inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleobase oligomer of the invention is likely to depend on such variables as the type and extent of the disorder, the overall health status of the particular patient, the formulation of the compound excipients, and its route of administration.

With respect to a subject having a neoplastic disease or disorder, an effective amount is sufficient to stabilize, slow, or reduce the proliferation of the neoplasm. Generally, doses of active polynucleotide compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the VPS4A, VPS4B, CHMP1A, CHMP1B, ULK3, VTA1, or IST1 polynucleotide or polypeptide compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Therapy

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest cancer cells that may have spread to other parts of the body from the original tumor, to relieve symptoms caused by the cancer, or to prevent cancer in the first place. As described above, if desired, treatment with an inhibitory nucleic acid molecule of the invention may be combined with therapies for the treatment of proliferative disease (e.g., radiotherapy, surgery, or chemotherapy). For any of the methods of application described above, an inhibitory nucleic acid molecule of the invention is desirably administered intravenously or is applied to the site of neoplasia (e.g., by injection).

After a subject is diagnosed as having a neoplasia (e.g., cancers, such as brain, bladder, bile, blood, breast, duct (e.g., bile duct or pancreatic duct), colon, colorectal, esophageal, gastric, germ cell, liver, ovarian, pancreatic, uterine, and lung cancer, renal cell carcinoma, pancreatic ductal adrenocarcinoma, and sarcomas, such as, osteosarcoma and rhabdomyosarcoma (e.g., pediatric rhabdomyosarcoma (RMS)) a method of treatment is selected. A number of standard treatment regimens are known to clinicians.

In one embodiment, a method for inducing cell death or reducing cell survival of a neoplastic cell or neoplasia (e.g., cancers, such as brain, bladder, bile, blood, breast, duct (e.g., bile duct or pancreatic duct), colon, colorectal, esophageal, gastric, germ cell, liver, ovarian, pancreatic, uterine, and lung cancer, renal cell carcinoma, pancreatic ductal adrenocarcinoma, and sarcomas, such as, osteosarcoma and rhabdomyosarcoma (e.g., pediatric rhabdomyosarcoma (RMS)) characterized by a loss of VPS4A expression may be provided where the cell is contacted with an agent that inhibits the expression or activity of VPS4A, thereby inducing cell death or reducing cell survival of the neoplastic cell or neoplasia.

Another embodiment provides for a method for inducing cell death or reducing cell survival of a neoplastic cell or neoplasia (e.g., bile duct or pancreatic duct), colon, colorectal, esophageal, gastric, germ cell, liver, ovarian, pancreatic, uterine, and lung cancer, renal cell carcinoma, pancreatic ductal adrenocarcinoma, and sarcomas, such as, osteosarcoma and rhabdomyosarcoma (e.g., pediatric rhabdomyosarcoma (RMS)) characterized by a loss of VPS4A expression may be provided where the cell is contacted with an agent that inhibits the expression or activity of VPS4B, thereby inducing cell death or reducing cell survival of the neoplastic cell or neoplasia.

A further embodiment provides for a method inducing cell death or reducing cell survival of a neoplastic cell characterized by a loss of VPS4A and/or VPS4B and/or SMAD family member 4 (SMAD4) also known as Mothers against decapentaplegic homolog 4, and/or cadherin-1 (CDH1), where the method has a step of contacting the cell with an agent that inhibits the expression or activity of VPS4B (if the cell has a loss of VPS4A), VPS4A (if the cell has a loss of VPS4B), Ulk3 kinase, chromatin modifying protein (CHMP) 1A (CHMP1A) and/or CHMP1B, thereby inducing cell death or reducing cell survival of the neoplasia cell. An exemplary Unc-51 Like Kinase 3 (ULK3) inhibitor may include SU6668. An exemplary VSP4B inhibitor may include MSC1094308 or any of those inhibitors described in Pohler, et al.,” A Non-Competitive Inhibitor of VCP/p97 and VPS4 Reveals Conserved Allosteric Circuits in Type I and II AAA ATPases”, Angew. Chem. Int. Ed., 57:1576-1580 (2018). Other agents may include a small molecular, polypeptide, or polynucleotide. Non-limiting examples of agents useful in the methods of the disclosure include: an anti-VPS4A antibody, a VPS4A siRNA, a VPS4A shRNA, a VPS4A miRNA, a VPS4A ribozyme, a VPS4A antisense RNA, a nucleic acid that decreases VPS4A expression, a vector expressing at least one nucleic acid that decreases VPS4A nucleic acid expression; an anti-VPS4B antibody, a VPS4B siRNA, a VPS4B shRNA, a VPS4B miRNA, a VPS4B ribozyme, a VPS4B antisense RNA, a nucleic acid that decreases VPS4B expression, a vector expressing at least one nucleic acid that decreases VPS4B nucleic acid expression; an anti-ULK3 antibody, an ULK3 siRNA, an ULK3 shRNA, an ULK3 miRNA, an ULK3 ribozyme, an ULK3 antisense RNA, a nucleic acid that decreases ULK3 expression, a vector expressing at least one nucleic acid that decreases ULK3 nucleic acid expression; an anti-CHMP1A antibody, a CHMP1A siRNA, a CHMP1A shRNA, a CHMP1A miRNA, a CHMP1A ribozyme, a CHMP1A antisense RNA, a nucleic acid that decreases CHMP1A expression, a vector expressing at least one nucleic acid that decreases CHMP1A nucleic acid expression; an anti-CHMP1B antibody, a CHMP1B siRNA, a CHMP1B shRNA, a CHMP1B miRNA, a CHMP1B ribozyme, a CHMP1B antisense RNA, a nucleic acid that decreases CHMP1B expression, a vector expressing at least one nucleic acid that decreases CHMP1B nucleic acid expression; an anti-IST1 antibody, a IST1 siRNA, a IST1 shRNA, a IST1 miRNA, a IST1 ribozyme, a IST1 antisense RNA, a nucleic acid that decreases IST1 expression, a vector expressing at least one nucleic acid that decreases IST1 nucleic acid expression; an anti-VTA1 antibody, a VTA1 siRNA, a VTA1 shRNA, a VTA1 miRNA, a VTA1 ribozyme, a VTA1 antisense RNA, a nucleic acid that decreases VTA1 expression, a vector expressing at least one nucleic acid that decreases VTA1 nucleic acid expression; an anti-IST1 antibody, a IST1 siRNA, a IST1 shRNA, a IST1 miRNA, a IST1 ribozyme, a IST1 antisense RNA, a nucleic acid that decreases IST1 expression, a vector expressing at least one nucleic acid that decreases IST1 nucleic acid expression and any combinations thereof. Further non-limiting examples of agents include a guide RNA targeting VPS4A, a guide RNA targeting VPS4B, a guide RNA targeting ULK3, a guide RNA targeting CHMP1A, a guide RNA targeting CHMP1B, a guide RNA targeting VTA1, a guide RNA targeting IST1, polypeptides or polynucleotides encoding polypeptides for targeted gene editing or for CRISPR interference, and various combinations thereof.

Any of the methods of the disclosure may further comprise contacting the neoplastic cell or neoplasia with an interferon (e.g., interferon-β, interferon-γ). Accordingly, combination therapies (e.g., VPS4 inhibitor and/or ULK3 inhibitor and/or interferon) may be utilized in the methods for inducing cell death or reducing cell survival of a neoplastic cell or neoplasia, as well as the methods of treating a subject suffering from a neoplasia and/or has a VPS4 copy number deficiency.

### Therapy Selection

In one embodiment, cancer therapy is selected by measuring markers in a biological sample from a patient having or at risk for developing a neoplasia and detecting an alteration in the expression of a test marker molecule relative to the sequence or expression of a reference molecule. The markers can be selected from VPS4A, VPS4B, SMAD family member 4 (SMAD4) also known as Mothers against decapentaplegic homolog 4, cadherin-1 (CDH1), CHMP4B, ITCH, and ISG15 polypeptides or polynucleotides. While the following approaches describe diagnostic methods featuring VPS4A, VPS4B, SMAD4, CDH1, CHMP4B, ITCH, and ISG15, the skilled artisan will appreciate that any one or more of the markers delineated herein is useful in such methods.

Loss of the expression of one or more of a VPS4A, VPS4B, SMAD4, or CDH1 nucleic acid molecule or polypeptide is correlated with neoplasia. Further, the Examples provided herein demonstrate that expression levels (e.g., as determined by measuring mRNA levels) of VPS4B, CHMP4B, ITCH, and/or ISG15 can be used as biomarkers (e.g., for determining VPS4A dependency). Accordingly, the invention provides compositions and methods (e.g., use of a multivariate model) for characterizing a neoplasia in a subject to select a therapy. The present invention provides a number of assays that are useful for the identification or characterization of a neoplasia. Alterations in gene expression are detected using methods known to the skilled artisan and described herein. Such information can be used to diagnose a neoplasia.

Methods for creating a multivariate model are known in the art (see, e.g., H. Joe, Multivariate Models and Dependence Concepts, Chapman & Hall, 1997; F. Harrell, Regression Modeling Strategies, Springer, 2001; and Stephan, et al., “PSA and new biomarkers within multivariate models to improve early detection of prostate cancer”, Cancer Letters, 249:18-29 (2007)).

In one approach, diagnostic methods of the invention are used to assay the expression of a VPS4A, VPS4B, SMAD4, CDH1, CHMP4B, ITCH, or ISG15 polypeptide in a biological sample relative to a reference (e.g., the level of such polypeptide present in a corresponding control tissue). In one embodiment, the level of a VPS4A, VPS4B, SMAD4, CDH1, CHMP4B, ITCH, or ISG15 polypeptide is detected using an antibody that specifically binds a VPS4A, VPS4B, SMAD4, CDH1, CHMP4B, ITCH, or ISG15 polypeptide. Exemplary antibodies that specifically bind such a polypeptide are known in the art. By “antibody” is meant any immunoglobulin polypeptide, or fragment thereof, having immunogen binding ability. Such antibodies are useful for characterizing a neoplasia. Methods for measuring an antibody-VPS4A, VPS4B, SMAD4, CDH1, CHMP4B, ITCH, or ISG15 complex include, for example, detection of fluorescence, luminescence, chemiluminescence, absorbance, reflectance, transmittance, birefringence or refractive index. Optical methods include microscopy (both confocal and non-confocal), imaging methods and non-imaging methods. Methods for performing these assays are readily known in the art. Other useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra. Immunoassays can be used to determine the quantity of VPS4A, VPS4B, SMAD4, CDH1, CHMP4B, ITCH, or ISG15 in a sample, where a decrease in the level of such polypeptide characterizes the neoplasia.

In one approach, quantitative PCR methods are used to identify a decrease in the expression of a VPS4A, VPS4B, SMAD4, CDH1, CHMP4B, ITCH, or ISG15 nucleic acid molecule. In another approach, PCR methods are used to identify an alteration in the sequence of a VPS4A, VPS4B, SMAD4, CDH1, CHMP4B, ITCH, or ISG15 nucleic acid molecule. In one embodiment, a probe capable of detecting a VPS4A, VPS4B, SMAD4, CDH1, CHMP4B, ITCH, or ISG15 nucleic acid molecule, including genomic sequences, or closely related molecules is used. Such probes may be used to hybridize to a nucleic acid sequence derived from a patient having a neoplasia. The specificity of the probe determines whether the probe hybridizes to a naturally occurring sequence, allelic variants, or other related sequences. Hybridization techniques may be used to identify mutations indicative of a neoplasia or may be used to monitor expression levels of these genes (for example, by Northern analysis (Ausubel et al., supra).

Another embodiment encompasses a method of characterizing a subject as having, or having a propensity to develop, a neoplasia. The method involves sequencing the VPS4A, VPS4B, SMAD4, CDH1, CHMP4B, ITCH, or ISG15 gene in a subject sample, wherein loss of or a mutation in VPS4A, VPS4B, SMAD4, CDH1, CHMP4B, ITCH, or ISG15 relative to a reference characterizes the neoplasia.

In general, the measurement of a nucleic acid molecule in a subject sample is compared with an amount present in a reference. A diagnostic amount distinguishes between a neoplastic tissue and a control tissue. The skilled artisan appreciates that the particular diagnostic amount used can be adjusted to increase sensitivity or specificity of the diagnostic assay depending on the preference of the diagnostician. In general, any significant decrease (e.g., at least about 10%, 15%, 30%, 50%, 60%, 75%, 80%, or 90%) in the level of test nucleic acid molecule or test polypeptide in the subject sample relative to a reference may be used to diagnose a neoplasia. Test molecules include VPS4A, VPS4B, SMAD4, CDH1, CHMP4B, ITCH, or ISG15. In one embodiment, the reference is the level of test polypeptide or nucleic acid molecule present in a control sample obtained from a patient that does not have a neoplasia. In another embodiment, the reference is a baseline level of test molecule present in a biologic sample derived from a patient prior to, during, or after treatment for a neoplasia. In yet another embodiment, the reference can be a standardized curve.

### Types of Biological Samples

The level of markers in a biological sample from a patient having or at risk for developing a neoplasia can be measured, and an alteration in the expression of test marker molecules relative to the sequence or expression of a reference molecule, can be determined in different types of biologic samples. Test markers include VPS4A, VPS4B, SMAD4, CDH1, CHMP4B, ITCH, or ISG15. The biological samples are generally derived from a patient, preferably as a bodily fluid (such as tissue sample, blood, feces, cerebrospinal fluid, phlegm, saliva, or urine) or tissue sample (e.g. a tissue sample obtained by biopsy).

### Hardware and Software

The present invention also relates to a computer system involved in carrying out the methods of the invention relating to both computations (e.g., calculations associated with a multivariate model) and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

The invention provides kits for the characterizing a neoplasia. In one embodiment, the kit detects an alteration in the expression of a Marker (e.g., VPS4A, VPS4B, SMAD4, CDH1, CHMP4B, ITCH, or ISG15) nucleic acid molecule or polypeptide relative to a reference level of expression. In related embodiments, the kit includes reagents for monitoring the expression of a VPS4A, VPS4B, SMAD4, CDH1, CHMP4B, ITCH, or ISG15 nucleic acid molecules, such as primers or probes that hybridize said nucleic acid molecule. In other embodiments, the kit includes an antibody that binds to a VPS4A, VPS4B, SMAD4, CDH1, CHMP4B, ITCH, or ISG15 polypeptide. In some embodiments, the kit includes an agent that alters expression or activity of VPS4A, VPS4B, CHMP1A, CHMP1B, ULK3, VTA1, or IST1.

Optionally, the kit includes directions for monitoring the nucleic acid molecule or polypeptide levels of a Marker in a biological sample derived from a subject. In other embodiments, the kit comprises a sterile container which contains the primer, probe, antibody, or other detection regents; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a neoplasia. Preferably, the kit further comprises any one or more of the reagents described in the diagnostic assays described herein. In other embodiments, the instructions include at least one of the following: description of the primer or probe; methods for using the enclosed materials for the diagnosis of a neoplasia; precautions; warnings; indications; clinical or research studies; and/or references. The instructions may be printed directly on the container (when present), or as a label applied to the container, or as a separate sheet, pamphlet, card, or folder supplied in or with the container.

### Patient Monitoring

The disease state or treatment of a patient having a neoplasia can be monitored using the methods and compositions of the invention. Therapeutics that alter the expression of a VPS4A, VPS4B, CHMP1A, CHMP1B, ULK3, VTA1, or IST1 polypeptide are taken as particularly useful in the invention.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Discovery of Synthetic Lethal Interactions with Genomic Loss of Established Tumor suppressors

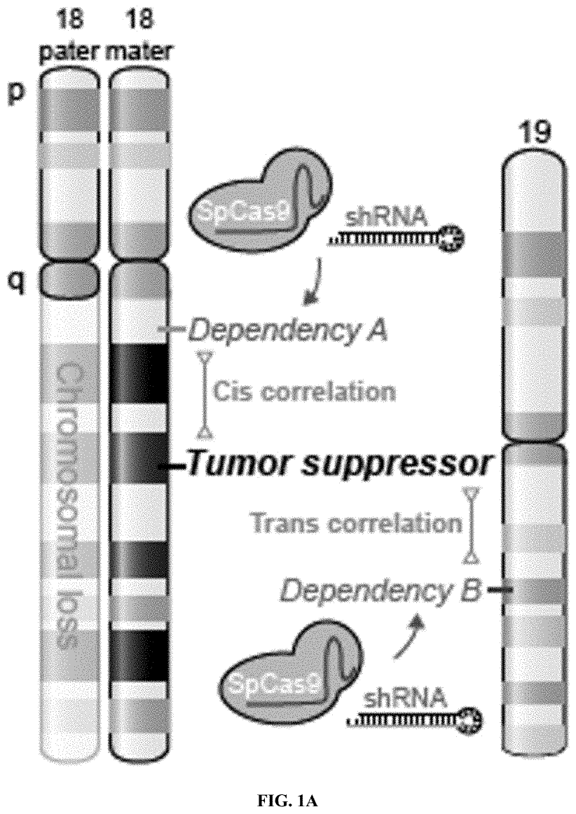

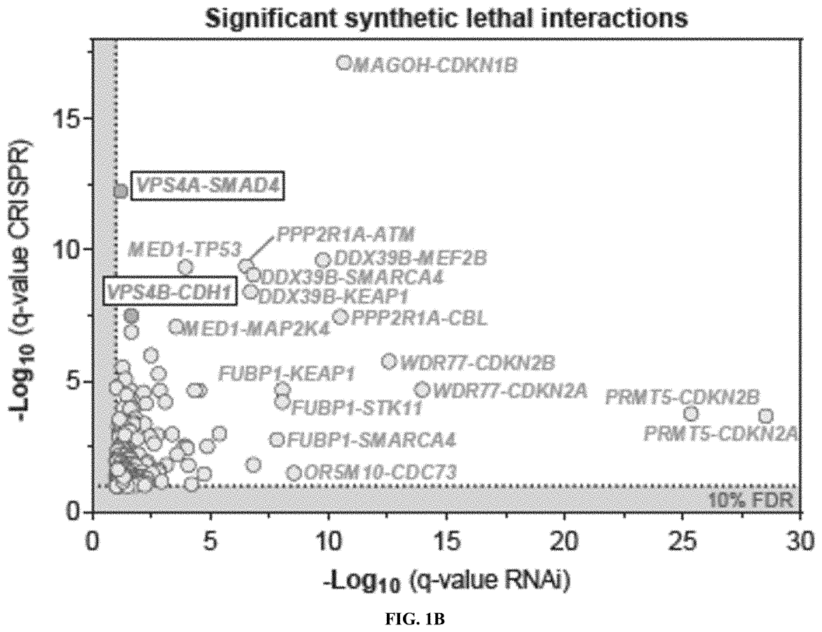

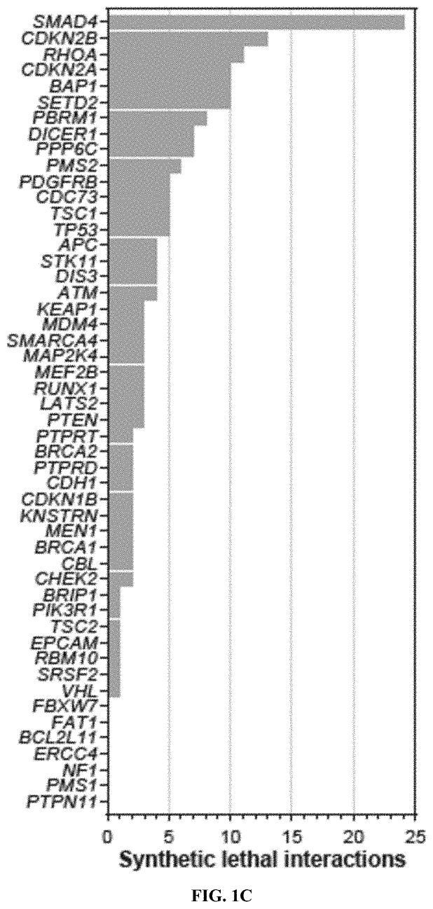

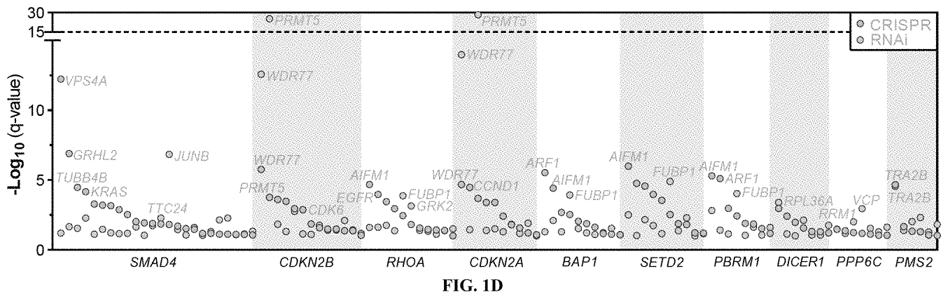

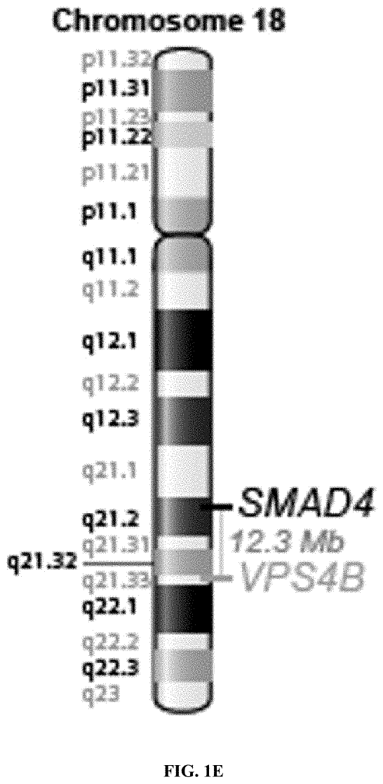

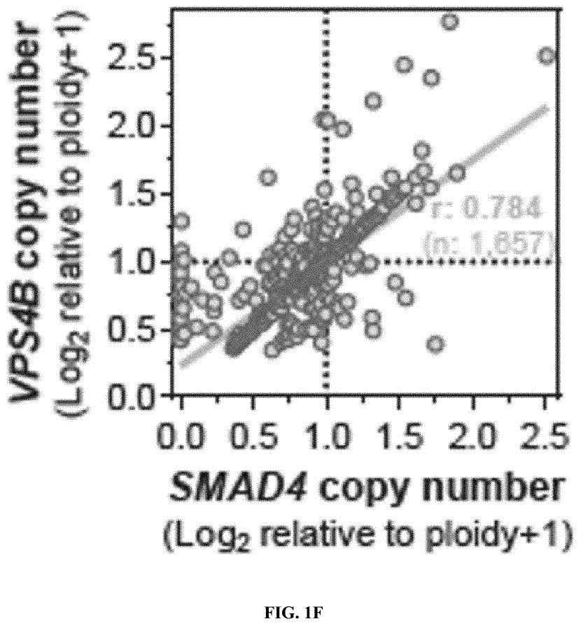

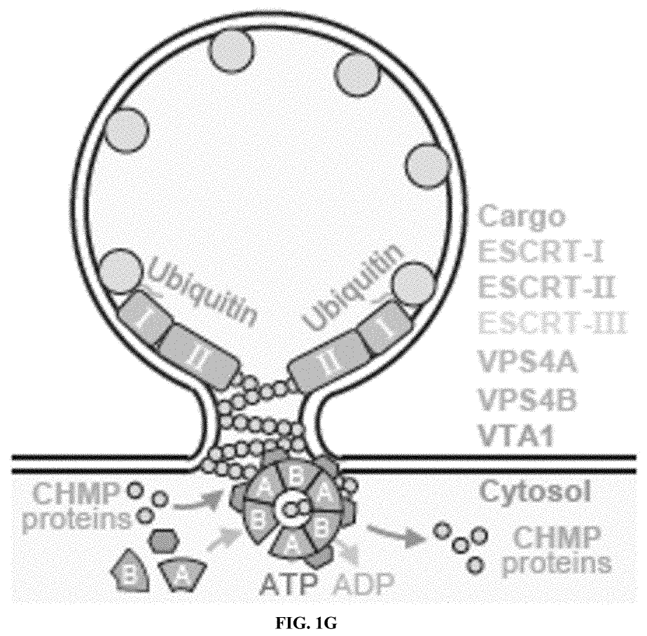

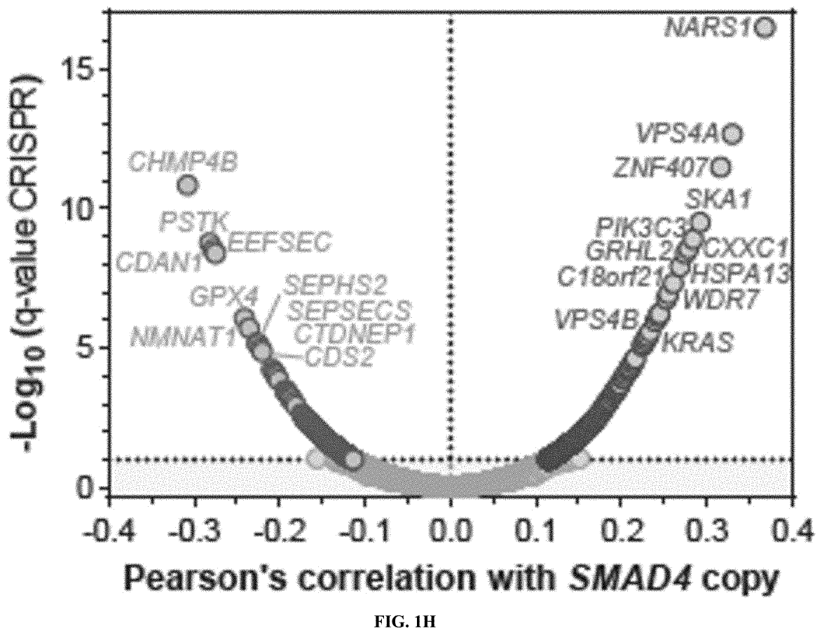

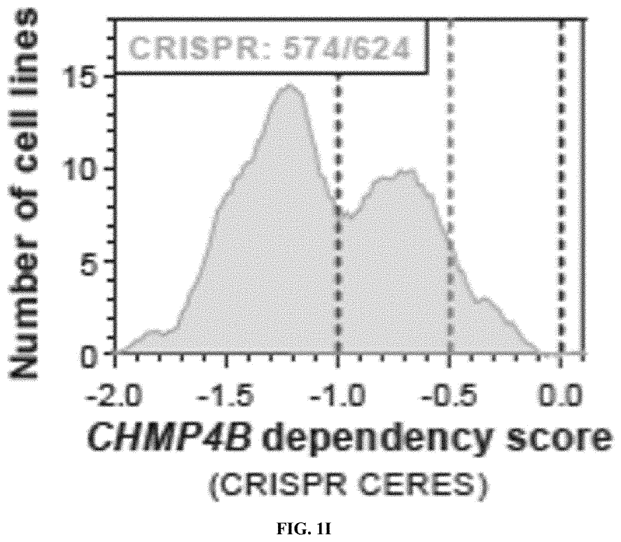

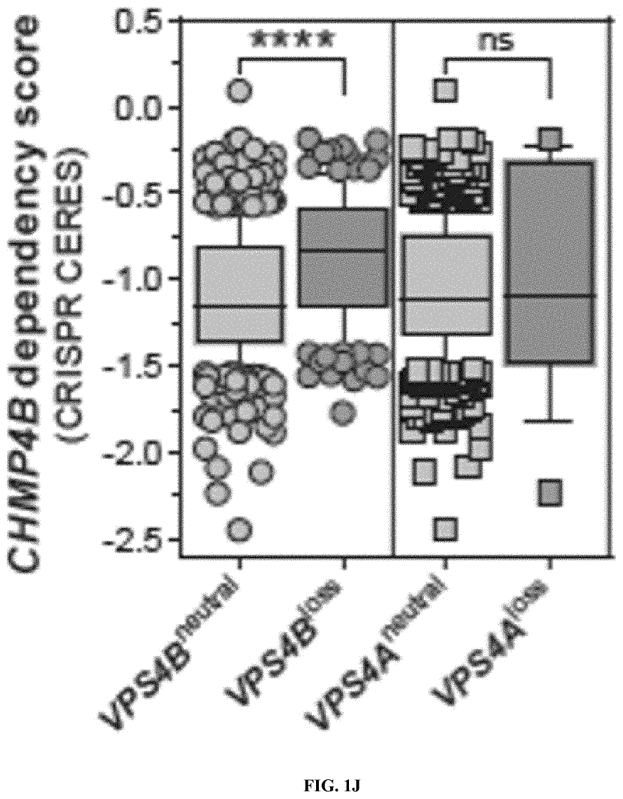

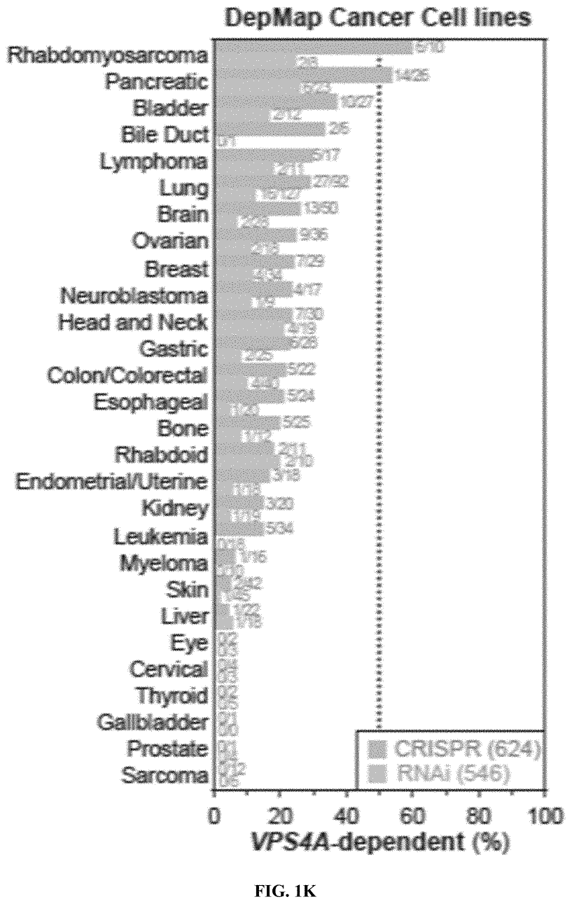

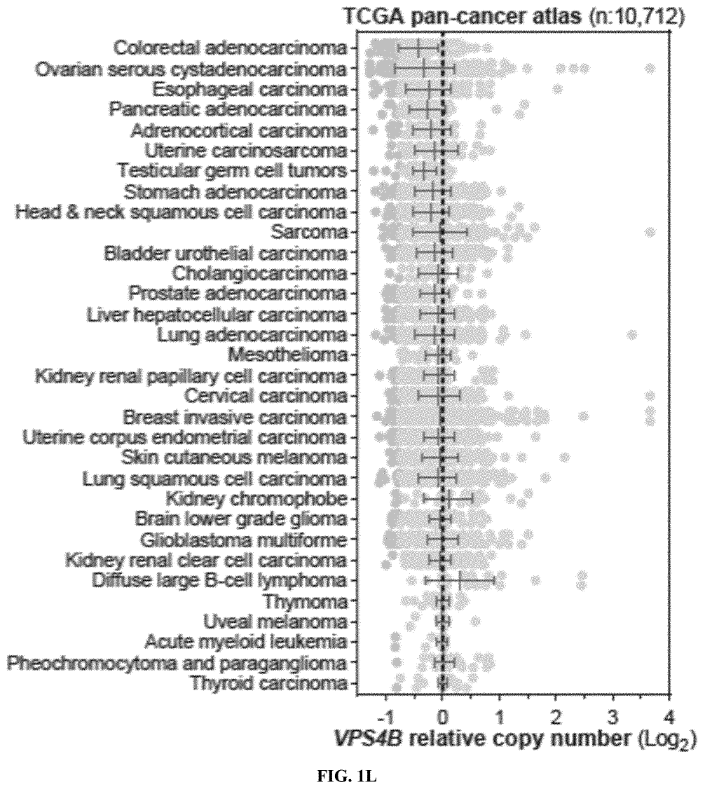

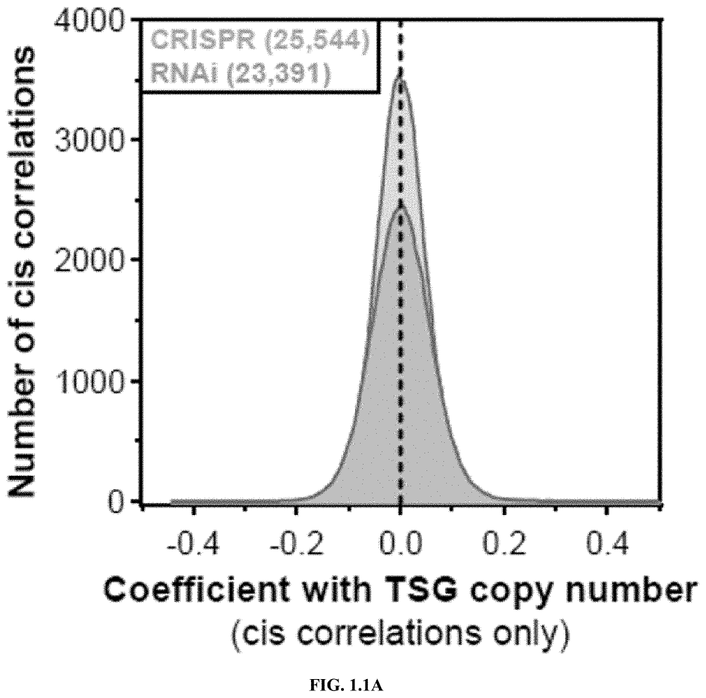

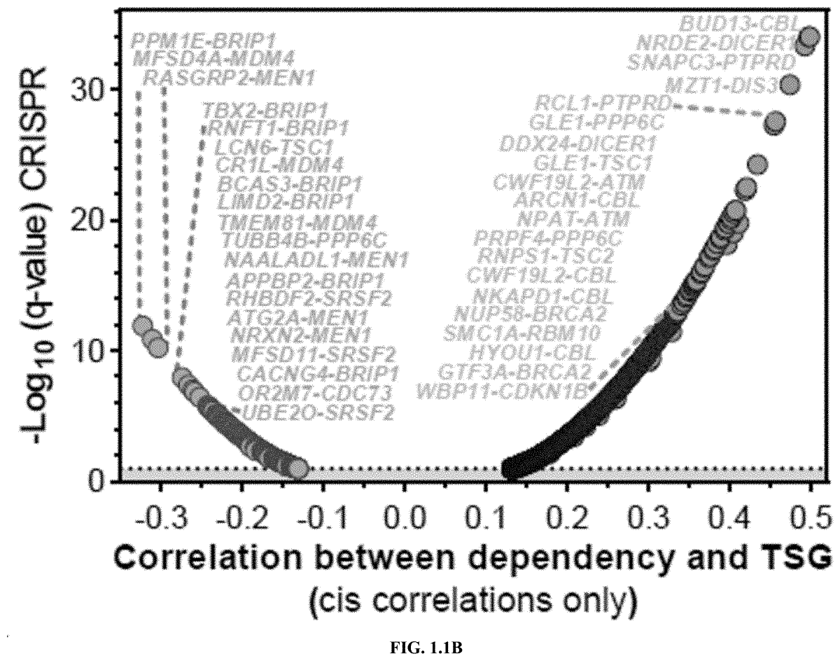

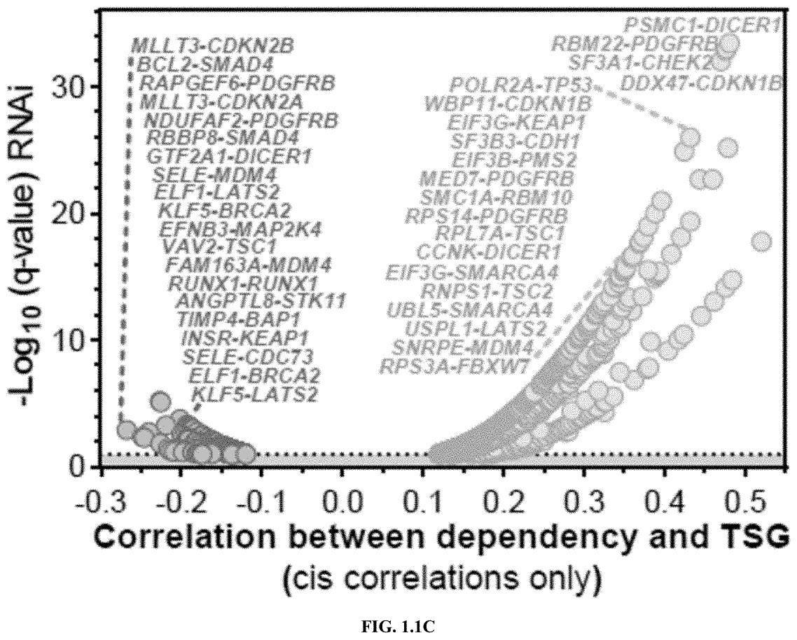

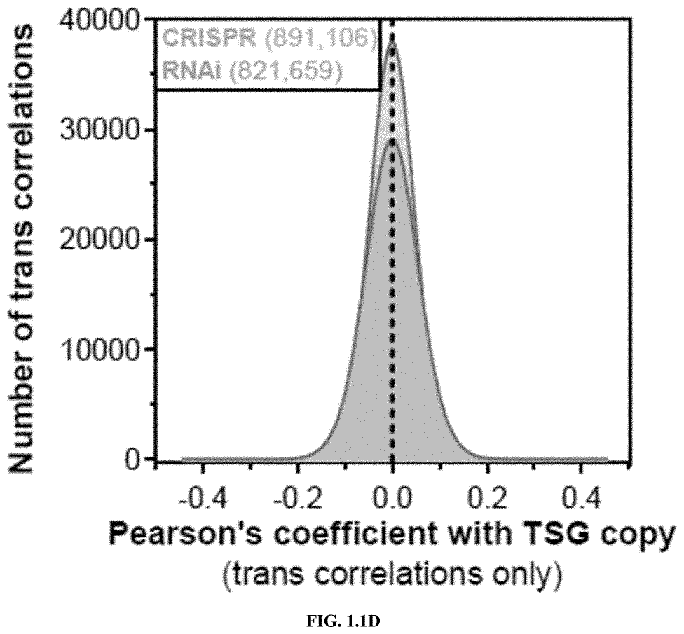

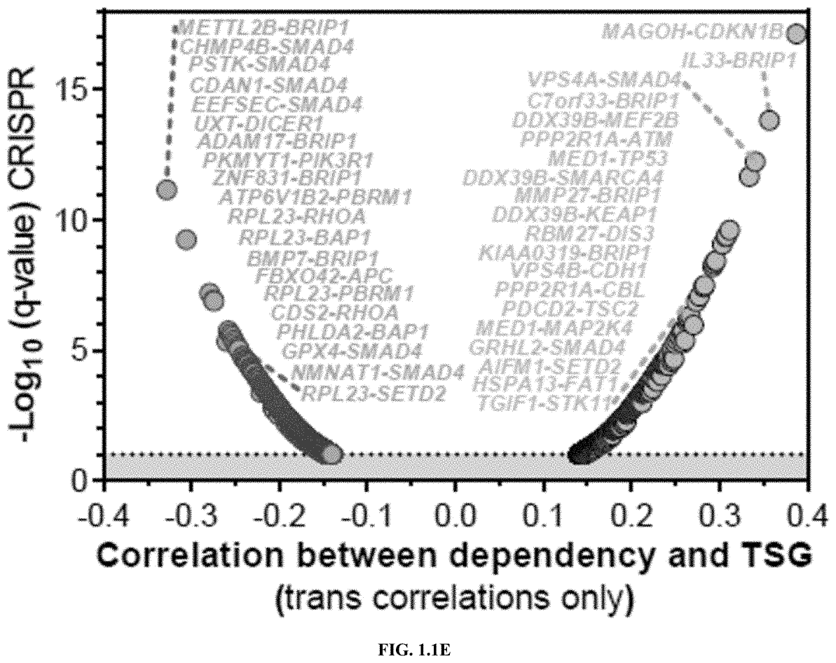

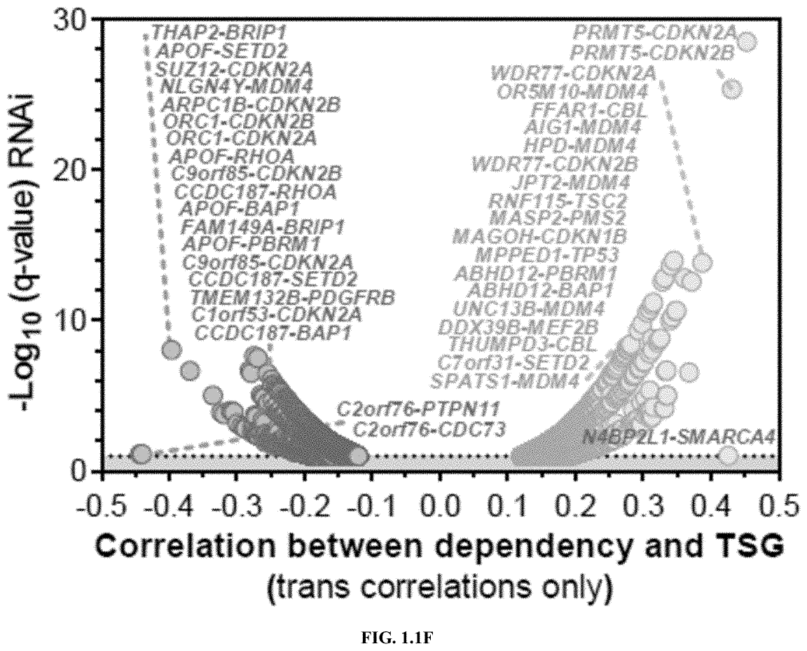

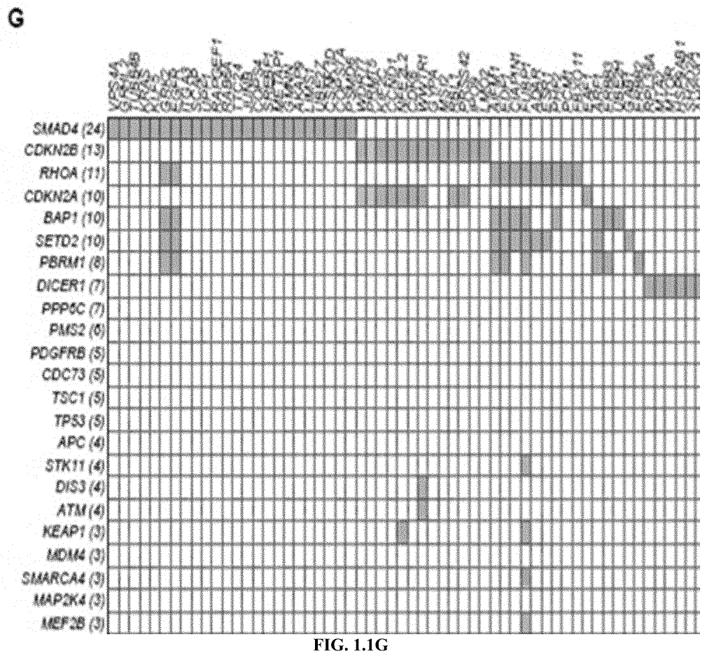

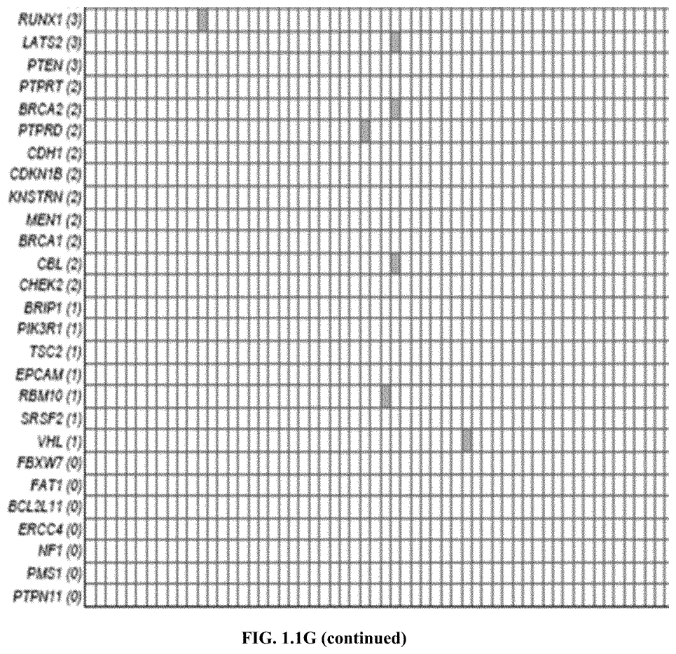

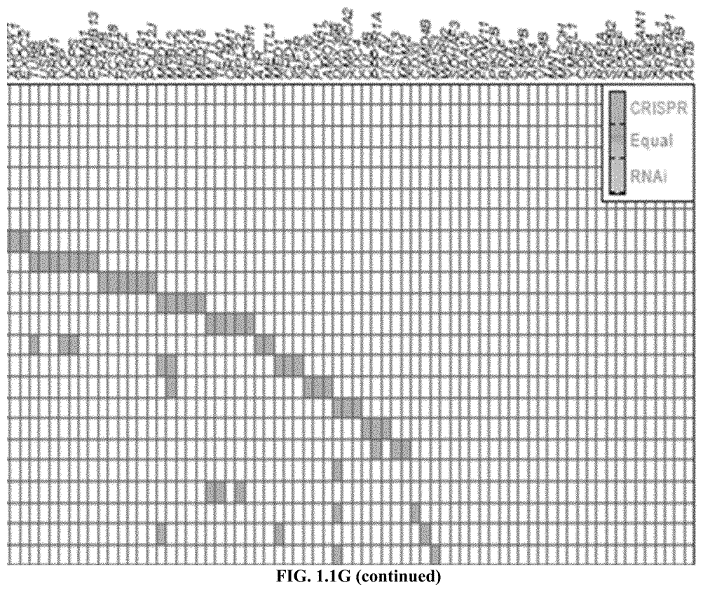

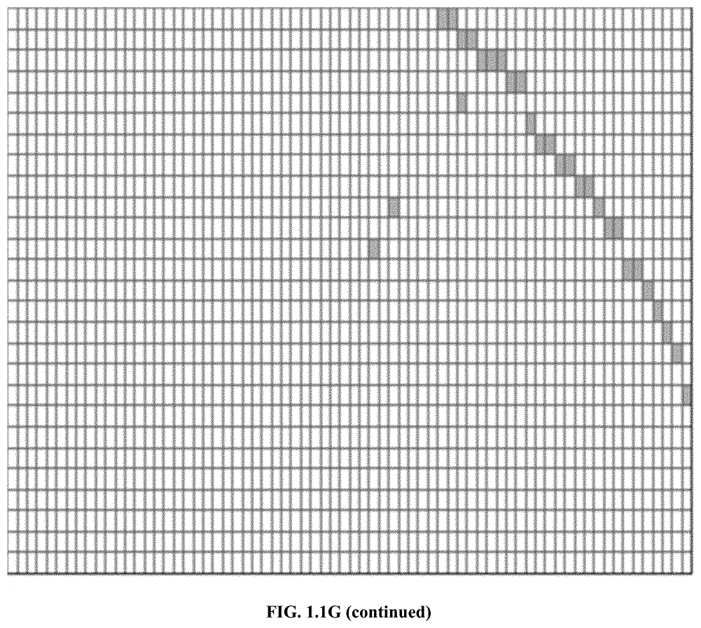

To uncover synthetic lethal interactions with genomic loss of tumor suppressor genes (TSGs), genome-scale RNA interference (RNAi) and CRISPR-SpCas9 cancer dependency datasets were analyzed (depmap.org). The analysis was focused on 50 commonly lost TSGs (Table 5) to identify synthetic lethal relationships that could be relevant to a large fraction of human cancers. Log 2-normalized copy number calls were correlated for each of these tumor suppressors with normalized, gene-level CRISPR-SpCas9 (622 cell lines, 18,333 genes) and RNAi (669 cell lines, 16,905 genes) dependency scores (FIG. 1A). Only 1.0% of CRISPR-SpCas9 (9,142/916,650) and 1.9% of RNAi (16,435/845,250) dependency-TSG interactions were significant at a 10% false discovery rate. Interestingly, a sizeable fraction of significant CRISPR-SpCas9 (31.5%; 2,877/9,142) and RNAi correlations (7.1%; 1,174/16,435) represented cis correlations in which the dependency gene localized to the same chromosomal arm as the TSG (FIGS. 1.1A-1.1C). As TSG loss is mainly driven by deletion of entire chromosomal arms, collateral (partial) loss of essential genes adjacent to TSGs often occurs, resulting in enhanced dependency on expression of these genes. Such cis occurring gene dependencies have previously been described as “CYCLOPS” (copy-number alterations yielding cancer liabilities owing to partial loss) https://paperpile.com/c/lzErcE/4HUEj%2BtwRRf..

To expand the understanding of synthetic lethal interactions with common TSGs beyond CYCLOPS genes, focus was placed on gene dependencies located on a different chromosome, i.e. in trans to the TSG (FIG. 1A). After filtering out cis correlations, 3,897 CRISPR-SpCas9 (0.4%) and 14,525 RNAi (1.8%) significant correlations were discovered at a 10% false discovery rate (FDR) (FIGS. 1.1D-1.1F). A total of 337 of these trans correlations were significant in both the CRISPR-SpCas9 and RNAi analysis (Table 4). Table 4 describes significant trans correlations with tumor suppressor gene copy number that scored in both the CRISPR-SpCas9 and RNAi analysis synthetic lethal analysis.

Some synthetic lethal interactions were unique to one dataset, partially due to incomplete overlap in the probed genes and screened cell lines between both datasets. To identify synthetic lethal interactions, positive correlations for which TSG loss (lower copy number) confers increased dependency (lower dependency score) were used to uncover 193 significant interactions across 127 genes (FIGS. 1B and 1.1G). Copy loss of the SMAD4, CDKN2B, RHOA, CDKN2A and BAP1 TSGs showed the largest number of synthetic lethal relationships (FIGS. 1C and 1.1G). For example, the SMAD4 copy number in 476 cases of pancreatic ductal adenocarcinoma (PDAC) cancer showed a gain (3.8%), loss (16.8%), neutral (22.9%), and shallow (56.5%). Whereas, in all cancers, the SMAD4 copy number showed a gain (4.7%), loss (1.2%), neutral (78.2%), and shallow (16.0%) in 28,231 cases.

Several paralog and collateral synthetic lethal interactions were among the most significant in the analysis, including those previously reported for MAGOH-CDKN1B and PRMT5/WDR77 with CDKN2A (FIGS. 1B and 1D) (Kryukov et al. Journal of Experimental Med, 214(10):2933-2946, 2016; Viswanathan et al., Nat Genet, 50(7):937-943, 2018). Dependency on the splicing-related DExD-box helicase 39B (DDX39B) correlated strongly with copy number of the MEF2B, SMARCA4, KEAP1 and STK11 tumor suppressor genes, which all map to chromosome 19p (FIG. 1B and Table 4). DDX39A, the main paralog of DDX39B, is located on chromosome 19p, suggesting that loss of 19p leads to loss of DDX39A, which in turn sensitizes cells to depletion of DDX39B. Similarly, dependency on the essential serine/threonine-protein phosphatase 2A regulatory subunit A alpha (PPP2R1A) correlated strongly with copy number of the ATM and CBL tumor suppressors on chromosome 11q which are adjacent to PPP2R1B, the main paralog of PPP2R1A (FIG. 1B and Table 4). Strong correlations were also identified between gene dependencies and copy loss of other members of the same pathway or protein complex. For example, dependency on the mediator complex subunit 1 (MED1) strongly correlated with loss of TP53 and MAP2K4, both of which map to chromosome 17p, along with MED9, MED11 and MED31, indicating that loss of these mediator subunits sensitizes cells to loss of MED1 https://paperpile.com/c/lzEreE/m9KwA. Moreover, dependency on FUBP1 correlated with copy number of TSGs (SMARCA4, KEAP1, STK11) located on chromosome 19p (FIG. 1B and Table 4). As reported previously, the gene encoding FUBP1's binding partner KHSRP (also known as FUBP2) localizes to chromosome 19p (Paolella et al. Elife, 6:e23268, 2017; Viswanathan et al. Nat Genet, 50(7):937-943, 2018).

### Example 2: The ESCRT Enzymes VPS4A and VPS4B are Paralog Synthetic Lethal Vulnerabilities in Cancers Harboring SMAD4 or CDH1 Loss

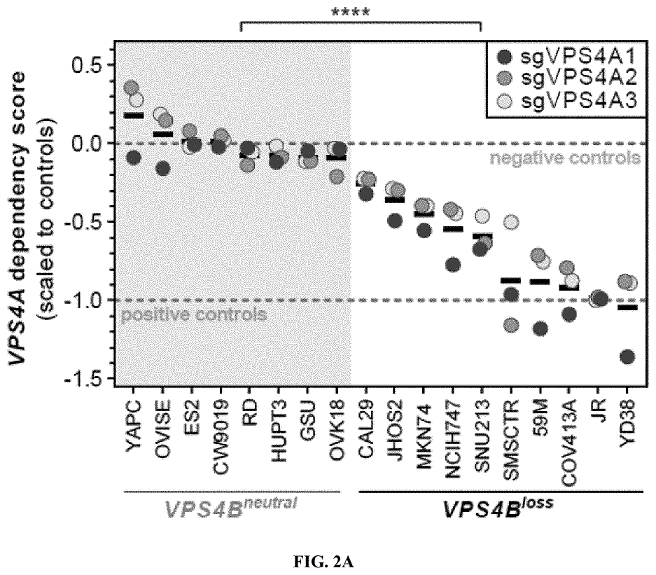

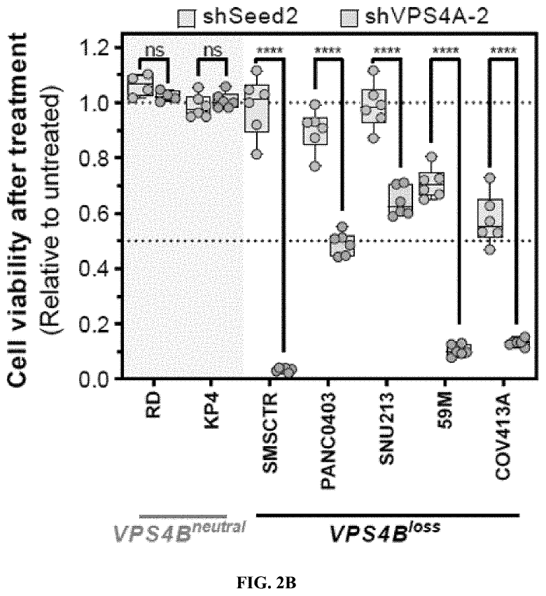

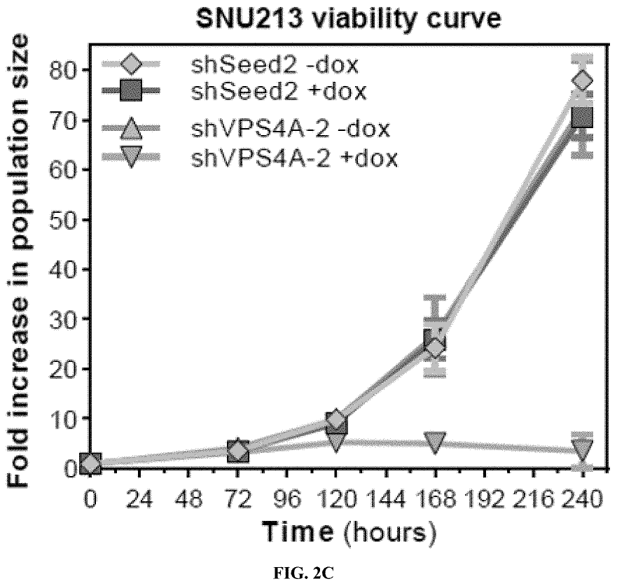

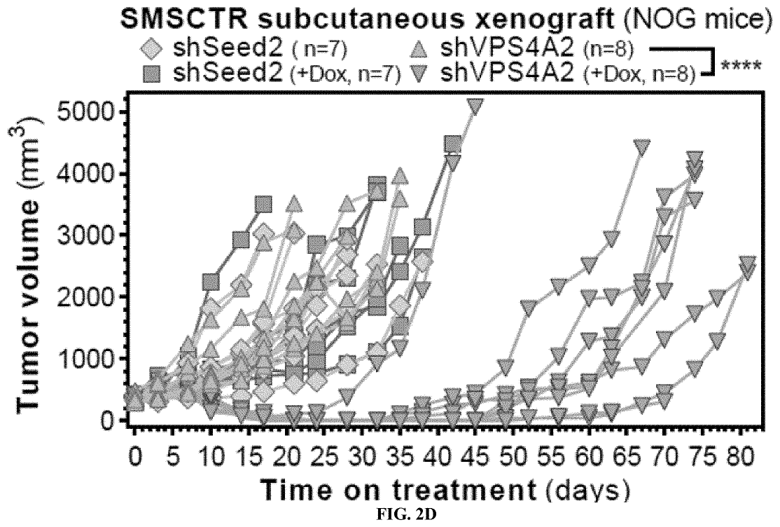

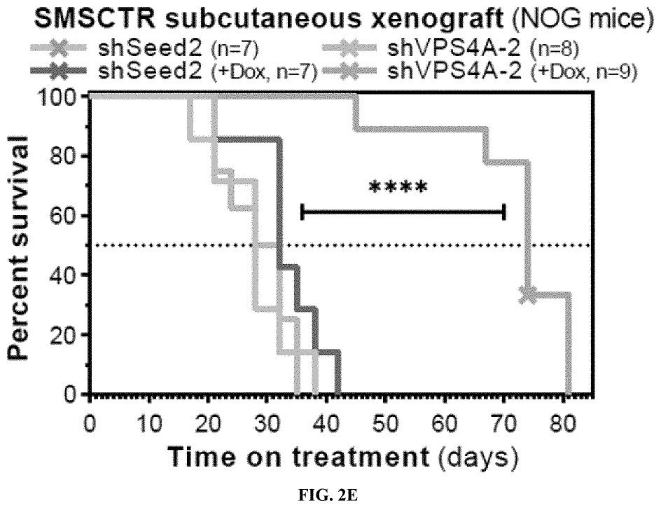

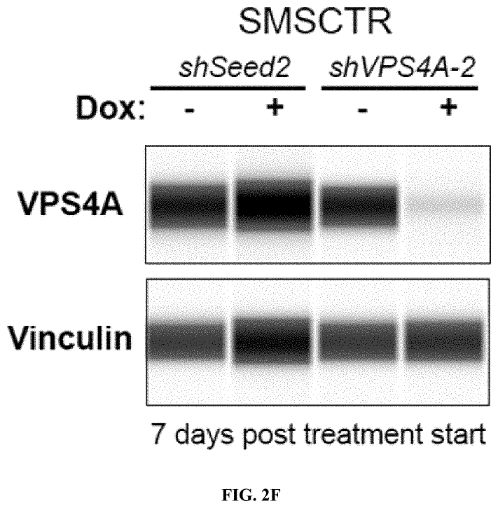

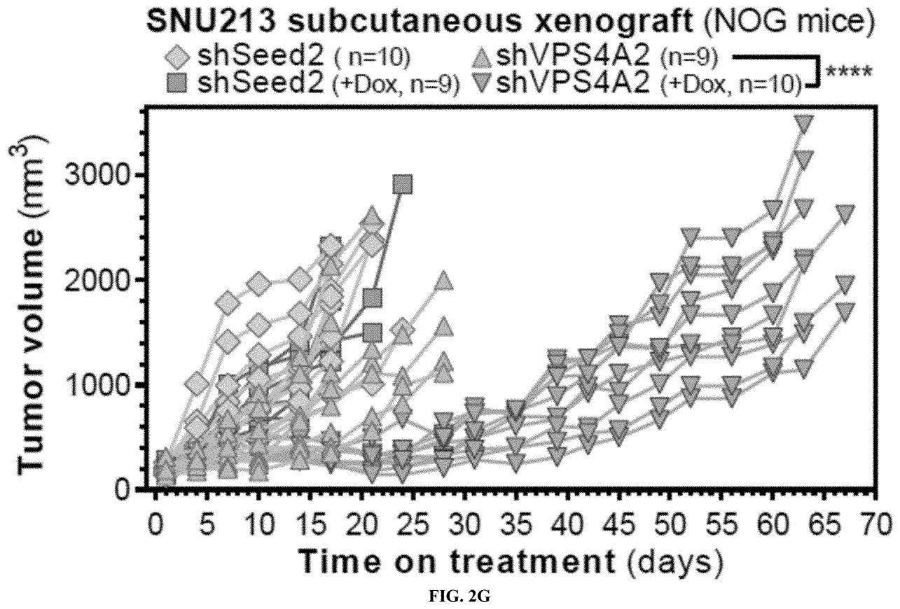

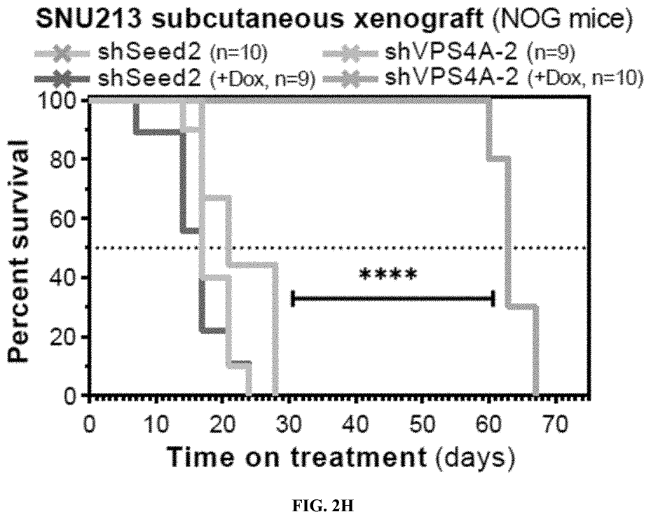

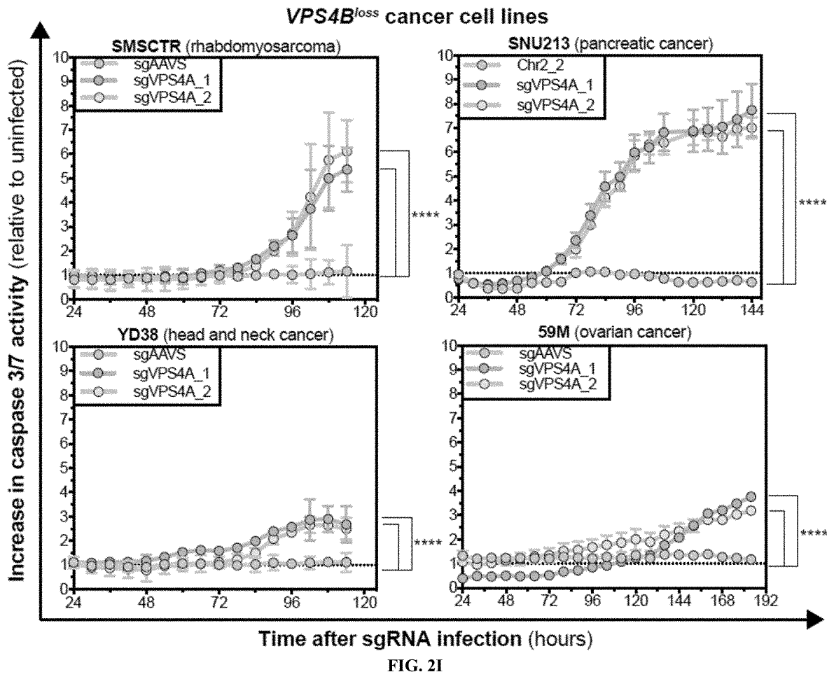

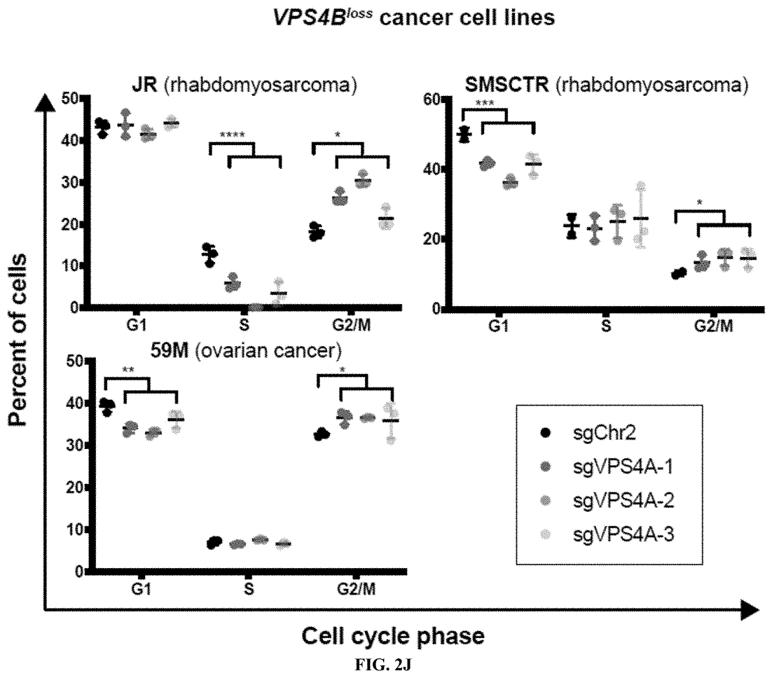

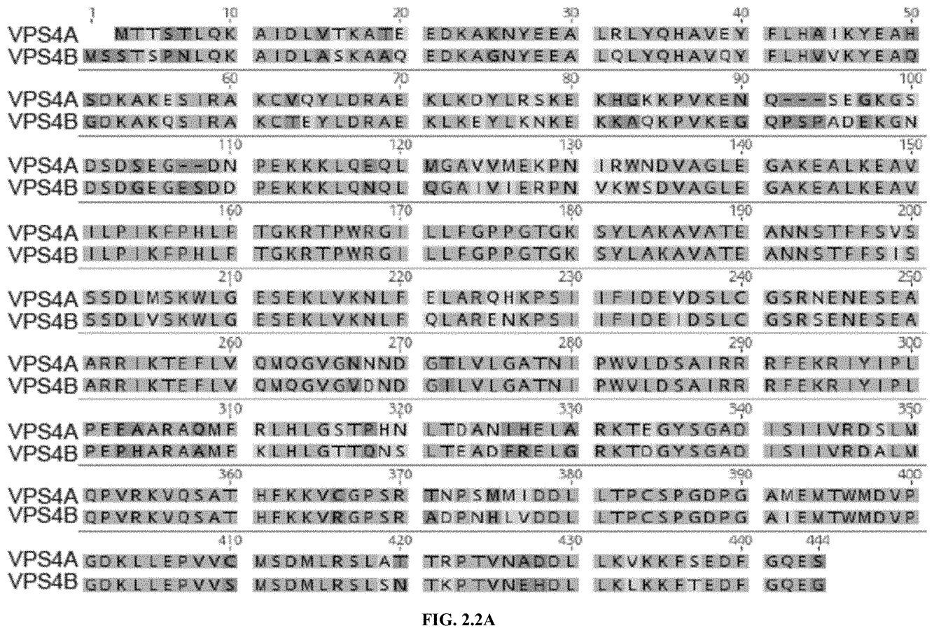

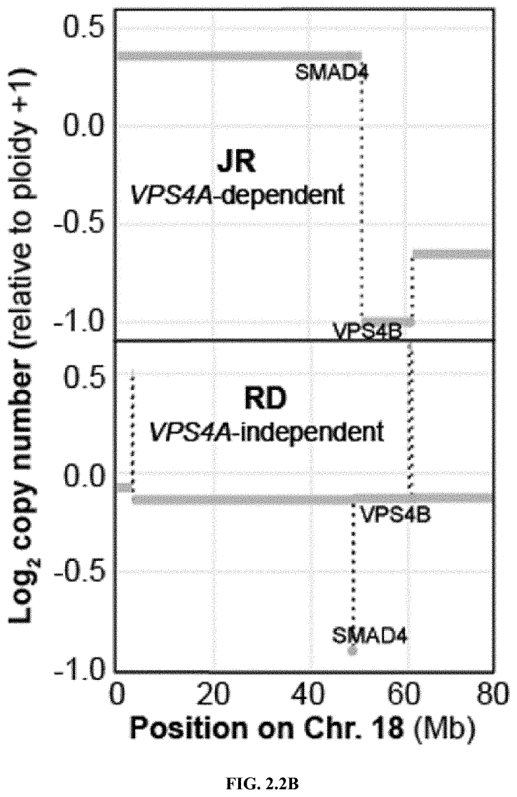

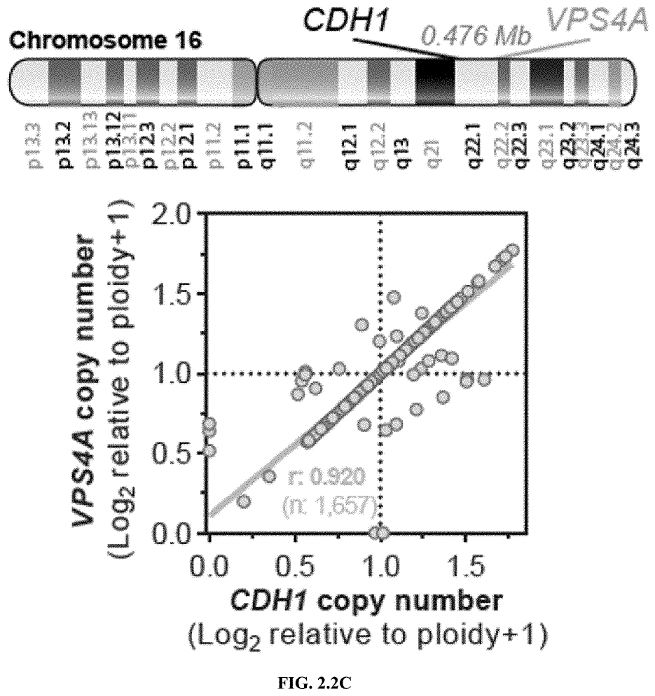

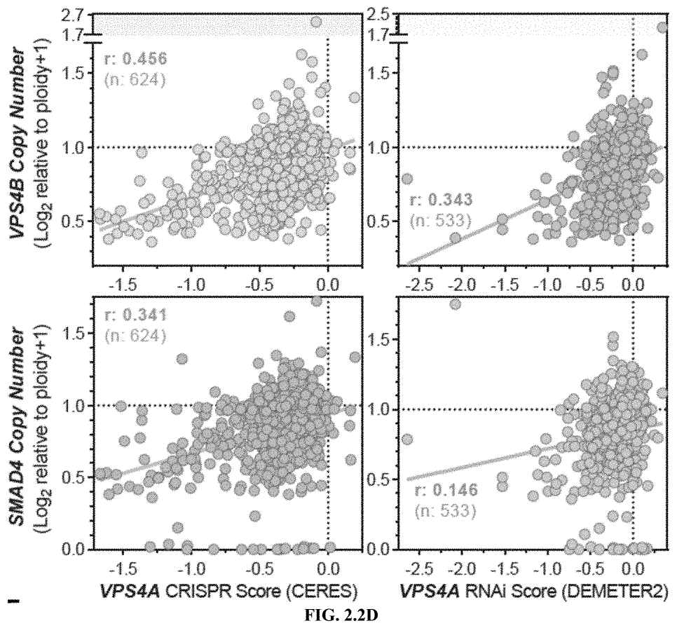

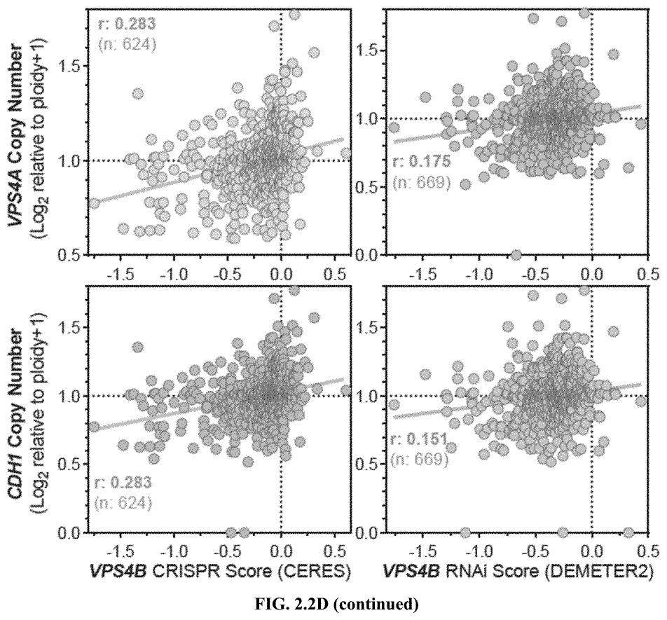

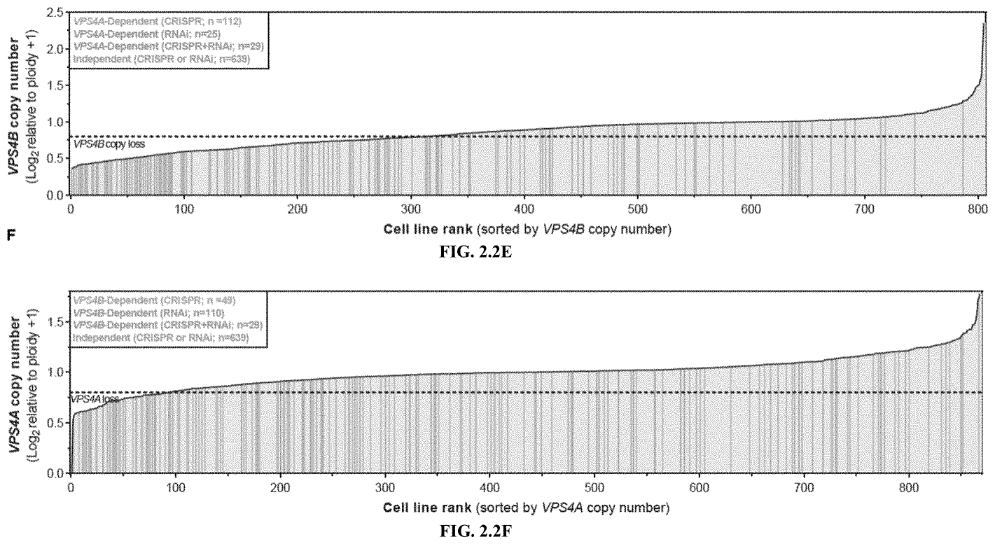

A striking number of trans dependency correlations were observed with SMAD4 deletion, one of the most commonly lost tumor suppressor genes in human cancer (FIGS. 1C and 1.1G). VPS4A scored as the strongest correlated gene dependency with copy loss of SMAD4/18q (FIGS. 1B-1D, Table 4). Notably, VPS4B, aparalog of VPS4A (FIG. 2.2A), resides 12.3 Mb adjacent to SMAD4 (FIG. 1E). Across cancer cell lines, VPS4B copy loss was frequent and strongly correlated with SMAD4 loss (FIG. 1F; R²: 0.615). Although SMAD4 was homozygously lost in some cell lines, homozygous loss of VPS4B was not observed (FIG. 1F). Interestingly, some VPS4A-dependent cell lines harbored normal SMAD4 copy number but showed focal loss of VPS4B, while some VPS4A-independent cell lines harbored loss of SMAD4 but retained normal copy number for VPS4B (FIG. 2.2B). These results indicate that dependency on VPS4A is driven by loss of VPS4B rather than SMAD4.

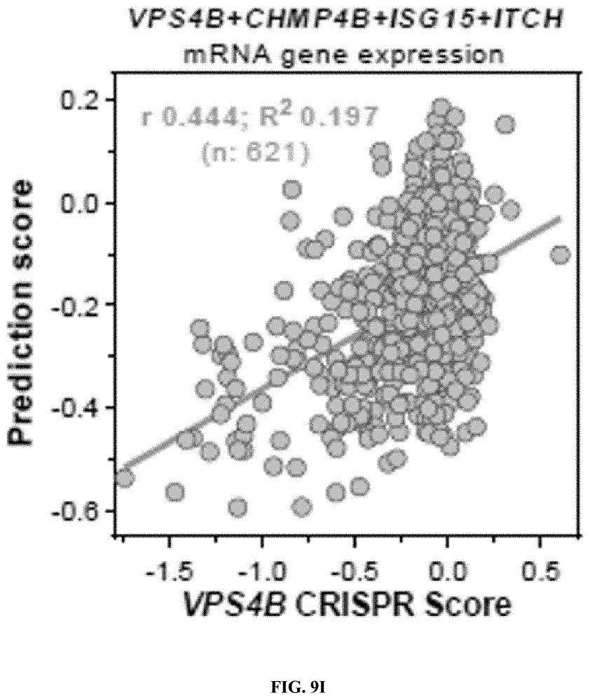

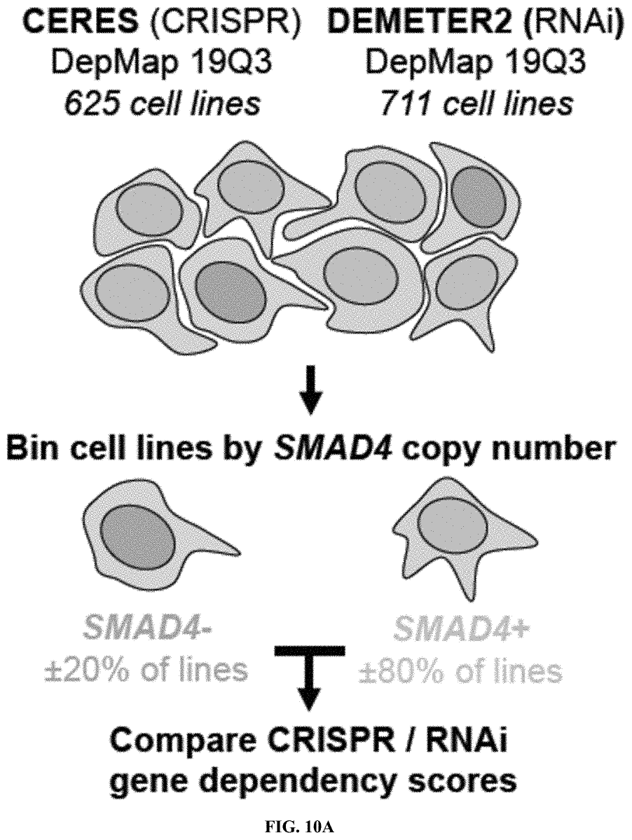

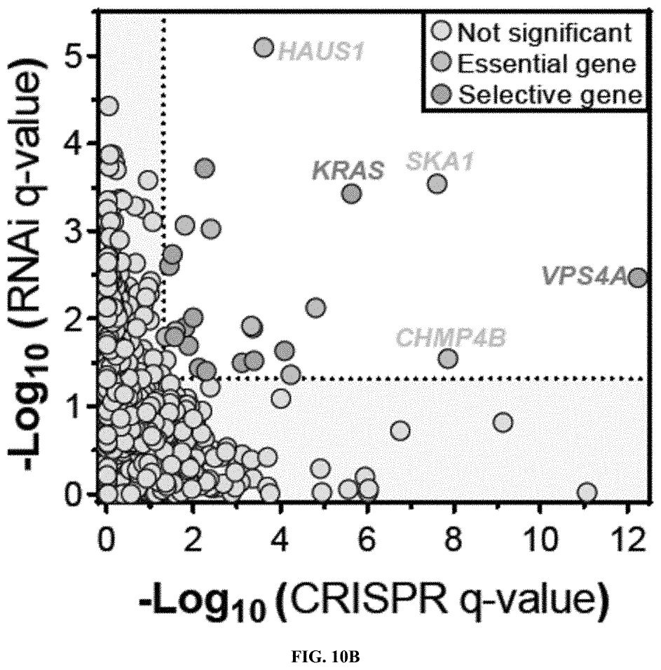

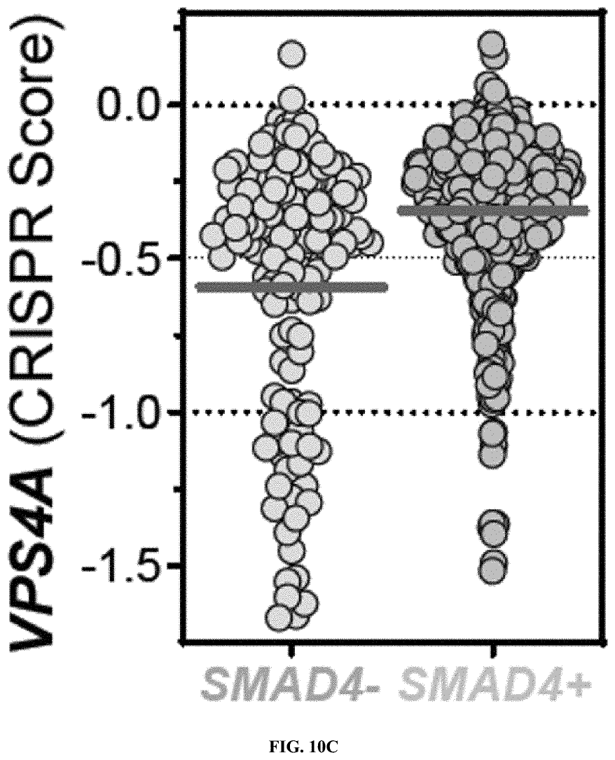

It was shown that SMAD4-deficient cancer cell lines selectively depend on VPS4A. The CERES (CRISPR) and DEMETER2 (RNAi) gene dependency scores were compared between cancer cell lines with neutral SMAD4 copy and cancer lines harboring SMAD4 copy loss to identify genes which are essential in SMAD4-deficient cancer cells. Gene dependencies were then classified as commonly essential genes (essential), selective essential genes (selective), and not significant genes (FIGS. 10A-10C). Statistical significance was determined using a 10%-false discovery rate-corrected (Benjamini-Hocberg) two-tailed unpaired T-test between the average gene dependency scores for each gene in SMAD4 neutral vs. SMAD4-deficient cancer cell lines.

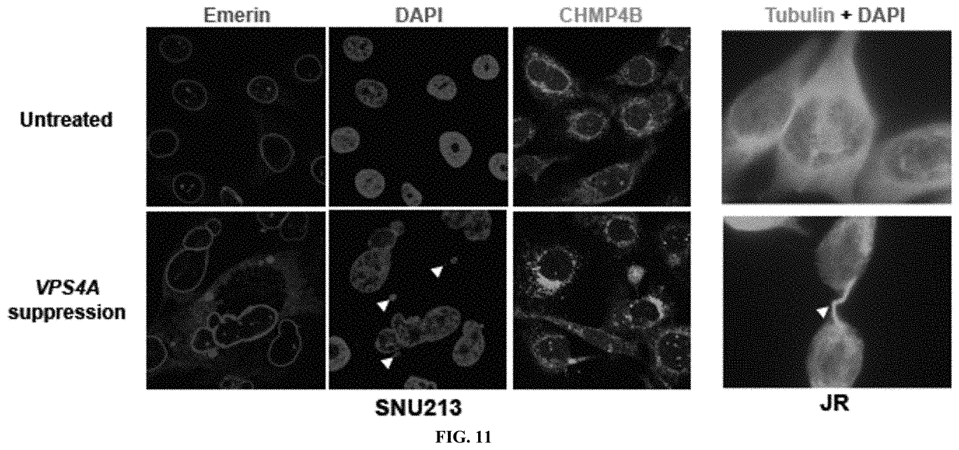

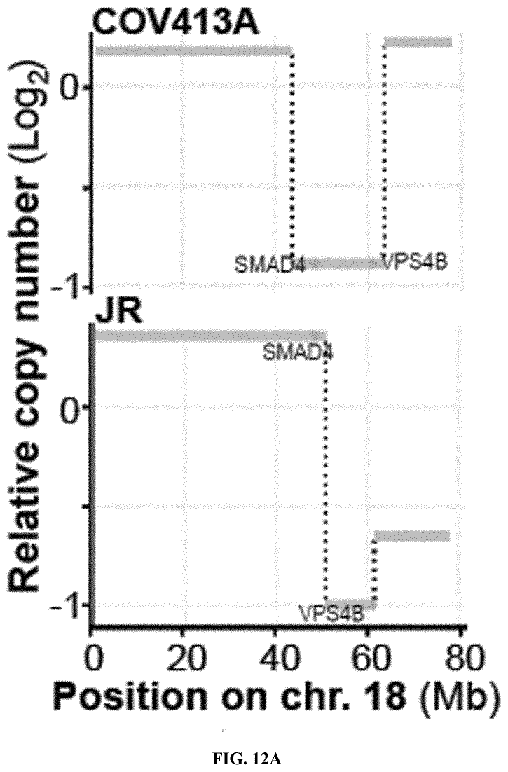

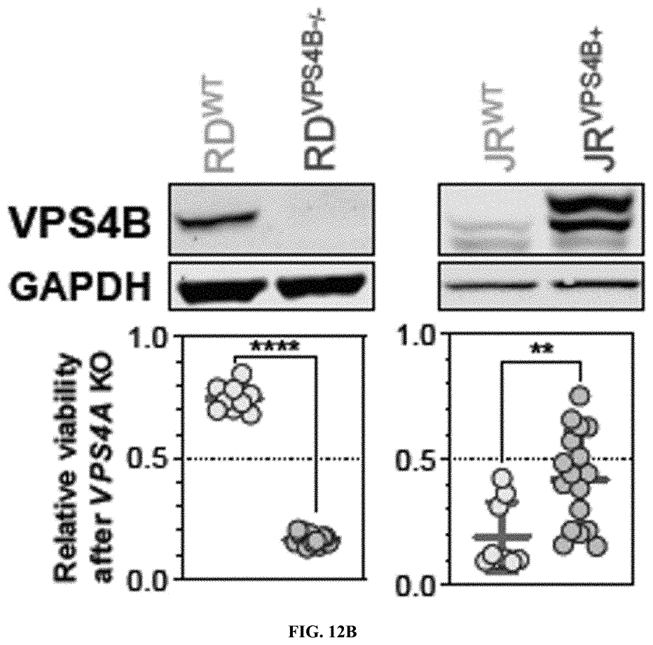

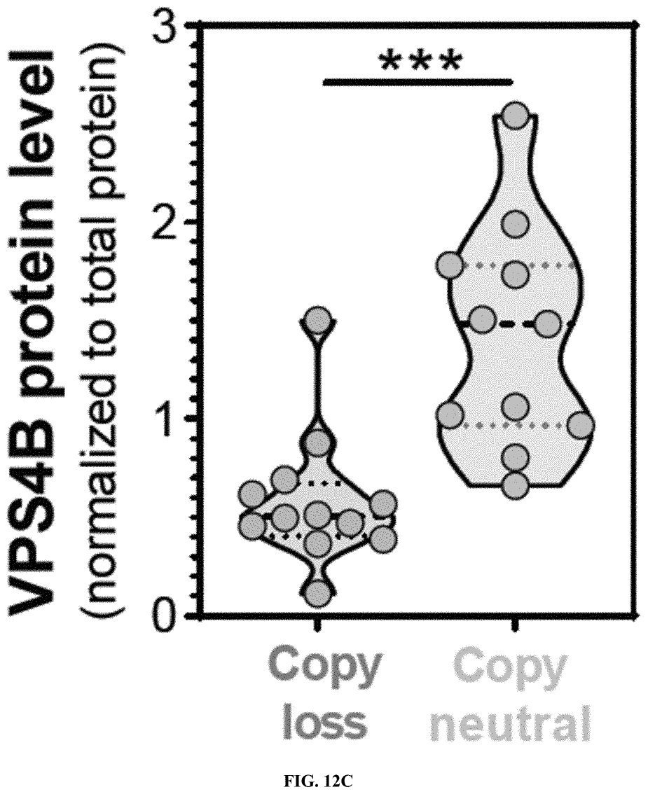

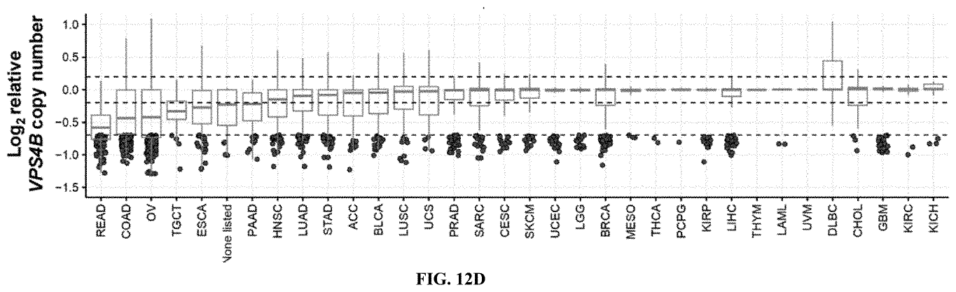

FIG. 12A shows the results in a plot of the log 2-normalized relative copy number of genetic probes (y-axis) across chromosome 18 (x-axis) highlighting SMAD4 and VPS4B copy number (dots) in the COV413A (top panel) ovarian cancer and JR (bottom panel) rhabdomyosarcoma cancer cell lines. **** p-value <0.0001, ** p-value <0.005; two-tailed unpaired T-test.

Furthermore, VPS4B copy loss correlated better with VPS4A dependency than did SMAD4 copy loss in both CRISPR-SpCas9 (R²:0.208 vs. R²: 0.116) and RNAi datasets (R2: 0.118 vs. R²: 0.021) (FIG. 2.2D, left panels). Moreover, of all VPS4A-dependent cancer cell lines, 63.9% (106/166) demonstrated at least partial genomic loss of VPS4B copies (FIG. 2.2E).

It was next evaluated whether a reciprocal relationship existed between VPS4B dependency and VPS4A copy number. VPS4B dependency significantly correlated with loss of the CDH1 tumor suppressor locus on chromosome 16q22.1 (FIG. 1B, Table 4). Strikingly, VPS4A localizes only 476 kilobases downstream of the CDH1 tumor suppressor gene (FIG. 2.2C). As a result, VPS4A copy number strongly correlated with CDH1 copy number (FIG. 2.2C, R²: 0.846). As expected, dependency on VPS4B correlated with VPS4A copy number in both CRISPR-SpCas9 and RNAi datasets (FIGS. 2.2D and 2.2F), although this correlation was less profound than that observed between VPS4A dependency and VPS4B copy loss (Compare FIGS. 2.2E and 2.2F).

VPS4A and VPS4B encode 49 kDa AAA ATPases and these paralog proteins are 81% identical (FIG. 2.2A). They form multimeric complexes with the ESCRT machinery to regulate reverse topology membrane remodeling and fission across many cellular processes (FIG. 1G). CHMP4B is a core filament-forming ESCRT-III protein that is essential for ESCRT mediated membrane remodeling https://paperpile.com/c/lzEreE/xcjnT. While VPS4A dependency scores positively correlated with SMAD4/VPS4B copy number (i.e., lower copy number correlates with lower gene dependency scores), a strong anticorrelation was observed between CHMP4B dependency and SMAD4 copy number (FIG. 1H, Table 4). Whereas CHMP4B is strongly essential for proliferation and survival in cells with euploid VPS4B copy number, cells that harbor loss of VPS4B are less sensitive to CHMP4B knockout (FIGS. 11 and 1J).

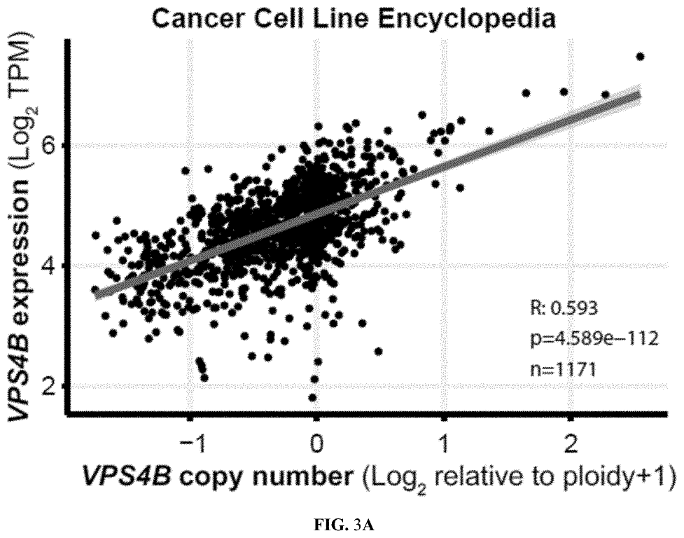

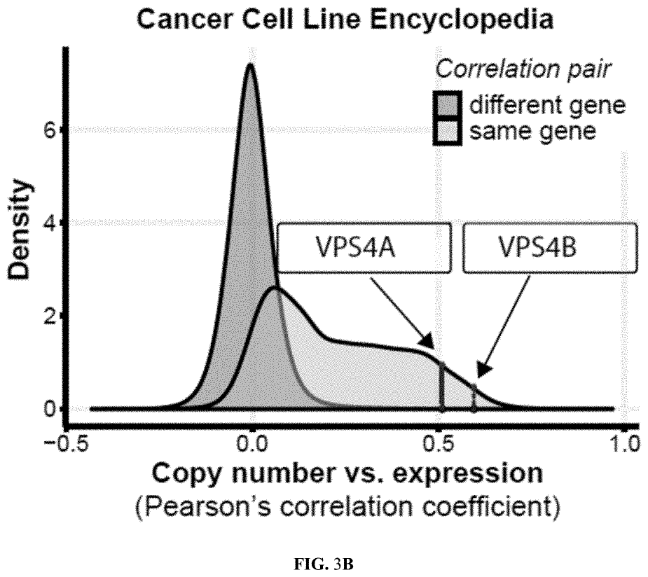

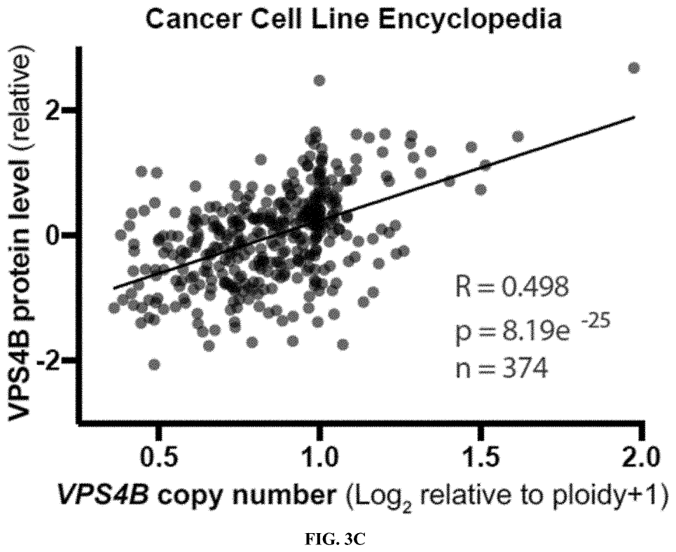

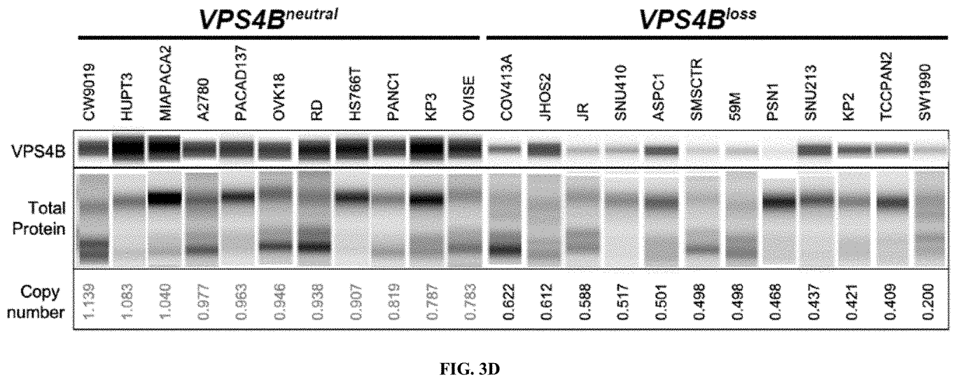

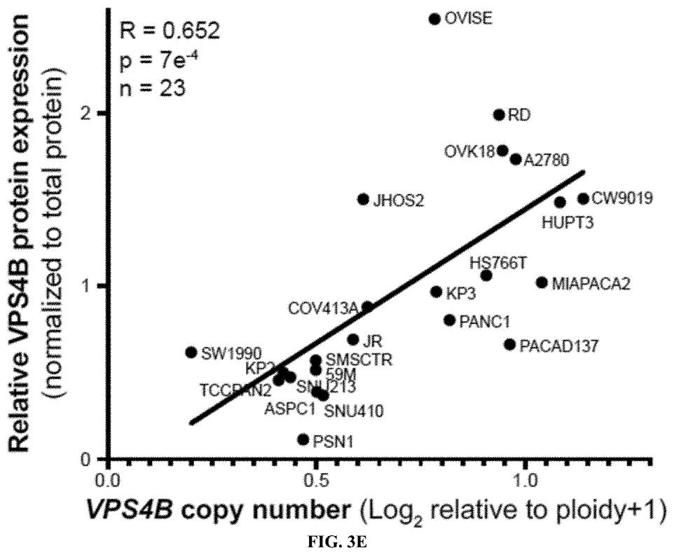

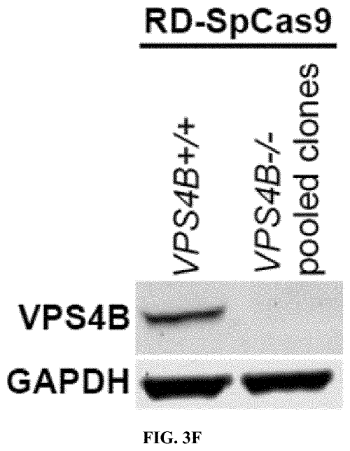

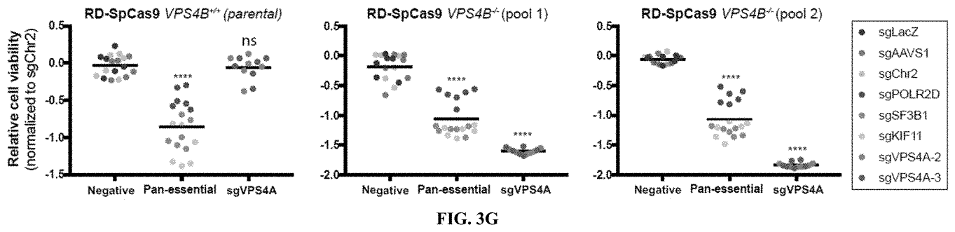

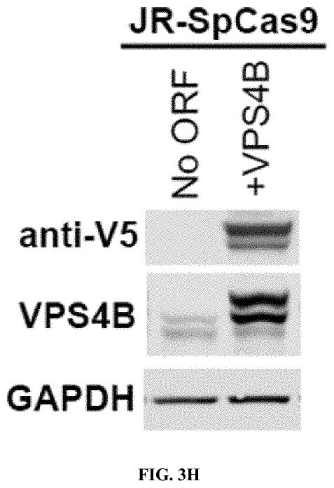

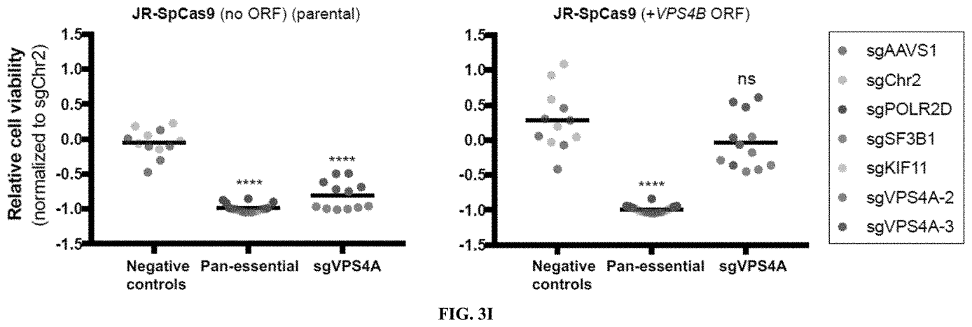

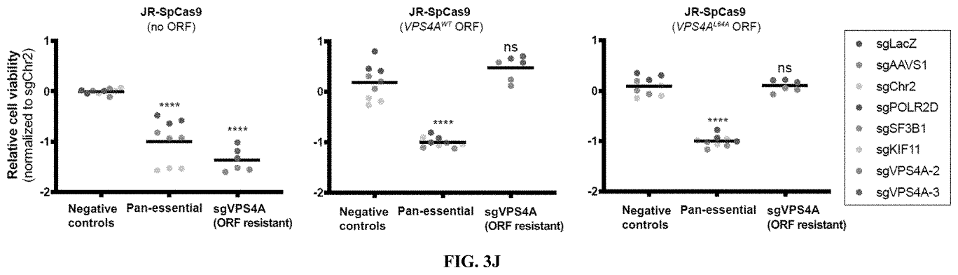

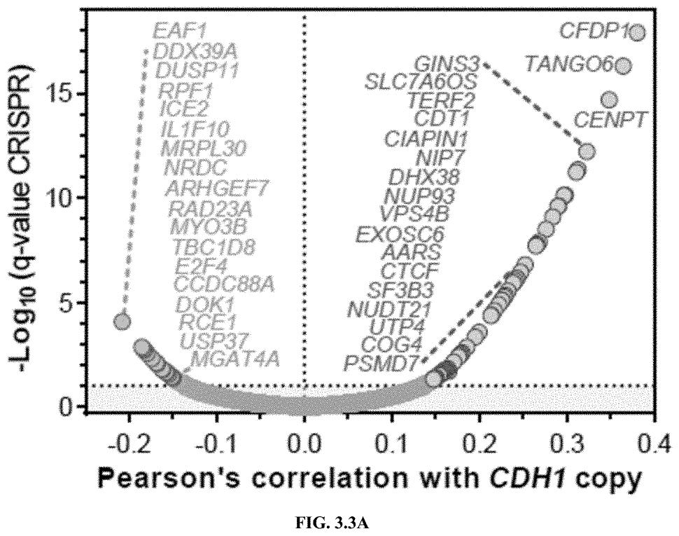

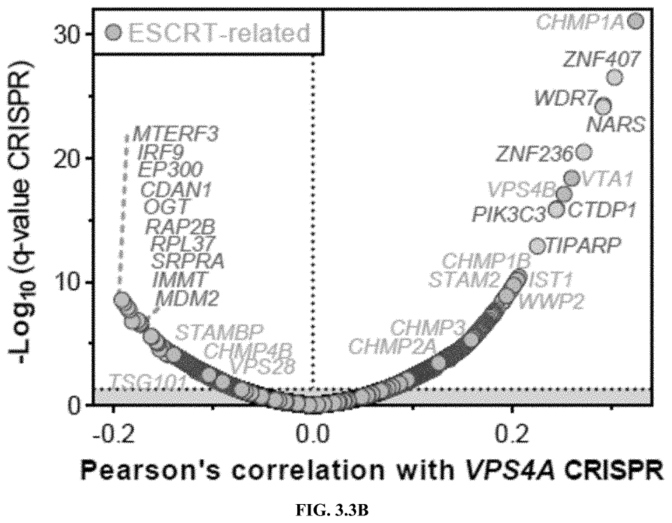

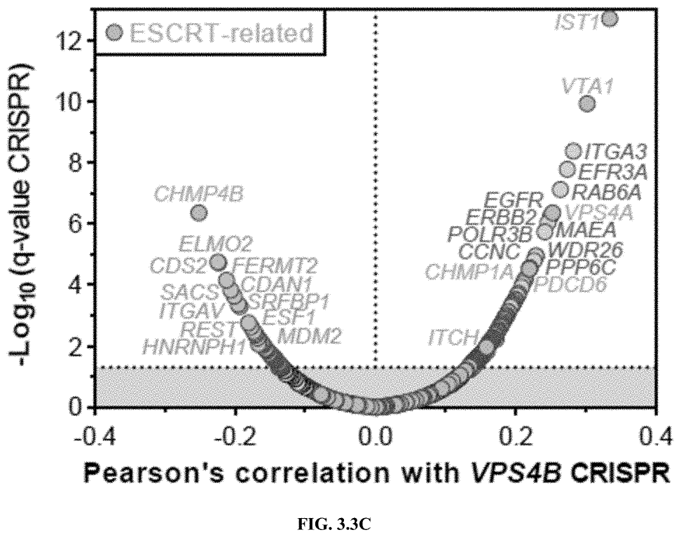

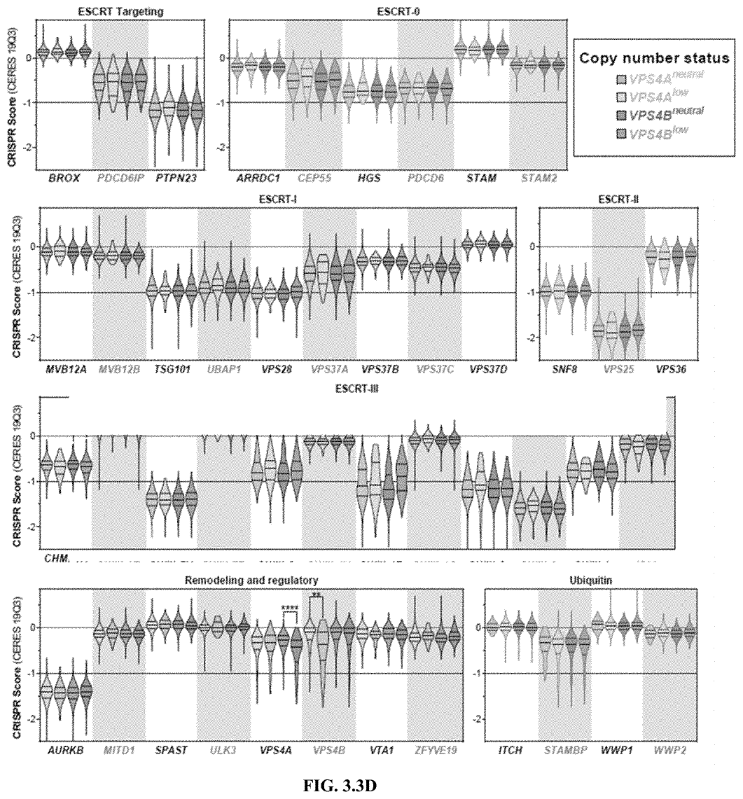

Correlation of VPS4A and VPS4B CRISPR dependency scores with CRISPR dependency scores for all other genes highlighted that VPS4A and VPS4B are co-essential with other specialized ESCRT genes such as CHMP1A, VTA1 and IST1. Conversely, dependency on CHMP4B anticorrelated with VPS4A/VPS4B dependency (FIGS. 3.3A-3.3D) (Vietri et al. Nature Review Molecular Cell Biology, 21:25-42, 2020). As expected, the CHMP1A and CHMP4B dependency scores also demonstrated significant interactions with VPS4A/B copy number loss (FIG. 3.3D). In summary, these results demonstrate a critical role for the ESCRT pathway in maintaining cancer cell survival and highlight synthetic lethal vulnerabilities in the context of genomic loss of ESCRT pathway members.

### Example 3: VPS4A and VPS4B Undergo Frequent Copy Loss Across Both Adult and Pediatric Cancer Types

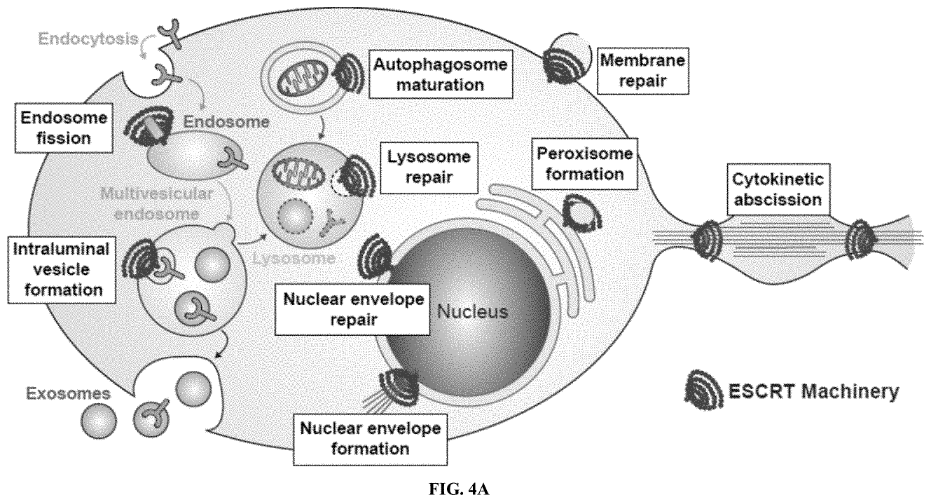

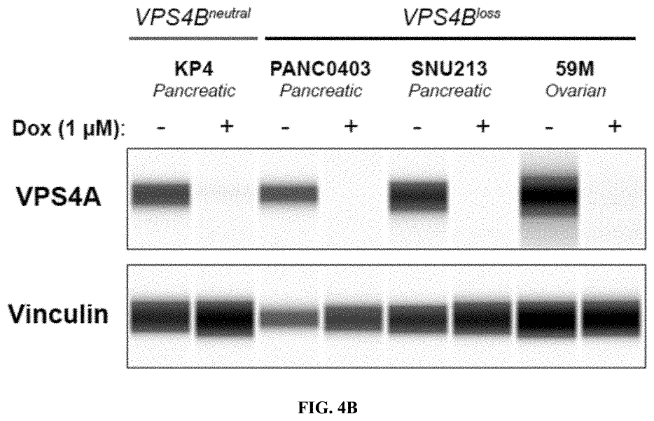

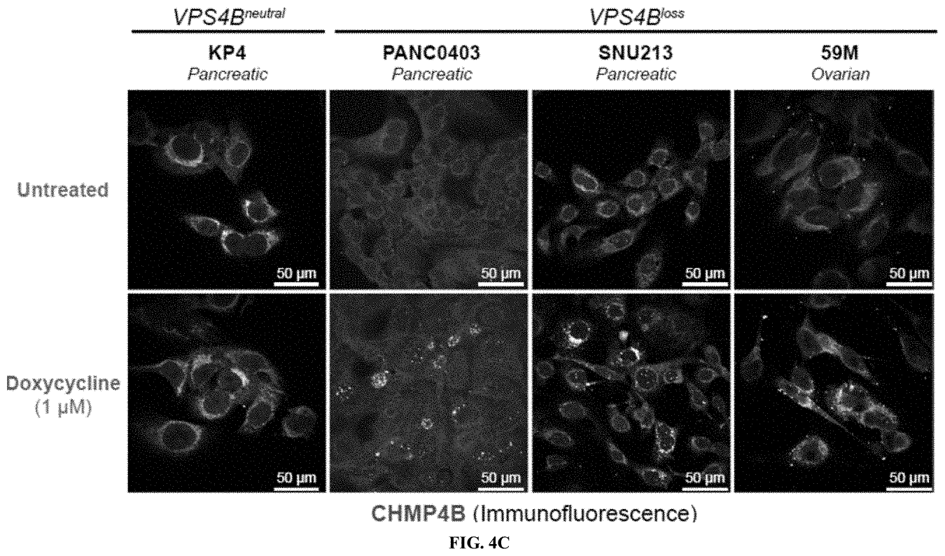

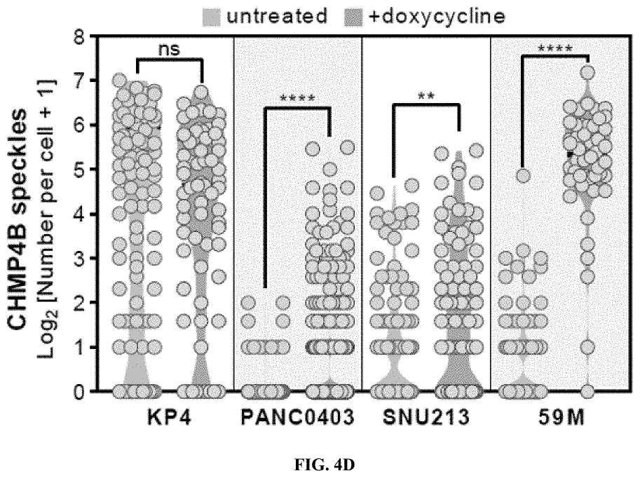

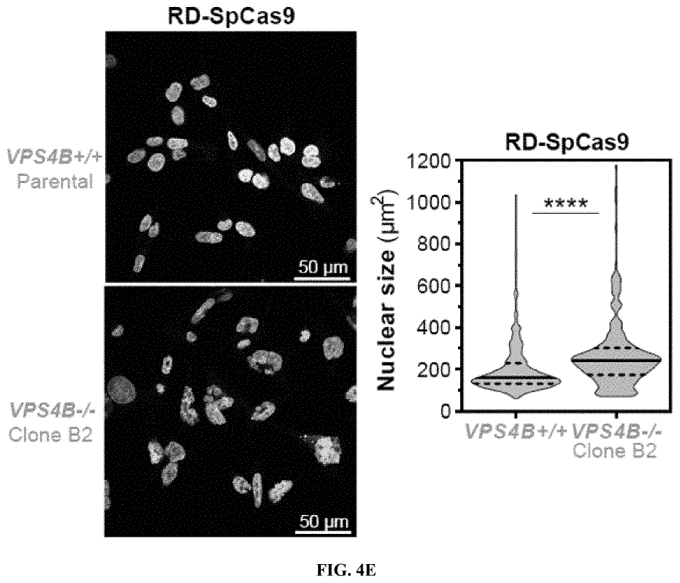

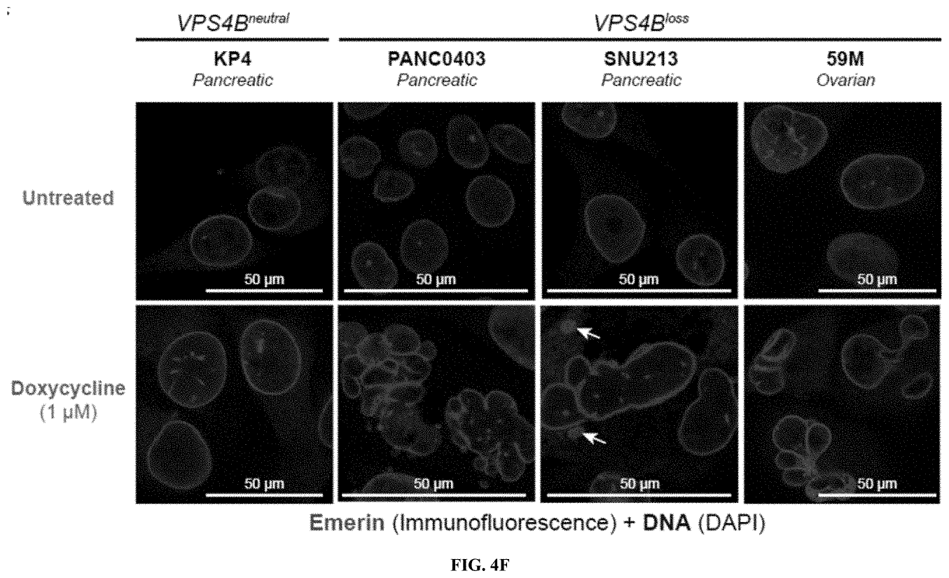

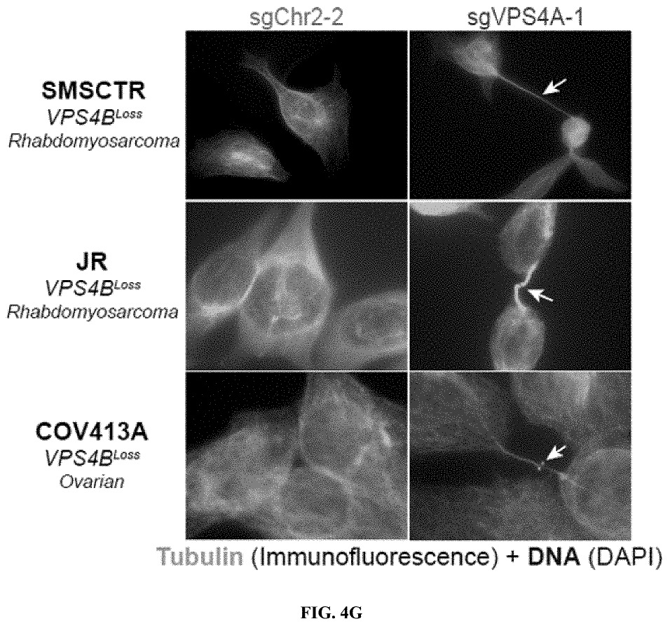

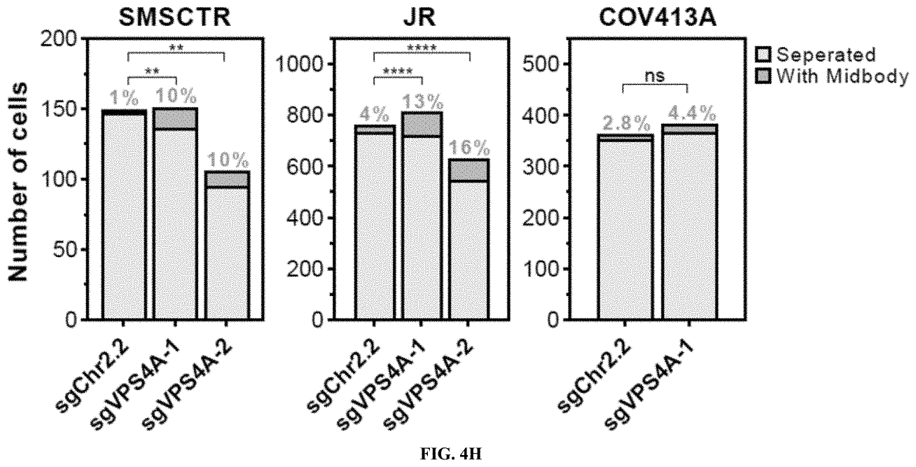

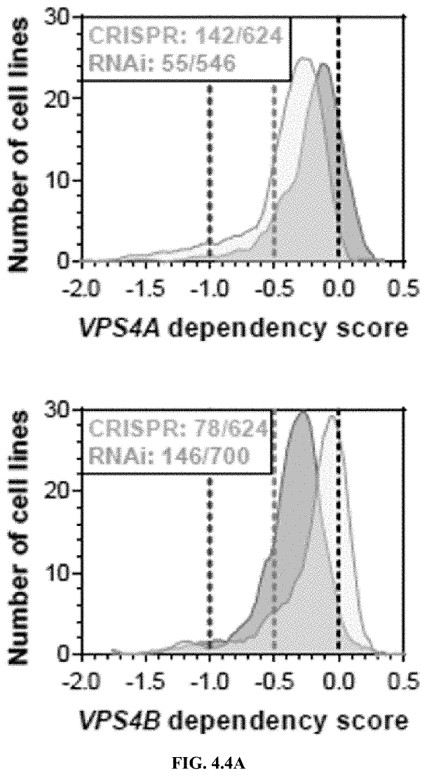

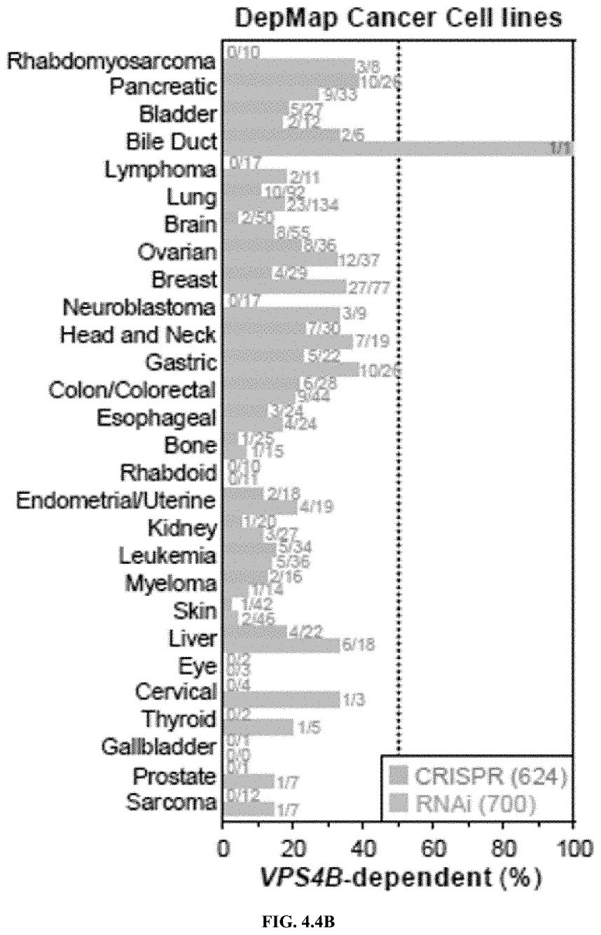

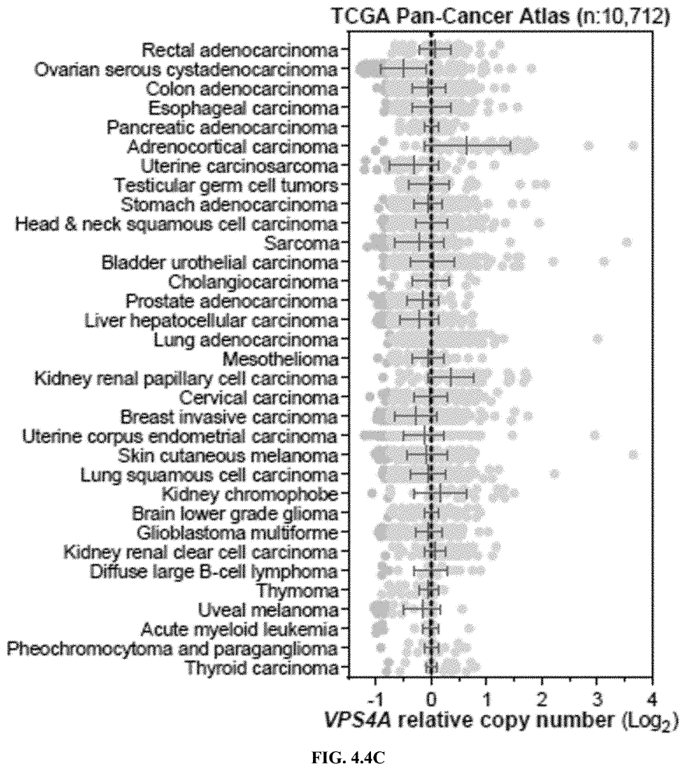

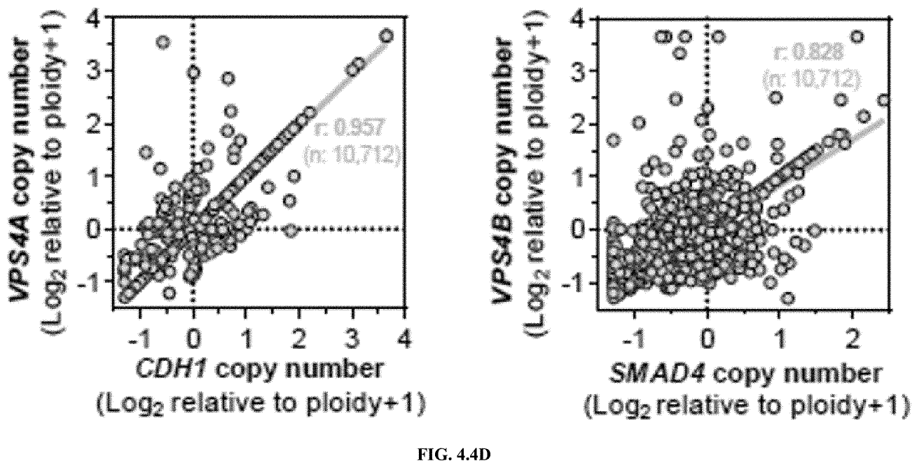

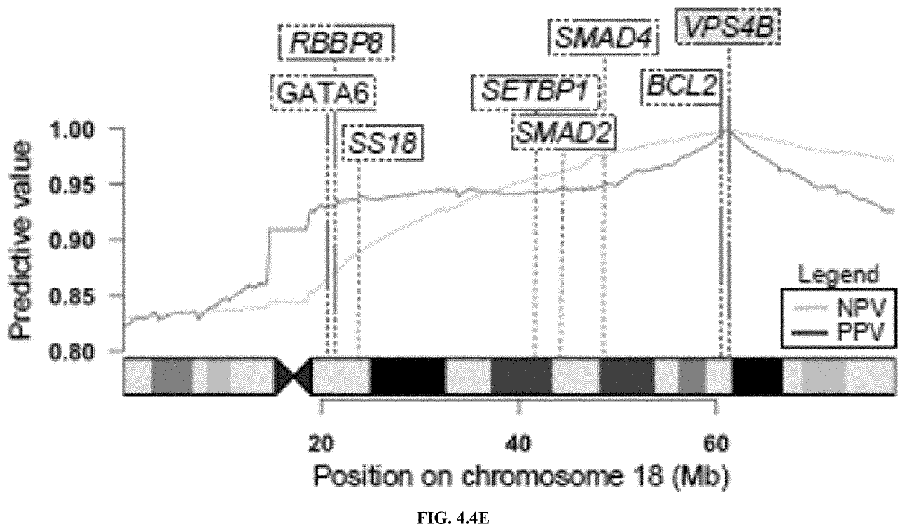

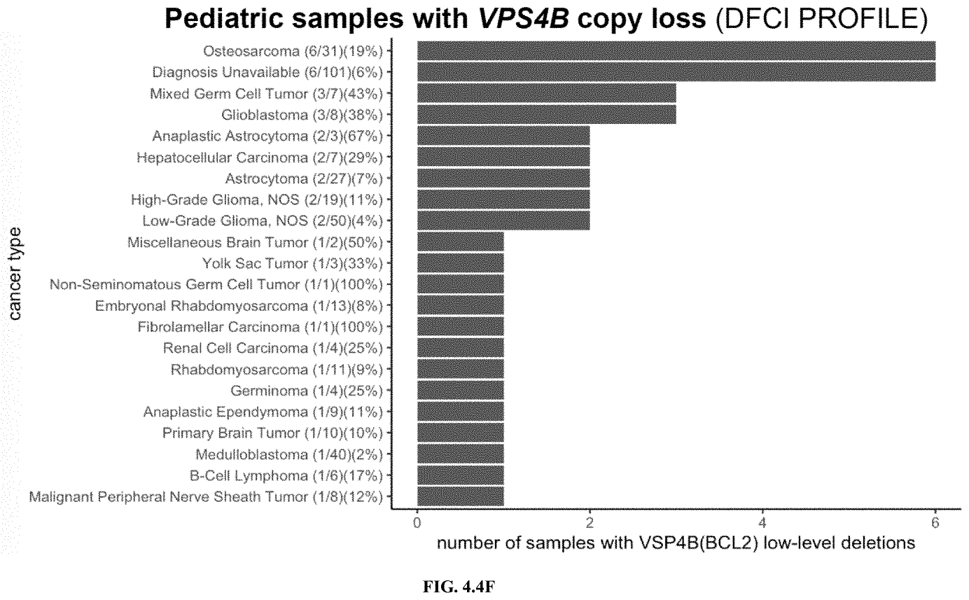

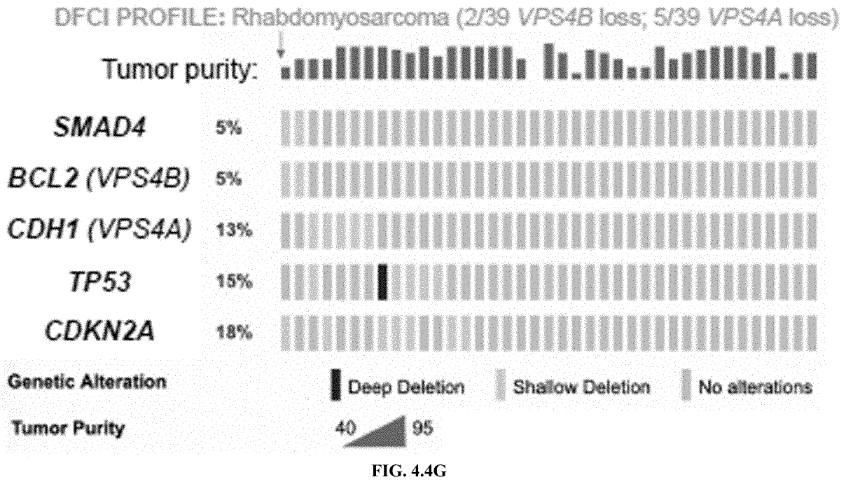

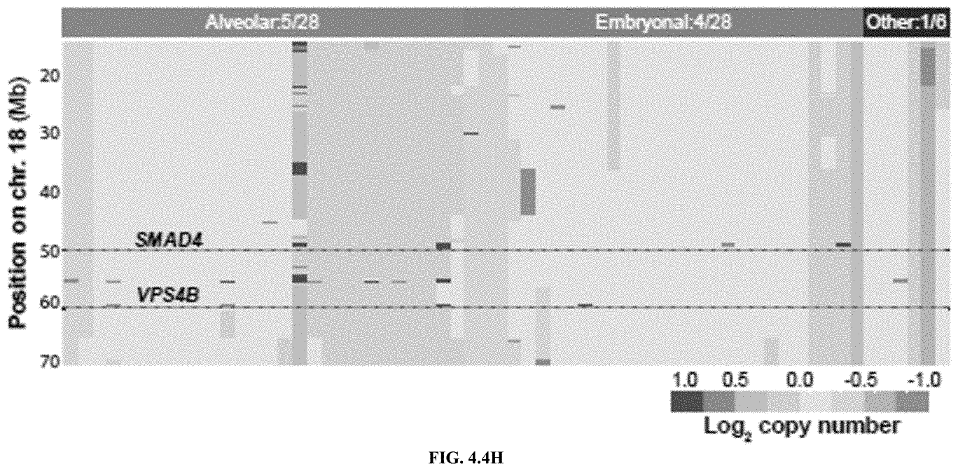

Across datasets, 22.7% (142/624) of cancer cell lines screened by CRISPR-SpCas9 and 10.0% (55/546) of those screened by RNAi depended on VPS4A for proliferation and survival (FIG. 4.4A upper panel), with over 40% of pancreatic cancer and pediatric rhabdomyosarcoma (RMS) cancer cell lines demonstrating robust dependence (FIG. 1K). Additionally, VPS4A was essential in a substantial fraction of bladder, bile duct, lung, ovarian, colon and esophageal cancer cell lines (FIG. 1K). For VPS4B, 12.5% (78/624) of cancer cell lines screened by CRISPR-SpCas9 and 20.9% (146/700) of those screened by RNAi were dependent on VPS4B (FIG. 4.4A, lower panel), with over 25% of ovarian, breast, pancreatic, liver, gastric and bile duct cancer cell lines demonstrating strong dependency (FIG. 4.4B).

To confirm the relevance of these findings beyond cancer cell lines, the frequency of VPS4A/B copy loss was examined in patient tumor samples from The Cancer Genome Atlas (TCGA) Pan-Cancer copy number dataset (Taylor et al. Cancer Cell, 33(4):676-689, 2018). In 10,712 adult cancers, VPS4B copy loss occurred in 33% (3,546/10,712) of cancers (FIG. 1L). In particular cancers known to exhibit SMAD4 loss, such as esophageal, colorectal and pancreatic cancers, showed frequent VPS4B loss. For example, VPS4B was lost in 47.5% of pancreatic ductal adenocarcinoma samples. Across the same TCGA dataset, VPS4A copy loss occurred in 27.1% of tumors, with common loss in ovarian, uterine and sarcoma samples (FIG. 4.4C). As expected, strong correlations were observed across the TCGA samples between VPS4B and SMAD4 copy number, as well as for VPS4A and CDH1 copy number (FIG. 4.4D).

To expand the analysis to pediatric samples, the Dana-Farber Cancer Institute patient targeted sequencing database was surveyed (Oncopanel/PROFILE) (Sanchez-Vega et al. Cell, 173(2):321-337, 2018). VPS4B copy number was inferred from copy calls through targeted sequencing of the neighboring BCL2 gene, ˜70 Kb away (FIG. 4.4E). 955 pediatric cancer samples were identified with a minimum neoplastic cellularity of 20% and 42 samples were defined by clear VPS4B copy loss (3.9%) (FIG. 4.4F). Notable VPS4B copy loss was observed in 40% of germ-cell tumors (6/15), 19% of osteosarcomas (6/31) and 9% of brain tumors (16/176). Given that pediatric RMS cancer cell lines were the most enriched for VPS4A dependency (FIG. 1K), the DFCI PROFILE RMS cohort and a published RMS cohort of 41 pediatric patients were investigated. It was observed that 5% (2/39; DFCI PROFILE RMS) and 16% (10/62; published cohort (Chen et al. Cancer Cell 24(6):710-724, 2013) of RMS tumors harbored partial VPS4B copy loss, which was not subtype-specific (FIGS. 4.4G and 4.4H).

Taken together, these data indicate that VPS4A and VPS4B copy loss occur in both adult and pediatric tumors from many different lineages and suggest that over a third of all human cancers may depend on VPS4A or VPS4B for survival. Given the robust synthetic lethal interaction between VPS4A and VPS4B/SMAD4 loss and the prominence of VPS4B/SMAD4 loss across cancer, focus was placed on subsequent validation and mechanistic studies on VPS4A as a vulnerability in the context of VPS4B copy loss.

### Example 4: VPS4A Validated as a Strong Genetic Dependency in Cancer Cells with Copy Loss of VPS4B

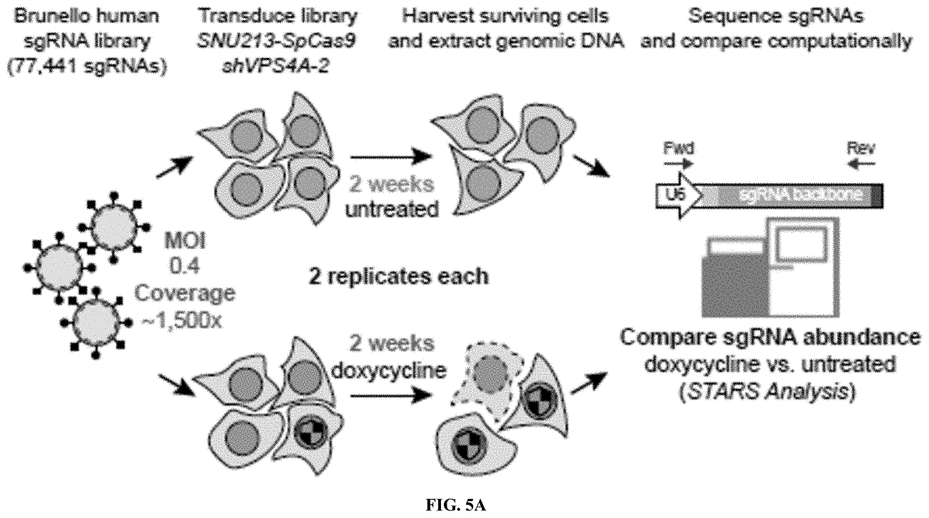

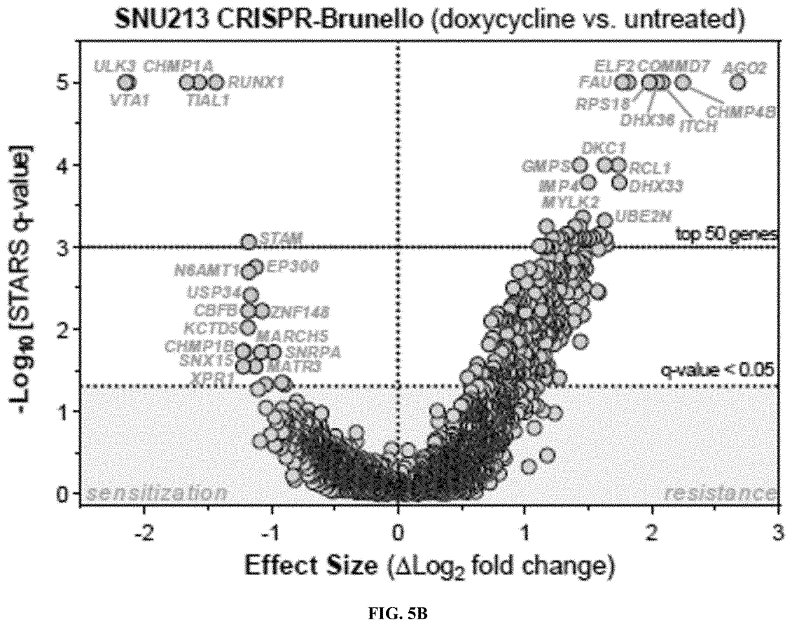

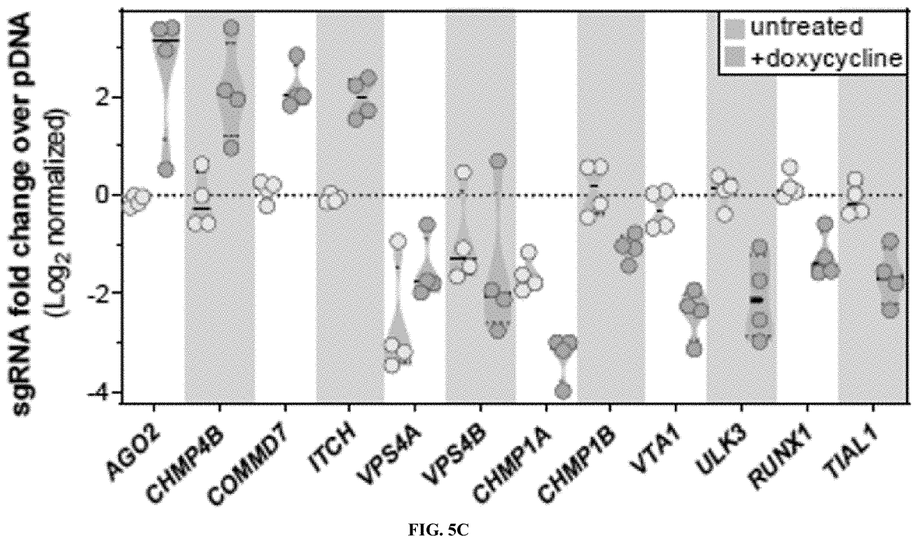

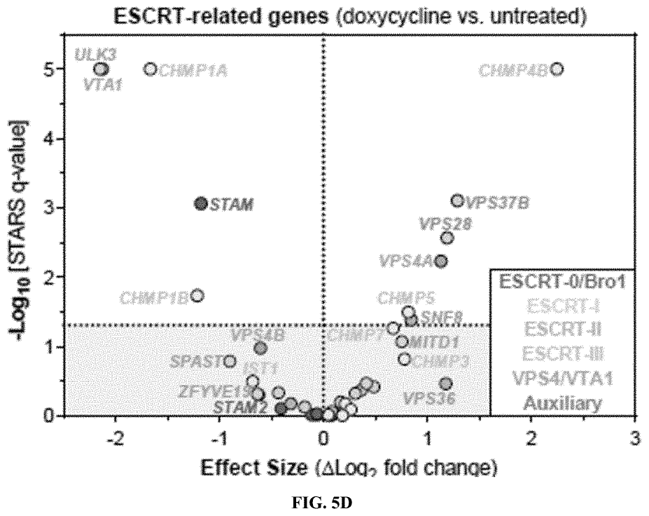

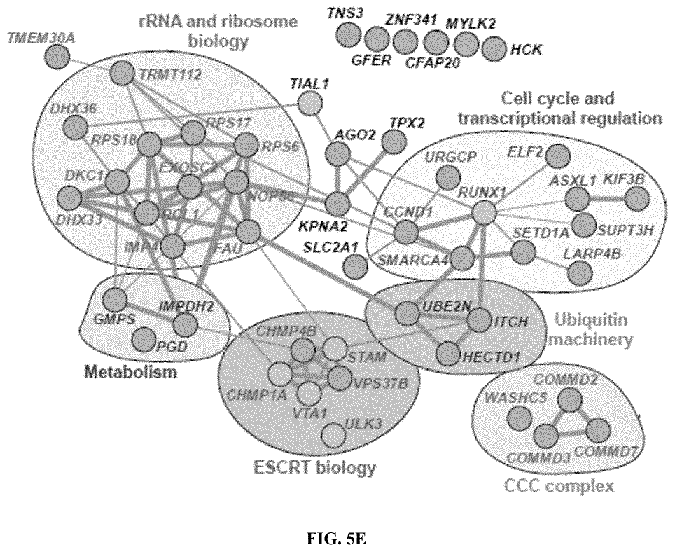

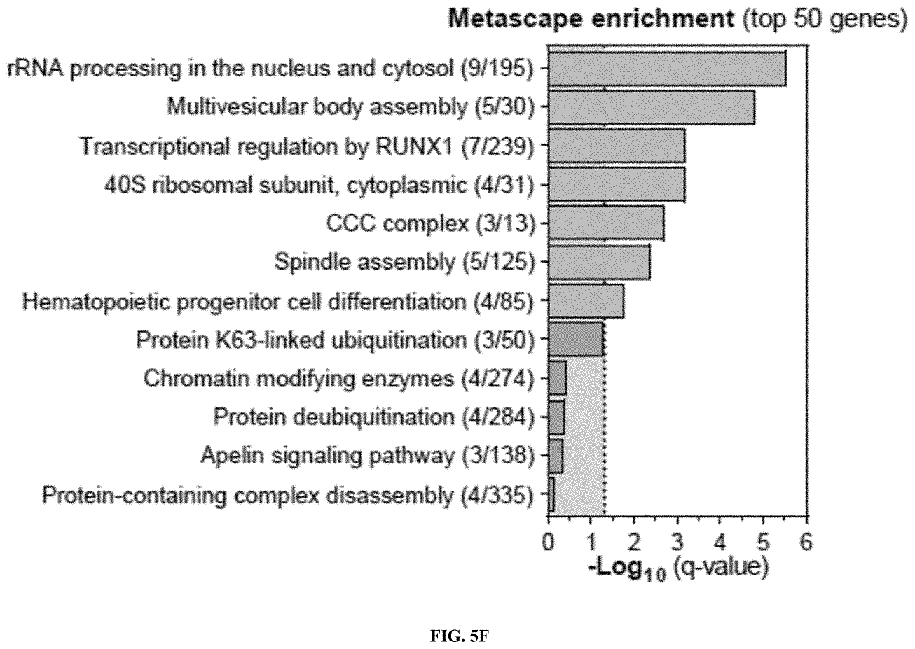

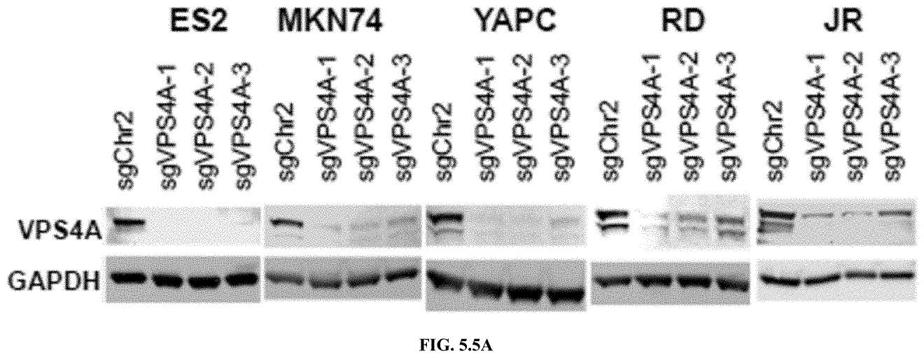

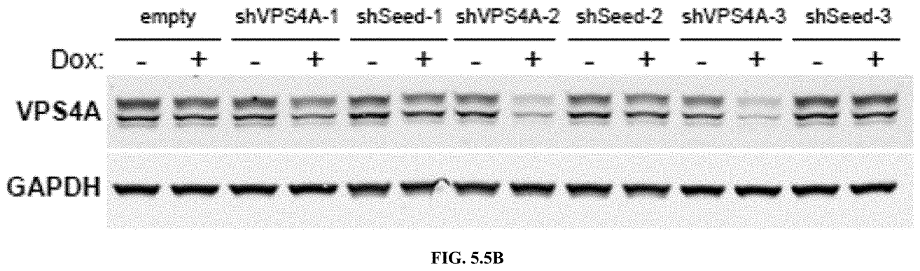

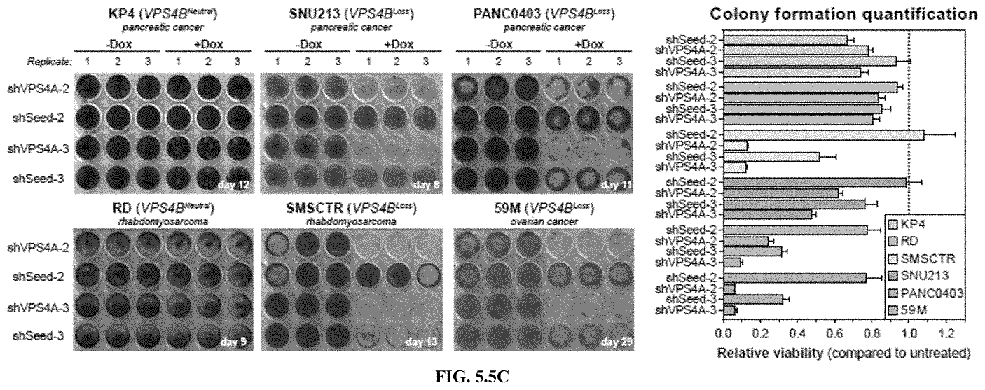

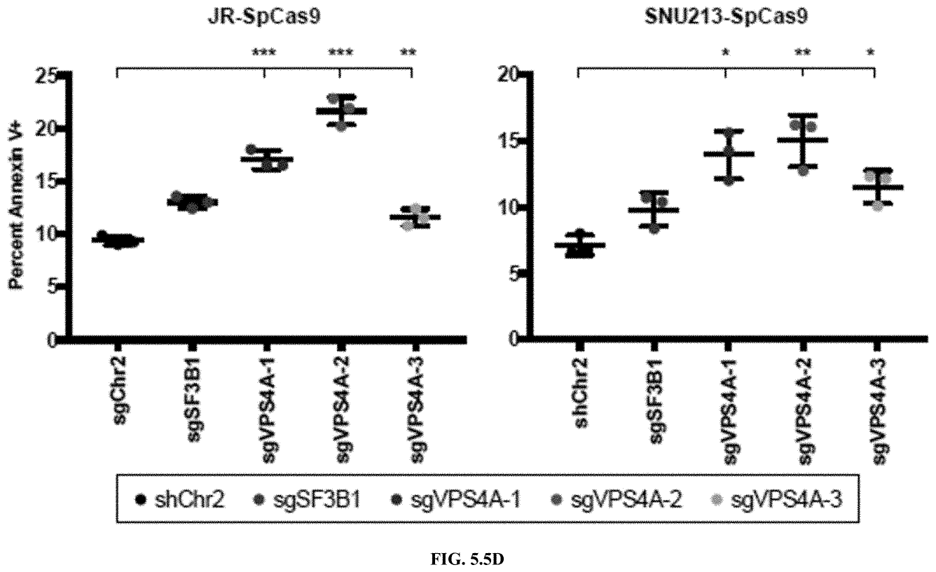

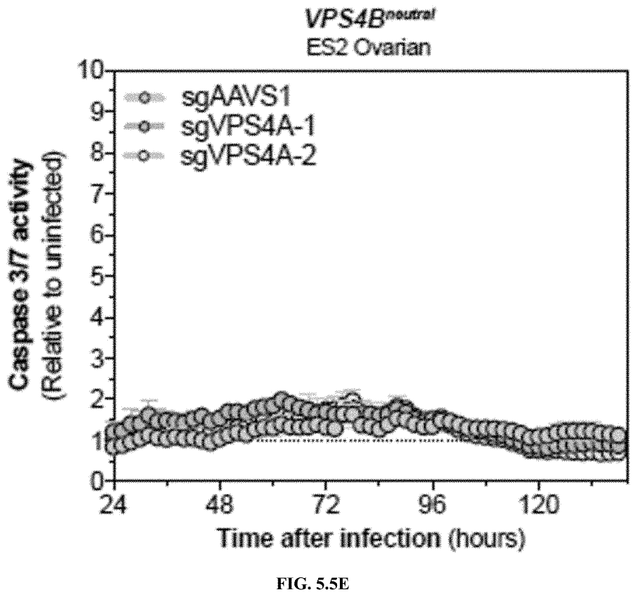

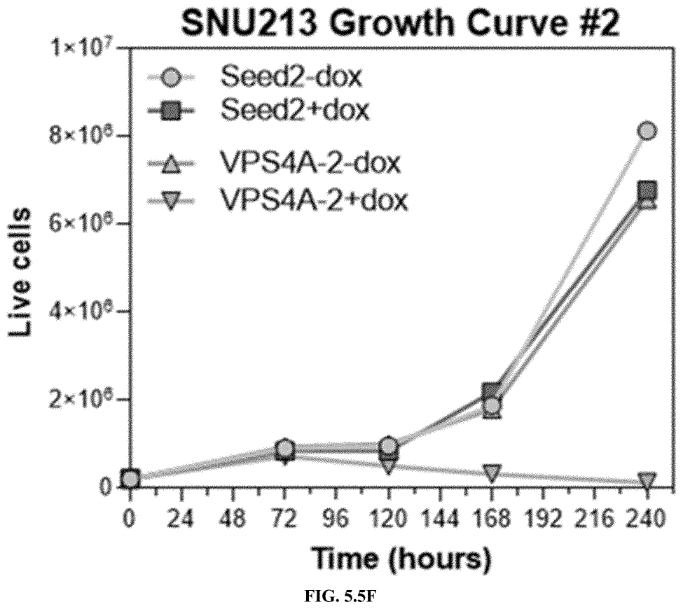

To confirm whether VPS4A inactivation can selectively kill cells with copy loss of VPS4B, it was examined if cancer cells with partial VPS4B copy loss (VPS4B^{loss} cells) were more sensitive to VPS4A ablation than cells without VPS4B copy number alterations (VPS4B^{neutral} cells). CRISPR-SpCas9-mediated knockout of VPS4A by 3 different sgRNAs were first evaluated, which confirmed their ability to ablate VPS4A expression by immunoblotting (FIG. 5.5A). Next, the effect of CRISPR-SpCas9-mediated VPS4A knockout on viability was measured for 8 VPS4B^{neutral} and 10 VPS4B^{loss} cell lines using a luminescence-based readout of cellular ATP seven days after lentiviral transduction. As expected, cell viability was significantly decreased in VPS4B^{loss} cells infected with any of the 3 sgRNAs targeting VPS4A (FIG. 2A). To corroborate these results, RNAi-mediated suppression of VPS4A expression was used. 3 VPS4A shRNAs and their paired C9-11 seed controls were evaluated for their ability to selectively suppress VPS4A expression using a doxycycline inducible RNAi system. C9-11 seed controls carry mutations at positions 9-11 of the shRNA to abrogate “on target” knockdown but retain “off-target” RNAi seed effects. shVPS4A-2 and the corresponding shSeed2 control were selected for use in further experiments based on their optimal knockdown effects and minimal off-target seed effects (FIG. 5.5B). Using this pair, RNAi-mediated suppression of VPS4A profoundly reduced proliferation of VPS4B^{loss}, but not VPS4B^{neutral}, cancer cell lines in cell viability and long-term colony formation assays (FIGS. 2B and 2C, FIG. 5.5C).

Based on the reduction of cell viability after VPS4A suppression in vitro, it was next asked whether VPS4A suppression could impair the growth of established tumor xenografts. Subcutaneous mouse xenografts were established using human VPS4B^{loss} cancer cell lines for Rhabdomyosarcoma (SMSCTR) and pancreatic ductal adenocarcinoma (SNU213). These cell lines were stably transduced with doxycycline-inducible shVPS4A-2 or negative control C9-11 RNAi systems. In response to induction of VPS4A suppression, but not that of the C9-11 control, established tumor xenografts exhibited near-complete regression (FIGS. 2D, 2E, 2G and 2H), resulting in marked prolongation of survival in both models (FIG. 2E, median survival 30 vs. 74 days, SMSCTR; FIG. 2H, median survival 21 vs. 63 days, SNU213). Immunoblotting confirmed VPS4A knockdown in shVPS4A-2 tumors, and not in C9-11 or untreated controls (FIG. 2F).

To better understand what drives loss of cell viability in VPS4A-dependent cancer cells, in vitro assays were performed to characterize apoptosis and cell cycle distribution (FIG. 2H-2J and FIG. 5.5D). Upon CRISPR-SpCas9-mediated disruption of VPS4A, significant induction of Caspase 3/7 activity, indicative of apoptosis, across RMS, head and neck, pancreatic and ovarian cancer cell lines with VPS4B^{loss} loss (FIG. 2I and FIG. 5.5D) were observed. In contrast, no apoptosis induction was observed in the VPS4B^{neutral} ovarian cancer cell line ES2 after VPS4A disruption (FIG. 5.5E). Besides apoptosis, consistent G2/M arrest upon VPS4A ablation in VPS4B^{loss} cancer cells was also observed (FIG. 2H).

Combined, these CRISPR and RNAi validation experiments demonstrate that VPS4A is critical for proliferation and survival of cancer cells with genomic copy loss of VPS4B.

Overexpression of VPS4B was found to rescue the dependent JR rhabdomyosarcoma cell line from CRISPR-mediated knockout of VPS4A (FIG. 12B). Points in the viability plot indicate technical replicates. Asterisks indicate a p-value <0.005 (unpaired Welch's t-test). CRISPR-mediated knockout of VPS4B sensitizes the non-dependent RD rhabdomyosarcoma cell line to suppression of VPS4A. Points in the viability plot indicate technical replicates. Asterisks indicate a p-value <0.0001 (unpaired Welch's t-test).

### Example 5: Altered VPS4B Expression Modulates VPS4A Dependency in Cancer Cells

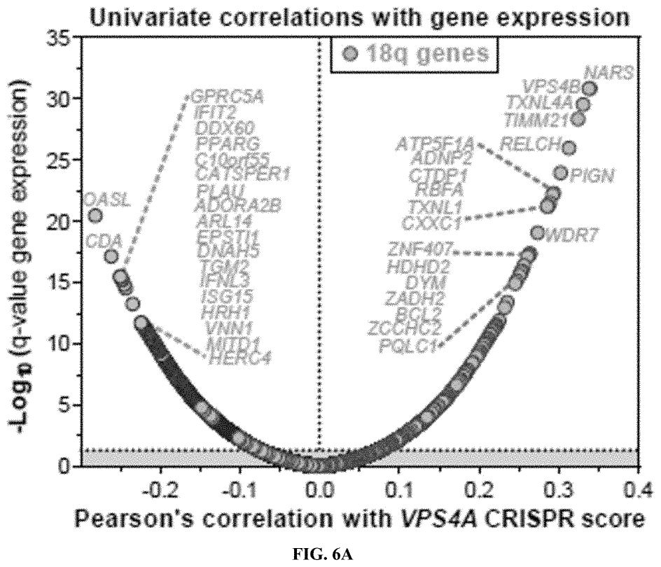

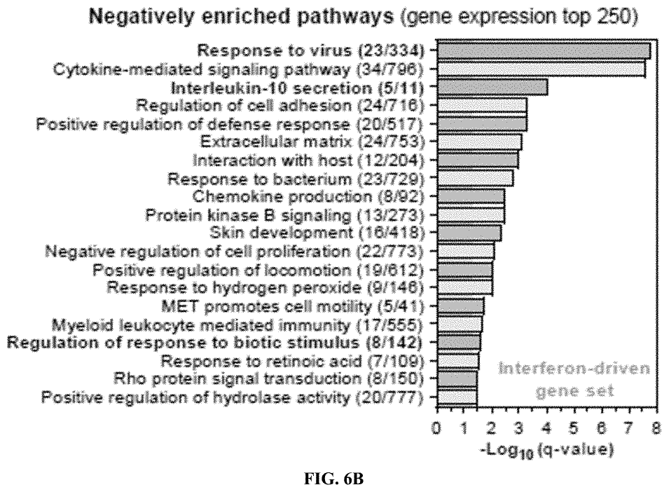

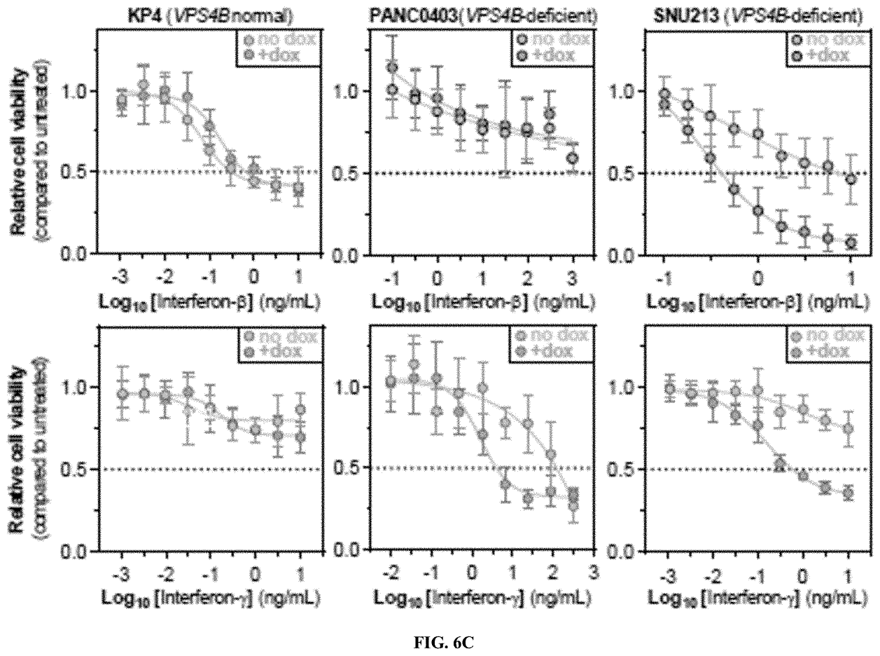

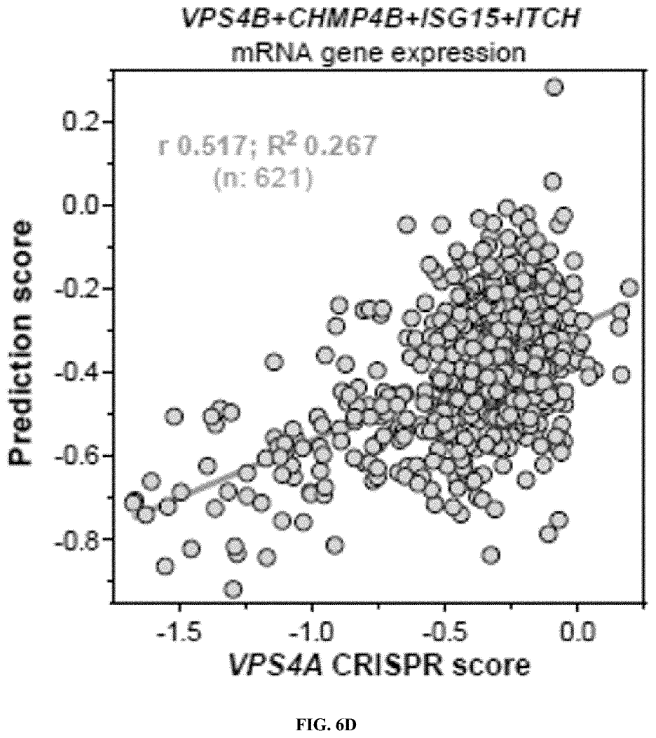

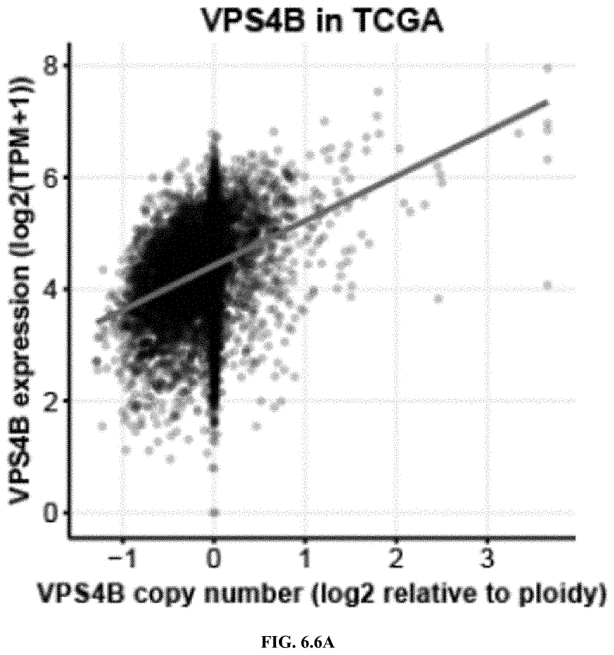

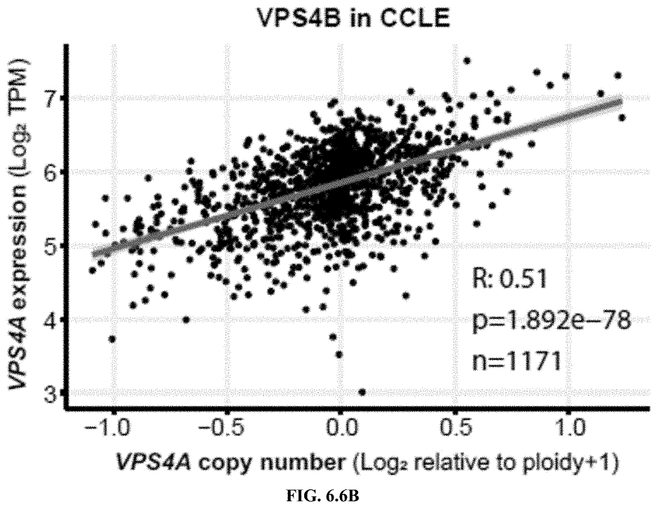

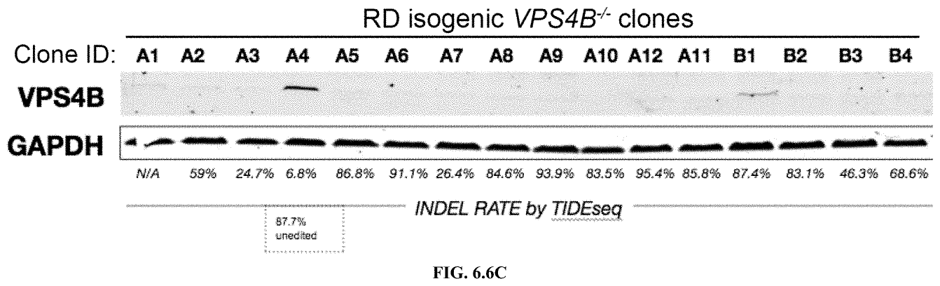

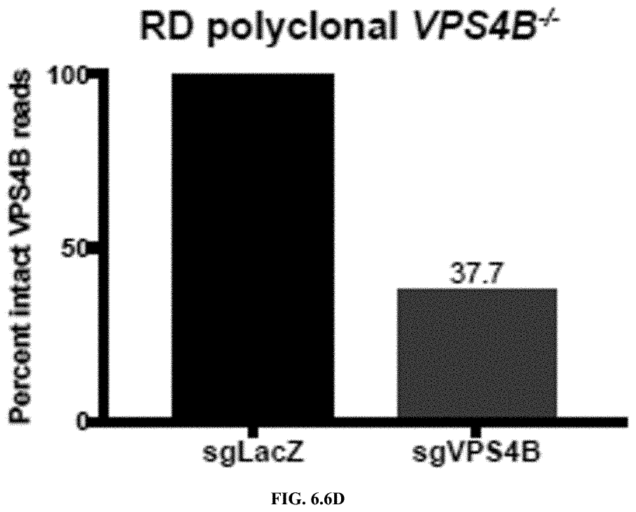

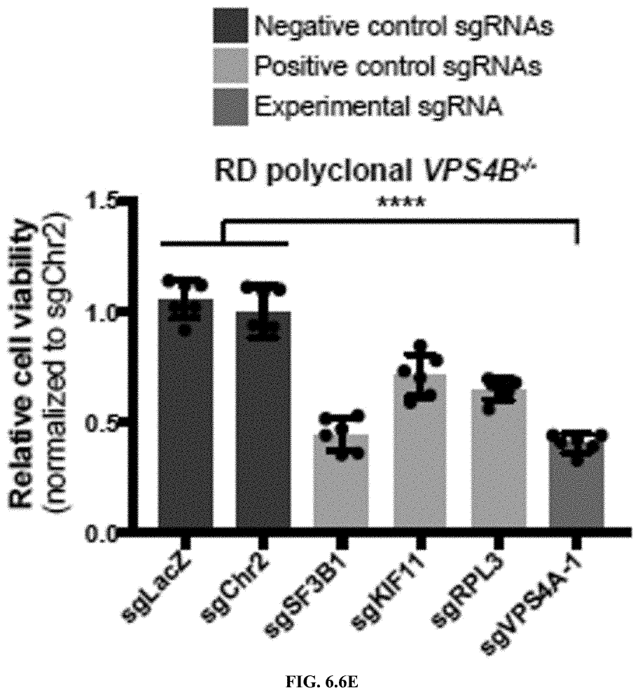

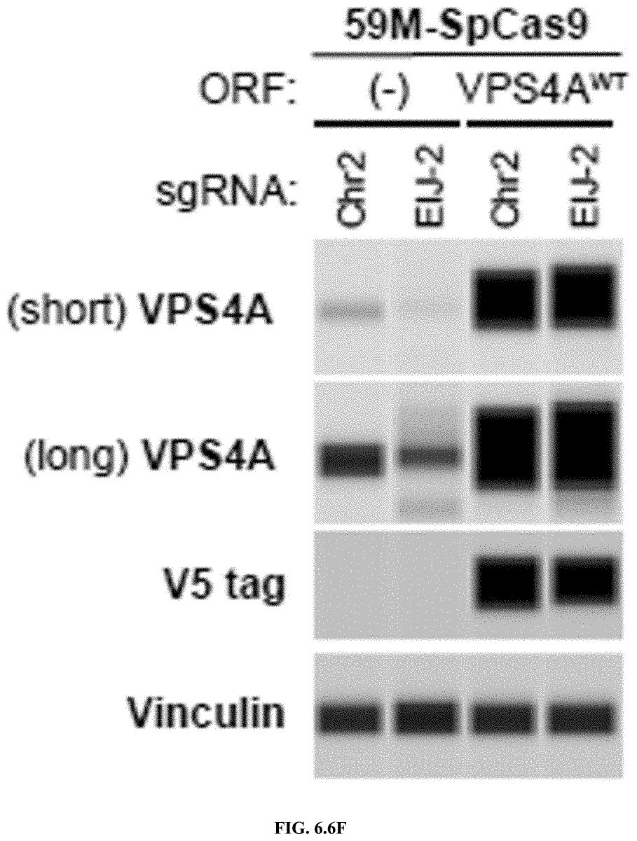

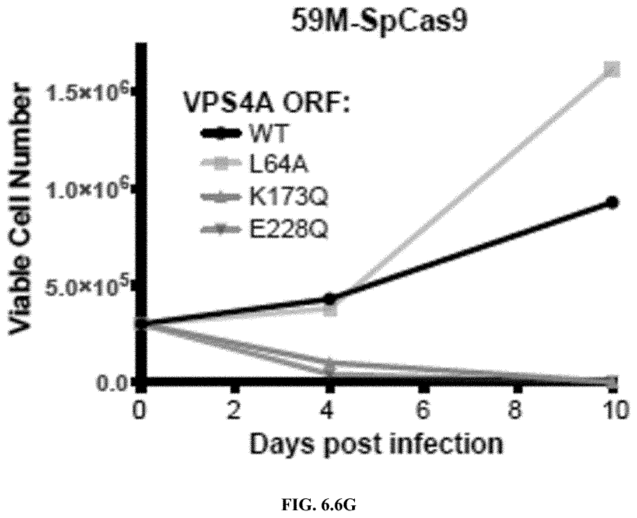

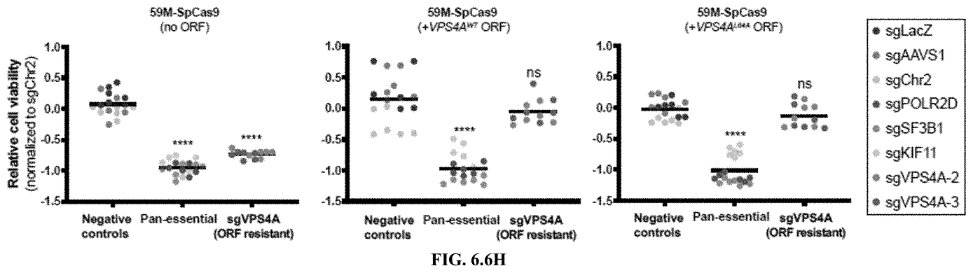

To investigate whether VPS4B copy loss reduces VPS4B mRNA and protein levels, VPS4B expression (RNAseq) was first correlated with copy number across 1,171 cell lines from the Cancer Cell Line Encyclopedia (CCLE) and 10,712 TCGA patient samples. In both datasets, VPS4B expression strongly correlated with VPS4B copy number (FIGS. 3A, 3B and 6.6A), indicating that gene dosage drives VPS4B expression and that VPS4B^{loss} cells express less VPS4B than VPS4B^{neutral} cells. Similar findings were obtained for VPS4A (FIGS. 3B and 6.6B). Next, quantitative proteomics data from 374 cancer cell lines was examined, and a significant decrease was observed in VPS4B protein expression in VPS4B^{loss} cells (FIG. 3C). Reduced VPS4B protein levels in VPS4B^{loss} cells were independently confirmed by quantitative Protein Simple capillary-based immunodetection of VPS4B in 23 cell lines (FIGS. 3D and 3E; n: 11 VPS4B^{neutral} and n: 12 VPS4B^{loss}).

To ascertain if reduction in VPS4B expression sensitizes VPS4B^{neutral} cells to VPS4A depletion, CRISPR-SpCas9 was used to knockout VPS4B in the VPS4B^{neutral} non-dependent RMS cancer cell line RD. 16 single-cell derived monoclonal cultures were screened for VPS4B ablation using immunoblot and TIDEseq, achieving knockout in 14/16 (FIG. 3F and FIG. 6.6C) https://paperpile.com/c/lzErE/u0oaa. Eight VPS4B^{-/-} monoclonal cultures were mixed into two distinct pools of four clones and tested each pool's tolerability to CRISPR-SpCas9-mediated knockout of VPS4A. Upon VPS4A ablation, VPS4B^{-/-} clone pools showed substantially reduced viability compared to negative controls, indicating that loss of VPS4B was enough to confer dependency on VPS4A in these cells (FIG. 3G). Similar results were observed with the original polyclonal VPS4B knockout cultures (FIGS. 6.6D and 6.6E). To determine if enhanced VPS4B expression can rescue cells from VPS4A dependency, VPS4B was stably overexpressed in the VPS4B^{loss} cell line JR (FIG. 3H). VPS4B overexpression was sufficient to rescue cells from VPS4A suppression (FIG. 3I). Combined, these results demonstrate that VPS4B expression levels modulate dependency on VPS4A.

Exogenous rescue experiments were performed to evaluate the ability of wild-type and loss-of-function VPS4A alleles to rescue VPS4B^{loss} cancer cells from VPS4A dependency and to confirm the specificity of the VPS4A sgRNAs. sgVPS4A-2 and sgVPS4A-3, which targeted intron-exon junctions, were used to inactivate endogenously, but not exogenously, expressed VPS4A variants (FIG. 6.6F). Stable expression was attempted of four different VPS4A variants: wild-type VPS4A^{WT}; VPS4A^{L64A}, which prevents binding of the microtubule interacting and transport (MIT) domain to ESCRT-III filaments https://paperpile.com/c/lzEreE/R0TCS; VPS4A^{K173Q}, which cannot bind ATP; and VPS4A^{E228Q}, which cannot hydrolyze ATP. Stable expression of either ATP mutant (VPS4A^{K173Q} or VPS4A^{E228Q}) was not compatible with long term cell culture (FIG. 6.6G). Toxicity associated with VPS4 ATPase mutants is consistent with reports of dominant-negative functions for these mutants (Fujita, 2003) and indicates that impairment of ATP binding or hydrolysis functionally inactivates not only the mutant VPS4A protein, but also co-expressed wild-type VPS4A or VPS4B proteins. Interestingly, both VPS4A^{WT} and VPS4A^{L64A} constructs could fully rescue cell viability upon disruption of endogenous VPS4A by CRISPR-SpCas9 in both JR and 59M cell lines (FIG. 3J and FIG. 6.6H). Rescue by the VPS4A^{L64A} mutant suggests that MIT domain interactions with ESCRT-III filaments are not required to rescue viability after VPS4A depletion.

### Example 6: VPS4A Suppression Leads to ESCRT-III Filament Accumulation, Deformed Nuclei and Abscission Defects in VPS4B^{loss} Cancer Cells

Suppression of VPS4 has been shown to affect ESCRT-III-mediated membrane remodeling and fission in human cells and results in impairment of several cellular functions, including mitotic spindle formation, maturation of autophagosomes, secretion of extracellular vesicles, DNA damage and cellular abscission. How VPS4A suppression alters ESCRT function in insensitive (VPS4B^{neutral}) and sensitive (VPS4B^{loss}) cancer cell lines were investigated using immunofluorescence to study known ESCRT-dependent cellular processes (FIG. 4A). First, the core ESCRT-III subunit CHMP4B was visualized by confocal immunofluorescence imaging after suppression of VPS4A. After 6 days of VPS4A suppression (FIG. 4B), significantly increased formation of bright CHMP4B speckles in the cytoplasm and the nucleus of sensitive cell lines (PANC0403, SNU213 and 59M) was observed, but not in the VPS4B^{neutral} cell line KP4 (FIGS. 4C-4D), indicating that loss of VPS4A/B function leads to accumulation of CHMP4B filaments.

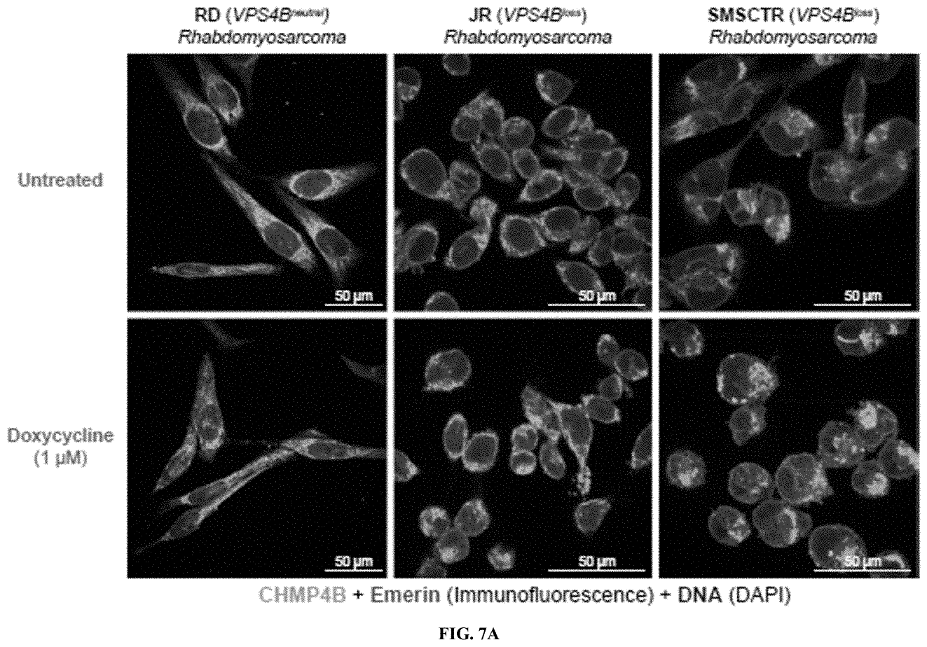

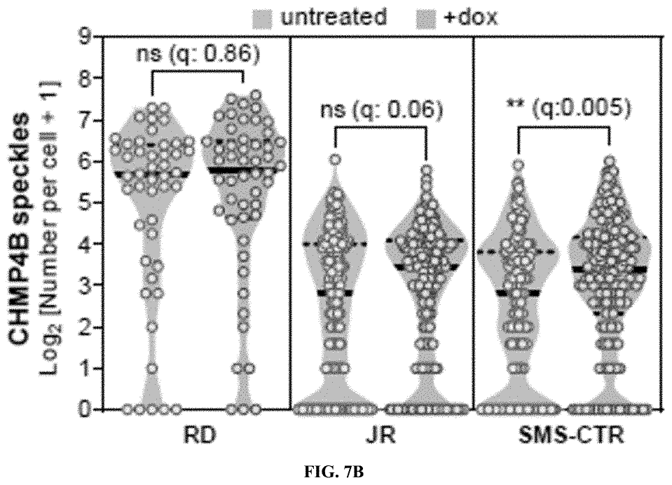

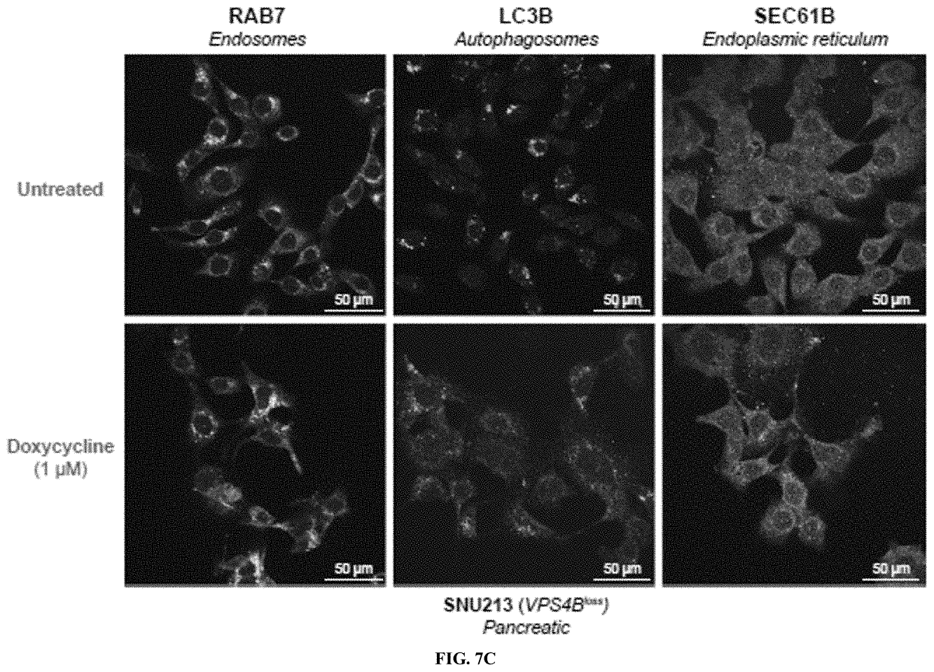

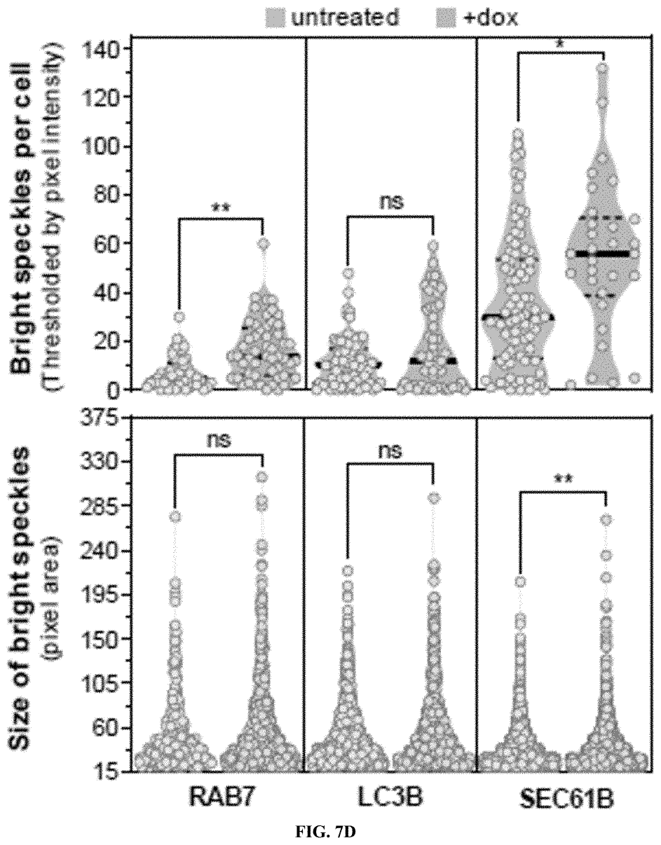

Upon VPS4A suppression, nuclear deformation and enlargement in VPS4B^{loss} cancer cells was also noticed. This phenotype was also observed in the VPS4B^{-/-} monoclonal RD cell lines previously created (FIGS. 3F-3G, 6.6C), even without VPS4A suppression (FIG. 4E). To further investigate this phenotype, the nuclear DNA was visualized using DAPI and the inner nuclear membrane by Emerin staining. Sustained VPS4A suppression led to pronounced deformation of the nuclear compartment in VPS4B^{loss} pancreatic and ovarian cancer cells, as evidenced by the presence of multi-lobed, fragmented nuclear structures and the presence of micronuclei and multinucleation (FIG. 4F). When an additional panel of 3 rhabdomyosarcoma lines (RD, JR and SMSCTR) were examined, these skeletal muscle-derived cells showed higher baseline CHMP4B expression with clear filament formation and some nuclear deformation and multinucleation (FIG. 7A). After 5-day suppression of VPS4A, a slight increase was observed in cytoplasmic CHMP4B speckles and nuclear deformation in VPS4B^{loss} JR and SMSCTR cells, while no obvious changes were observed in the VPS4B^{neutral} RD cells (FIG. 7B).

As the ESCRT machinery is required for cytoplasmic vesicle trafficking, it also investigated whether VPS4A suppression induced alterations in cytoplasmic membrane structures in the VPS4B^{loss} SNU213 cancer cell line. By immunofluorescence visualization of RAB7 (endosomes), LC3B (autophagosomes) and SEC61B (endoplasmic reticulum), it was observed that sustained VPS4A suppression for 7 days induced changes in endosomal and endoplasmic reticulum structures but did not significantly alter the amount or size of autophagosomes (FIGS. 7C and 7D).

Finally, cancer cells undergoing VPS4A suppression were observed still attached to other cells through long cytokinetic bridges, indicative of abscission defects. As the ESCRT and VPS4 machinery play a crucial role in cellular abscission, this phenotype was further investigated by immunofluorescence staining of tubulin four days after CRISPR-SpCas9-mediated knockout of VPS4A (FIG. 4G). Cytokinetic bridging was evident upon visual inspection in VPS4B^{loss} cancer cells of multiple lineages, and quantification revealed a significantly increased fraction of VPS4B cancer cells were still connected to a neighboring cell by a cytokinetic bridge (FIG. 4H).

FIG. 11 identifies the downstream effects of VPS4A suppression on a VPS4A-dependent cancer cell line, SNU213. VPS4A suppression by an inducible shRNA against VPS4A lead to deformation of the nuclear envelope (Emerin) and nucleus (DAPI) and to cytoplasmic accumulation of ESCRT-III CHMP4B-containing filaments in pancreatic SNU213 cancer cells. While a CRISPR knockout of VPS4A triggered abscission defects in the VPS4A-dependent rhabdomyosarcoma JR cancer cell line (Tubulin+DAPI).

### Example 7: CRISPR-SpCas9 Screening for Modifiers of VPS4A Dependency Reveals an Important Role for ESCRT Proteins and the ULK3 Abscission Checkpoint Kinase

Since the ESCRT pathway is involved in several processes essential for cancer cell survival, a genome-scale CRISPR-SpCas9 loss-of-function screen was performed in combination with RNAi-mediated silencing of VPS4A to map suppressors and enhancers of cancer cell death elicited upon VPS4A suppression. For this purpose, the SNU213 pancreatic cancer cell line was utilized, which harbors VPS4B copy loss and is dependent on VPS4A (FIGS. 2A-2C and FIG. 5.5C). The Brunello sgRNA library (Doench et al. Nature Biotechnol, 34(2):184-191, 2016) was tonsduced into SNU213 cells stably expressing SpCas9 and the dox-inducible shVPS4A-2 RNAi system and conducted the screen in the presence or absence of doxycycline (FIG. 5A). After passaging, gene-level scores were calculated using the STARS algorithm (Doench et al., 2016) and the values were compared between screens to identify genes that promote resistance or enhanced sensitivity to VPS4A suppression (FIG. 5B, Table 5). Table 5 describes the results from the CRISPR-SpCas9 modifier screen with untreated and doxycycline treated SNU213 cells expressing the inducible shVPS4A-2 RNAi system. Included are sgRNA normalized read counts, sgRNA and gene-level log 2 fold changes, results of the STARS analysis.

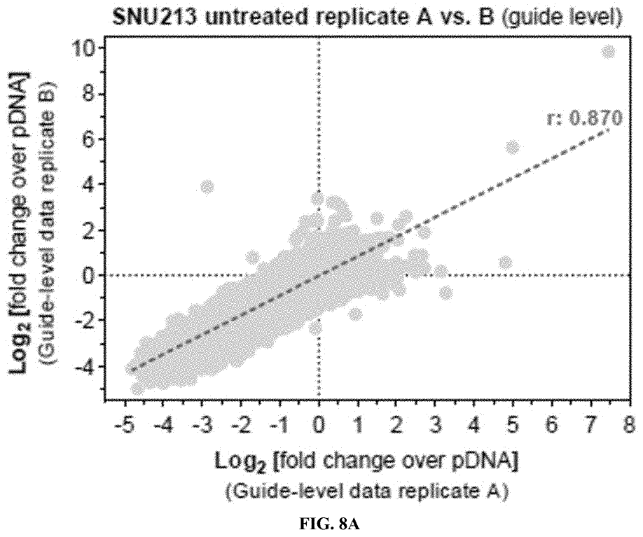

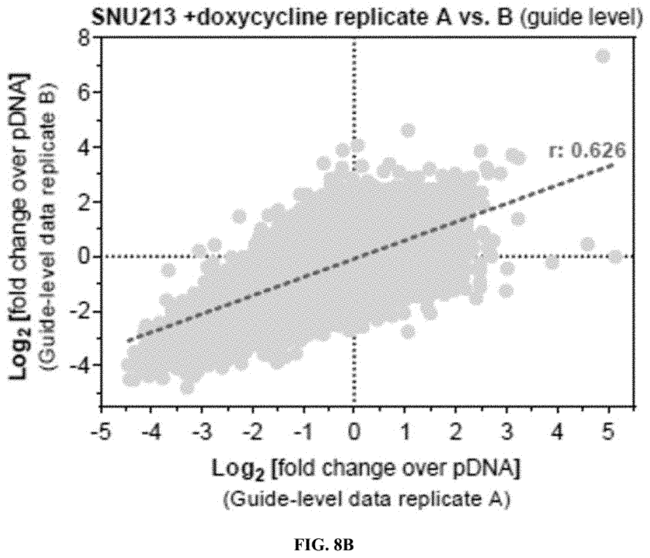

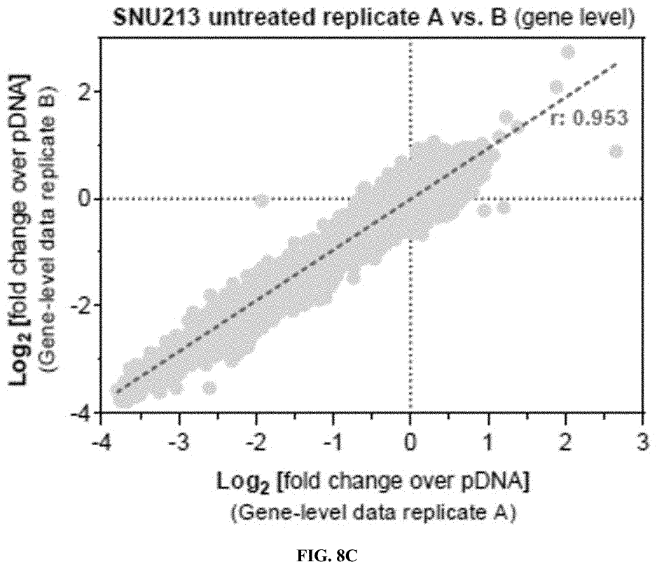

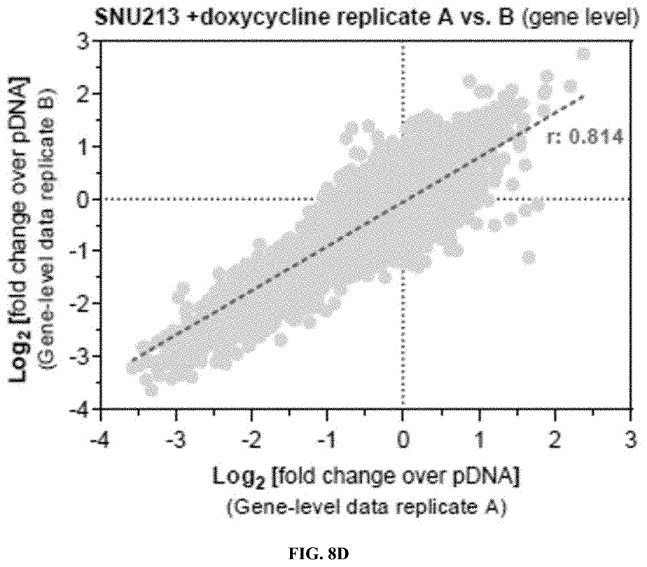

Replicate guide and gene-level scores strongly correlated, indicating robust screening performance (FIGS. 8A-8D).

sgRNAs enriched in doxycycline-treated (VPS4A-suppressed) cells indicate resistance genes for which knockout promotes cell survival in the presence of RNAi-mediated VPS4A suppression (FIG. 5B, points to the right of the center dotted line and above the grey-shaded area). The genes promoting cell proliferation and viability during VPS4A suppression (e.g., ELF2, COMMD7, AGO2, FAU, RPS18, CHMP4B, ITCH, DKC1, RL1, MYLK2) are found above the grey-shaded area and to the left of the central dotted line in FIG. 5B. Argonaute 2 protein (AGO2), an essential protein for RNAi-mediated gene silencing, was identified as the most highly enriched gene, further validating the robustness of the screen (FIGS. 5B and 5C). sgRNAs targeting CHMP4B scored as the second most enriched set of sgRNAs (FIGS. 5B-5E), supporting the notion that CHMP4B ESCRT-III filaments play a crucial role in mediating the mechanism of antiproliferation conferred by VPS4A suppression. sgRNAs targeting additional members of the ESCRT machinery were also significantly enriched and included sgRNAs targeting the ESCRT-I VPS28 and VPS37B, ESCRT-II SNF8, and ESCRT-III CHMP5 (FIGS. 5C-5E). Other top enriched target genes included the Itchy E3 ubiquitin ligase encoding ITCH, the endosome-associated and uncharacterized COMMD7 gene, the RNA G-quadruplex unfolding DEAH/RHA helicase DHX36, and the ETS family transcription factor ELF2 (FIGS. 5B and 5E). Enrichment was also observed of additional sgRNAs targeting various ribosomal and nucleoli genes, the endosome-associated COMMD2 and COMMD3 genes, and various metabolism-related genes such as GMPS, IMPDH2, and PGD (FIG. 5E, Table 5).

Strong depletion was observed of sgRNAs targeting numerous genes in doxycycline-treated (VPS4A suppressed) cells relative to the untreated cells (VPS4A expressed), indicating genes for which knockout results in a synthetic lethal interaction or a selective enhancement of the antiproliferative effect of VPS4A-suppression (FIG. 5B, see points found in the top and middle panels on the left of the effect size at 0, i.e., the vertical dotted line (e.g., ULK3, CHMP1A, VTA1, RUNX1, TIAL1)). Most notably, sgRNAs targeting the VPS4A/VPS4B complex cofactor VTA1, as well as the two accessory ESCRT-III filament genes CHMP1A and CHMP1B scored as strong sensitizers to VPS4A suppression (FIGS. 5B-5E). Next to VTA1, the ULK3 gene, which encodes an abscission checkpoint kinase, scored as the top synthetic lethal gene. Knockout of TIAL1, which encodes a splicing and apoptosis-related regulatory protein, and RUNX1, which encodes a transcriptional complex-core binding factor, also scored as potent sensitization mechanisms (FIGS. 5B and 5E). Gene-set enrichment analysis of the top 50 resistance and sensitizer genes from the screen defined many common pathways enriched among the top scoring genes from the screen (FIG. 5F).

### Example 8: Interferon Signaling and CHMP4B Expression Modulate VPS4A Dependency

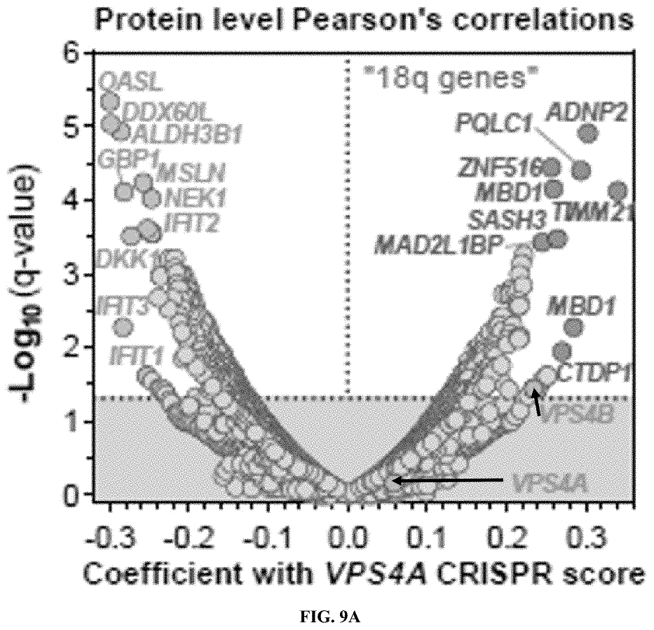

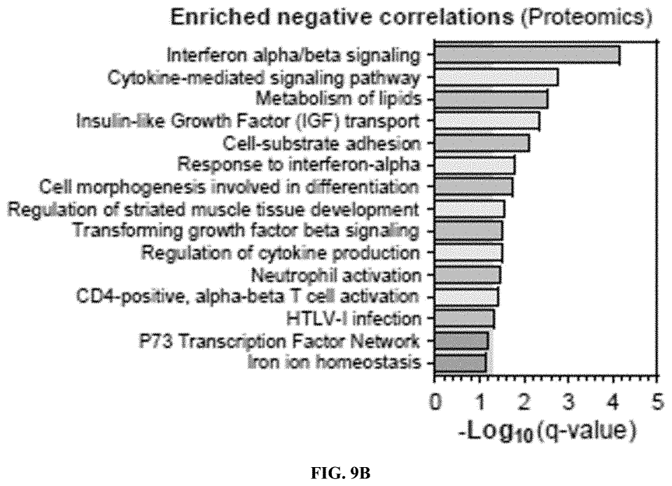

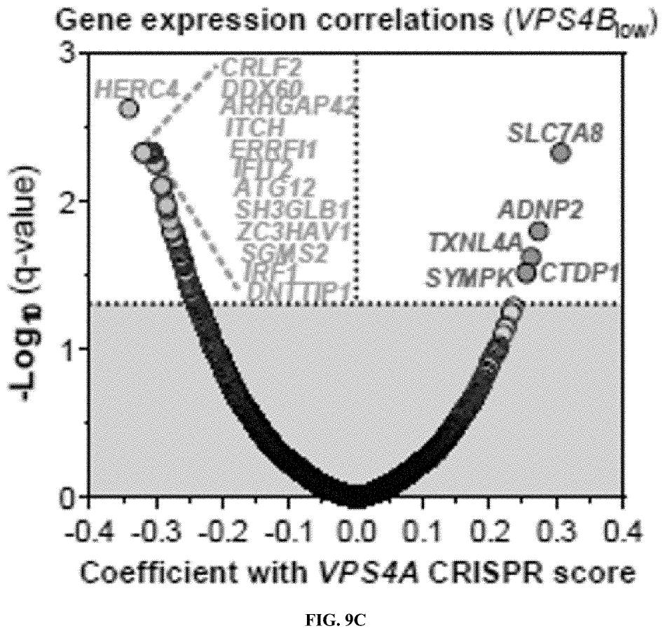

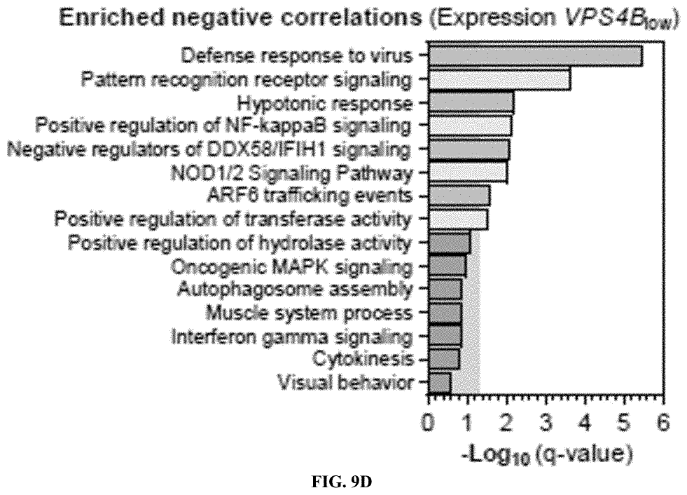

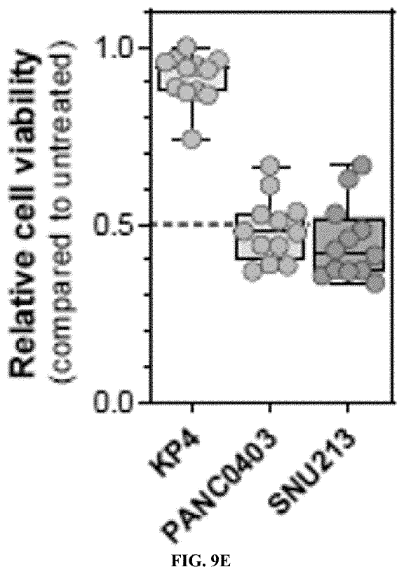

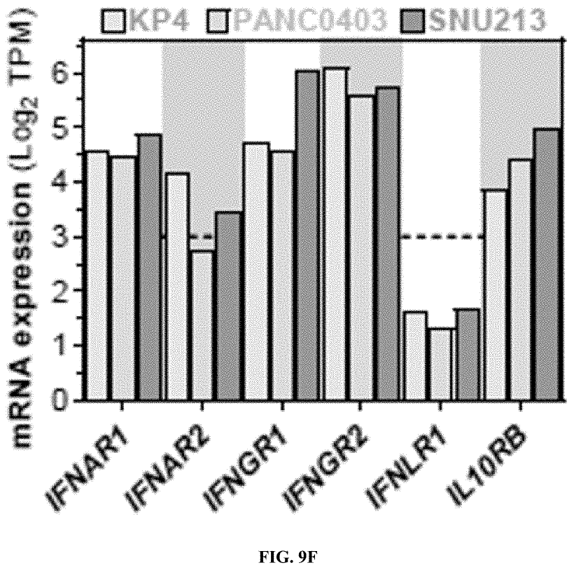

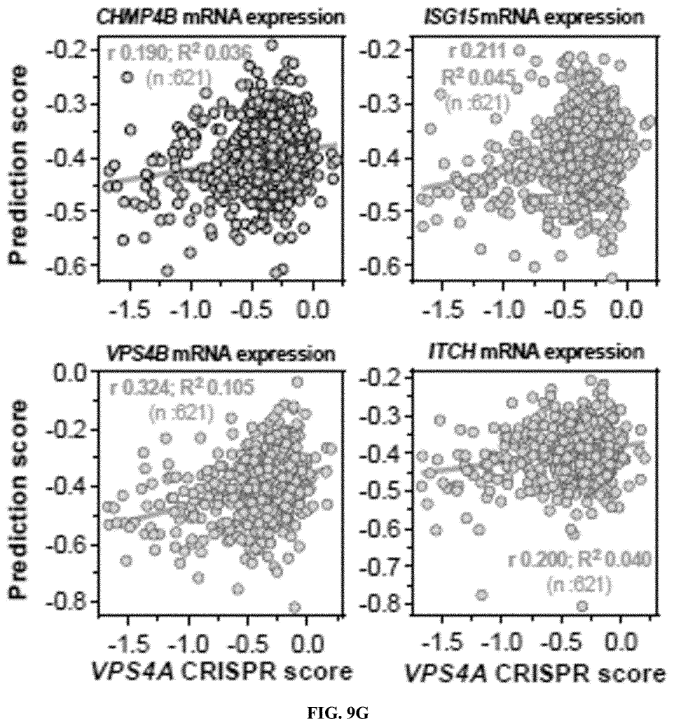

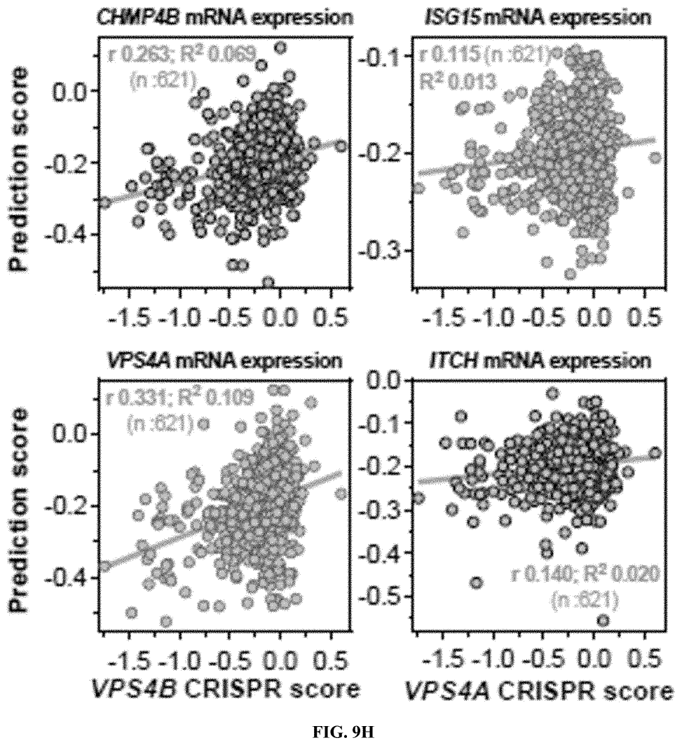

While VPS4A dependency correlates strongly with VPS4B copy number and expression (FIGS. 3F-3I, and FIGS. 2.2D, 2.2F), not every cancer cell line showing VPS4B copy loss is sensitive to VPS4A suppression (FIGS. 2.2D and 2.2F). To search for additional biomarkers to predict VPS4A dependency besides VPS4B loss, VPS4A CRISPR dependency scores were correlated with gene-level RNAseq expression values across cancer cell lines (FIG. 6A). As expected, VPS4A dependency correlated with lower expression of genes located on 18q, confirming the synthetic lethal relationship with SMAD4/VPS4B copy number. Gene-set enrichment analysis on the top 250 anticorrelated genes with VPS4A dependency indicated enrichment of the cellular response to viral infection, cytokine (interleukin) signaling, cell adhesion pathways, and cytoskeletal organization (FIG. 6B). Top enriched pathways were driven by interferon type 1 and 2 signaling pathways and complementary analysis of quantitative proteomic data (FIG. 9A) further pointed to a strong anticorrelation between VPS4A dependency and innate immune response genes, including type 1 interferon and interleukin signaling (FIG. 9B). This anticorrelation between VPS4A CRISPR dependency and the innate response against virus remained even after controlling for VPS4B loss (FIGS. 9C-9D), indicating that increased expression of interferon response genes may enhance cellular sensitivity to VPS4A ablation. To test this hypothesis, a set of 3 pancreatic cancer cell lines expressing the shVPS4A-2 RNAi system were treated with or without doxycycline and in combination with varying doses of interferon-β and interferon-γ. Strikingly, co-treatment of the type II interferon-γ with doxycycline strongly sensitized the VPS4B^{loss} cell lines PANC0403 and SNU213, but not the VPS4B^{neutral} cell line KP4, to VPS4A suppression (FIGS. 6C, 9E). Interferon-β strongly sensitized SNU213 cells to VPS4A suppression but did not alter sensitivity of PANC0403 cells (FIG. 6C), possibly due to a lowered expression of the IFNAR2 receptor (FIG. 9F).

Given that interferon response genes correlate with VPS4A dependency, it was next investigated whether a multivariate model incorporating additional features along with VPS4B expression could yield an improved biomarker for VPS4A dependency. For this purpose, a 10-fold cross-validated four-parameter linear model was generated to predict VPS4A dependency by incorporating gene mRNA expression levels of VPS4B, CHMP4B and ITCH, the two top-scoring modifiers of CRISPR VPS4A dependency screen (FIG. 5B), and the interferon response gene ISG15. This combined model significantly improved the correlation between predicted and observed VPS4A dependency scores over models based on each gene independently or any combination of pairs or triplets of the 4 genes (FIGS. 6D, 9G). This model also outperformed any univariate correlation between VPS4A dependency scores and other gene expression, copy number or gene dependency features. Applying a similar modeling strategy to VPS4B dependency prediction showed that addition of CHMP4B expression to VPS4A expression also strongly increased the predictive power over univariate models and correlations (FIGS. 9H-9I). In summary, these data indicate that the VPS4A-VPS4B synthetic lethal interaction is driven by expression levels of the associated VPS4 paralog and CHMP4B, with expression of interferon response genes serving to further modulate VPS4A or VPS4B dependency.

### Discussion

A systematic analysis was performed of genome-scale CRISPR-SpCas9 and RNAi screening data from the Cancer Dependency Map (www.depmap.org) and genetic vulnerabilities were mapped that correlate with copy number loss of one of 50 common TSGs (see Tables 4 and 5). This compendium of synthetic lethal interactions for cancer nominates multiple known and novel targets for potential therapeutic development and further mechanistic study. A striking number of synthetic lethal interactions with copy loss of the SMAD4 tumor suppressor on chromosome 18q, one of the most frequent genomic alterations in human cancer, are described herein. Most notably, it was observed that a large subset of SMAD4-deficient cancer cells selectively requires expression of the ESCRT-related ATPase VPS4A for survival due to genomic loss of its paralog VPS4B, located 12.3 Mb downstream of SMAD4 on 18q. Furthermore, VPS4A suppression induces apoptosis and cell cycle arrest in in vitro cancer models with reduced copy number of VPS4B and results in profound in vivo tumor regression in subcutaneous cancer xenograft mouse models. Reciprocally, cancer cells with loss of CDH1 (encoding E-cadherin) on chromosome 16q show collateral loss of VPS4A, which sensitizes these cells to depletion of VPS4B. Moreover, cells with VPS4B loss are desensitized to depletion of the ESCRT-III core filament forming protein CHMP4B. These results demonstrate a critical role for the ESCRT machinery in maintaining cancer cell survival and highlight dose-dependent relationships between ESCRT proteins as well as synthetic lethal vulnerabilities in the context of genomic copy loss of one or more pathway components.

The VPS4A-VPS4B paralog dependency is an example of collateral synthetic lethality, where deletion of a neighboring bystander gene leads to cancer dependence on another related gene. Collateral lethality was first described for loss of ENO1 on chromosome 1p36 resulting in dependence on the paralog ENO2 (Muller et al. Nature, 488(7411):337-42, 2012). The ENO1-ENO2 paralog dependency was not verified as the analysis provided herein did not include any tumor suppressor gene located on chromosome 1p. More recently, a collateral lethality relationship was identified for ME2, which is adjacent to SMAD4, leading to dependence on the paralog gene ME3. However, ME3 did not score as a dependency in any of the cancer cell lines in the CRISPR-SpCas9 or RNAi screening datasets evaluated in this study or in the Sanger Institute's CRISPR-SpCas9 Cancer Dependency Map. Possible reasons include technical differences in reagent quality or experimental differences. It has been reported that dependence on PRMT is linked to the co-deletion of WMTAP with the tumor suppressor CDKN2A, and this collateral lethality relationship robustly scored in the present analysis (Kryukov et al. Journal of Experimental Med, 214(10):2933-2946, 2016; Marjon et al., Cell Rep, 15(3):574-58, 2016; Mavrakis et al., Science, 351(6278):1208-13, 2016). The VPS4A-VPS4B paralog dependency relationship has been previously reported in screening data without functional characterization or mechanistic study (McDonald, E. R., 3rd et al. Cell 170, 577-592 e510 (2017); Viswanathan et al. Nat Genet, 50(7):937-943, 2018), and a recent complementary study demonstrated the VPS4A-VPS4B synthetic lethal interaction in a mouse xenograft model of colon cancer (Szymantska et al., EMBO Mol Med e10812 (2020)).

The ESCRT machinery mediates inverse membrane involution, forming complexes on the cytosolic face of the involuting membrane neck (FIG. 1G). The VPS4A/B ATPases are believed to function as hexameric complexes to modulate ESCRT-III filament dynamics and drive ESCRT-mediated membrane fission and sealing to support a multitude of cellular processes (Vietri et al. Nature Review Molecular Cell Biology, 21:25-42, 2020) (FIG. 4A). In response to VPS4A suppression, cancer cells with genomic loss of VPS4B arrest in G2/M, accumulate CHMP4B ESCRT-III filaments, and demonstrate cytokinesis defects, nuclear deformation, and micronucleation, ultimately leading to apoptosis. Outside of anticancer therapeutic development, VPS4 suppression has also been shown to cause defective mitotic spindle formation, disrupted endocytic and vesicular trafficking, impaired maturation of autophagosomes, increased cell-surface accumulation of receptor tyrosine kinases, defective plasma membrane repair, and even DNA damage (Bishop and Woodman. Mol. Biol. Of the Cell, 11(1):227-239, 2000; Lin et al. Molecular and Cellular Bio, 32(6):1124-1138, 2012; Mierzwa et al. Nat Cell Biol., 19(7):787-798, 2017; Morita et al. Cancer Research, 70(1):257-265, 2010; Scheffer et al. Nat Commun, 5:5646, 2014; Szymanska et al., EMBO Mol Med e10812 (2020); Takahashi et al. Nature Communications, 9:1249, 2018; Vietri et al. Nature, 2015, Jun. 11; 522(7555):231-5, 2015; Zheng et al. Oncogene, 31(43):4630-8, 2012). Combined with the results presented herein, these reports indicate that VPS4 ablation impacts a multitude of cellular processes that could contribute to profound anticancer activity. On top of that, combined depletion of VPS4A and VPS4B was shown to cause cell-autonomous activation of inflammatory signaling mediated by NF-κB signaling and expression of immunomodulatory cytokines in colorectal HCT-116 cancer cells, leading to immunogenic cell death and potential activation of M1 macrophages in vitro (Szymantska et al., 2020).

CHMP4B is the main filament-forming ESCRT-III protein which requires nucleation and activation to form multimeric filament structures (Christ et al. Trends in biochemical sciences, 43(1):42-56, 2017). An anticorrelation was observed between CHMP4B dependency and VPS4B loss in genome-scale screening data (FIGS. 1H-1J), indicating that VPS4B-deficient cells harbor reduced fitness from CHMP4B accumulation and that depletion of CHMP4B supports the proliferation of these cells. In line with this hypothesis, CRISPR-SpCas9 screening to identify modifiers of VPS4A dependency revealed that knockout of CHMP4B conferred resistance to VPS4A suppression in VPS4B-deficient cells. Furthermore, integration of CHMP4B mRNA expression levels with VPS4B mRNA expression or gene copy number in a multiple linear model resulted in significantly improved prediction of VPS4A dependency. Collectively, these data support the paradigm that cancer cells with increased levels of CHMP4B more strongly require VPS4 activity to maintain viability and that VPS4 suppression induces cell death in part due to excessive CHMP4B accumulation. This paradigm could also help explain why a subset of cell lines with partial VPS4B or VPS4A copy loss do not exhibit strong dependency on the reciprocal paralog.

Cancer cell sensitivity to VPS4A suppression was also potently enhanced by disruption of regulators of the abscission checkpoint, including genes encoding the ULK3 kinase and the ESCRT-III proteins CHMP1A and CHMP1B. The abscission checkpoint is a genome protection mechanism that relies on Aurora B kinase (AURKB) and ESCRT-III subunits to delay abscission in response to chromosome mis-segregation to avoid DNA damage and aneuploidy. ULK3 is regulated by AURKB and binds to and phosphorylates ESCRT-III proteins including CHMP1A, CHMP1B, CHMP2A, and IST1, resulting in inhibition of ESCRT-III polymerization and VPS4 activity. Consequently, knockout of ULK3, CHMP1A or CHMP1B would be expected to further disrupt the abscission checkpoint, leading to further impairment in cytokinesis beyond that observed with VPS4A suppression alone. Moreover, as CHMP1A and CHMP1B are regulatory ESCRT-III proteins with among the strongest affinity for VPS4 proteins https://paperpile.com/c/lzErE/R0TCS, knockout of these genes might also impair recruitment of remaining VPS4 proteins to ESCRT-III filaments, further enhancing excessive accumulation of CHMP4B or other ESCRT-III filaments and contributing to further disruption of ESCRT-mediated cellular processes. Thus, key components of the ESCRT machinery regulate cancer cell survival and specifically modulate sensitivity and resistance to VPS4A suppression. These findings indicate that inhibition of the ESCRT pathway and blockade of the abscission checkpoint could provide strategies to further enhance sensitivity of cancer cells to VPS4A suppression.

Integrative transcriptomic and proteomic analysis also identified a strong correlation between baseline interferon response gene expression and VPS4A dependency. Notably, the ubiquitin-like protein interferon-stimulated gene 15 (ISG15) has been reported to be upregulated by the interferon response upon viral infection to block viral release by preventing VPS4 enzymes from interacting with ESCRT-III filaments at the site of the budding viral particle (Kuang et al. J Virology, 85(14):7153-7161, 2011; Pincetic et al. J Virol, 84(9):4725-36, 2010). Furthermore, the cellular interferon response has also been shown to downregulate VPS4 expression to further halt viral maturation (Cabrera et al. mBio, 10(2):e02567-18, 2019). When included in the presently described multiple linear model to predict VPS4A dependency, the interferon response gene ISG15 and the immune-related E3 ubiquitin-protein ligase ITCH improved the predictive power of the model over expression of VPS4B or CHMP4B, alone or in combination. Moreover, interferon treatment of VPS4A-dependent cell lines enhanced sensitivity to VPS4A knockdown. Thus, these data collectively indicate that baseline interferon response signaling potently modulates VPS4A dependency. Combined with the observation that VPS4A+VPS4B depletion leads to immunogenic cell death in colorectal cancer cells, the presently described results indicate that the modulation of inflammatory signaling in the context of VPS4 depletion forms a potential combinatorial therapeutic strategy for future consideration.

The SMAD4 tumor suppressor on chromosome 18q21.33 is lost in approximately 33% of human cancer, with particularly high rates of loss in pancreatic cancers (68%), colorectal (71%) and renal cell carcinomas (17%) (Zack, T. I. et al. Nat Genet 45, 1134-1140 (2013)). Given its proximity to SMIAD4, VPS4B is often co-deleted with SMAD4, thereby sensitizing cells with 18q loss to VPS4A suppression. Conversely, VPS4A is adjacent to CDH1 and is also lost in other tumor types, including cancer lineages in which VPS4B is not commonly deleted, thus sensitizing those tumor cells to VPS4B depletion. Interestingly, complete genomic loss of either VPS4A or VPS4B was almost never observed, even though SMAD4 and CDH1 are sometimes lost completely. In aggregate, over one-third of cancers harbor partial copy loss of VPS4A or VPS4B and a diverse spectrum of tumors showing VPS4A or VPS4B loss will be sensitive to depletion or inhibition of the associated paralog.

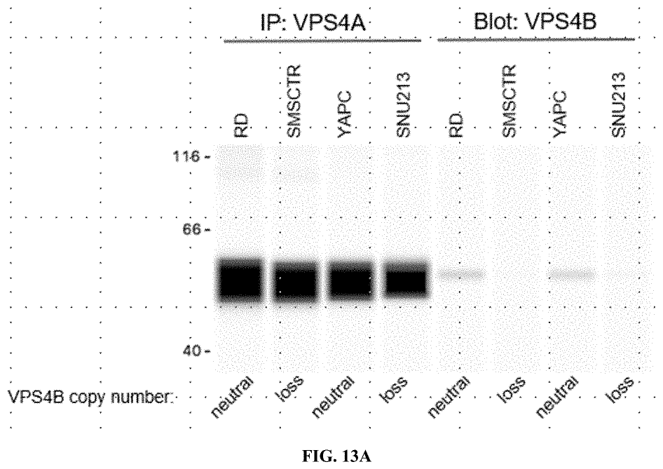

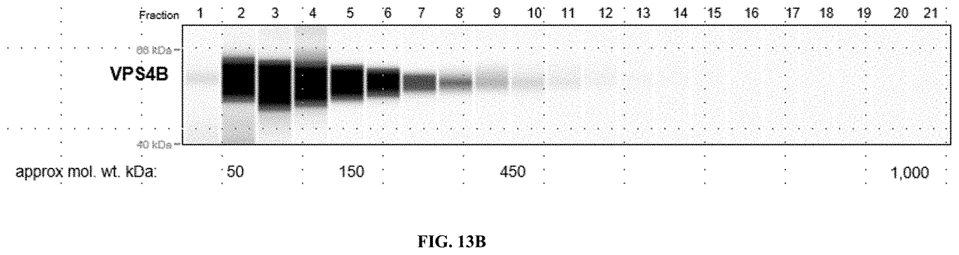

Finally, the mutant rescue experiments showed that the ATPase domain is critical for the function of VPS4A in mediating survival of cells with partial copy loss of VPS4B. Although VPS4A and B demonstrate 80.5% homology, the development of small molecules that differentially target VPS4A in cells with VPS4B loss or VPS4B in cells with VPS4A loss remains a tractable possibility due to small structural differences near the ATP-binding pocket. Moreover, combined inhibition of VPS4A and VPS4B may also prove effective and clinically tolerable given a potential therapeutic window arising from gene dosage alterations and differences in total VPS4A/B levels in tumor versus normal cells. Although currently no specific VPS4A/B inhibitor has been developed, non-specific inhibitors of AAA ATPases have been reported to bind VPS4 proteins (Pöhler et al. Angew Chem Int Ed Engl, 57(6):1576-1580, 2018; Zhang et al. Mycopathologia, 181(5-6):329-39, 2016). For example, although the findings described herein support functional redundancy of VPS4A and VPS4B, distinct functions of each paralog protein may also exist given the wide range of cellular processes regulated by the ESCRT machinery. Moreover, various studies using in vitro experiments or yeast cells (which normally express only a single VPS4 protein) have demonstrated that VPS4A and VPS4B could interact (Huttlin et al. Cell, 162(2):425-440, 2015; Scheuring, S. et al. J Mol Biol 312, 469-480 (2001)). The degree to which VPS4A and VPS4B cooperate and form functional homomeric versus heteromeric complexes in living human (cancer) cells remains to be fully elucidated. However, the immunoprecipitation with VPS4A in various cancer cell lines (RD, SMSCTR, YAPC, SNU213) with either a neutral or loss of VPS4B copy number and immunoblots in the rhabdomyosarcoma (RD) and pancreatic (YAPC) cell lines with a neutral VPS4B copy number suggests that there is a weak interaction between VPS4A and VPS4B and that most of VPS4A and VPS4B are not in a heteromeric state (FIG. 13A). Glycerol gradient fractionation and immunoblot for VPS4B suggested that VPS4A and VPS4B exist predominantly in low molecular weight pools (FIG. 13B). VPS4A and VPS4B were found to rarely colocalize in cells as evidenced by confocal fluorescence visualization in the pancreatic cancer (SNU213) cell line (VPS4B^{loss}) of DNA (DAPI), VPS4A (Alexa Fluor 488) and VPS4B (Alexa Fluor 568. An overlay of these three vizualizations demonstrated that VPS4A and VPS4B mainly localize to the cytoplasm and do not overlap significantly. This indicates that VPS4A and VPS4B likely have a higher affinity for homomeric complexes over heteromeric complexes (FIG. 13C).

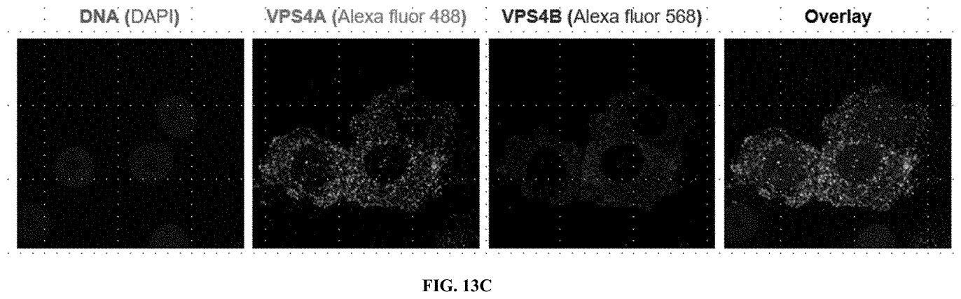

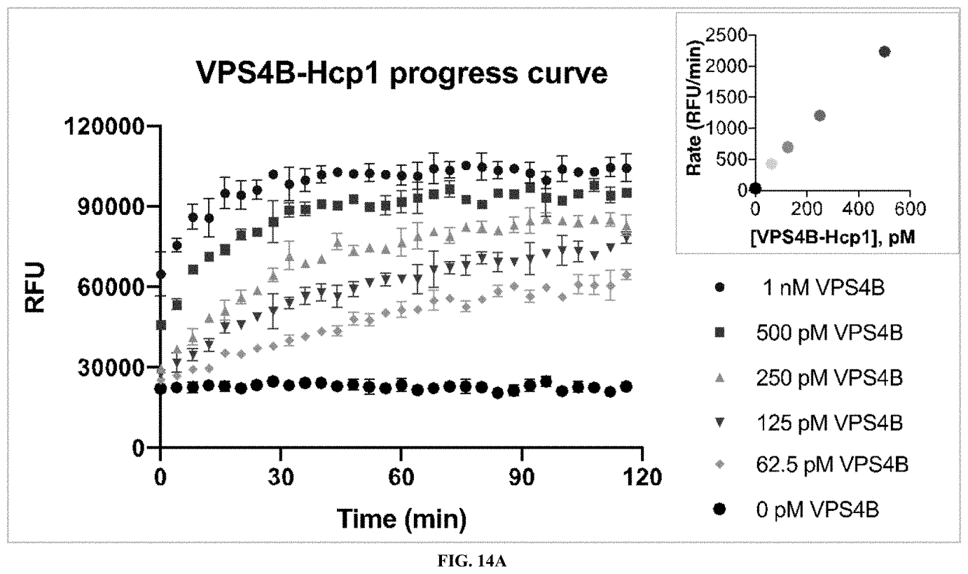

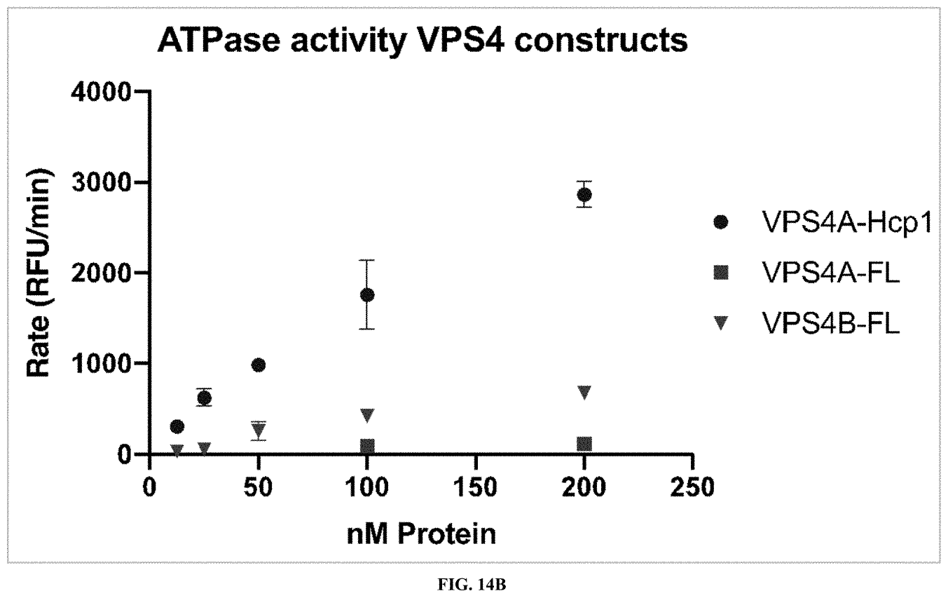

Active oligomers of VPS4A and VPS4B were achieved through recombinant production and an in vitro assay was used to measure ATPas activity. VPS4 activity was detected and confirmed to be both time and concentration dependent for VPS4B as demonstrated by the VPS4B-Hcp1 tagged construct progress curve (FIG. 14A). ATPase activity of other VPS4 constructs, including VPS4A-Hcp1, VPS4A-FL, and VPS4B-FL, similarly demonstrated concentration dependency (FIG. 14B). Hcp1 (also known as His6 tag (SEQ ID NO: 32)) and FL (also known as FLAG tag) were used. The preliminary turnover number that was calculated for the VPS4A and VPS4B tagged constructs showed over a 600 fold change between the VPS4A-Hcp1 and VPS4B-Hcp1 constructs and just over a 1 fold change between VPSrA-FL and VPS4B-FL (Table 1). The data demonstrate that the hexameric proteins showed the highest specific activity.

TABLE 1
The preliminary turnover number calculated
for the VPS4A and VPS4B constructs.

| | VPS4A-Hcp1 | VPS4B-Hcp1 | VPS4A-FL | VPS4B-FL |
|---|---|---|---|---|
| Turnover | 53 | 33280 | 10 | 21 |
| (sec⁻¹) | | | | |

Given the genomic biomarker prevalence and the potent synthetic lethal relationships demonstrated here, the development of small molecule inhibitors of VPS4 proteins may prove an important advance in the treatment of cancer.

### Methods and Materials

The results reported herein above were obtained using the following materials and methods.

Discovery of Synthetic Lethal Interactions with Genomic Loss of Tumor Suppressors

To uncover synthetic lethal interactions with somatic CNAs of established TSGs, data was analyzed and integrated from pooled, genome-scale RNA interference (RNAi) and CRISPR-SpCas9 loss-of-function screening for effects on cell proliferation from over 600 well annotated cancer cell lines within the Broad's Institute Cancer Dependency Map Public 19Q3 release (depmap.org) (McFarland, J. M. et al. Nat Commun 9, 4610 (2018); Meyers, R. M. et al. Nature genetics 49, 1779-1784 (2017); Tsherniak, A. et al. Cell 170, 564-576 e516 (2017)). The analysis was limited to 50 common TSGs (Table 5) and correlated Log 2-normalized copy number calls for each of these tumor suppressors with normalized, gene-level CRISPR (622 cell lines, 18,333 genes) and RNAi (669 cell lines, 16,905 genes) dependency scores. These correlations were performed in R, a language and environment for statistical computing and graphics, using the inbuilt cor.test function. For each pair of tumor-suppressor gene and dependency gene, the Pearson correlation was calculated with its p-value. A 10% false-discovery rate (FDR, Benjamini-Hochberg) was applied using the p.adjust function in R. Gene chromosomal location information was obtained from the Atlas of Genetics and Cytogenetics in Oncology and Haematology (downloaded june 2019). When the chromosomal arm-level location of a dependency gene was the same as the arm-level location of the correlated tumor suppressor gene, this interaction was classified as a cis interaction. If the chromosomal arm housing the dependency gene was different from the location of the tumor suppressor gene, the interaction was classified as a trans interaction instead. For synthetic lethal interaction analysis, only trans dependency genes were incorporated that showed a positive correlation with copy loss of tumor suppressor genes. Significant synthetic lethal interactions were then cross-referenced between the RNAi and CRISPR datasets to obtain a list of highly confident synthetic lethal interactions (Table 4). Finalized results were visualized with GraphPad Prism v8.3.0.

### Code, Data and Materials Availability

All code will be made available on a public repository and is available on request. The Public 19Q3 Broad Institute's Cancer Dependency Map and Cancer Cell Line Encyclopedia datasets (depmap.org), are also available on figshare: doi.org/10.6084/m9.figshare.9201770.v3; doi.org/10.6084/m9.figshare.9170975.v1.

### Analyses of Cancer Patient Sample Copy Number Data

Copy Number Analysis from TCGA PanCan Cohort

Copy number data from 10,712 TCGA patient samples (Sanchez-Vega et al. Cell, 173(2):321-337, 2018; Taylor et al. Cancer Cell, 33(4):676-689, 2018) were downloaded from the NIH Genomic Data Commons at gdc.cancer.gov/about-data/publications/pancan-aneuploidy. GISTIC thresholded copy number calls (Mermel et al. Genome Biol., 12(4):R41, 2011) were used to determine copy number status of VPS4B. Samples with copy number values of “−1” or “−2” were called having at least partial copy loss or deeper deletions, respectively.

VPS4B Copy Number Analysis from RMS Patient Sample Data

Illumina whole exome and whole genome paired end sequences were downloaded from RMS patient samples published by (Chen et al., Cancer Cell 24(6):710-724, 2013). Copy numbers were called using GATK4 (DePristo et al. Nat. Genet, 43(5):491-498, 2011; Van der Auwera et al. Curr Protoc Bioinformatics, 43(1110):11.10.1-11.10.33, 2013) to obtain relative copy number values, which were log 2 transformed with a pseudocount of 1. In their study, multiple samples from each patient were possible from primary, metastatic or patient-derived xenografts. For those cases VPS4B copy number calls were highly concordant and therefore the additional samples were removed to prevent double counting.

Copy Number Analysis from Dana-Farber Cancer Institute (DFCI) Profile Project Using OncoPanel

DFCI's database of all pediatric, adult pancreatic, adult ovarian, and adult sarcoma patient samples were analyzed using the OncoPanel targeted sequencing assay (Garcia et al. Archives of Pathology and Laboratory Medicine, 141(6):751-758, 2017; Sholl et al. JCI Insight, 1(19):e87062, 2016), in accordance with DFCI's IRB approval. samples were chosen with a known/annotated primary tumor type and over 20% histological tumor purity. Though VPS4B is not covered on OncoPanel, it was investigated whether a neighboring gene's copy number status could be used as a surrogate. First, TCGA PanCancerAtlas copy number calls (10,967 samples in cBioPortal (Cerami et al. Cancer Discov, 2(5):401-404, 2012; Gao et al. Sci. Signal, 6(269):p11-p11, 2013) were used to assess the positive and negative predictive values for 240 genes on chromosome 18 to predict a concurrent deletion for VPS4B, where both gene's copy number status was known. In particular, a given gene's “Shallow deletion” copy call was used to infer whether the VPS4B copy call in that sample is also “Shallow deletion.” Of the chromosome 18 genes that are covered on Oncopanel, BCL2 shallow deletion is the best predictor of VPS4B shallow deletion, with 99.7% positive predictive value and 99.9% negative predictive value (see FIG. 2.2E). Next, the frequency of BCL2/VPS4B-inferred shallow deletions across the DFCI PROFILE were calculated.

### Cell Culture

All cell lines were from validated sources and procured through the Broad Institute's Dependency Map project cell banks. Parental cell lines were obtained from the Cancer Cell Line Encyclopedia and SpCas9-expressing cell lines were obtained from the Broad Institute's Genetic Perturbation Platform. All cell lines were originally obtained from authorized cell line banks including the American Type Culture Collection (ATCC), Korean Cell Line Bank (KCLB), Deutsche Sammlung von Mikroorganismen und Zellkulturen (DSMZ) and the Japanese Riken cell line bank. All cells were cultured in RPMI-1640 with 10% fetal bovine serum with additional supplements when indicated. Cells were initially thawed and expanded in their native media, however if their native media was not RPMI, then they were adapted and maintained in RPMI for all experiments. Cell lines were validated by STR profiling and routinely tested for mycoplasma.

### Generation of Cell Lines for Validation Experiments

CRISPR-SpCas9 and shRNA validation experiments were performed using lentiviral transduction of sgRNAs into cancer cell lines that stably express the SpCas9 nuclease. Lentiviral transduction was used to generate stable cell lines expressing SpCas9 or the inducible RNAi systems. Lentiviral particles for SpCas9, shRNAs and sgRNAs were created by co-transfection into HEK293T cells with a packaging (psPAX2) and VSV-G envelope plasmid (pMD2.G).

CRISPR-Based Cell Viability Assays with CellTiter-Glo

### Overall Assay Design and Cell Line Optimization

For FIG. 2A, CellTiter-Glo viability assays were performed with stably expressing SpCas9 cell lines in 96 well plates. Cells were seeded and infected with sgRNA expressing lentivirus in wells on day 0, and selected with puromycin 24 hours later. Cell titer-Glo viability was read out 7 days after plating and infection. Prior to CellTiter-Glo viability assays, all cell lines were individually optimized for the assay including titrating both cell seeding density and volume of virus used for infection. All lentiviral preps for each sgRNA described below were batch controlled and titrated on three different cell lines representing cell lines with low, medium and high transduction efficiency. Optimal cell seeding densities and viral volumes for infections were then used for all subsequent viability assays using CRISPR.

sgRNAs Design and Rationale

For viability assays, 3 negative control guides (sgLacZ, sgChr2 and sgAAVS1), 3 positive control guides targeting pan-essential genes (sgPOLR2D, sgSF3B1 and sgKIF11), and 3 guides targeting VPS4A (sgVPS4A-1, sgVPS4A-2 and sgVPS4A-3) were used. For negative control guides, two “cutting control” guides were designed that allow CRISPR-SpCas9 to cleave safe regions of the human genome to control for effects of DNA double strand breaks. For sgChr2, the sgRNA targets a gene desert on chromosome 2, which is also the least copy number altered chromosome across cancer (Beroukhim et al. Nature, 463(7283):899-905, 2010). For sgAAVS1, the sgRNA targets the safe harbor AAVS1 integration locus which is an intronic region in PPP1R12C. sgLacZ represents a non-targeting sequence not found in the human genome. The 20 bp targeting sequences for each sgRNA were:

| | sgLacZ: |
|---|---|
| (SEQ ID NO: 33) | |
| | 5′-AACGGCGGATTGACCGTAAT |
| | sgChr2: |
| (SEQ ID NO: 34) | |
| | 5′-GGTGTGCGTATGAAGCAGTG |
| | sgAAVS1: |
| (SEQ ID NO: 35) | |
| | 5′-AGGGAGACATCCGTCGGAGA |
| | sgPOLR2D: |
| (SEQ ID NO: 36) | |
| | 5′-AGAGACTGCTGAGGAGTCCA |
| | sgSF3B1: |
| (SEQ ID NO: 37) | |
| | 5′-AAGGGTATCCGCCAACACAG |
| | sgKIF11: |
| (SEQ ID NO: 38) | |
| | 5′-CAGTATAGACACCACAGTTG |
| | sgVPS4A-1: |
| (SEQ ID NO: 39) | |
| | 5′-ACTCACACTTGATAGCGTGG |
| | sgVPS4A-2: |
| (SEQ ID NO: 40) | |
| | 5′-GGGCCGCACGAAGTACCTGG |
| | (intron/exon junction, |
| | also used in ORF rescue) |
| | sgVPS4A-3: |
| (SEQ ID NO: 41) | |
| | 5′-ATTGTTATTCCCCACCCCTG |
| | (intron/exon junction, |
| | also used in ORF rescue) |

### Viability Assay Data Quality Control

Each assay was required to meet specific quality control metrics. There were 10 unique conditions, one for each of the 9 guides described above, plus a no infection control. There were 6 replicate wells per sgRNA infection, 3 were selected with puromycin and 3 were not. For quality control of raw luminescence from CellTiter-Glo, infection efficiency (puro/no puro selection for each sgRNA) was required to be at least 80%, and all replicate wells had to be within 2 standard deviations of the mean for that sgRNA infection. Viability reduction from cutting controls, corresponding to DNA double strand breaks, was to be no more than 30% of the non-targeting sgLacZ. SpCas9 activity from each cell line was also required to be greater than 50%, determined by the percent viability reduction of the average of the 3 pan-essential genes (sgPOLR2D, sgSF3B1 and sgKIF11) to negative controls (sgLacZ, sgChr2 and sgAAVS1).

### Data Normalization

The viability data was normalized and scaled in a manner comparable to the DepMap dependency scores (CERES for CRISPR (Meyers, R. M. et al. Nature genetics 49, 1779-1784 (2017) and DEMETER2 for RNAi (McFarland, J. M. et al. Nat Commun 9, 4610 (2018)). Viability scores were normalized on a scale from 0 (the average effect of negative sgRNA cutting controls) to −1 (the average effect of knockout from 3 different pan-essential genes). The distance of each well to the average of the two cutting control sgRNAs (sgChr2 and sgAAVS1) was first calculated.

Cutting control normalized values individual well value −(AVERAGE(cutting control wells))

For each assay well, these values were then scaled from 0 to −1.0 represents the average viability effect of the cutting controls and −1 represents the average viability effect knockout of the 3 pan-essential genes run in the assay.

$$Scaled viability : \frac{Cutting control normalized well value}{\begin{matrix}AVERAGE ( cutting control normalized wells ) - \\ AVERAGE ( pan essential control normalized wells )\end{matrix}}$$

Scaling the cell viability effect in this way allows for one to compare across cells lines that have differential responses to “off target” effects of CRISPR e.g. DNA double strand breaks, and differential Cas9 activity when cell lines exhibit differences in maximum number of cells killed by pan essential gene ablation.

### Generation of Doxycycline Induced RNAi Reagents

VPS4A shRNA sequences were selected from project DRIVE (McDonald, E. R., 3rd et al. Cell 170, 577-592 e510 (2017)) and cloned into the pRSITEP-U6Tet-(shRNA)-EF1-TetRep-2A-Puro vector (Cellecta #SVSHU6TEP-L) for doxycycline inducible shRNA expression. Negative control shRNA seed sequences were generated for each on-target shRNA. Seed sequences contain mutations in base pair positions 9-11 of the shRNA that are intended to remove on-target knockdown, but retain the same seed sequence (bp positions 2-8) and off-target effects (Buehler et al. PLoS One, 7(12):e51942, 2012). shRNA target sequences are provided below:

| | shVPS4A-1: |
|---|---|
| (SEQ ID NO: 42) | |
| | 5′-GCAAGAAGCCAGTCAAAGAGA |
| | shSeed-1: |
| (SEQ ID NO: 43) | |
| | 5′-GCAAGAAGCCTCACAAAGAGA |
| | shVPS4A-2: |
| (SEQ ID NO: 44) | |
| | 5′-CGAGAAGCTGAAGGATTATTT |
| | shSeed-2: |
| (SEQ ID NO: 45) | |
| | 5′-CGAGAAGCTGTTCGATTATTT |
| | shVPS4A-3: |
| (SEQ ID NO: 46) | |
| | 5′-GCCGAGAAGCTGAAGGATTAT |
| | shSeed-3: |
| (SEQ ID NO: 47) | |
| | 5′-GCCGAGAAGCACTAGGATTAT |

RNAi Based Cell Viability Assays with CellTiter-Glo

For FIG. 2B, cell lines stably expressing doxycycline-inducible shVPS4A-2 or sequenced match shSeed-2 control were plated in 96 well plates in the presence or absence of 1 uM doxycycline. Cells were cultured for 7 days and then assayed for cell number using CellTiter-Glo. Relative cell viability was calculated by dividing the doxycycline condition luminescence values by the no doxycycline treatment for each cell line.

Subcutaneous Xenograft Study with SMSCTR

Animal studies were done in accordance with Dana Farber Cancer Institute's IACUC approved protocol (DFCI 16-015). Rhabdomyosarcoma SMSCTR cells stably transduced with the CRISPR-SpCas9 endonuclease and the shVPS4A-2 or shSeed2 tetracycline-inducible RNAi system were maintained in log phase growth in RPMI-1640 with 10% FBS and 300 μg/mL hygromycin. They were confirmed as mycoplasma free and prepared for subcutaneous injection into female CIEA NOG mice (NOD. Cg-Prkdcscid Il2rgtm1Sug/JicTac) (Taconic labs). A total of 38 mice were injected once in the flank with 8e⁶ cells resuspended in 100 μL PBS without matrigel. Tumor size was monitored at least biweekly by calliper measurement after shaving and 3-5 weeks after injection, mice were randomized to doxycycline containing diet (625 ppm) or control diet when tumors reached ˜300 mm³. Once tumors reached >2000 mm³, mice were sacrificed, and tumors were harvested and stored at −80° C. To assess on-target knockdown of VPS4A, one mouse for each treatment arm and for both shSeed2 and shVPS4A-2 tumors were selected (total of four mice) and sacrificed 7 days after treatment randomization. Early time point tumors were harvested, weighted and lysed in 15×tumor-weight in volume RIPA lysis buffer using 2 mL microcentrifuge tubes coupled to Precellys® Evolution bead-mill homogenization at 7500 rpm for 3×30 seconds. After homogenization, tubes were spun down at 4° C. and the lysis supernatant was collected and stored at −20° C. until immunoblotting. This experiment was repeated a second time using a cohort of eight NRG mice (NOD.Cg-RagltmlMom Il2rgtm1Wjl/SzJ, 007799; The Jackson Laboratory), with two flank tumors per mouse and achieved similar results.

### Immuno-Based Detection of Proteins

Immunoblots were carried out on RIPA-generated lysates following either standard wet-transfer protocols imaged using LI-COR fluorescent secondary antibodies on an Odyssey CLx Imager (LI-COR Biosciences) or by automated capillary-based detection of chemiluminescent signal generated by HRP-conjugated secondary antibodies with a Wes system (Protein Simple).

### Western Blotting

Whole cell protein lysates were collected in cold RIPA buffer (150 mM NaCl, 1.0% IGEPAL® CA-630, 0.5% sodium deoxycholate, 0.1% SDS, 50 mM Tris, pH 8.0) supplemented with cOmplete, Mini Protease Inhibitor Cocktail Tablets (Roche). Cell extracts were cleared by spinning at max speed in tabletop centrifuge at 4° C. Protein concentrations were quantified using BCA Protein Assay Kit (Pierce) and diluted to equivalent concentrations. Lysates were run on NuPAGE™ 4-12% Bis-Tris Protein Gels and transferred to PVDF membranes. Licor fluorescent secondary antibodies were used to detect proteins using an Odyssey CLx Imager. Antibodies used in Western blotting are listed in Table 2.

### Protein Simple Capillary Based Detection

Cell lysates were prepared similarly using cold RIPA buffer supplemented with proteinase inhibitors. Protein concentrations were determined using the BCA Protein Assay Kit (Pierce). Samples were then diluted to 0.125 mg/mL total protein and prepared according to the instructions of the ProteinSimple Wes System. Briefly, lysates were denatured by 5 minutes of boiling at 95° C. in sample buffer with 1% SDS and 40 mM DTT. 3 μL of denatured sample was then separated and detected using chemiluminescence generated by anti-mouse and anti-rabbit secondary antibodies conjugated to horseradish peroxidase using standard settings and volumes for the 12-230 kDa 25-capillary separation module used with a Wes System (SM-W004; ProteinSimple). Antibodies used in the ProteinSimple Wes System are listed in Table 2.

TABLE 2
Antibodies used for Western blotting and the ProteinSimple Wes System.

| | | | | Protein | | |
|---|---|---|---|---|---|---|
| | | | Western | Simple | | |
| Target | Species | Clonality | Dilution | Dilution | Vendor | Catalog # |
| VPS4A | Mouse | Monoclonal | 1:500 | 1:20 | Santa Cruz | sc-133122 |
| | | | | | Biotechnology | |
| VPS4B | Rabbit | Monoclonal | 1:1000 | 1:33 | Abcam | ab137027 |
| V5 | Rabbit | Monoclonal | 1:1000 | 1:50 | Cell Signaling | 13202S |
| | | | | | Technology | |
| GAPDH | Rabbit | Monoclonal | 1:1000 | — | Cell Signaling | 2118 |
| | | | | | Technology | |
| Vinculin | Rabbit | Monoclonal | — | 1:75 | Abcam | ab129002 |
| anti- | Goat | NA | 1:10000 | — | LI-COR | 926-68020 |
| mouse- | | | | | Biosciences | |
| IRDye | | | | | | |
| 680LT | | | | | | |
| Anti- | Goat | NA | 1:10000 | — | LI-COR | 926-32211 |
| rabbit- | | | | | Biosciences | |
| IRDye | | | | | | |
| 800CW | | | | | | |
| Anti- | Goat | NA | — | undiluted | ProteinSimple | DM-002 |
| mouse- | | | | | | |
| HRP | | | | | | |
| anti- | Goat | NA | — | undiluted | ProteinSimple | DM-001 |
| rabbit- | | | | | | |
| HRP | | | | | | |

### Incucyte-Based Apoptosis by Caspase 3/7 Fluorescent Reporter Dye

### Cell Seeding

Stable SpCas9 expressing cells were plated and infected in a manner similar to CRISPR Cell-Titer Glo viability assay described above. Six replicate wells per sgRNA were seeded in clear bottom 96-well plates with EMEM media supplemented with 10% Fetal Bovine Serum and 1× Penicillin-Streptomycin-Glutamine. Standard RPMI-1640 contains riboflavin which can generate fluorescent background with caspase 3/7 signal in the Incucyte assay and was therefore not used. On the same day as cell seeding, cells were infected with sgRNAs expression vectors.

### Antibiotic Selection and Caspase Dye Treatment:

24 hours after plating, three of the six replicates received fresh media with 1 μg/mL of puromycin and three of the six replicates received fresh media without puromycin. All media conditions contained 5 μM of IncuCyte Caspase-3/7 Green Apoptosis Assay Reagent (catalog #: 4440). Media selection was performed in the dark due to the light-sensitive nature of the apoptosis reagent.

Following selection, the plate was transferred into the IncuCyte® S3 Live-Cell Analysis System (catalog #: 4647) for imaging. Phase contrast images and green fluorescent channel images were captured using the 10× objective magnification every two hours for a total of 46 time-points. For each well, four images containing both phase contrast and green channel data were obtained.

### Incucyte Data Analysis:

Using the IncuCyte® S3 Analysis System software, cell confluence over time was quantified along with the total area of green (apoptosis positive) objects in μm²/well. Computer generated masks for confluence and green area, trained on a sample set of images across timepoints and confluency levels, were manually checked for accuracy. The ruleset generated by the training image set was then applied to all images and all time points.

Each metric was averaged over the four quadrants per well. First, the green object area metric for each well was divided by the confluence metric for each well, yielding a quantitation of the percent field-of-view positive for apoptosis. These values for each well at each time point were subsequently normalized to well average time-matched no-infection control, no-puromycin condition, which represent unperturbed cell growth. Standard error was computed and plotted using the 3 resulting values per condition, each representing a single well of a 96 well assay plate.

$$Normalized {Signal}_{W , T} = \frac{( {G}_{W , T} - {P}_{W , T} )}{( {G}_{N , T} / {P}_{N , T} )}$$

W=well

T=timepoint

G=total green object area

P=phase contrast confluence

N=no-infection control well average

### Edu and DAPI Stain-Based Cell Cycle Analysis by Flow Cytometry

Cell lines stably expressing SpCas9 cells were plated in 6 well plates and infected with sgRNA expressing vectors. Cells were selected with puromycin 24 hours after infection. Four days after infection, cells were labeled with EdU for 1-3 hours and stained with the Click-iT™ Plus EdU Flow Cytometry Assay Kit (ThermoFisher, Catalog #: C10632). Cells were co-stained with DAPI and then analyzed by flow cytometry and analyzed with FlowJo v10.

### Long-Term Colony Formation Assays

Cell lines stably expressing doxycycline inducible shVPS4A or seed matched control RNAi reagents were plated in 24 well plates in triplicate with or without 1 μM doxycycline. Three different plating densities (18,000, 9,000 or 4,500 cells/well) were used to determine the optimal plating density. Plates with optimal density were selected as the plating density that generated negative control wells that reached confluence after 14 days of plating. For staining, 24 well plates were fixed with 10% buffered formalin 15 min, washed deionized water, stained with 0.1% crystal violet for 20 min, and washed with deionized water again. For quantification, crystal violet dye was extracted using 1 mL of 10% acetic acid for 20 min, diluted 4-fold with water and 50 μL were plated in triplicate in 96 well plates. Absorbance was quantified at 590 nm.

### Apoptosis Induction by Annexin Vflow Cytometry

Cas9 stable cell lines were plated and infected with the indicated sgRNAs in 6 well plates (cell plating range 2e⁵ to 5e⁵ cells per well). Cells were selected with puromycin 24 hours after plating and infection and assayed 5 days after infection by flow cytometry. Inactivation of pan essential gene SF3B1 was used as a positive control for apoptosis induction. Cells were stained using the BD Pharmingen FITC Annexin V Apoptosis Detection Kit (catalog #: 556547) according to the manufacturer's specifications and analyzed by flow cytometry with FloJo version 10.

### Quantification of VPS4B Protein Levels

VPS4B protein levels were examined using two data sources. First VPS4B quantitative proteomics from a subset of 375 cell lines (Nusinow et al. Cell 180(2):387-402.e16, 2020) from the CCLE (data available on depmap.org).

Briefly, tandem mass tagged (TMT) signal-to-noise values from MS3 scans were exported and paired with their MS2 peptide identities. Filtered TMT values were summed and normalized for loading within a ten-plex. Normalized protein abundance values were log 2-transformed and mean protein expression per cell line was centered at 0. Second, quantification of VPS4B from cell line lysates by Protein Simple were calculated by extracting the VPS4B luminescent peak signal intensity and dividing it by the sum of all total protein peak intensities.

FIG. 12C quantifies VPS4B protein level in cells with copy number loss or copy number neutral, showing a statistically significant difference. FIG. 12D demonstrates the relative VPS4B copy number in a multitude of TCGA Pan-Cancer Atlas samples.

### Generation of Isogenic VPS4B−/− Cell Lines

VPS4A^{neutral} RD cells, stably expressing SpCas9, were infected with a lentivirus expressing sgRNA targeting the sixth exon of VPS4B (sgVPS4B: 5′-CCACTTAGAAACAAGATCAG (SEQ ID NO: 48)). Due to the variable enzymatic activity of SpCas9 across single cells, the infected cells were serially diluted into clear bottom 96-well plates and examined for the presence of single cells. Wells containing single cells were expanded. Sixteen of the resulting clonal populations were interrogated for VPS4B knockout by western blot. DNA extracts from the isogenic cell clones were Sanger sequenced and presence of indels were assessed by the TIDEseq method of deconvolution (tide.deskgen.com/) using VPS4B exon 6 targeting primers (VPS4B-For: 5′-GCCTAATCATGTTTCAGGTACAGA (SEQ ID NO: 49), VPS4B-Rev: 5′-GGCAAGAGAACACCTTGGAG (SEQ ID NO: 50)). Cell lines that were both null by western blot and contained >80% indels by TIDEseq were selected for further experimentation and pooled into 2 groups of 4 to mitigate the effects of clonal variation.

### Generation of VPS4A and VPS4B Overexpression Plasmids and Cell Lines

A pLX313 ORF expression vector containing VPS4B was procured from the Broad Genetic Perturbation Platform (portals.broadinstitute.org/gpp/public/). For VPS4B over expression, the VPS4B^{loss} JR cell line stably expressing SpCas9 was infected with pLX313-VPS4B lentiviral particles and selected with 200 ug/mL hygromycin. Cells were expanded and examined for increased VPS4B expression by western blot. Cells were then placed in 7 day Cell-Titer Glo viability assays as described above in the “CRISPR based cell viability assays with CellTiter-glo” Methods section.

VPS4A Site-Directed Mutagenesis and Quantification Viability after VPS4A ORF Expression

A pDONR223 VPS4A vector was procured from the Broad Genetic Perturbation Platform. Three mutations in VPS4A reported to alter function were selected from the literature to interrogate their ability to rescue cell viability in VPS4A-dependent cell lines following endogenous VPS4A inactivation. VPS4A^{L64A} was reported to prevent MIT domain binding of ESCRT-III filament CHMP1B without disrupting MIT domain folding (Scott et al. Proc Natl Acad Sci USA, 102(39):13813-8, 2005). Whereas VPS4A^{K173Q} exbited dominant negative activity that aborgates ATP binding (Stuchell et al. J Biol Chem, 279(34):36059-71, 2004). Lastly, a VPS4A^{E228Q} mutant was engineered that prevents ATP hydrolysis (Scheuring, S. et al. J Mol Biol 312, 469-480 (2001); Tanaka et al. J Biol Chem, 277(42):40142-7), 2002). Primers for site-directed mutagenesis were designed using the NEBasechanger tool (nebasechanger.neb.com/) and site-directed mutagenesis was performed with the Q5® Site-Directed Mutagenesis kit (catalog #: E0554S). Following confirmation by Sanger sequencing, the mutant constructs were Gateway cloned, as well as a wild-type VPS4A construct, into the pLX_TRC313 expression vector. Following confirmation by Sanger sequencing, lentivirus was generated in HEK 293T cells. SpCas9-expressing cell lines JR, and 59M were transduced with the mutant expression lentivirus in 6 well dishes. 24 hours after transduction, the culture media was replaced with media containing 200 μg/mL hygromycin. The growth kinetics of the 59M cultures were tracked by repeated cell counts using a Vi-CELL XR (Beckman-Coulter).

### Cellular Immunostains and Confocal Microscopy

Immunostains were performed on cells plated in 8-well chamber slides and grown for 5-6 days. Cells were fixed and permeabilized using standard paraformaldehyde and triton-based protocols. Immunostaining was performed with validated primary antibodies (Table 3) with alexa-fluor-conjugated secondary antibodies. DNA was visualized with DAPI and images were obtained with either an upright epifluorescence microscope, or a Nikon Eclipse Ti inverted microscope equipped with a Yokogawa Life Sciences CSU-W1 spinning disc confocal system. Images were quantified using CellProfiler v3.1.9 and ImageJ where indicated.

For plating, cells were seeded into 8-well chamber slides (either Nunc Lab-Tek II Chamber Slides, or Ibidi tissue culture treated g-Slide 8-Well). For Lab-Tek slides, chambers were coated with 1:50 dilutions of collagen I (Corning Collagen I, catalog #: 354249) and laminin (Sigma, catalog #: L2020) in 1×PBS for 1-3 hours at 37° C. Depending on the cell line, cells were seeded at a range of densities from 5,000 to 30,000 cells per chamber. For experiments with doxycycline induced RNAi, cells were first plated in small T25 flasks in the presence or absence of 1 μM doxycycline and treated for 4-5 days and then harvested using 0.25% trypsin and then plated into chamber slides which were incubated for another 1-2 days. For CRISPR-based gene inactivation, SpCas9 stable cell lines were first seeded and infected in 6-well plates. Media was changed 24 hours later and selected with puromycin for an additional 24 hours. Selected cells were then trypsinized from 6-wells plates and moved to chamber slides in media that lacked puromycin and cultured for an additional 3 days.

After incubation, cells were washed with 1×PBS and fixed with fresh 4% paraformaldehyde in 1×PBS for 15 minutes. Fixation was quenched with two washes of0.2 M glycine in 1×PBS. Cells were perleabilized with 0.10.2% (v/v) Triton X-100 in 1×PBS for 1015 minutes at room temperature and blocked with blocking buffer (1% (w/v) bovine serum albumin (BSA) in 1×PBS, or 10% normal goat serum (w/v) in 1×PBS). All primary antibodies were diluted in blocking buffer and incubated overnight at 4° C. (see below for antibody sources and dilutions). Cells were washed three times with 1×PBS and stained with Alexa Fluor conjugated secondary antibodies (Molecular Probes, ThermoFisher) diluted 1:500˜1:1000 in blocking buffer. Cells were counterstained with 5 μg/mL DAPI in 1×PBS. Cells plated in Nunc Lab-Tek II plates were then incubated for 20 minutes and washed twice with deionized water, and coverslipped with ProLong Gold Antifade Mountant (ThermoFisher). Images of cellular abscission were collected with an upright epifluorescence microscope; other images were captured on a Nikon Eclipse Ti inverted microscope with a Yokogawa Life Sciences CSU-W1 spinning disc confocal.

TABLE 3
Antibodies used for immunofluorescence.

| Target | Species | Clonality | Dilution | | Vendor | Lot | Catalog # |
|---|---|---|---|---|---|---|---|
| CHMP4B | Rabbit | Polyclonal | 4 | μg/mL | Proteintech | | 13683-1-AP |
| Emerin | Mouse | Clone 0203 | 0.5 | μg/mL | Novus | 2602 | NBP2-52877 |
| | | | | | Biologicals | | |
| RAB7 | Rabbit | Clone D95F2 | | | Cell Signaling | 1 | #9367S |
| | | | | | Technology | | |
| LC3B | Rabbit | Clone D11 | 0.5 | μg/mL | Cell Signaling | 13 | #3868P |
| | | | | | Technology | | |
| SEC61B | Rabbit | Polyclonal | 1 | μg/mL | Sigma-Aldrich | A113820 | HPA049407 |
| Tubulin | Mouse | Clone DM1A | | | Sigma-Aldrich | | T6199 |
| anti-rabbit | Goat | Polyclonal | 2 | μg/mL | ThermoFisher | 2018309 | A-11008 |
| Alexa Fluor | | | | | Scientific | | |
| 488 | | | | | | | |
| anti-mouse | Goat | Polyclonal | 4 | μg/mL | ThermoFisher | 2014175 | A-11004 |
| Alexa Fluor | | | | | Scientific | | |
| 568 | | | | | | | |

### Quantification of Cellular Immunostains

Confocal fluorescence images of CHMP4B3 speckles and other punctate stains of LC3B3, RAB7 and SEC61B were analyzed and quantified after control and doxycycline treatment using a custom image analysis pipeline in CellProfiler (Kamentsky et al. Bioinformatics, 27(8):1179-1180, 2011). Briefly, cells and background were identified using background controlled nuclear (DAPI) and cytoplasmic (CHMP4B-alexa fluor 488/561 or Cellmask Deep Red Stain—ThermoFisher Scientific—H32721) immunofluorescent signals. Fluorescent signals above an adaptive background-controlled threshold were quantified and counted as speckles of >3 μm and assigned to background or cellular areas. Cell speckle counts were then log 2-normalized after adding a value of 1. Data was plotted using GraphPad Prism v8.3.0 and statistical significance was determined using GraphPad's inbuilt ANOVA test with false-discovery correction using the corrected method of Benjamini and Yekutieli.

### CRISPR-SpCas9 Loss-of-Function Screen for Modifiers of VPS4A Dependency

SNU213 pancreatic cancer cells stably transduced with the CRISPR-SpCas9 endonuclease and the shVPS4A-2 inducible RNAi system were infected with a genome-scale Brunello lentiviral sgRNA library (Addgene—73179). To prepare for infection, SNU213 cells were passaged and upscaled to 300×10⁶ cells at 37° C., 5% CO₂ for 1.5 weeks in T75, T175 and then 500 cm² bioassay plates (Nunc Nunclon Delta Treated Square BioAssay Dish, ThermoFisher Scientific—166508) with standardized screening medium; L-glutamine containing RPMI-1640 medium supplemented with 10% FBS (Sigma-Aldrich—F4135), 2 μg/mL blasticidin S (SpCas9 selection; Gibco ThermoFisher Scientific—A1113903) and 300 μg/mL hygromycin B (RNAi system selection; Gibco ThermoFisher Scientific—10687010). Cells were then harvested with trypsin and counted using a Vi-CELL XR and trypan blue exclusion (Beckman Coulter).

For infection, cells were diluted to 1×10⁶ cells/mL in 300 mL L-glutamine containing RPMI-1640 medium without antibiotics. Polybrene (MilliPore-Sigma—TR-1003-G) was added to a final concentration of 8 μg/mL, followed by 16 mL of previously titrated Brunello lentiviral particles to obtain an multiplicity of infection (MOI) of 0.4 and a coverage of ˜1.500 cells/sgRNA. The cell suspension was mixed by manual pipetting and then plated into twelve 12-well plates at 2×10⁶ cells per well. Plates were spinfected for 1.5 hours at 750×g, 35° C. and then further incubated overnight at 37° C., 5% CO₂. The following morning, all infected cells were collected by trypsinization and combined into 1 pool. Cells were then diluted in standardized screening medium supplemented with 2 μg/mL puromycin (Gibco ThermoFisher Scientific—A1113803) and plated in twelve 500 cm² bioassay plates with 120 mL of cell suspension per plate. Cells were then grown and selected with puromycin for 5 days to allow for CRISPR-SpCas9 mutagenesis.

Following counting, all cells were harvested by trypsin collection and counted. Then, 40×10⁶ cells were plated for each treatment arm in four 500 cm² bioassay plates (10×10⁶ cells per plate, ˜500× coverage) with 120 mL medium per plate. Each treatment arm was replicated (4 plates per replicate, 16 plates total) and cells were treated with either standardized screening medium with 2 μg/mL puromycin or standardized screening medium with 2 μM doxycycline and 2 μg/mL puromycin for a period of 2 weeks. During this period, the medium was refreshed every 3 days and cells were monitored and passaged to maintain 40×10⁶ cells per replicate.

After treatment, surviving cells were collected by mild trypsinization and collected by centrifugation at 500×g, supernatant was removed and cell pellets were frozen at −80° C. Genomic DNA from cell pellets (at least 30 million cells per replicate) was purified using silica-membrane-based nucleic acid extraction with the QiaAMP DNA Mini kit (QiaGen 51304). For this purpose, cell pellets for each replicate were suspended in PBS at a concentration of 25×106 cells/mL and divided into 1.5 mL tubes containing 5×10⁶ cells each. These were then processed according to the QiaAMP DNA Mini kit protocol with 2 modifications. During the proteinase K incubation step, 1 mg/mL RNase A (QiaGen 19101) was added to degrade contaminating cellular RNA. For gDNA elution, spin columns were incubated with 125 μL elution buffer at 56° C. for 1 hour before elution by centrifugation, this step was repeated once and both 125 μL fractions were combined. Following gDNA extraction, gDNA concentrations were measured using a NanoDrop 8000 (ThermoFisher Scientific ND-8000-GL).

To determine the sgRNA sequences present in the gDNA of surviving cells, a total of 240 μg of gDNA for each replicate was subjected to PCR amplification using primers with Illumina P5 and P7 adapters. Each PCR reaction was performed in 100 μL using 10 μg of input gDNA. PCR was carried out over 28 cycles using the ExTaq hot-start DNA polymerase (Clontech RR001C). Amplified sgRNAs were purified using the AMPure XP magnetic bead purification system (Beckman Coulter, A63880). Amplified products were sequenced by next-generation single short-read 50-cycle Illumina-based sequencing on a HiSeq 2500. Individual sgRNA read counts were sample normalized to read counts per million, adjusted by a value of 1 and then log 2 transformed. Log 2-normalized sgRNA scores were then compared to the plasmid input library to determine sgRNA fold changes. Fold change scores were then sorted and collapsed into a single gene score with statistical significance values using the STARS v1.3 algorithm as a python script (Doench et al. Nature Biotechnol, 34(2):184-191, 2016). For this purpose, a threshold of 50% was used with a 1000× null distribution. The analysis was performed in both a negative (depletion) and positive (enrichment) direction and the lowest FDR q-value of these two directions was taken for each gene. Q-values below 0.05 were regarded as significant. The top 50 most significant genes were then clustered and visualized using functional associations predicted with STRING v11.0 (string-db.org).

Integrated gene-set enrichment analysis was performed on the top 50 significant genes using metascape's human-standardized express analysis (GO, Reactome, KEGG, CORUM gene sets) (metascape.org, update 2019-08-14). Data was visualized and plotted using Graphpad Prism v8.3.0.

### Interferon Dose-Response Curves

To determine whether interferon treatment cooperate with VPS4A suppression, pancreatic cancer cell lines (KP4, PANC0403 and SNU213) stably expressing the doxycycline-inducible RNAi system against VPS4A (shVPS4A-2) were plated in white-walled 96-well plates at 100˜400 cells per well in 100 μL of 10% FBS-supplemented RPMI-1640 with L-glutamine with or without 1 μM doxycycline. Plates were incubated at 37° C., 5% CO₂ for 3 days. A 9-point log 10 titration of a stock solution (5 μg/mL) of purified recombinant human interferon-β1 or interferon-γ (PeproTech 300-02BC; 300-02) dissolved in 1% BSA containing PBS with 0.3% Tween-20 was then added to the cells using a D300e Digital Dispenser (Tecan) using T8+ and D4+ dispense cassette heads (HP). At the same time, the doxycycline was refreshed. Cells were then incubated for an additional 3 days at 37° C., 5% CO₂. Afterwards, 100 μL of premixed CellTiter-Glo (Promega) reagent was added to the wells to lyse cells by shaking the plates at 500 RPM for 10 minutes at room temperature. Luminescence for each well was then measured using an Envision plate reader (Perkin Elmer) to measure cell viability using the ATP-based readout. Luminescence signal for each well was normalized to the average signal from 6 wells treated without interferon for each cell line. These normalized values were then visualized using GraphPad Prism 8.3.0 fitted with a four-parameter log-based non-linear dose-response curve. For each dose point, 3 replicate wells were used and the experiment was repeated once after two weeks (total of 6 values for each dose point). The 6-day timing of the assay was optimized to reach 50% inhibition in cell viability through VPS4A suppression with doxycycline treatment in the PANC0403 and SNU213 cell lines. Doxycycline was refreshed at day 3 to maintain stringent VPS4A suppression during the experiment.

### Correlation Analysis for VPS4A and VPS4B Dependency Scores

CRISPR VPS4A or VPS4B dependency scores across 622 cell lines from the Broad Institute's public 19Q3 dependency map were correlated with gene expression, copy number, proteomics, and other CRISPR dependency scores from the Broad Institute's 19Q3 Dependency Map/CCLE release. Pearson's correlations were performed in R using the cor.test function. P-values were then corrected for false discovery using the Benjamini-Hochberg method of the p.adjust function in R and q-values were −log 10-normalized. The results were plotted using GraphPad Prism v8.3.0. Some of the results were used for gene-set enrichment analysis. For this purpose, symbols for the top significantly (5% FDR) correlating genes were uploaded to Metascape (metascape.org, update 2019-08-14) and analyzed for Homo sapiens by incorporating GO, KEGG and Reactome gene sets.

For the multiple linear models, VPS4A and VPS4B CRISPR dependency scores together with RNAseq-determined mRNA expression values for the indicated genes were binned into 10 equal-sized groups of cell lines. A multiple linear model was then trained in R using the specified features using the in-built lm function. For this purpose, the model was first trained on 9 of the bins and then utilized to predict the last bin. This process was repeated 10 times to predict all 10 bins (10-fold cross validation) and all prediction scores were then collected and correlated with the real observed values using Pearson correlation. The results of the 4-parameter multiple linear models were plotted using GraphPad Prism v8.3.0 and statistics for each of the models were extracted and saved into a table using a simple R script.

TABLE 4
Gene dependencies that correlated with tumor suppressor
gene copy loss in the CRISPR-SpCas9 and RNAi analysis.

| ID | Rank | Pair | Dep_Gene | Chr_Dep | KB_dep | TSG_Gene | Chr_TSG | KB_TSG |
|---|---|---|---|---|---|---|---|---|
| 1 | 1 | VPS4A- | VPS4A | 16q | 69311.38 | SMAD4 | 18q | 51030.21 |
| | | SMAD4 | | | | | | |
| 2 | 1 | CHMP4B- | CHMP4B | 20q | 33811.3 | SMAD4 | 18q | 51030.21 |
| | | SMAD4 | | | | | | |
| 3 | 1 | GRHL2- | GRHL2 | 8q | 101492.44 | SMAD4 | 18q | 51030.21 |
| | | SMAD4 | | | | | | |
| 4 | 1 | TUBB4B- | TUBB4B | 9q | 137241.26 | SMAD4 | 18q | 51030.21 |
| | | SMAD4 | | | | | | |
| 5 | 1 | KRAS- | KRAS | 12p | 25204.79 | SMAD4 | 18q | 51030.21 |
| | | SMAD4 | | | | | | |
| 6 | 1 | KLF5- | KLF5 | 13q | 73058.79 | SMAD4 | 18q | 51030.21 |
| | | SMAD4 | | | | | | |
| 7 | 1 | GRK2- | GRK2 | 11q | 67266.4 | SMAD4 | 18q | 51030.21 |
| | | SMAD4 | | | | | | |
| 8 | 1 | EGFR- | EGFR | 7p | 55019.03 | SMAD4 | 18q | 51030.21 |
| | | SMAD4 | | | | | | |
| 9 | 1 | EP300- | EP300 | 22q | 41092.61 | SMAD4 | 18q | 51030.21 |
| | | SMAD4 | | | | | | |
| 10 | 1 | PRKAR1A- | PRKAR1A | 17q | 68512.38 | SMAD4 | 18q | 51030.21 |
| | | SMAD4 | | | | | | |
| 11 | 1 | ITGA3- | ITGA3 | 17q | 50055.97 | SMAD4 | 18q | 51030.21 |
| | | SMAD4 | | | | | | |
| 12 | 1 | IRF4-SMAD4 | IRF4 | 6p | 391.74 | SMAD4 | 18q | 51030.21 |
| 13 | 1 | SCAP- | SCAP | 3p | 47413.69 | SMAD4 | 18q | 51030.21 |
| | | SMAD4 | | | | | | |
| 14 | 1 | ACSL5- | ACSL5 | 10q | 112375.27 | SMAD4 | 18q | 51030.21 |
| | | SMAD4 | | | | | | |
| 15 | 1 | UPF1- | UPF1 | 19p | 18831.94 | SMAD4 | 18q | 51030.21 |
| | | SMAD4 | | | | | | |
| 16 | 1 | RAPGEF1- | RAPGEF1 | 9q | 131576.77 | SMAD4 | 18q | 51030.21 |
| | | SMAD4 | | | | | | |
| 17 | 1 | RAB6A- | RAB6A | 11q | 73675.64 | SMAD4 | 18q | 51030.21 |
| | | SMAD4 | | | | | | |
| 18 | 1 | TTC24- | TTC24 | 1q | 156581.37 | SMAD4 | 18q | 51030.21 |
| | | SMAD4 | | | | | | |
| 19 | 1 | JUNB- | JUNB | 19p | 12791.5 | SMAD4 | 18q | 51030.21 |
| | | SMAD4 | | | | | | |
| 20 | 1 | COPS4- | COPS4 | 4q | 83035.09 | SMAD4 | 18q | 51030.21 |
| | | SMAD4 | | | | | | |
| 21 | 1 | SREBF1- | SREBF1 | 17p | 17811.35 | SMAD4 | 18q | 51030.21 |
| | | SMAD4 | | | | | | |
| 22 | 1 | METAP1- | METAP1 | 4q | 98995.64 | SMAD4 | 18q | 51030.21 |
| | | SMAD4 | | | | | | |
| 23 | 1 | EED-SMAD4 | EED | 11q | 86244.38 | SMAD4 | 18q | 51030.21 |
| 24 | 1 | PRAG1- | PRAG1 | 8p | 8317.74 | SMAD4 | 18q | 51030.21 |
| | | SMAD4 | | | | | | |
| 25 | 1 | RPS21- | RPS21 | 20q | 62387.07 | SMAD4 | 18q | 51030.21 |
| | | SMAD4 | | | | | | |
| 26 | 1 | PKP3- | PKP3 | 11p | 394.18 | SMAD4 | 18q | 51030.21 |
| | | SMAD4 | | | | | | |
| 27 | 1 | GMNN- | GMNN | 6p | 24774.93 | SMAD4 | 18q | 51030.21 |
| | | SMAD4 | | | | | | |
| 28 | 1 | LMO7- | LMO7 | 13q | 75760.66 | SMAD4 | 18q | 51030.21 |
| | | SMAD4 | | | | | | |
| 29 | 1 | AKAP9- | AKAP9 | 7q | 91940.88 | SMAD4 | 18q | 51030.21 |
| | | SMAD4 | | | | | | |
| 30 | 1 | ULK3- | ULK3 | 15q | 74836.12 | SMAD4 | 18q | 51030.21 |
| | | SMAD4 | | | | | | |
| 31 | 1 | LIMS1- | LIMS1 | 2q | 108588.3 | SMAD4 | 18q | 51030.21 |
| | | SMAD4 | | | | | | |
| 32 | 1 | UBE2Z- | UBE2Z | 17q | 48908.37 | SMAD4 | 18q | 51030.21 |
| | | SMAD4 | | | | | | |
| 33 | 1 | CSNK1D- | CSNK1D | 17q | 82242.66 | SMAD4 | 18q | 51030.21 |
| | | SMAD4 | | | | | | |
| 34 | 1 | COPS7A- | COPS7A | 12p | 6723.98 | SMAD4 | 18q | 51030.21 |
| | | SMAD4 | | | | | | |
| 35 | 1 | PSMD6- | PSMD6 | 3p | 64010.55 | SMAD4 | 18q | 51030.21 |
| | | SMAD4 | | | | | | |
| 36 | 1 | RPS27A- | RPS27A | 2p | 55232.41 | SMAD4 | 18q | 51030.21 |
| | | SMAD4 | | | | | | |
| 37 | 2 | WDR77- | WDR77 | 1p | 111439.89 | CDKN2B | 9p | 22002.9 |
| | | CDKN2B | | | | | | |
| 38 | 2 | PRMT5- | PRMT5 | 14q | 22920.51 | CDKN2B | 9p | 22002.9 |
| | | CDKN2B | | | | | | |
| 39 | 2 | KIF2C- | KIF2C | 1p | 44739.82 | CDKN2B | 9p | 22002.9 |
| | | CDKN2B | | | | | | |
| 40 | 2 | CCND1- | CCND1 | 11q | 69641.11 | CDKN2B | 9p | 22002.9 |
| | | CDKN2B | | | | | | |
| 41 | 2 | CCNE1- | CCNE1 | 19q | 29811.99 | CDKN2B | 9p | 22002.9 |
| | | CDKN2B | | | | | | |
| 42 | 2 | NFE2L2- | NFE2L2 | 2q | 177230.3 | CDKN2B | 9p | 22002.9 |
| | | CDKN2B | | | | | | |
| 43 | 2 | CDK6- | CDK6 | 7q | 92604.92 | CDKN2B | 9p | 22002.9 |
| | | CDKN2B | | | | | | |
| 44 | 2 | MARCH5- | MARCH5 | 10q | 92291.16 | CDKN2B | 9p | 22002.9 |
| | | CDKN2B | | | | | | |
| 45 | 2 | EZH2- | EZH2 | 7q | 148807.37 | CDKN2B | 9p | 22002.9 |
| | | CDKN2B | | | | | | |
| 46 | 2 | CDKN1A- | CDKN1A | 6p | 36676.49 | CDKN2B | 9p | 22002.9 |
| | | CDKN2B | | | | | | |
| 47 | 2 | EFTUD2- | EFTUD2 | 17q | 44850.29 | CDKN2B | 9p | 22002.9 |
| | | CDKN2B | | | | | | |
| 48 | 2 | ORC6- | ORC6 | 16q | 46689.65 | CDKN2B | 9p | 22002.9 |
| | | CDKN2B | | | | | | |
| 49 | 2 | SMARCD1- | SMARCD1 | 12q | 50085.2 | CDKN2B | 9p | 22002.9 |
| | | CDKN2B | | | | | | |
| 50 | 2 | WWTR1- | WWTR1 | 3q | 149517.23 | CDKN2B | 9p | 22002.9 |
| | | CDKN2B | | | | | | |
| 51 | 2 | SKP2- | SKP2 | 5p | 36152.04 | CDKN2B | 9p | 22002.9 |
| | | CDKN2B | | | | | | |
| 52 | 2 | KEAP1- | KEAP1 | 19p | 10486.12 | CDKN2B | 9p | 22002.9 |
| | | CDKN2B | | | | | | |
| 53 | 2 | GYPA- | GYPA | 4q | 144109.3 | CDKN2B | 9p | 22002.9 |
| | | CDKN2B | | | | | | |
| 54 | 2 | SNRPC- | SNRPC | 6p | 34757.09 | CDKN2B | 9p | 22002.9 |
| | | CDKN2B | | | | | | |
| 55 | 2 | MSI2- | MSI2 | 17q | 57257.01 | CDKN2B | 9p | 22002.9 |
| | | CDKN2B | | | | | | |
| 56 | 2 | RBL1- | RBL1 | 20q | 37003.57 | CDKN2B | 9p | 22002.9 |
| | | CDKN2B | | | | | | |
| 57 | 2 | ORC1- | ORC1 | 1p | 52372.83 | CDKN2B | 9p | 22002.9 |
| | | CDKN2B | | | | | | |
| 58 | 2 | CDC6- | CDC6 | 17q | 40287.89 | CDKN2B | 9p | 22002.9 |
| | | CDKN2B | | | | | | |
| 59 | 2 | PRSS42- | PRSS42 | 3p | 46830.4 | CDKN2B | 9p | 22002.9 |
| | | CDKN2B | | | | | | |
| 60 | 2 | IPO9- | IPO9 | 1q | 201829.16 | CDKN2B | 9p | 22002.9 |
| | | CDKN2B | | | | | | |
| 61 | 2 | BCOR- | BCOR | Xp | 40051.25 | CDKN2B | 9p | 22002.9 |
| | | CDKN2B | | | | | | |
| 62 | 2 | MYBL2- | MYBL2 | 20q | 43667.02 | CDKN2B | 9p | 22002.9 |
| | | CDKN2B | | | | | | |
| 63 | 2 | SDHA- | SDHA | 5p | 218.22 | CDKN2B | 9p | 22002.9 |
| | | CDKN2B | | | | | | |
| 64 | 2 | TP53BP1- | TP53BP1 | 15q | 43403.06 | CDKN2B | 9p | 22002.9 |
| | | CDKN2B | | | | | | |
| 65 | 2 | CDK2- | CDK2 | 12q | 55966.77 | CDKN2B | 9p | 22002.9 |
| | | CDKN2B | | | | | | |
| 66 | 2 | RING1- | RING1 | 6p | 33208.51 | CDKN2B | 9p | 22002.9 |
| | | CDKN2B | | | | | | |
| 67 | 2 | SUZ12- | SUZ12 | 17q | 31937.01 | CDKN2B | 9p | 22002.9 |
| | | CDKN2B | | | | | | |
| 68 | 2 | LMO2- | LMO2 | 11p | 33858.58 | CDKN2B | 9p | 22002.9 |
| | | CDKN2B | | | | | | |
| 69 | 3 | WDR77- | WDR77 | 1p | 111439.89 | CDKN2A | 9p | 21967.75 |
| | | CDKN2A | | | | | | |
| 70 | 3 | CCND1- | CCND1 | 11q | 69641.11 | CDKN2A | 9p | 21967.75 |
| | | CDKN2A | | | | | | |
| 71 | 3 | PRMT5- | PRMT5 | 14q | 22920.51 | CDKN2A | 9p | 21967.75 |
| | | CDKN2A | | | | | | |
| 72 | 3 | CCNE1- | CCNE1 | 19q | 29811.99 | CDKN2A | 9p | 21967.75 |
| | | CDKN2A | | | | | | |
| 73 | 3 | CDK6- | CDK6 | 7q | 92604.92 | CDKN2A | 9p | 21967.75 |
| | | CDKN2A | | | | | | |
| 74 | 3 | KIF2C- | KIF2C | 1p | 44739.82 | CDKN2A | 9p | 21967.75 |
| | | CDKN2A | | | | | | |
| 75 | 3 | EZH2- | EZH2 | 7q | 148807.37 | CDKN2A | 9p | 21967.75 |
| | | CDKN2A | | | | | | |
| 76 | 3 | CDKN1A- | CDKN1A | 6p | 36676.49 | CDKN2A | 9p | 21967.75 |
| | | CDKN2A | | | | | | |
| 77 | 3 | WWTR1- | WWTR1 | 3q | 149517.23 | CDKN2A | 9p | 21967.75 |
| | | CDKN2A | | | | | | |
| 78 | 3 | NFE2L2- | NFE2L2 | 2q | 177230.3 | CDKN2A | 9p | 21967.75 |
| | | CDKN2A | | | | | | |
| 79 | 3 | ORC1- | ORC1 | 1p | 52372.83 | CDKN2A | 9p | 21967.75 |
| | | CDKN2A | | | | | | |
| 80 | 3 | SKP2- | SKP2 | 5p | 36152.04 | CDKN2A | 9p | 21967.75 |
| | | CDKN2A | | | | | | |
| 81 | 3 | RBL1- | RBL1 | 20q | 37003.57 | CDKN2A | 9p | 21967.75 |
| | | CDKN2A | | | | | | |
| 82 | 3 | MARCH5- | MARCH5 | 10q | 92291.16 | CDKN2A | 9p | 21967.75 |
| | | CDKN2A | | | | | | |
| 83 | 3 | ORC6- | ORC6 | 16q | 46689.65 | CDKN2A | 9p | 21967.75 |
| | | CDKN2A | | | | | | |
| 84 | 3 | MYBL2- | MYBL2 | 20q | 43667.02 | CDKN2A | 9p | 21967.75 |
| | | CDKN2A | | | | | | |
| 85 | 3 | PPP1R15B- | PPP1R15B | 1q | 204403.36 | CDKN2A | 9p | 21967.75 |
| | | CDKN2A | | | | | | |
| 86 | 3 | BMI1- | BMI1 | 10p | 22321.21 | CDKN2A | 9p | 21967.75 |
| | | CDKN2A | | | | | | |
| 87 | 3 | PRSS42- | PRSS42 | 3p | 46830.4 | CDKN2A | 9p | 21967.75 |
| | | CDKN2A | | | | | | |
| 88 | 3 | RING1- | RING1 | 6p | 33208.51 | CDKN2A | 9p | 21967.75 |
| | | CDKN2A | | | | | | |
| 89 | 3 | CDK2- | CDK2 | 12q | 55966.77 | CDKN2A | 9p | 21967.75 |
| | | CDKN2A | | | | | | |
| 90 | 3 | SUZ12- | SUZ12 | 17q | 31937.01 | CDKN2A | 9p | 21967.75 |
| | | CDKN2A | | | | | | |
| 91 | 3 | SNRPC- | SNRPC | 6p | 34757.09 | CDKN2A | 9p | 21967.75 |
| | | CDKN2A | | | | | | |
| 92 | 3 | GRPEL1- | GRPEL1 | 4p | 7058.9 | CDKN2A | 9p | 21967.75 |
| | | CDKN2A | | | | | | |
| 93 | 3 | TRRAP- | TRRAP | 7q | 98878.49 | CDKN2A | 9p | 21967.75 |
| | | CDKN2A | | | | | | |
| 94 | 3 | EAF1- | EAF1 | 3p | 15427.56 | CDKN2A | 9p | 21967.75 |
| | | CDKN2A | | | | | | |
| 95 | 4 | ARF1-BAP1 | ARF1 | 1q | 228082.66 | BAP1 | 3p | 52401 |
| 96 | 4 | AIFM1-BAP1 | AIFM1 | Xq | 130129.36 | BAP1 | 3p | 52401 |
| 97 | 4 | RPS15A- | RPS15A | 16p | 18782.96 | BAP1 | 3p | 52401 |
| | | BAP1 | | | | | | |
| 98 | 4 | EDF1-BAP1 | EDF1 | 9q | 136862.67 | BAP1 | 3p | 52401 |
| 99 | 4 | FUBP1-BAP1 | FUBP1 | 1p | 77948.4 | BAP1 | 3p | 52401 |
| 100 | 4 | EGFR-BAP1 | EGFR | 7p | 55019.03 | BAP1 | 3p | 52401 |
| 101 | 4 | ERBB3- | ERBB3 | 12q | 56080.03 | BAP1 | 3p | 52401 |
| | | BAP1 | | | | | | |
| 102 | 4 | RPS27A- | RPS27A | 2p | 55232.41 | BAP1 | 3p | 52401 |
| | | BAP1 | | | | | | |
| 103 | 4 | CIAPIN1- | CIAPIN1 | 16q | 57428.17 | BAP1 | 3p | 52401 |
| | | BAP1 | | | | | | |
| 104 | 4 | CBFB-BAP1 | CBFB | 16q | 67029.15 | BAP1 | 3p | 52401 |
| 105 | 4 | SEM1-BAP1 | SEM1 | 7q | 96688.77 | BAP1 | 3p | 52401 |
| 106 | 4 | GRK2-BAP1 | GRK2 | 11q | 67266.4 | BAP1 | 3p | 52401 |
| 107 | 4 | PHF2-BAP1 | PHF2 | 9q | 93576.63 | BAP1 | 3p | 52401 |
| 108 | 4 | UBE2H- | UBE2H | 7q | 129830.73 | BAP1 | 3p | 52401 |
| | | BAP1 | | | | | | |
| 109 | 4 | RPL18A- | RPL18A | 19p | 17859.88 | BAP1 | 3p | 52401 |
| | | BAP1 | | | | | | |
| 110 | 4 | KIF2A-BAP1 | KIF2A | 5q | 62306.16 | BAP1 | 3p | 52401 |
| 111 | 5 | AIFM1- | AIFM1 | Xq | 130129.36 | PBRM1 | 3p | 52545.39 |
| | | PBRM1 | | | | | | |
| 112 | 5 | ARF1- | ARF1 | 1q | 228082.66 | PBRM1 | 3p | 52545.39 |
| | | PBRM1 | | | | | | |
| 113 | 5 | RPS15A- | RPS15A | 16p | 18782.96 | PBRM1 | 3p | 52545.39 |
| | | PBRM1 | | | | | | |
| 114 | 5 | EDF1- | EDF1 | 9q | 136862.67 | PBRM1 | 3p | 52545.39 |
| | | PBRM1 | | | | | | |
| 115 | 5 | FUBP1- | FUBP1 | 1p | 77948.4 | PBRM1 | 3p | 52545.39 |
| | | PBRM1 | | | | | | |
| 116 | 5 | ERBB2- | ERBB2 | 17q | 39699.98 | PBRM1 | 3p | 52545.39 |
| | | PBRM1 | | | | | | |
| 117 | 5 | EGFR- | EGFR | 7p | 55019.03 | PBRM1 | 3p | 52545.39 |
| | | PBRM1 | | | | | | |
| 118 | 5 | RPS27A- | RPS27A | 2p | 55232.41 | PBRM1 | 3p | 52545.39 |
| | | PBRM1 | | | | | | |
| 119 | 5 | SEM1- | SEM1 | 7q | 96688.77 | PBRM1 | 3p | 52545.39 |
| | | PBRM1 | | | | | | |
| 120 | 5 | CBFB- | CBFB | 16q | 67029.15 | PBRM1 | 3p | 52545.39 |
| | | PBRM1 | | | | | | |
| 121 | 5 | ERBB3- | ERBB3 | 12q | 56080.03 | PBRM1 | 3p | 52545.39 |
| | | PBRM1 | | | | | | |
| 122 | 5 | GRK2- | GRK2 | 11q | 67266.4 | PBRM1 | 3p | 52545.39 |
| | | PBRM1 | | | | | | |
| 123 | 5 | RPL18A- | RPL18A | 19p | 17859.88 | PBRM1 | 3p | 52545.39 |
| | | PBRM1 | | | | | | |
| 124 | 5 | KIF2A- | KIF2A | 5q | 62306.16 | PBRM1 | 3p | 52545.39 |
| | | PBRM1 | | | | | | |
| 125 | 6 | AIFM1- | AIFM1 | Xq | 130129.36 | RHOA | 3p | 49359.14 |
| | | RHOA | | | | | | |
| 126 | 6 | EGFR-RHOA | EGFR | 7p | 55019.03 | RHOA | 3p | 49359.14 |
| 127 | 6 | EDF1-RHOA | EDF1 | 9q | 136862.67 | RHOA | 3p | 49359.14 |
| 128 | 6 | RPS15A- | RPS15A | 16p | 18782.96 | RHOA | 3p | 49359.14 |
| | | RHOA | | | | | | |
| 129 | 6 | CIAPIN1- | CIAPIN1 | 16q | 57428.17 | RHOA | 3p | 49359.14 |
| | | RHOA | | | | | | |
| 130 | 6 | FUBP1- | FUBP1 | 1p | 77948.4 | RHOA | 3p | 49359.14 |
| | | RHOA | | | | | | |
| 131 | 6 | GRK2-RHOA | GRK2 | 11q | 67266.4 | RHOA | 3p | 49359.14 |
| 132 | 6 | ADAR- | ADAR | 1q | 154582.06 | RHOA | 3p | 49359.14 |
| | | RHOA | | | | | | |
| 133 | 6 | ESR1-RHOA | ESR1 | 6q | 151809.11 | RHOA | 3p | 49359.14 |
| 134 | 6 | PHF2-RHOA | PHF2 | 9q | 93576.63 | RHOA | 3p | 49359.14 |
| 135 | 6 | PCM1-RHOA | PCM1 | 8p | 17922.86 | RHOA | 3p | 49359.14 |
| 136 | 6 | CBFB-RHOA | CBFB | 16q | 67029.15 | RHOA | 3p | 49359.14 |
| 137 | 6 | FBXO11- | FBXO11 | 2p | 47806.92 | RHOA | 3p | 49359.14 |
| | | RHOA | | | | | | |
| 138 | 7 | ITGAV- | ITGAV | 2q | 186590.06 | PDGFRB | 5q | 150113.84 |
| | | PDGFRB | | | | | | |
| 139 | 7 | MED1- | MED1 | 17q | 39404.29 | PDGFRB | 5q | 150113.84 |
| | | PDGFRB | | | | | | |
| 140 | 7 | MYBL2- | MYBL2 | 20q | 43667.02 | PDGFRB | 5q | 150113.84 |
| | | PDGFRB | | | | | | |
| 141 | 7 | WWTR1- | WWTR1 | 3q | 149517.23 | PDGFRB | 5q | 150113.84 |
| | | PDGFRB | | | | | | |
| 142 | 7 | MED12- | MED12 | Xq | 71118.56 | PDGFRB | 5q | 150113.84 |
| | | PDGFRB | | | | | | |
| 143 | 7 | PPP2R1A- | PPP2R1A | 19q | 52189.8 | PDGFRB | 5q | 150113.84 |
| | | PDGFRB | | | | | | |
| 144 | 7 | FERMT2- | FERMT2 | 14q | 52857.27 | PDGFRB | 5q | 150113.84 |
| | | PDGFRB | | | | | | |
| 145 | 7 | RUNX1- | RUNX1 | 21q | 34787.8 | PDGFRB | 5q | 150113.84 |
| | | PDGFRB | | | | | | |
| 146 | 7 | PTK2- | PTK2 | 8q | 140658.38 | PDGFRB | 5q | 150113.84 |
| | | PDGFRB | | | | | | |
| 147 | 7 | ZFP36L1- | ZFP36L1 | 14q | 68787.66 | PDGFRB | 5q | 150113.84 |
| | | PDGFRB | | | | | | |
| 148 | 7 | TLN1- | TLN1 | 9p | 35697.34 | PDGFRB | 5q | 150113.84 |
| | | PDGFRB | | | | | | |
| 149 | 7 | MED16- | MED16 | 19p | 867.96 | PDGFRB | 5q | 150113.84 |
| | | PDGFRB | | | | | | |
| 150 | 8 | AIFM1- | AIFM1 | Xq | 130129.36 | SETD2 | 3p | 47016.41 |
| | | SETD2 | | | | | | |
| 151 | 8 | ARF1-SETD2 | ARF1 | 1q | 228082.66 | SETD2 | 3p | 47016.41 |
| 152 | 8 | EGFR- | EGFR | 7p | 55019.03 | SETD2 | 3p | 47016.41 |
| | | SETD2 | | | | | | |
| 153 | 8 | EDF1-SETD2 | EDF1 | 9q | 136862.67 | SETD2 | 3p | 47016.41 |
| 154 | 8 | CIAPIN1- | CIAPIN1 | 16q | 57428.17 | SETD2 | 3p | 47016.41 |
| | | SETD2 | | | | | | |
| 155 | 8 | RPS15A- | RPS15A | 16p | 18782.96 | SETD2 | 3p | 47016.41 |
| | | SETD2 | | | | | | |
| 156 | 8 | FUBP1- | FUBP1 | 1p | 77948.4 | SETD2 | 3p | 47016.41 |
| | | SETD2 | | | | | | |
| 157 | 8 | ADAR- | ADAR | 1q | 154582.06 | SETD2 | 3p | 47016.41 |
| | | SETD2 | | | | | | |
| 158 | 8 | GRK2- | GRK2 | 11q | 67266.4 | SETD2 | 3p | 47016.41 |
| | | SETD2 | | | | | | |
| 159 | 8 | CBFB- | CBFB | 16q | 67029.15 | SETD2 | 3p | 47016.41 |
| | | SETD2 | | | | | | |
| 160 | 8 | ESR1-SETD2 | ESR1 | 6q | 151809.11 | SETD2 | 3p | 47016.41 |
| 161 | 8 | GAB1- | GAB1 | 4q | 143336.83 | SETD2 | 3p | 47016.41 |
| | | SETD2 | | | | | | |
| 162 | 9 | MYT1- | MYT1 | 20q | 64164.47 | CDC73 | 1q | 193121.96 |
| | | CDC73 | | | | | | |
| 163 | 9 | NPAT- | NPAT | 11q | 108157.39 | CDC73 | 1q | 193121.96 |
| | | CDC73 | | | | | | |
| 164 | 9 | TEAD1- | TEAD1 | 11p | 12674.42 | CDC73 | 1q | 193121.96 |
| | | CDC73 | | | | | | |
| 165 | 9 | OR5M10- | OR5M10 | 11q | 56576.77 | CDC73 | 1q | 193121.96 |
| | | CDC73 | | | | | | |
| 166 | 9 | RPL22- | RPL22 | 1p | 6185.02 | CDC73 | 1q | 193121.96 |
| | | CDC73 | | | | | | |
| 167 | 9 | SMARCE1- | SMARCE1 | 17q | 40627.73 | CDC73 | 1q | 193121.96 |
| | | CDC73 | | | | | | |
| 168 | 9 | ZMYND11- | ZMYND11 | 10p | 134.48 | CDC73 | 1q | 193121.96 |
| | | CDC73 | | | | | | |
| 169 | 9 | SFPQ-CDC73 | SFPQ | 1p | 35183.6 | CDC73 | 1q | 193121.96 |
| 170 | 9 | MIB1-CDC73 | MIB1 | 18q | 21741.33 | CDC73 | 1q | 193121.96 |
| 171 | 9 | SOD2- | SOD2 | 6q | 159679.06 | CDC73 | 1q | 193121.96 |
| | | CDC73 | | | | | | |
| 172 | 9 | ZFC3H1- | ZFC3H1 | 12q | 71609.6 | CDC73 | 1q | 193121.96 |
| | | CDC73 | | | | | | |
| 173 | 10 | BIRC6- | BIRC6 | 2p | 32357.03 | DICER1 | 14q | 95086.23 |
| | | DICER1 | | | | | | |
| 174 | 10 | RPL36A- | RPL36A | Xq | 101390.89 | DICER1 | 14q | 95086.23 |
| | | DICER1 | | | | | | |
| 175 | 10 | MTA2- | MTA2 | 11q | 62593.2 | DICER1 | 14q | 95086.23 |
| | | DICER1 | | | | | | |
| 176 | 10 | MTOR- | MTOR | 1p | 11106.53 | DICER1 | 14q | 95086.23 |
| | | DICER1 | | | | | | |
| 177 | 10 | UBA6- | UBA6 | 4q | 67615.76 | DICER1 | 14q | 95086.23 |
| | | DICER1 | | | | | | |
| 178 | 10 | HSP90AB1- | HSP90AB1 | 6p | 44246.96 | DICER1 | 14q | 95086.23 |
| | | DICER1 | | | | | | |
| 179 | 10 | SH2D2A- | SH2D2A | 1q | 156806.24 | DICER1 | 14q | 95086.23 |
| | | DICER1 | | | | | | |
| 180 | 10 | AHCYL1- | AHCYL1 | 1p | 109984.77 | DICER1 | 14q | 95086.23 |
| | | DICER1 | | | | | | |
| 181 | 10 | EXOC2- | EXOC2 | 6p | 485.14 | DICER1 | 14q | 95086.23 |
| | | DICER1 | | | | | | |
| 182 | 10 | POLDIP3- | POLDIP3 | 22q | 42583.72 | DICER1 | 14q | 95086.23 |
| | | DICER1 | | | | | | |
| 183 | 10 | RBBP7- | RBBP7 | Xp | 16844.65 | DICER1 | 14q | 95086.23 |
| | | DICER1 | | | | | | |
| 184 | 11 | ITGAV-APC | ITGAV | 2q | 186590.06 | APC | 5q | 112737.86 |
| 185 | 11 | RAB1A-APC | RAB1A | 2p | 65086.85 | APC | 5q | 112737.86 |
| 186 | 11 | DDX21-APC | DDX21 | 10q | 68956.12 | APC | 5q | 112737.86 |
| 187 | 11 | ZFP36-APC | ZFP36 | 19q | 39406.85 | APC | 5q | 112737.86 |
| 188 | 11 | PTK2-APC | PTK2 | 8q | 140658.38 | APC | 5q | 112737.86 |
| 189 | 11 | RAC1-APC | RAC1 | 7p | 6374.5 | APC | 5q | 112737.86 |
| 190 | 11 | PLXNA1- | PLXNA1 | 3q | 126988.59 | APC | 5q | 112737.86 |
| | | APC | | | | | | |
| 191 | 11 | TLN1-APC | TLN1 | 9p | 35697.34 | APC | 5q | 112737.86 |
| 192 | 11 | AMOTL2- | AMOTL2 | 3q | 134355.35 | APC | 5q | 112737.86 |
| | | APC | | | | | | |
| 193 | 11 | MYBL2-APC | MYBL2 | 20q | 43667.02 | APC | 5q | 112737.86 |
| 194 | 12 | DDX39B- | DDX39B | 6p | 31530.22 | KEAP1 | 19p | 10486.12 |
| | | KEAP1 | | | | | | |
| 195 | 12 | FUBP1- | FUBP1 | 1p | 77948.4 | KEAP1 | 19p | 10486.12 |
| | | KEAP1 | | | | | | |
| 196 | 12 | MYB-KEAP1 | MYB | 6q | 135181.32 | KEAP1 | 19p | 10486.12 |
| 197 | 12 | PRKAR1A- | PRKAR1A | 17q | 68512.38 | KEAP1 | 19p | 10486.12 |
| | | KEAP1 | | | | | | |
| 198 | 12 | GCLC- | GCLC | 6p | 53497.34 | KEAP1 | 19p | 10486.12 |
| | | KEAP1 | | | | | | |
| 199 | 12 | GNG5- | GNG5 | 1p | 84498.32 | KEAP1 | 19p | 10486.12 |
| | | KEAP1 | | | | | | |
| 200 | 12 | CBFB- | CBFB | 16q | 67029.15 | KEAP1 | 19p | 10486.12 |
| | | KEAP1 | | | | | | |
| 201 | 12 | STK17B- | STK17B | 2q | 196133.58 | KEAP1 | 19p | 10486.12 |
| | | KEAP1 | | | | | | |
| 202 | 12 | NFE2L2- | NFE2L2 | 2q | 177230.3 | KEAP1 | 19p | 10486.12 |
| | | KEAP1 | | | | | | |
| 203 | 12 | LMO2- | LMO2 | 11p | 33858.58 | KEAP1 | 19p | 10486.12 |
| | | KEAP1 | | | | | | |
| 204 | 13 | TRA2B- | TRA2B | 3q | 185914.57 | PMS2 | 7p | 5970.93 |
| | | PMS2 | | | | | | |
| 205 | 13 | WDR1-PMS2 | WDR1 | 4p | 10074.34 | PMS2 | 7p | 5970.93 |
| 206 | 13 | ZC3H18- | ZC3H18 | 16q | 88570.38 | PMS2 | 7p | 5970.93 |
| | | PMS2 | | | | | | |
| 207 | 13 | MED26- | MED26 | 19p | 16574.91 | PMS2 | 7p | 5970.93 |
| | | PMS2 | | | | | | |
| 208 | 13 | H2AFZ- | H2AFZ | 4q | 99948.09 | PMS2 | 7p | 5970.93 |
| | | PMS2 | | | | | | |
| 209 | 13 | SRRT-PMS2 | SRRT | 7q | 100875.08 | PMS2 | 7p | 5970.93 |
| 210 | 13 | BIRC6-PMS2 | BIRC6 | 2p | 32357.03 | PMS2 | 7p | 5970.93 |
| 211 | 13 | NCKAP1- | NCKAP1 | 2q | 182924.85 | PMS2 | 7p | 5970.93 |
| | | PMS2 | | | | | | |
| 212 | 13 | ACTG1- | ACTG1 | 17q | 81509.97 | PMS2 | 7p | 5970.93 |
| | | PMS2 | | | | | | |
| 213 | 13 | POLR2J- | POLR2J | 7q | 102473.1 | PMS2 | 7p | 5970.93 |
| | | PMS2 | | | | | | |
| 214 | 14 | NPAT- | NPAT | 11q | 108157.39 | MDM4 | 1q | 204516.38 |
| | | MDM4 | | | | | | |
| 215 | 14 | RPL22- | RPL22 | 1p | 6185.02 | MDM4 | 1q | 204516.38 |
| | | MDM4 | | | | | | |
| 216 | 14 | MYT1- | MYT1 | 20q | 64164.47 | MDM4 | 1q | 204516.38 |
| | | MDM4 | | | | | | |
| 217 | 14 | TEAD1- | TEAD1 | 11p | 12674.42 | MDM4 | 1q | 204516.38 |
| | | MDM4 | | | | | | |
| 218 | 14 | TRPS1- | TRPS1 | 8q | 115408.5 | MDM4 | 1q | 204516.38 |
| | | MDM4 | | | | | | |
| 219 | 14 | TBP-MDM4 | TBP | 6q | 170554.33 | MDM4 | 1q | 204516.38 |
| 220 | 14 | SF1-MDM4 | SF1 | 11q | 64764.6 | MDM4 | 1q | 204516.38 |
| 221 | 14 | SMARCE1- | SMARCE1 | 17q | 40627.73 | MDM4 | 1q | 204516.38 |
| | | MDM4 | | | | | | |
| 222 | 14 | ZMYND11- | ZMYND11 | 10p | 134.48 | MDM4 | 1q | 204516.38 |
| | | MDM4 | | | | | | |
| 223 | 15 | RPAP3- | RPAP3 | 12q | 47661.93 | PPP6C | 9q | 125146.57 |
| | | PPP6C | | | | | | |
| 224 | 15 | TUBB-PPP6C | TUBB | 6p | 30720.2 | PPP6C | 9q | 125146.57 |
| 225 | 15 | USP5-PPP6C | USP5 | 12p | 6852.13 | PPP6C | 9q | 125146.57 |
| 226 | 15 | RRM1- | RRM1 | 11p | 4116.08 | PPP6C | 9q | 125146.57 |
| | | PPP6C | | | | | | |
| 227 | 15 | VCP-PPP6C | VCP | 9p | 35056.07 | PPP6C | 9q | 125146.57 |
| 228 | 15 | DCPS-PPP6C | DCPS | 11q | 126303.75 | PPP6C | 9q | 125146.57 |
| 229 | 15 | PSMA1- | PSMA1 | 11p | 14504.88 | PPP6C | 9q | 125146.57 |
| | | PPP6C | | | | | | |
| 230 | 15 | PCDHB13- | PCDHB13 | 5q | 141213.92 | PPP6C | 9q | 125146.57 |
| | | PPP6C | | | | | | |
| 231 | 16 | TUBB-TSC1 | TUBB | 6p | 30720.2 | TSC1 | 9q | 132891.35 |
| 232 | 16 | VCP-TSC1 | VCP | 9p | 35056.07 | TSC1 | 9q | 132891.35 |
| 233 | 16 | DCPS-TSC1 | DCPS | 11q | 126303.75 | TSC1 | 9q | 132891.35 |
| 234 | 16 | ATR-TSC1 | ATR | 3q | 142449.24 | TSC1 | 9q | 132891.35 |
| 235 | 16 | EGFR-TSC1 | EGFR | 7p | 55019.03 | TSC1 | 9q | 132891.35 |
| 236 | 16 | METTL1- | METTL1 | 12q | 57768.57 | TSC1 | 9q | 132891.35 |
| | | TSC1 | | | | | | |
| 237 | 16 | RPAP3-TSC1 | RPAP3 | 12q | 47661.93 | TSC1 | 9q | 132891.35 |
| 238 | 16 | TM9SF2- | TM9SF2 | 13q | 99501.37 | TSC1 | 9q | 132891.35 |
| | | TSC1 | | | | | | |
| 239 | 17 | ESR1-BRIP1 | ESR1 | 6q | 151809.11 | BRIP1 | 17q | 61679.19 |
| 240 | 17 | ZMYM2- | ZMYM2 | 13q | 19958.67 | BRIP1 | 17q | 61679.19 |
| | | BRIP1 | | | | | | |
| 241 | 17 | GATA3- | GATA3 | 10p | 8054.7 | BRIP1 | 17q | 61679.19 |
| | | BRIP1 | | | | | | |
| 242 | 17 | FOXA1- | FOXA1 | 14q | 37589.55 | BRIP1 | 17q | 61679.19 |
| | | BRIP1 | | | | | | |
| 243 | 17 | RPL27A- | RPL27A | 11p | 8682.45 | BRIP1 | 17q | 61679.19 |
| | | BRIP1 | | | | | | |
| 244 | 17 | SAFB-BRIP1 | SAFB | 19p | 5623.04 | BRIP1 | 17q | 61679.19 |
| 245 | 17 | SF3B4-BRIP1 | SF3B4 | 1q | 149923.32 | BRIP1 | 17q | 61679.19 |
| 246 | 18 | MED1-TP53 | MED1 | 17q | 39404.29 | TP53 | 17p | 7668.4 |
| 247 | 18 | MED10-TP53 | MED10 | 5p | 6371.93 | TP53 | 17p | 7668.4 |
| 248 | 18 | KIF2C-TP53 | KIF2C | 1p | 44739.82 | TP53 | 17p | 7668.4 |
| 249 | 18 | CHD4-TP53 | CHD4 | 12p | 6570.08 | TP53 | 17p | 7668.4 |
| 250 | 18 | METAP2- | METAP2 | 12q | 95474.05 | TP53 | 17p | 7668.4 |
| | | TP53 | | | | | | |
| 251 | 18 | IGF1R-TP53 | IGF1R | 15q | 98648.54 | TP53 | 17p | 7668.4 |
| 252 | 18 | MYBL2- | MYBL2 | 20q | 43667.02 | TP53 | 17p | 7668.4 |
| | | TP53 | | | | | | |
| 253 | 19 | DDX39B- | DDX39B | 6p | 31530.22 | SMARCA4 | 19p | 10960.92 |
| | | SMARCA4 | | | | | | |
| 254 | 19 | PRKAR1A- | PRKAR1A | 17q | 68512.38 | SMARCA4 | 19p | 10960.92 |
| | | SMARCA4 | | | | | | |
| 255 | 19 | FUBP1- | FUBP1 | 1p | 77948.4 | SMARCA4 | 19p | 10960.92 |
| | | SMARCA4 | | | | | | |
| 256 | 19 | MYB- | MYB | 6q | 135181.32 | SMARCA4 | 19p | 10960.92 |
| | | SMARCA4 | | | | | | |
| 257 | 19 | CD33- | CD33 | 19q | 51225.08 | SMARCA4 | 19p | 10960.92 |
| | | SMARCA4 | | | | | | |
| 258 | 19 | GCLC- | GCLC | 6p | 53497.34 | SMARCA4 | 19p | 10960.92 |
| | | SMARCA4 | | | | | | |
| 259 | 20 | FUBP1- | FUBP1 | 1p | 77948.4 | STK11 | 19p | 1205.8 |
| | | STK11 | | | | | | |
| 260 | 20 | DDX39B- | DDX39B | 6p | 31530.22 | STK11 | 19p | 1205.8 |
| | | STK11 | | | | | | |
| 261 | 20 | SMARCA2- | SMARCA2 | 9p | 2015.22 | STK11 | 19p | 1205.8 |
| | | STK11 | | | | | | |
| 262 | 20 | POT1-STK11 | POT1 | 7q | 124822.39 | STK11 | 19p | 1205.8 |
| 263 | 20 | CDC40- | CDC40 | 6q | 110180.42 | STK11 | 19p | 1205.8 |
| | | STK11 | | | | | | |
| 264 | 20 | PPM1D- | PPM1D | 17q | 60600.18 | STK11 | 19p | 1205.8 |
| | | STK11 | | | | | | |
| 265 | 21 | CUL4B-DIS3 | CUL4B | Xq | 120524.59 | DIS3 | 13q | 72755.4 |
| 266 | 21 | PPP2R1A- | PPP2R1A | 19q | 52189.8 | DIS3 | 13q | 72755.4 |
| | | DIS3 | | | | | | |
| 267 | 21 | ITGAV-DIS3 | ITGAV | 2q | 186590.06 | DIS3 | 13q | 72755.4 |
| 268 | 21 | NAA10-DIS3 | NAA10 | Xq | 153929.83 | DIS3 | 13q | 72755.4 |
| 269 | 21 | WWTR1- | WWTR1 | 3q | 149517.23 | DIS3 | 13q | 72755.4 |
| | | DIS3 | | | | | | |
| 270 | 22 | MED1- | MED1 | 17q | 39404.29 | MAP2K4 | 17p | 12020.82 |
| | | MAP2K4 | | | | | | |
| 271 | 22 | MED10- | MED10 | 5p | 6371.93 | MAP2K4 | 17p | 12020.82 |
| | | MAP2K4 | | | | | | |
| 272 | 22 | METAP2- | METAP2 | 12q | 95474.05 | MAP2K4 | 17p | 12020.82 |
| | | MAP2K4 | | | | | | |
| 273 | 22 | KIF2C- | KIF2C | 1p | 44739.82 | MAP2K4 | 17p | 12020.82 |
| | | MAP2K4 | | | | | | |
| 274 | 22 | SAMD4B- | SAMD4B | 19q | 39342.46 | MAP2K4 | 17p | 12020.82 |
| | | MAP2K4 | | | | | | |
| 275 | 23 | DDX39B- | DDX39B | 6p | 31530.22 | MEF2B | 19p | 19145.57 |
| | | MEF2B | | | | | | |
| 276 | 23 | PRKAR1A- | PRKAR1A | 17q | 68512.38 | MEF2B | 19p | 19145.57 |
| | | MEF2B | | | | | | |
| 277 | 23 | SRSF11- | SRSF11 | 1p | 70205.68 | MEF2B | 19p | 19145.57 |
| | | MEF2B | | | | | | |
| 278 | 23 | MEIS3- | MEIS3 | 19q | 47403.12 | MEF2B | 19p | 19145.57 |
| | | MEF2B | | | | | | |
| 279 | 23 | FUBP1- | FUBP1 | 1p | 77948.4 | MEF2B | 19p | 19145.57 |
| | | MEF2B | | | | | | |
| 280 | 24 | PPP6C- | PPP6C | 9q | 125146.57 | PTPRT | 20q | 42072.75 |
| | | PTPRT | | | | | | |
| 281 | 24 | SBDS-PTPRT | SBDS | 7q | 66987.68 | PTPRT | 20q | 42072.75 |
| 282 | 24 | CMA1- | CMA1 | 14q | 24505.36 | PTPRT | 20q | 42072.75 |
| | | PTPRT | | | | | | |
| 283 | 24 | UFC1-PTPRT | UFC1 | 1q | 161153.74 | PTPRT | 20q | 42072.75 |
| 284 | 24 | SNRPB- | SNRPB | 20p | 2461.64 | PTPRT | 20q | 42072.75 |
| | | PTPRT | | | | | | |
| 285 | 25 | CBFB- | CBFB | 16q | 67029.15 | RUNX1 | 21q | 34787.8 |
| | | RUNX1 | | | | | | |
| 286 | 25 | RAB6A- | RAB6A | 11q | 73675.64 | RUNX1 | 21q | 34787.8 |
| | | RUNX1 | | | | | | |
| 287 | 25 | WDR92- | WDR92 | 2p | 68130.15 | RUNX1 | 21q | 34787.8 |
| | | RUNX1 | | | | | | |
| 288 | 25 | SDHAF3- | SDHAF3 | 7q | 97116.59 | RUNX1 | 21q | 34787.8 |
| | | RUNX1 | | | | | | |
| 289 | 25 | NFAT5- | NFAT5 | 16q | 69565.97 | RUNX1 | 21q | 34787.8 |
| | | RUNX1 | | | | | | |
| 290 | 26 | PPP2R1A- | PPP2R1A | 19q | 52189.8 | ATM | 11q | 108223.53 |
| | | ATM | | | | | | |
| 291 | 26 | WWTR1- | WWTR1 | 3q | 149517.23 | ATM | 11q | 108223.53 |
| | | ATM | | | | | | |
| 292 | 26 | CSTF3-ATM | CSTF3 | 11p | 33084.58 | ATM | 11q | 108223.53 |
| 293 | 26 | MDM2-ATM | MDM2 | 12q | 68808.17 | ATM | 11q | 108223.53 |
| 294 | 27 | WWTR1- | WWTR1 | 3q | 149517.23 | BRCA2 | 13q | 32315.48 |
| | | BRCA2 | | | | | | |
| 295 | 27 | MYBL2- | MYBL2 | 20q | 43667.02 | BRCA2 | 13q | 32315.48 |
| | | BRCA2 | | | | | | |
| 296 | 27 | NAA15- | NAA15 | 4q | 139301.47 | BRCA2 | 13q | 32315.48 |
| | | BRCA2 | | | | | | |
| 297 | 27 | MED12- | MED12 | Xq | 71118.56 | BRCA2 | 13q | 32315.48 |
| | | BRCA2 | | | | | | |
| 298 | 28 | WWTR1- | WWTR1 | 3q | 149517.23 | LATS2 | 13q | 20973.04 |
| | | LATS2 | | | | | | |
| 299 | 28 | NAA15- | NAA15 | 4q | 139301.47 | LATS2 | 13q | 20973.04 |
| | | LATS2 | | | | | | |
| 300 | 28 | NR4A3- | NR4A3 | 9q | 99821.86 | LATS2 | 13q | 20973.04 |
| | | LATS2 | | | | | | |
| 301 | 28 | NONO- | NONO | Xq | 71283.19 | LATS2 | 13q | 20973.04 |
| | | LATS2 | | | | | | |
| 302 | 29 | CCND1- | CCND1 | 11q | 69641.11 | PTPRD | 9p | 8314.25 |
| | | PTPRD | | | | | | |
| 303 | 29 | SIRPD- | SIRPD | 20p | 1534.25 | PTPRD | 9p | 8314.25 |
| | | PTPRD | | | | | | |
| 304 | 29 | ARF4- | ARF4 | 3p | 57571.36 | PTPRD | 9p | 8314.25 |
| | | PTPRD | | | | | | |
| 305 | 29 | DYRK1A- | DYRK1A | 21q | 37420.3 | PTPRD | 9p | 8314.25 |
| | | PTPRD | | | | | | |
| 306 | 30 | VPS4B- | VPS4B | 18q | 63389.19 | CDH1 | 16q | 68737.29 |
| | | CDH1 | | | | | | |
| 307 | 30 | MNT-CDH1 | MNT | 17p | 2384.06 | CDH1 | 16q | 68737.29 |
| 308 | 30 | MED8-CDH1 | MED8 | 1p | 43383.91 | CDH1 | 16q | 68737.29 |
| 309 | 31 | MAGOH- | MAGOH | 1p | 53226.89 | CDKN1B | 12p | 12717.27 |
| | | CDKN1B | | | | | | |
| 310 | 31 | GSK3B- | GSK3B | 3q | 119821.96 | CDKN1B | 12p | 12717.27 |
| | | CDKN1B | | | | | | |
| 311 | 31 | YME1L1- | YME1L1 | 10p | 27110.11 | CDKN1B | 12p | 12717.27 |
| | | CDKN1B | | | | | | |
| 312 | 32 | CDK2- | CDK2 | 12q | 55966.77 | KNSTRN | 15q | 40382.72 |
| | | KNSTRN | | | | | | |
| 313 | 32 | SKP2- | SKP2 | 5p | 36152.04 | KNSTRN | 15q | 40382.72 |
| | | KNSTRN | | | | | | |
| 314 | 32 | RBBP4- | RBBP4 | 1p | 32651.15 | KNSTRN | 15q | 40382.72 |
| | | KNSTRN | | | | | | |
| 315 | 33 | CSTF3- | CSTF3 | 11p | 33084.58 | MEN1 | 11q | 64803.51 |
| | | MEN1 | | | | | | |
| 316 | 33 | RAB1A- | RAB1A | 2p | 65086.85 | MEN1 | 11q | 64803.51 |
| | | MEN1 | | | | | | |
| 317 | 33 | HNRNPD- | HNRNPD | 4q | 82353.31 | MEN1 | 11q | 64803.51 |
| | | MEN1 | | | | | | |
| 318 | 34 | FBXW11- | FBXW11 | 5q | 171861.55 | PTEN | 10q | 87863.44 |
| | | PTEN | | | | | | |
| 319 | 34 | PIK3CB- | PIK3CB | 3q | 138652.7 | PTEN | 10q | 87863.44 |
| | | PTEN | | | | | | |
| 320 | 34 | BRAF-PTEN | BRAF | 7q | 140732.56 | PTEN | 10q | 87863.44 |
| 321 | 35 | SNRPB2- | SNRPB2 | 20p | 16729.96 | BRCA1 | 17q | 43044.3 |
| | | BRCA1 | | | | | | |
| 322 | 35 | SPDEF- | SPDEF | 6p | 34537.8 | BRCA1 | 17q | 43044.3 |
| | | BRCA1 | | | | | | |
| 323 | 36 | PPP2R1A- | PPP2R1A | 19q | 52189.8 | CBL | 11q | 119206.28 |
| | | CBL | | | | | | |
| 324 | 36 | WWTR1- | WWTR1 | 3q | 149517.23 | CBL | 11q | 119206.28 |
| | | CBL | | | | | | |
| 325 | 37 | DDX5- | DDX5 | 17q | 64498.25 | CHEK2 | 22q | 28687.74 |
| | | CHEK2 | | | | | | |
| 326 | 37 | ELMSAN1- | ELMSAN1 | 14q | 73715.12 | CHEK2 | 22q | 28687.74 |
| | | CHEK2 | | | | | | |
| 327 | 38 | PRIM1- | PRIM1 | 12q | 56731.58 | FBXW7 | 4q | 152321.26 |
| | | FBXW7 | | | | | | |
| 328 | 38 | GLE1- | GLE1 | 9q | 128504.69 | FBXW7 | 4q | 152321.26 |
| | | FBXW7 | | | | | | |
| 329 | 39 | GRPEL1- | GRPEL1 | 4p | 7058.9 | PIK3R1 | 5q | 68288.42 |
| | | PIK3R1 | | | | | | |
| 330 | 39 | RPL30- | RPL30 | 8q | 98041.71 | PIK3R1 | 5q | 68288.42 |
| | | PIK3R1 | | | | | | |
| 331 | 40 | ATP6AP1- | ATP6AP1 | Xq | 154428.63 | TSC2 | 16p | 2048.2 |
| | | TSC2 | | | | | | |
| 332 | 40 | PCBP1-TSC2 | PCBP1 | 2p | 70087.45 | TSC2 | 16p | 2048.2 |
| 333 | 41 | ARID1B- | ARID1B | 6q | 156777.93 | EPCAM | 2p | 47369.15 |
| | | EPCAM | | | | | | |
| 334 | 42 | COPB2-FAT1 | COPB2 | 3q | 139357.59 | FAT1 | 4q | 186587.78 |
| 335 | 43 | CDK6- | CDK6 | 7q | 92604.92 | RBM10 | Xp | 47145.22 |
| | | RBM10 | | | | | | |
| 336 | 44 | ACTB-SRSF2 | ACTB | 7p | 5527.15 | SRSF2 | 17q | 76734.12 |
| 337 | 45 | AIFM1-VHL | AIFM1 | Xq | 130129.36 | VHL | 3p | 10141.64 |

| | | | | −Log(q- |
|---|---|---|---|---|
| ID | Pearson_r_CRISPR | P_value_CRISPR | P_adjusted_BH_CRISPR | value_CRISPR) |
| 1 | 0.340726124 | 1.99E−18 | 5.92E−13 | 12.22762695 |
| 2 | −0.305793654 | 5.66E−15 | 5.60E−10 | 9.251440728 |
| 3 | 0.27396377 | 3.32E−12 | 1.34E−07 | 6.87162696 |
| 4 | 0.234989116 | 2.82E−09 | 3.44E−05 | 4.462896383 |
| 5 | 0.228756095 | 7.48E−09 | 7.10E−05 | 4.149041515 |
| 6 | 0.210904485 | 1.05E−07 | 0.000538013 | 3.269207161 |
| 7 | 0.209356941 | 1.31E−07 | 0.000643521 | 3.191436961 |
| 8 | 0.208733417 | 1.43E−07 | 0.000690966 | 3.160543335 |
| 9 | −0.206339433 | 1.99E−07 | 0.000887539 | 3.051812769 |
| 10 | −0.203993771 | 2.75E−07 | 0.00112701 | 2.948072339 |
| 11 | 0.202346169 | 3.45E−07 | 0.00133491 | 2.874548035 |
| 12 | −0.197292089 | 6.79E−07 | 0.002207088 | 2.656180337 |
| 13 | 0.194116561 | 1.03E−06 | 0.002989131 | 2.524455099 |
| 14 | −0.187655319 | 2.36E−06 | 0.005369054 | 2.270102215 |
| 15 | 0.180687696 | 5.57E−06 | 0.009578729 | 2.018692132 |
| 16 | 0.178247872 | 7.48E−06 | 0.011710341 | 1.931430445 |
| 17 | 0.176929388 | 8.75E−06 | 0.013171258 | 1.880372733 |
| 18 | 0.176286784 | 9.44E−06 | 0.013794658 | 1.860289059 |
| 19 | 0.174614531 | 1.15E−05 | 0.015315092 | 1.814880377 |
| 20 | 0.171157318 | 1.72E−05 | 0.020065148 | 1.697557638 |
| 21 | 0.164890423 | 3.49E−05 | 0.030111185 | 1.521272146 |
| 22 | 0.162898546 | 4.34E−05 | 0.033484936 | 1.475150524 |
| 23 | −0.162596654 | 4.49E−05 | 0.033946191 | 1.469208943 |
| 24 | −0.160075116 | 5.90E−05 | 0.038803357 | 1.411130703 |
| 25 | −0.153111717 | 0.000123173 | 0.0558895 | 1.252669778 |
| 26 | −0.150663595 | 0.000158338 | 0.063585566 | 1.19664146 |
| 27 | 0.149893231 | 0.000171225 | 0.065909055 | 1.181054915 |
| 28 | −0.149757362 | 0.000173597 | 0.066278305 | 1.17862861 |
| 29 | −0.149376964 | 0.000180405 | 0.067365987 | 1.171559321 |
| 30 | −0.149327564 | 0.000181307 | 0.067491181 | 1.170752973 |
| 31 | 0.147174361 | 0.000225004 | 0.074249801 | 1.129304704 |
| 32 | 0.146613055 | 0.000237918 | 0.076400157 | 1.116905747 |
| 33 | 0.146139025 | 0.000249361 | 0.078214533 | 1.106712546 |
| 34 | 0.145925843 | 0.000254674 | 0.079018575 | 1.102270806 |
| 35 | 0.144101709 | 0.000304658 | 0.085572462 | 1.067665974 |
| 36 | −0.142871517 | 0.00034339 | 0.089394249 | 1.048690422 |
| 37 | 0.258367363 | 5.65E−11 | 1.80E−06 | 5.745594914 |
| 38 | 0.221394252 | 2.29E−08 | 0.000174104 | 3.759190459 |
| 39 | 0.217915015 | 3.82E−08 | 0.000257144 | 3.589824148 |
| 40 | 0.215224455 | 5.66E−08 | 0.000350145 | 3.455751742 |
| 41 | −0.209518821 | 1.28E−07 | 0.000632522 | 3.19892406 |
| 42 | 0.203980137 | 2.76E−07 | 0.00112701 | 2.948072339 |
| 43 | 0.201893714 | 3.66E−07 | 0.001383346 | 2.859069235 |
| 44 | −0.195401905 | 8.71E−07 | 0.002647674 | 2.577135452 |
| 45 | −0.19459518 | 9.67E−07 | 0.002835971 | 2.547298194 |
| 46 | −0.191201582 | 1.50E−06 | 0.003911158 | 2.407694637 |
| 47 | −0.187288634 | 2.47E−06 | 0.005551501 | 2.255589542 |
| 48 | −0.186802408 | 2.62E−06 | 0.005798314 | 2.23669828 |
| 49 | −0.176363355 | 9.36E−06 | 0.013737736 | 1.862084824 |
| 50 | 0.175909043 | 9.87E−06 | 0.014111329 | 1.850432096 |
| 51 | −0.170317659 | 1.89E−05 | 0.021258408 | 1.672469253 |
| 52 | −0.168810217 | 2.25E−05 | 0.023550064 | 1.628007906 |
| 53 | 0.167386024 | 2.64E−05 | 0.025866861 | 1.58725627 |
| 54 | −0.166836597 | 2.81E−05 | 0.027038191 | 1.568022374 |
| 55 | 0.163429558 | 4.10E−05 | 0.032661932 | 1.485958128 |
| 56 | 0.16304416 | 4.28E−05 | 0.033313781 | 1.477376068 |
| 57 | −0.161644491 | 4.98E−05 | 0.035748532 | 1.446741792 |
| 58 | −0.15871151 | 6.84E−05 | 0.041952128 | 1.377246006 |
| 59 | 0.158334082 | 7.12E−05 | 0.042684958 | 1.369725145 |
| 60 | 0.158250205 | 7.18E−05 | 0.042717413 | 1.369395061 |
| 61 | −0.163812832 | 7.28E−05 | 0.042918696 | 1.367353485 |
| 62 | −0.1544012 | 0.000107746 | 0.05243735 | 1.280359264 |
| 63 | −0.151934777 | 0.000139046 | 0.059338738 | 1.226661696 |
| 64 | −0.148537654 | 0.00019632 | 0.069920757 | 1.155393876 |
| 65 | −0.147539457 | 0.00021696 | 0.072975081 | 1.136825417 |
| 66 | −0.14378794 | 0.000314129 | 0.086449143 | 1.063239305 |
| 67 | −0.143409255 | 0.000325926 | 0.087585801 | 1.057566296 |
| 68 | 0.142950342 | 0.000340776 | 0.089150727 | 1.049875111 |
| 69 | 0.23955577 | 1.36E−09 | 2.16E−05 | 4.665859599 |
| 70 | 0.235241822 | 2.71E−09 | 3.40E−05 | 4.468263709 |
| 71 | 0.219547514 | 3.01E−08 | 0.000212646 | 3.672342698 |
| 72 | −0.214356129 | 6.41E−08 | 0.000381055 | 3.41901202 |
| 73 | 0.213481121 | 7.27E−08 | 0.00041075 | 3.38642225 |
| 74 | 0.213429156 | 7.33E−08 | 0.00041075 | 3.38642225 |
| 75 | −0.207088933 | 1.80E−07 | 0.000820331 | 3.086011113 |
| 76 | −0.201856373 | 3.68E−07 | 0.001384498 | 2.85870779 |
| 77 | 0.191230303 | 1.50E−06 | 0.0039082 | 2.40802321 |
| 78 | 0.173960745 | 1.24E−05 | 0.016196336 | 1.790583233 |
| 79 | −0.173618932 | 1.29E−05 | 0.016636373 | 1.778941341 |
| 80 | −0.167910294 | 2.49E−05 | 0.024819477 | 1.605207376 |
| 81 | 0.166536691 | 2.90E−05 | 0.027399758 | 1.562253279 |
| 82 | −0.166108598 | 3.04E−05 | 0.028209598 | 1.549603109 |
| 83 | −0.163970454 | 3.86E−05 | 0.031753502 | 1.498208373 |
| 84 | −0.159237829 | 6.46E−05 | 0.040432188 | 1.393272751 |
| 85 | −0.158258678 | 7.17E−05 | 0.042717413 | 1.369395061 |
| 86 | −0.156020904 | 9.09E−05 | 0.048204501 | 1.31691241 |
| 87 | 0.151568607 | 0.000144364 | 0.060533916 | 1.21800123 |
| 88 | −0.147110829 | 0.000226432 | 0.07440571 | 1.128393736 |
| 89 | −0.147071185 | 0.000227328 | 0.074484938 | 1.127931539 |
| 90 | −0.146714097 | 0.000235544 | 0.075856261 | 1.120008568 |
| 91 | −0.146013043 | 0.000252488 | 0.078669132 | 1.104195639 |
| 92 | −0.145408733 | 0.000268003 | 0.081417241 | 1.089283621 |
| 93 | −0.145350942 | 0.000269532 | 0.081609605 | 1.088258724 |
| 94 | 0.14505219 | 0.00027757 | 0.082374561 | 1.084206887 |
| 95 | 0.254300945 | 1.15E−10 | 3.01E−06 | 5.521347953 |
| 96 | 0.242101744 | 3.40E−09 | 3.98E−05 | 4.39988336 |
| 97 | −0.219949887 | 2.83E−08 | 0.000204984 | 3.688279312 |
| 98 | 0.197746325 | 6.39E−07 | 0.002124642 | 2.672714146 |
| 99 | 0.194172893 | 1.02E−06 | 0.002986733 | 2.524803581 |
| 100 | 0.181508696 | 5.04E−06 | 0.008991606 | 2.046162725 |
| 101 | 0.17632932 | 9.40E−06 | 0.013770521 | 1.861049622 |
| 102 | −0.172650603 | 1.45E−05 | 0.017945124 | 1.746053527 |
| 103 | −0.168784459 | 2.25E−05 | 0.023591235 | 1.627249318 |
| 104 | −0.166618748 | 2.88E−05 | 0.027293367 | 1.563942886 |
| 105 | −0.158264643 | 7.17E−05 | 0.042717413 | 1.369395061 |
| 106 | 0.148644696 | 0.000194219 | 0.069466009 | 1.15822765 |
| 107 | 0.146919977 | 0.000230773 | 0.075134643 | 1.12415977 |
| 108 | 0.145305451 | 0.000270742 | 0.081609605 | 1.088258724 |
| 109 | −0.143582555 | 0.000320477 | 0.087008579 | 1.060437926 |
| 110 | −0.141936834 | 0.000375838 | 0.093420328 | 1.029558613 |
| 111 | 0.258759599 | 2.42E−10 | 5.27E−06 | 5.278601277 |
| 112 | 0.24687396 | 4.06E−10 | 8.23E−06 | 5.08460256 |
| 113 | −0.217847099 | 3.86E−08 | 0.000257144 | 3.589824148 |
| 114 | 0.204281277 | 2.65E−07 | 0.001101652 | 2.957955706 |
| 115 | 0.191945109 | 1.36E−06 | 0.003677361 | 2.434463771 |
| 116 | 0.177204544 | 8.47E−06 | 0.012855981 | 1.890894791 |
| 117 | 0.176075169 | 9.68E−06 | 0.014006565 | 1.853668345 |
| 118 | −0.166671766 | 2.86E−05 | 0.02726982 | 1.56431773 |
| 119 | −0.164975511 | 3.45E−05 | 0.03005907 | 1.522024458 |
| 120 | −0.164812065 | 3.52E−05 | 0.030227537 | 1.519597241 |
| 121 | 0.164706677 | 3.56E−05 | 0.030407796 | 1.517015063 |
| 122 | 0.14931028 | 0.000181623 | 0.067491181 | 1.170752973 |
| 123 | −0.144566069 | 0.00029113 | 0.084175004 | 1.074816853 |
| 124 | −0.143865723 | 0.000311756 | 0.086172646 | 1.064630573 |
| 125 | 0.248195902 | 1.32E−09 | 2.14E−05 | 4.669375082 |
| 126 | 0.225097862 | 1.31E−08 | 0.000110109 | 3.958175272 |
| 127 | 0.214783921 | 6.03E−08 | 0.000363088 | 3.439988536 |
| 128 | −0.20422229 | 2.67E−07 | 0.001105435 | 2.956466955 |
| 129 | −0.204115694 | 2.71E−07 | 0.001116515 | 2.952135621 |
| 130 | 0.192399728 | 1.29E−06 | 0.00356051 | 2.448487801 |
| 131 | 0.174602916 | 1.15E−05 | 0.015315092 | 1.814880377 |
| 132 | 0.166430289 | 2.94E−05 | 0.027611112 | 1.558916095 |
| 133 | 0.162435282 | 4.57E−05 | 0.034245789 | 1.465392822 |
| 134 | 0.16104016 | 5.32E−05 | 0.036690038 | 1.43545184 |
| 135 | 0.159431533 | 6.33E−05 | 0.040078637 | 1.397087055 |
| 136 | −0.153243719 | 0.000121503 | 0.055666925 | 1.254402772 |
| 137 | 0.140357842 | 0.000437227 | 0.099990678 | 1.000040488 |
| 138 | −0.206217554 | 2.03E−07 | 0.000893715 | 3.048800898 |
| 139 | 0.185195339 | 3.20E−06 | 0.006735104 | 2.171655724 |
| 140 | 0.17776086 | 7.93E−06 | 0.012238845 | 1.912259566 |
| 141 | −0.168891995 | 2.23E−05 | 0.023446144 | 1.629928564 |
| 142 | 0.17461963 | 2.31E−05 | 0.023887261 | 1.621833648 |
| 143 | −0.16317412 | 4.21E−05 | 0.033117852 | 1.479937843 |
| 144 | −0.161502476 | 5.06E−05 | 0.035937918 | 1.44444709 |
| 145 | 0.158332715 | 7.12E−05 | 0.042684958 | 1.369725145 |
| 146 | −0.155236683 | 9.87E−05 | 0.050032369 | 1.300748934 |
| 147 | −0.152053494 | 0.000137362 | 0.058876261 | 1.230059779 |
| 148 | −0.149339214 | 0.000181094 | 0.067491181 | 1.170752973 |
| 149 | 0.14043608 | 0.000433978 | 0.099798795 | 1.000874701 |
| 150 | 0.270751511 | 3.21E−11 | 1.06E−06 | 5.974555014 |
| 151 | 0.241000426 | 1.07E−09 | 1.82E−05 | 4.740466478 |
| 152 | 0.23707364 | 2.02E−09 | 2.73E−05 | 4.56350689 |
| 153 | 0.225256459 | 1.28E−08 | 0.000109799 | 3.959403133 |
| 154 | −0.21700415 | 4.37E−08 | 0.000284144 | 3.546460919 |
| 155 | −0.200356238 | 4.51E−07 | 0.001611824 | 2.792682283 |
| 156 | 0.19400965 | 1.04E−06 | 0.003021194 | 2.519821458 |
| 157 | 0.176762888 | 8.92E−06 | 0.013366494 | 1.873982502 |
| 158 | 0.17481665 | 1.12E−05 | 0.015088425 | 1.821356084 |
| 159 | −0.153896759 | 0.000113551 | 0.053565677 | 1.271113403 |
| 160 | 0.151960847 | 0.000138674 | 0.059253258 | 1.227287766 |
| 161 | 0.150564982 | 0.000159936 | 0.063938825 | 1.194235352 |
| 162 | −0.186189555 | 2.83E−06 | 0.006183747 | 2.208748314 |
| 163 | −0.183377635 | 4.01E−06 | 0.007772682 | 2.109429097 |
| 164 | 0.168254981 | 2.39E−05 | 0.024355222 | 1.613407902 |
| 165 | −0.1639382 | 3.87E−05 | 0.031753502 | 1.498208373 |
| 166 | 0.153973922 | 0.000112644 | 0.053387093 | 1.272563724 |
| 167 | −0.153706616 | 0.000115814 | 0.054085517 | 1.266919017 |
| 168 | −0.151905957 | 0.000139458 | 0.059397336 | 1.226233036 |
| 169 | −0.14795324 | 0.000208169 | 0.071899405 | 1.143274705 |
| 170 | −0.143031198 | 0.000338115 | 0.088850176 | 1.051341706 |
| 171 | −0.141830536 | 0.000379705 | 0.093883736 | 1.027409635 |
| 172 | 0.140717091 | 0.000422491 | 0.098556139 | 1.006316316 |
| 173 | −0.208466886 | 1.48E−07 | 0.000709551 | 3.149016539 |
| 174 | 0.211914562 | 2.53E−07 | 0.001067476 | 2.971641685 |
| 175 | 0.190962189 | 1.55E−06 | 0.003974946 | 2.400668813 |
| 176 | 0.179211321 | 6.66E−06 | 0.010812097 | 1.966090057 |
| 177 | −0.169875913 | 1.99E−05 | 0.022053505 | 1.656522383 |
| 178 | 0.166315334 | 2.98E−05 | 0.027851022 | 1.555158865 |
| 179 | 0.155693026 | 9.41E−05 | 0.048686467 | 1.312591742 |
| 180 | 0.154737769 | 0.000104029 | 0.051471962 | 1.288429274 |
| 181 | 0.151218011 | 0.000149634 | 0.061692389 | 1.209768411 |
| 182 | −0.144978827 | 0.000279578 | 0.082603979 | 1.082999034 |
| 183 | −0.147443206 | 0.000362726 | 0.091930524 | 1.036540264 |
| 184 | −0.172495056 | 1.47E−05 | 0.018195619 | 1.740033169 |
| 185 | −0.170746889 | 1.80E−05 | 0.02070813 | 1.68385911 |
| 186 | −0.170475374 | 1.86E−05 | 0.021037121 | 1.677013694 |
| 187 | −0.161458008 | 5.08E−05 | 0.035937918 | 1.44444709 |
| 188 | −0.160714468 | 5.51E−05 | 0.037514103 | 1.425805431 |
| 189 | −0.158411777 | 7.06E−05 | 0.042584214 | 1.370751366 |
| 190 | 0.146460459 | 0.000241547 | 0.077065583 | 1.113139534 |
| 191 | −0.145314899 | 0.00027049 | 0.081609605 | 1.088258724 |
| 192 | 0.145086508 | 0.000276636 | 0.082340267 | 1.084387731 |
| 193 | 0.141609987 | 0.000387845 | 0.094811493 | 1.023139013 |
| 194 | 0.295115774 | 5.25E−14 | 3.90E−09 | 8.408780831 |
| 195 | 0.229871218 | 6.30E−09 | 6.28E−05 | 4.202144412 |
| 196 | −0.187267361 | 2.47E−06 | 0.00555234 | 2.255523937 |
| 197 | −0.178633887 | 7.14E−06 | 0.011389797 | 1.943484001 |
| 198 | −0.170127828 | 1.93E−05 | 0.021620785 | 1.665128551 |
| 199 | −0.160561255 | 5.60E−05 | 0.03788062 | 1.421582916 |
| 200 | −0.157322302 | 7.93E−05 | 0.044559893 | 1.351055864 |
| 201 | −0.151495802 | 0.000145444 | 0.060738836 | 1.216533532 |
| 202 | 0.144270108 | 0.000299685 | 0.08515673 | 1.069781023 |
| 203 | −0.143356055 | 0.000327616 | 0.087784748 | 1.056580932 |
| 204 | 0.239058716 | 1.47E−09 | 2.24E−05 | 4.649005664 |
| 205 | −0.219492479 | 3.03E−08 | 0.000212694 | 3.672244571 |
| 206 | 0.169730739 | 2.02E−05 | 0.022283325 | 1.652020005 |
| 207 | −0.158831883 | 6.75E−05 | 0.041578975 | 1.381126216 |
| 208 | 0.156513288 | 8.63E−05 | 0.046971039 | 1.328169833 |
| 209 | 0.154986598 | 0.000101359 | 0.050828392 | 1.293893633 |
| 210 | −0.153468077 | 0.000118714 | 0.054841751 | 1.260888687 |
| 211 | −0.144147644 | 0.000303294 | 0.085462398 | 1.068224923 |
| 212 | 0.14346853 | 0.000324052 | 0.087451566 | 1.058232409 |
| 213 | 0.141771635 | 0.000381863 | 0.094052014 | 1.026631898 |
| 214 | −0.184315477 | 3.57E−06 | 0.007201821 | 2.142557677 |
| 215 | 0.183230554 | 4.09E−06 | 0.007846354 | 2.105332106 |
| 216 | −0.173819762 | 1.26E−05 | 0.016393215 | 1.785335865 |
| 217 | 0.164574287 | 3.61E−05 | 0.030562908 | 1.514805325 |
| 218 | −0.149508334 | 0.000178026 | 0.066960003 | 1.174184534 |
| 219 | −0.149260244 | 0.000182543 | 0.067578698 | 1.170190182 |
| 220 | −0.148090075 | 0.000205336 | 0.071352844 | 1.146588713 |
| 221 | −0.14640334 | 0.000242919 | 0.077309443 | 1.111767455 |
| 222 | −0.142931589 | 0.000341396 | 0.089161847 | 1.049820945 |
| 223 | −0.230570762 | 5.65E−09 | 5.92E−05 | 4.227429948 |
| 224 | 0.16293877 | 4.32E−05 | 0.033424313 | 1.475937507 |
| 225 | 0.158176564 | 7.24E−05 | 0.042853834 | 1.368010321 |
| 226 | 0.152220087 | 0.00013503 | 0.058269467 | 1.234558955 |
| 227 | 0.149686802 | 0.000174841 | 0.066536334 | 1.176941133 |
| 228 | −0.146898473 | 0.000231267 | 0.075267972 | 1.123389785 |
| 229 | 0.143106233 | 0.000335662 | 0.088758902 | 1.051788078 |
| 230 | 0.143005818 | 0.000338948 | 0.088960902 | 1.050800823 |
| 231 | 0.238844 | 1.52E−09 | 2.24E−05 | 4.649005664 |
| 232 | 0.191753955 | 1.40E−06 | 0.003719351 | 2.429532849 |
| 233 | −0.176898761 | 8.78E−06 | 0.01317475 | 1.880257629 |
| 234 | 0.173575175 | 1.30E−05 | 0.016673277 | 1.777979026 |
| 235 | −0.1627599 | 4.41E−05 | 0.033706038 | 1.4722923 |
| 236 | −0.157032706 | 8.17E−05 | 0.045302192 | 1.343880788 |
| 237 | −0.155905494 | 9.20E−05 | 0.048345939 | 1.315639997 |
| 238 | −0.143935738 | 0.000309634 | 0.085848399 | 1.066267801 |
| 239 | −0.225178978 | 1.29E−08 | 0.000109799 | 3.959403133 |
| 240 | −0.183085649 | 4.16E−06 | 0.007928647 | 2.100800908 |
| 241 | −0.158346894 | 7.11E−05 | 0.042677868 | 1.369797288 |
| 242 | −0.157803054 | 7.53E−05 | 0.043647918 | 1.360036468 |
| 243 | −0.156395467 | 8.74E−05 | 0.047150032 | 1.326518011 |
| 244 | −0.150240164 | 0.000165304 | 0.064805801 | 1.188386119 |
| 245 | 0.146785905 | 0.000233869 | 0.075426136 | 1.12247814 |
| 246 | 0.307341503 | 4.07E−15 | 4.53E−10 | 9.343890947 |
| 247 | 0.200762795 | 4.27E−07 | 0.001571833 | 2.803593646 |
| 248 | −0.179139173 | 6.72E−06 | 0.010866736 | 1.963900874 |
| 249 | 0.165821995 | 3.14E−05 | 0.028675647 | 1.542486769 |
| 250 | −0.159581124 | 6.23E−05 | 0.039891783 | 1.399116549 |
| 251 | 0.158578332 | 6.93E−05 | 0.042291523 | 1.37374667 |
| 252 | 0.14849055 | 0.000197251 | 0.070112263 | 1.154206013 |
| 253 | 0.303197223 | 9.81E−15 | 8.75E−10 | 9.058222606 |
| 254 | −0.200096371 | 4.67E−07 | 0.001657624 | 2.780513967 |
| 255 | 0.199662186 | 4.95E−07 | 0.001729488 | 2.762082472 |
| 256 | −0.151448197 | 0.000146154 | 0.060887589 | 1.21547122 |
| 257 | 0.148875976 | 0.000189752 | 0.068743127 | 1.162770717 |
| 258 | −0.148263827 | 0.000201791 | 0.070961667 | 1.148976188 |
| 259 | 0.239795405 | 1.30E−09 | 2.14E−05 | 4.669375082 |
| 260 | 0.186244071 | 2.81E−06 | 0.006171054 | 2.209640626 |
| 261 | 0.150547892 | 0.000160214 | 0.063992678 | 1.193869715 |
| 262 | −0.145151745 | 0.000274867 | 0.082137057 | 1.085460862 |
| 263 | 0.144221642 | 0.000301109 | 0.085339145 | 1.068851713 |
| 264 | −0.143046023 | 0.000337629 | 0.088821686 | 1.051480986 |
| 265 | 0.221604878 | 6.77E−08 | 0.000394025 | 3.404475942 |
| 266 | 0.202622788 | 3.32E−07 | 0.001302636 | 2.885177059 |
| 267 | 0.154108739 | 0.000111076 | 0.053072744 | 1.275128458 |
| 268 | −0.147129363 | 0.000373488 | 0.093095743 | 1.031070177 |
| 269 | 0.140532842 | 0.00042999 | 0.099293012 | 1.003081315 |
| 270 | 0.277109519 | 1.83E−12 | 8.59E−08 | 7.066103401 |
| 271 | 0.150686964 | 0.000157962 | 0.063491676 | 1.197283211 |
| 272 | −0.148246638 | 0.000202139 | 0.07103052 | 1.148555008 |
| 273 | −0.142993232 | 0.000339362 | 0.089021916 | 1.050503063 |
| 274 | 0.141362298 | 0.00039718 | 0.095604989 | 1.019519443 |
| 275 | 0.311407504 | 1.69E−15 | 2.51E−10 | 9.600252274 |
| 276 | −0.211047103 | 1.03E−07 | 0.000533374 | 3.272968303 |
| 277 | −0.190644758 | 1.61E−06 | 0.004092625 | 2.387997996 |
| 278 | 0.163513236 | 4.06E−05 | 0.032495278 | 1.488179741 |
| 279 | 0.162558639 | 4.51E−05 | 0.034029262 | 1.468147469 |
| 280 | −0.178465591 | 7.29E−06 | 0.011469504 | 1.940455362 |
| 281 | −0.160206236 | 5.82E−05 | 0.038715255 | 1.412117878 |
| 282 | 0.147007197 | 0.00022878 | 0.074731354 | 1.126497149 |
| 283 | −0.141218936 | 0.000402679 | 0.096226753 | 1.016704171 |
| 284 | 0.141169901 | 0.000404576 | 0.096367902 | 1.016067595 |
| 285 | −0.207645886 | 1.66E−07 | 0.000770957 | 3.112969926 |
| 286 | 0.206937034 | 1.83E−07 | 0.000833567 | 3.079059696 |
| 287 | 0.180975836 | 5.38E−06 | 0.009348363 | 2.029264414 |
| 288 | 0.144526534 | 0.000292259 | 0.084296193 | 1.074192038 |
| 289 | −0.141689227 | 0.000384901 | 0.094513088 | 1.024508047 |
| 290 | 0.308346467 | 3.28E−15 | 4.17E−10 | 9.379592156 |
| 291 | 0.16875776 | 2.26E−05 | 0.023635024 | 1.626443943 |
| 292 | 0.155480748 | 9.62E−05 | 0.04937657 | 1.306479083 |
| 293 | 0.144150788 | 0.000303201 | 0.085462398 | 1.068224923 |
| 294 | 0.168869942 | 2.23E−05 | 0.023446144 | 1.629928564 |
| 295 | −0.164925152 | 3.47E−05 | 0.03005907 | 1.522024458 |
| 296 | 0.158651962 | 6.88E−05 | 0.042133042 | 1.375377182 |
| 297 | −0.158767293 | 0.000121501 | 0.055666925 | 1.254402772 |
| 298 | 0.196593802 | 7.44E−07 | 0.002377095 | 2.623953434 |
| 299 | 0.192083002 | 1.34E−06 | 0.003652269 | 2.437437295 |
| 300 | −0.155138764 | 9.98E−05 | 0.050308831 | 1.298355772 |
| 301 | 0.158507534 | 0.000124694 | 0.056118853 | 1.250891212 |
| 302 | 0.229997444 | 6.18E−09 | 6.28E−05 | 4.202144412 |
| 303 | −0.164066218 | 3.82E−05 | 0.031637721 | 1.499794809 |
| 304 | 0.15944999 | 6.32E−05 | 0.04002771 | 1.397639254 |
| 305 | −0.144035603 | 0.000306631 | 0.085709109 | 1.066973021 |
| 306 | 0.283460558 | 5.39E−13 | 3.20E−08 | 7.494777984 |
| 307 | 0.148112879 | 0.000204867 | 0.071320175 | 1.146787599 |
| 308 | −0.143640788 | 0.000318665 | 0.086869013 | 1.061135114 |
| 309 | 0.387646532 | 8.30E−24 | 7.39E−18 | 17.13108438 |
| 310 | −0.155903482 | 9.21E−05 | 0.048345939 | 1.315639997 |
| 311 | 0.143118752 | 0.000335255 | 0.088701716 | 1.052067977 |
| 312 | 0.189164807 | 1.95E−06 | 0.004700855 | 2.327823177 |
| 313 | 0.158603858 | 6.91E−05 | 0.042250959 | 1.374163425 |
| 314 | −0.155172344 | 9.94E−05 | 0.050233079 | 1.299010199 |
| 315 | 0.212579552 | 8.28E−08 | 0.000447019 | 3.349673672 |
| 316 | 0.184992989 | 3.29E−06 | 0.006826014 | 2.16583284 |
| 317 | −0.15789999 | 7.45E−05 | 0.043499145 | 1.361519276 |
| 318 | 0.225451183 | 1.24E−08 | 0.000108452 | 3.964763339 |
| 319 | 0.159290966 | 6.42E−05 | 0.04028984 | 1.394804458 |
| 320 | 0.151941395 | 0.000138951 | 0.059329407 | 1.22672999 |
| 321 | 0.213004659 | 7.79E−08 | 0.000428436 | 3.368114206 |
| 322 | 0.166465419 | 2.93E−05 | 0.027531795 | 1.560165476 |
| 323 | 0.282334844 | 6.71E−13 | 3.63E−08 | 7.440443257 |
| 324 | 0.154679414 | 0.000104665 | 0.051569259 | 1.287609105 |
| 325 | 0.205030213 | 2.39E−07 | 0.001012836 | 2.994460739 |
| 326 | 0.140462297 | 0.000432894 | 0.09965242 | 1.001512148 |
| 327 | −0.163383412 | 4.12E−05 | 0.032740241 | 1.484918128 |
| 328 | −0.148287671 | 0.000201309 | 0.070876077 | 1.149500329 |
| 329 | 0.149773652 | 0.000173311 | 0.066254245 | 1.178786293 |
| 330 | −0.141173214 | 0.000404447 | 0.096367902 | 1.016067595 |
| 331 | 0.17701165 | 1.78E−05 | 0.020522186 | 1.687776384 |
| 332 | −0.141498912 | 0.000392006 | 0.09515622 | 1.021562819 |
| 333 | 0.171133689 | 1.72E−05 | 0.020067193 | 1.697513367 |
| 334 | −0.146111188 | 0.000250049 | 0.078347513 | 1.105974785 |
| 335 | 0.163290175 | 4.16E−05 | 0.03293063 | 1.482399965 |
| 336 | 0.186144973 | 2.85E−06 | 0.006193548 | 2.208060526 |
| 337 | 0.247238709 | 1.54E−09 | 2.24E−05 | 4.649005664 |

| | | | | −Log(q- |
|---|---|---|---|---|
| ID | Pearson_r_RNAi | P_value_RNAi | P_adjusted_BH_RNAi | value_RNAi) |
| 1 | 0.146885577 | 0.000662059 | 0.062152192 | 1.206543553 |
| 2 | −0.205170786 | 0.000577094 | 0.058355548 | 1.233917845 |
| 3 | 0.172047261 | 8.70E−05 | 0.022157456 | 1.654480104 |
| 4 | 0.166496317 | 0.000147199 | 0.029073964 | 1.536495751 |
| 5 | 0.171928581 | 7.51E−06 | 0.00536186 | 2.270684538 |
| 6 | 0.126772671 | 0.001015796 | 0.07693937 | 1.113851374 |
| 7 | 0.14281313 | 0.000210582 | 0.034793251 | 1.458504986 |
| 8 | 0.131250356 | 0.000653735 | 0.061785203 | 1.20911552 |
| 9 | −0.136567235 | 0.000396624 | 0.048307035 | 1.315989617 |
| 10 | −0.167797497 | 1.25E−05 | 0.00713101 | 2.146848976 |
| 11 | 0.12934233 | 0.000784205 | 0.06774776 | 1.169105057 |
| 12 | −0.186764629 | 1.15E−06 | 0.001508484 | 2.821459367 |
| 13 | 0.130056475 | 0.000746363 | 0.066162071 | 1.179390908 |
| 14 | −0.180867893 | 3.65E−05 | 0.013425679 | 1.872063727 |
| 15 | 0.171177441 | 9.46E−05 | 0.023242181 | 1.633723123 |
| 16 | 0.12350647 | 0.001370526 | 0.088453204 | 1.053286432 |
| 17 | 0.154118366 | 6.10E−05 | 0.018171137 | 1.740617897 |
| 18 | −0.229382074 | 7.43E−06 | 0.005324801 | 2.273696627 |
| 19 | 0.259944736 | 8.57E−12 | 1.50E−07 | 6.824292619 |
| 20 | 0.156003591 | 0.000215998 | 0.035269649 | 1.452598864 |
| 21 | 0.123872918 | 0.001325697 | 0.087067268 | 1.060145084 |
| 22 | 0.169634044 | 0.000109585 | 0.025011635 | 1.60185792 |
| 23 | −0.165209175 | 1.75E−05 | 0.008648353 | 2.063066567 |
| 24 | −0.150306215 | 9.75E−05 | 0.023645144 | 1.626258031 |
| 25 | −0.152770888 | 0.000281545 | 0.040344255 | 1.394218297 |
| 26 | −0.171586685 | 4.61E−05 | 0.015266662 | 1.816255918 |
| 27 | 0.133710913 | 0.001444798 | 0.090741388 | 1.042194583 |
| 28 | −0.134934131 | 0.000466016 | 0.052279018 | 1.281672576 |
| 29 | 0.136031113 | 0.000418265 | 0.049603949 | 1.304483748 |
| 30 | −0.148915127 | 0.000109297 | 0.024993655 | 1.602170231 |
| 31 | −0.167900206 | 1.31E−05 | 0.007339882 | 2.134310939 |
| 32 | 0.172188306 | 7.39E−06 | 0.005313421 | 2.274625756 |
| 33 | 0.125048412 | 0.001211339 | 0.083408359 | 1.078790426 |
| 34 | 0.139901318 | 0.000920862 | 0.073272717 | 1.135057705 |
| 35 | 0.199626987 | 0.000380194 | 0.047281588 | 1.325307943 |
| 36 | −0.128810208 | 0.000824674 | 0.069420558 | 1.158511899 |
| 37 | 0.32823565 | 2.87E−18 | 2.62E−13 | 12.5821316 |
| 38 | 0.431394941 | 1.06E−31 | 4.37E−26 | 25.35913251 |
| 39 | 0.157022178 | 4.46E−05 | 0.014991312 | 1.824160363 |
| 40 | 0.136463427 | 0.000392749 | 0.048186635 | 1.317073399 |
| 41 | −0.146130946 | 0.000148842 | 0.029187993 | 1.534795764 |
| 42 | 0.185014574 | 1.45E−06 | 0.001777244 | 2.750253057 |
| 43 | 0.127948526 | 0.000894327 | 0.07225136 | 1.141153976 |
| 44 | −0.144816589 | 0.000170927 | 0.031129333 | 1.506830191 |
| 45 | −0.248640663 | 6.95E−11 | 8.40E−07 | 6.075922048 |
| 46 | −0.131995015 | 0.000620122 | 0.060278006 | 1.21984112 |
| 47 | −0.138078033 | 0.001684108 | 0.09773713 | 1.009940419 |
| 48 | −0.176529524 | 2.75E−05 | 0.011318856 | 1.946197484 |
| 49 | −0.173019207 | 3.79E−05 | 0.013713391 | 1.862855153 |
| 50 | 0.125754725 | 0.001116025 | 0.08041676 | 1.094653431 |
| 51 | −0.194365057 | 4.06E−07 | 0.000735919 | 3.133169752 |
| 52 | −0.175828673 | 2.81E−05 | 0.011483339 | 1.939931807 |
| 53 | −0.225515853 | 5.68E−05 | 0.017363395 | 1.760365348 |
| 54 | −0.171893898 | 8.83E−05 | 0.022362355 | 1.650482453 |
| 55 | 0.140546839 | 0.000271486 | 0.039621431 | 1.402069845 |
| 56 | 0.141564056 | 0.000239507 | 0.037243267 | 1.428952226 |
| 57 | −0.278736363 | 2.04E−11 | 2.95E−07 | 6.530699282 |
| 58 | −0.144015164 | 0.000644635 | 0.061354157 | 1.212156004 |
| 59 | 0.166898358 | 1.47E−05 | 0.007817563 | 2.106928614 |
| 60 | 0.157262881 | 0.000250242 | 0.038064066 | 1.419484821 |
| 61 | −0.137607611 | 0.000357576 | 0.045792584 | 1.33920485 |
| 62 | −0.194801043 | 3.82E−07 | 0.000704886 | 3.15188108 |
| 63 | −0.137732018 | 0.000353154 | 0.045438833 | 1.342572833 |
| 64 | −0.17435937 | 5.73E−06 | 0.004521519 | 2.344715608 |
| 65 | −0.240817874 | 2.62E−10 | 2.66E−06 | 5.575293109 |
| 66 | −0.225729929 | 3.45E−09 | 2.01E−05 | 4.69611448 |
| 67 | −0.246858064 | 1.65E−10 | 1.75E−06 | 5.756762992 |
| 68 | −0.130005131 | 0.000763905 | 0.066882072 | 1.174690278 |
| 69 | 0.345057571 | 3.85E−20 | 1.06E−14 | 13.97646035 |
| 70 | 0.14236622 | 0.000215776 | 0.035251028 | 1.452828217 |
| 71 | 0.453005617 | 3.67E−35 | 3.01E−29 | 28.52074825 |
| 72 | −0.148856036 | 0.000111309 | 0.025202078 | 1.598563652 |
| 73 | 0.13995921 | 0.000276248 | 0.039947536 | 1.398510005 |
| 74 | 0.145061401 | 0.000164721 | 0.030600196 | 1.514275795 |
| 75 | −0.239371332 | 3.58E−10 | 3.51E−06 | 5.455264699 |
| 76 | −0.147330357 | 0.000131055 | 0.027442011 | 1.561584068 |
| 77 | 0.13598122 | 0.000420334 | 0.049686523 | 1.30376139 |
| 78 | 0.157105637 | 4.48E−05 | 0.015006243 | 1.82372802 |
| 79 | −0.28133337 | 1.31E−11 | 2.03E−07 | 6.693511957 |
| 80 | −0.172334925 | 7.37E−06 | 0.005312442 | 2.274705822 |
| 81 | 0.130037733 | 0.000747693 | 0.066229879 | 1.178946035 |
| 82 | −0.148559644 | 0.000114911 | 0.025594478 | 1.591853725 |
| 83 | −0.183126962 | 1.34E−05 | 0.007435432 | 2.128693784 |
| 84 | −0.168523409 | 1.18E−05 | 0.006890666 | 2.161738822 |
| 85 | −0.191470189 | 1.21E−05 | 0.007012268 | 2.154141475 |
| 86 | −0.132688495 | 0.000574492 | 0.058200843 | 1.235070725 |
| 87 | 0.160500943 | 3.12E−05 | 0.012167657 | 1.91479305 |
| 88 | −0.214303305 | 2.12E−08 | 8.50E−05 | 4.070747359 |
| 89 | −0.234819265 | 7.38E−10 | 6.19E−06 | 5.208260716 |
| 90 | −0.274402689 | 1.00E−12 | 2.22E−08 | 7.653648154 |
| 91 | −0.146643759 | 0.000844289 | 0.070213528 | 1.153579207 |
| 92 | −0.19289561 | 0.000600699 | 0.05934231 | 1.226635554 |
| 93 | −0.196565997 | 2.86E−07 | 0.000569012 | 3.244878632 |
| 94 | 0.132413063 | 0.001720513 | 0.098833942 | 1.005093883 |
| 95 | 0.13525022 | 0.000451761 | 0.051490256 | 1.288274949 |
| 96 | 0.167009808 | 1.45E−05 | 0.007778483 | 2.109105086 |
| 97 | −0.158216233 | 4.05E−05 | 0.01421938 | 1.847119343 |
| 98 | 0.135633505 | 0.00043502 | 0.050603598 | 1.295818604 |
| 99 | 0.211440243 | 3.37E−08 | 0.000117706 | 3.929201265 |
| 100 | 0.145210865 | 0.000160326 | 0.03032331 | 1.518223387 |
| 101 | 0.131684552 | 0.000632928 | 0.060796236 | 1.21612331 |
| 102 | −0.126622974 | 0.001012164 | 0.076809373 | 1.114585779 |
| 103 | 0.183858199 | 0.001084371 | 0.079438585 | 1.099968499 |
| 104 | −0.168021158 | 1.61E−05 | 0.008215311 | 2.085376013 |
| 105 | −0.160599037 | 3.00E−05 | 0.011906785 | 1.924205496 |
| 106 | 0.133155022 | 0.000554376 | 0.057195937 | 1.242634823 |
| 107 | 0.146387283 | 0.00014486 | 0.02884857 | 1.539875713 |
| 108 | 0.125045205 | 0.001191198 | 0.082851418 | 1.081700055 |
| 109 | −0.184022905 | 0.001073016 | 0.079028895 | 1.10221409 |
| 110 | −0.143053188 | 0.000906088 | 0.072788837 | 1.137935222 |
| 111 | 0.186612476 | 1.22E−06 | 0.001574654 | 2.802814793 |
| 112 | 0.140488413 | 0.000267356 | 0.039386899 | 1.404648215 |
| 113 | −0.164621893 | 1.93E−05 | 0.009120928 | 2.039960964 |
| 114 | 0.128609726 | 0.000855666 | 0.070695423 | 1.150608701 |
| 115 | 0.213475821 | 2.47E−08 | 9.56E−05 | 4.019589456 |
| 116 | 0.12315717 | 0.001391297 | 0.089080613 | 1.050216805 |
| 117 | 0.149988083 | 9.62E−05 | 0.023452465 | 1.629811498 |
| 118 | −0.137485497 | 0.000354654 | 0.045546212 | 1.341547738 |
| 119 | −0.153135206 | 6.98E−05 | 0.019644879 | 1.706750637 |
| 120 | −0.149230794 | 0.00013097 | 0.02743129 | 1.561753774 |
| 121 | 0.124040773 | 0.001294696 | 0.086019109 | 1.06540506 |
| 122 | 0.149308419 | 0.000106016 | 0.024633893 | 1.608466948 |
| 123 | −0.18384364 | 0.00108538 | 0.079473139 | 1.099779633 |
| 124 | −0.143373602 | 0.000882027 | 0.071712598 | 1.144404541 |
| 125 | 0.149526781 | 0.000106057 | 0.024633893 | 1.608466948 |
| 126 | 0.14936757 | 0.000102853 | 0.024284632 | 1.614668478 |
| 127 | 0.154592369 | 5.94E−05 | 0.017907637 | 1.746961729 |
| 128 | −0.181416915 | 2.41E−06 | 0.002517691 | 2.598997539 |
| 129 | 0.202782649 | 0.000305227 | 0.042128787 | 1.37542105 |
| 130 | 0.209944639 | 4.22E−08 | 0.00013492 | 3.869922583 |
| 131 | 0.194407816 | 4.03E−07 | 0.000734785 | 3.13383944 |
| 132 | 0.140093681 | 0.000278314 | 0.040101323 | 1.396841296 |
| 133 | 0.137185062 | 0.000365478 | 0.046313755 | 1.334290004 |
| 134 | 0.126782996 | 0.001014824 | 0.076921857 | 1.113950241 |
| 135 | 0.151900648 | 0.000316908 | 0.042926232 | 1.367277233 |
| 136 | −0.157468157 | 5.38E−05 | 0.016823893 | 1.774073505 |
| 137 | 0.144831245 | 0.000170665 | 0.031117098 | 1.507000908 |
| 138 | −0.210225615 | 4.05E−08 | 0.000131963 | 3.879546537 |
| 139 | 0.127130423 | 0.000982593 | 0.075662392 | 1.121119935 |
| 140 | 0.174446378 | 5.67E−06 | 0.004489765 | 2.347776389 |
| 141 | −0.127505505 | 0.00094886 | 0.074354064 | 1.128695291 |
| 142 | 0.145483026 | 0.000159371 | 0.030200315 | 1.519988533 |
| 143 | −0.160033834 | 3.21E−05 | 0.012335544 | 1.908841686 |
| 144 | −0.143383896 | 0.000630074 | 0.060639385 | 1.217245212 |
| 145 | 0.125532678 | 0.001139067 | 0.081109687 | 1.090927275 |
| 146 | −0.186266398 | 1.27E−06 | 0.001621798 | 2.790003196 |
| 147 | −0.134085952 | 0.000506365 | 0.054569677 | 1.263048614 |
| 148 | −0.15996082 | 0.00014789 | 0.029115963 | 1.535868836 |
| 149 | 0.129492021 | 0.00078737 | 0.067862243 | 1.168371791 |
| 150 | 0.178807116 | 3.37E−06 | 0.003177062 | 2.497974317 |
| 151 | 0.121903039 | 0.001583559 | 0.094751243 | 1.023415086 |
| 152 | 0.168435089 | 1.15E−05 | 0.006804692 | 2.167191557 |
| 153 | 0.153994332 | 6.35E−05 | 0.018548728 | 1.731685878 |
| 154 | 0.188417189 | 0.000807652 | 0.068643441 | 1.163400952 |
| 155 | −0.18317727 | 1.91E−06 | 0.002136991 | 2.670197367 |
| 156 | 0.229195222 | 2.01E−09 | 1.31E−05 | 4.882665718 |
| 157 | 0.138835268 | 0.000316118 | 0.042873704 | 1.367808992 |
| 158 | 0.172682922 | 7.06E−06 | 0.005142361 | 2.28883743 |
| 159 | −0.168736817 | 1.48E−05 | 0.007838531 | 2.105765317 |
| 160 | 0.12129975 | 0.001644588 | 0.096589739 | 1.015069008 |
| 161 | 0.129215732 | 0.000793663 | 0.068114011 | 1.166763545 |
| 162 | 0.204811597 | 9.08E−08 | 0.000246944 | 3.607402189 |
| 163 | −0.143511377 | 0.00065151 | 0.061693677 | 1.209759348 |
| 164 | 0.18691761 | 1.12E−06 | 0.001489772 | 2.826880312 |
| 165 | 0.321267839 | 7.90E−14 | 2.82E−09 | 8.549472545 |
| 166 | 0.131001933 | 0.000694905 | 0.063896051 | 1.194525979 |
| 167 | −0.135785552 | 0.00042854 | 0.050238914 | 1.298959759 |
| 168 | −0.225512893 | 3.68E−09 | 2.07E−05 | 4.684241088 |
| 169 | −0.18314228 | 1.86E−06 | 0.002098925 | 2.678003108 |
| 170 | −0.200750977 | 1.71E−07 | 0.000402867 | 3.394838118 |
| 171 | −0.124957336 | 0.001200826 | 0.083129684 | 1.08024387 |
| 172 | 0.225991168 | 5.47E−05 | 0.016965286 | 1.770438825 |
| 173 | −0.162468398 | 2.38E−05 | 0.010418328 | 1.982201973 |
| 174 | 0.290017777 | 1.76E−07 | 0.000406712 | 3.390713341 |
| 175 | 0.127723184 | 0.000929777 | 0.073606324 | 1.13308487 |
| 176 | 0.120634131 | 0.001745212 | 0.09942708 | 1.002495317 |
| 177 | −0.15062837 | 9.42E−05 | 0.023176082 | 1.634959977 |
| 178 | 0.167633665 | 1.35E−05 | 0.007455552 | 2.127520193 |
| 179 | −0.136270197 | 0.001251725 | 0.084799157 | 1.071608467 |
| 180 | 0.124378152 | 0.001287667 | 0.085799894 | 1.066513248 |
| 181 | −0.154857366 | 5.77E−05 | 0.017572987 | 1.755154421 |
| 182 | −0.142212478 | 0.000224057 | 0.035907636 | 1.444813191 |
| 183 | −0.121450334 | 0.001648989 | 0.096758301 | 1.014311766 |
| 184 | −0.164931119 | 1.81E−05 | 0.008799263 | 2.055553718 |
| 185 | −0.137835315 | 0.001097578 | 0.079836667 | 1.097797602 |
| 186 | −0.131482942 | 0.000651388 | 0.061693677 | 1.209759348 |
| 187 | 0.126008318 | 0.001080844 | 0.079335824 | 1.100530662 |
| 188 | −0.157079483 | 4.61E−05 | 0.015265721 | 1.816282676 |
| 189 | −0.160767283 | 2.94E−05 | 0.011786538 | 1.928613751 |
| 190 | 0.162050884 | 2.61E−05 | 0.01094119 | 1.960935426 |
| 191 | −0.14294822 | 0.000707744 | 0.06455439 | 1.19007422 |
| 192 | 0.181261029 | 0.00127867 | 0.085577179 | 1.067642036 |
| 193 | 0.170481118 | 9.26E−06 | 0.006052306 | 2.21807914 |
| 194 | 0.258502679 | 1.21E−11 | 1.99E−07 | 6.702098059 |
| 195 | 0.276363757 | 3.41E−13 | 8.90E−09 | 8.05042931 |
| 196 | −0.140435407 | 0.000268803 | 0.039433672 | 1.404132779 |
| 197 | −0.209843425 | 4.09E−08 | 0.000132933 | 3.876367369 |
| 198 | −0.173945211 | 6.23E−06 | 0.004708488 | 2.327118576 |
| 199 | −0.131636054 | 0.000641889 | 0.061270203 | 1.212750678 |
| 200 | −0.180331867 | 3.59E−06 | 0.003324837 | 2.478229647 |
| 201 | −0.126103154 | 0.001099629 | 0.07989392 | 1.097486268 |
| 202 | 0.141777972 | 0.000234303 | 0.036829065 | 1.433809304 |
| 203 | −0.160608898 | 3.08E−05 | 0.01206874 | 1.918338081 |
| 204 | 0.222992731 | 5.53E−09 | 2.93E−05 | 4.533215724 |
| 205 | −0.273869484 | 6.53E−08 | 0.00019014 | 3.720927283 |
| 206 | 0.159660665 | 0.00027494 | 0.039870551 | 1.399347765 |
| 207 | −0.165132024 | 1.74E−05 | 0.008628216 | 2.064079016 |
| 208 | 0.163982534 | 2.02E−05 | 0.009368586 | 2.028325949 |
| 209 | 0.197224104 | 6.51E−06 | 0.004855894 | 2.313730788 |
| 210 | −0.169574242 | 1.02E−05 | 0.006342314 | 2.197752276 |
| 211 | −0.166725193 | 7.58E−05 | 0.020543806 | 1.687319098 |
| 212 | 0.134489089 | 0.000482058 | 0.053193049 | 1.274145113 |
| 213 | 0.15715858 | 4.57E−05 | 0.015195022 | 1.818298662 |
| 214 | −0.15995683 | 0.000141963 | 0.028601455 | 1.543611871 |
| 215 | 0.148480994 | 0.000118658 | 0.026023936 | 1.584627011 |
| 216 | 0.214505686 | 2.10E−08 | 8.50E−05 | 4.070747359 |
| 217 | 0.186325283 | 1.22E−06 | 0.001574683 | 2.802806951 |
| 218 | −0.122001503 | 0.001569647 | 0.09437397 | 1.025147777 |
| 219 | −0.142930829 | 0.000208033 | 0.034601574 | 1.460904141 |
| 220 | −0.165820261 | 0.000156754 | 0.029917649 | 1.524072536 |
| 221 | −0.135628235 | 0.000435246 | 0.050603598 | 1.295818604 |
| 222 | −0.229239371 | 1.99E−09 | 1.31E−05 | 4.882665718 |
| 223 | −0.137962807 | 0.001085826 | 0.07947435 | 1.099773013 |
| 224 | 0.143599349 | 0.00019409 | 0.033295416 | 1.477615551 |
| 225 | 0.130035406 | 0.000734251 | 0.065690775 | 1.182495612 |
| 226 | 0.163704637 | 2.15E−05 | 0.009739927 | 2.01144429 |
| 227 | 0.189917314 | 7.78E−07 | 0.001160774 | 2.935252452 |
| 228 | 0.166724498 | 0.0001441 | 0.028775537 | 1.540976569 |
| 229 | 0.154249695 | 0.000443038 | 0.051032678 | 1.292151638 |
| 230 | 0.170518213 | 0.000100746 | 0.024042734 | 1.619016145 |
| 231 | 0.219468104 | 9.69E−09 | 4.63E−05 | 4.334365319 |
| 232 | 0.139276861 | 0.000308729 | 0.042405475 | 1.372578069 |
| 233 | 0.195281623 | 8.05E−06 | 0.005549064 | 2.255780256 |
| 234 | 0.139000319 | 0.000304489 | 0.042062243 | 1.376107569 |
| 235 | −0.141201788 | 0.000243288 | 0.037533044 | 1.425586208 |
| 236 | 0.150552277 | 0.000345759 | 0.044909234 | 1.347664348 |
| 237 | −0.1460043 | 0.000540698 | 0.05649081 | 1.248022199 |
| 238 | −0.134317403 | 0.000504805 | 0.054518599 | 1.263455309 |
| 239 | −0.162079407 | 2.46E−05 | 0.010595787 | 1.974866792 |
| 240 | −0.164389629 | 9.58E−05 | 0.023418895 | 1.630433607 |
| 241 | −0.188187514 | 9.48E−07 | 0.001335378 | 2.874395918 |
| 242 | −0.219696821 | 9.35E−09 | 4.50E−05 | 4.346903561 |
| 243 | −0.196326585 | 0.000476672 | 0.052920075 | 1.276379546 |
| 244 | −0.135084996 | 0.000450188 | 0.051423668 | 1.288836951 |
| 245 | 0.155825859 | 5.31E−05 | 0.016698409 | 1.777324909 |
| 246 | 0.212014567 | 3.08E−08 | 0.000110192 | 3.957848508 |
| 247 | 0.17863675 | 3.24E−05 | 0.012434547 | 1.905370032 |
| 248 | −0.164831934 | 1.80E−05 | 0.008792833 | 2.055871173 |
| 249 | 0.141404038 | 0.000243471 | 0.037539875 | 1.42550718 |
| 250 | −0.146446403 | 0.000147309 | 0.029077374 | 1.536444819 |
| 251 | 0.148830526 | 0.000108996 | 0.024967314 | 1.602628175 |
| 252 | 0.144738245 | 0.000172336 | 0.031241269 | 1.505271334 |
| 253 | 0.260487808 | 8.31E−12 | 1.48E−07 | 6.828588142 |
| 254 | −0.237172812 | 4.93E−10 | 4.51E−06 | 5.34629499 |
| 255 | 0.273606569 | 5.96E−13 | 1.48E−08 | 7.82881682 |
| 256 | −0.129272694 | 0.00080386 | 0.068510528 | 1.164242685 |
| 257 | −0.130197558 | 0.000736428 | 0.065792375 | 1.181824434 |
| 258 | −0.152614229 | 7.58E−05 | 0.020543806 | 1.687319098 |
| 259 | 0.276903472 | 3.06E−13 | 8.67E−09 | 8.062140935 |
| 260 | 0.162849474 | 2.38E−05 | 0.010411441 | 1.982489148 |
| 261 | 0.164171325 | 1.98E−05 | 0.009257844 | 2.033490147 |
| 262 | −0.140381951 | 0.000270271 | 0.039507293 | 1.403322732 |
| 263 | 0.21716391 | 1.39E−08 | 6.29E−05 | 4.201565083 |
| 264 | −0.123458084 | 0.001365134 | 0.088286071 | 1.05410781 |
| 265 | 0.180822076 | 3.67E−05 | 0.013463783 | 1.870832884 |
| 266 | 0.177079665 | 4.06E−06 | 0.003598493 | 2.443879372 |
| 267 | 0.153364304 | 6.81E−05 | 0.01934571 | 1.713415316 |
| 268 | −0.157585636 | 4.24E−05 | 0.014567987 | 1.836600443 |
| 269 | 0.124437687 | 0.001259245 | 0.085025059 | 1.070453058 |
| 270 | 0.203928714 | 1.03E−07 | 0.000272117 | 3.565243838 |
| 271 | 0.162244323 | 0.000163939 | 0.030558601 | 1.514866526 |
| 272 | −0.124078691 | 0.001323181 | 0.087018039 | 1.06039071 |
| 273 | −0.128509743 | 0.000856021 | 0.070695849 | 1.150606086 |
| 274 | 0.125679155 | 0.001104633 | 0.08005217 | 1.096626892 |
| 275 | 0.298178536 | 3.66E−15 | 1.67E−10 | 9.777069594 |
| 276 | −0.195734205 | 3.22E−07 | 0.00061989 | 3.207685433 |
| 277 | −0.156420377 | 4.84E−05 | 0.015787411 | 1.801689096 |
| 278 | −0.204243608 | 2.97E−06 | 0.002891896 | 2.538817283 |
| 279 | 0.226756066 | 3.00E−09 | 1.80E−05 | 4.744696179 |
| 280 | −0.213143812 | 2.72E−08 | 0.000101686 | 3.992737814 |
| 281 | −0.19055326 | 5.82E−06 | 0.004542825 | 2.342674005 |
| 282 | 0.144618858 | 0.000997371 | 0.07637454 | 1.117051394 |
| 283 | −0.149070977 | 0.000108764 | 0.024948874 | 1.602949057 |
| 284 | 0.130938489 | 0.000699119 | 0.064100446 | 1.19313895 |
| 285 | −0.247014572 | 1.61E−10 | 1.74E−06 | 5.75935369 |
| 286 | 0.149340999 | 0.000103149 | 0.024287483 | 1.614617485 |
| 287 | 0.124512854 | 0.001250636 | 0.084771176 | 1.071751793 |
| 288 | −0.157637852 | 0.000177642 | 0.031827504 | 1.497197416 |
| 289 | −0.132737792 | 0.000577239 | 0.058357884 | 1.233900461 |
| 290 | 0.255082021 | 2.14E−11 | 3.03E−07 | 6.519258058 |
| 291 | 0.130069857 | 0.000745416 | 0.06613514 | 1.179567723 |
| 292 | 0.144894447 | 0.000975128 | 0.075399762 | 1.122630024 |
| 293 | 0.125892174 | 0.001083106 | 0.079402578 | 1.100165399 |
| 294 | 0.145716664 | 0.000155496 | 0.029782028 | 1.526045738 |
| 295 | −0.214743903 | 2.03E−08 | 8.38E−05 | 4.07696038 |
| 296 | 0.123351551 | 0.001389893 | 0.089046265 | 1.050384293 |
| 297 | −0.228620739 | 2.21E−09 | 1.39E−05 | 4.858331956 |
| 298 | 0.181692228 | 2.24E−06 | 0.002391393 | 2.621349022 |
| 299 | 0.144373702 | 0.000179037 | 0.031945157 | 1.495594973 |
| 300 | −0.138929289 | 0.000313136 | 0.042698657 | 1.369585785 |
| 301 | 0.122393002 | 0.001515437 | 0.092688152 | 1.032975776 |
| 302 | 0.193226478 | 4.57E−07 | 0.000797523 | 3.098256702 |
| 303 | −0.127029762 | 0.000991833 | 0.076085171 | 1.118699981 |
| 304 | 0.124411831 | 0.001262219 | 0.085101311 | 1.070063748 |
| 305 | −0.132811708 | 0.000573126 | 0.058138132 | 1.235538924 |
| 306 | 0.151232116 | 8.60E−05 | 0.021996582 | 1.657644799 |
| 307 | −0.140566189 | 0.000265245 | 0.039226751 | 1.406417662 |
| 308 | −0.149246425 | 0.000104209 | 0.024380584 | 1.612955902 |
| 309 | 0.349042378 | 3.36E−16 | 2.12E−11 | 10.67350396 |
| 310 | −0.12383057 | 0.001353286 | 0.087879541 | 1.056112221 |
| 311 | 0.158013714 | 4.15E−05 | 0.014413474 | 1.841231334 |
| 312 | 0.123959912 | 0.00129337 | 0.08597973 | 1.065603924 |
| 313 | 0.151202754 | 8.63E−05 | 0.022041807 | 1.656752797 |
| 314 | −0.126327829 | 0.001058527 | 0.078501301 | 1.105123148 |
| 315 | 0.176560443 | 5.61E−05 | 0.017235731 | 1.76357028 |
| 316 | 0.15333247 | 0.000277527 | 0.040062009 | 1.397267276 |
| 317 | −0.139793857 | 0.000293026 | 0.041170846 | 1.385410211 |
| 318 | 0.137188288 | 0.000372861 | 0.046788465 | 1.329861203 |
| 319 | 0.121420676 | 0.001640071 | 0.096460226 | 1.015651726 |
| 320 | 0.142182407 | 0.000219916 | 0.035500129 | 1.449770074 |
| 321 | 0.17052418 | 9.21E−06 | 0.006036554 | 2.219210932 |
| 322 | 0.164835457 | 0.000171719 | 0.031201848 | 1.505819684 |
| 323 | 0.306088916 | 5.62E−16 | 3.08E−11 | 10.5118654 |
| 324 | 0.123323462 | 0.001393432 | 0.089123821 | 1.0500062 |
| 325 | 0.238167148 | 4.41E−10 | 4.12E−06 | 5.385057427 |
| 326 | 0.188847764 | 0.001561292 | 0.094127425 | 1.026283824 |
| 327 | −0.135048409 | 0.001743747 | 0.099404161 | 1.002595437 |
| 328 | −0.159060971 | 0.000290088 | 0.040933129 | 1.387925053 |
| 329 | 0.192030541 | 0.000636378 | 0.061006337 | 1.214625049 |
| 330 | −0.26661177 | 1.71E−06 | 0.00198434 | 2.702383912 |
| 331 | 0.147346344 | 0.000130832 | 0.027417186 | 1.561977121 |
| 332 | −0.151141655 | 0.000578702 | 0.058386963 | 1.233684117 |
| 333 | 0.126273986 | 0.001063811 | 0.078683935 | 1.104113931 |
| 334 | −0.123655379 | 0.001352143 | 0.087846989 | 1.056273118 |
| 335 | 0.151920662 | 7.79E−05 | 0.020827293 | 1.68136717 |
| 336 | 0.142680233 | 0.000211171 | 0.034841523 | 1.457902865 |
| 337 | 0.188366863 | 9.60E−07 | 0.001340589 | 2.872704397 |

TABLE 5
Listing of the 50 human tumor suppressor genes used in
a synthetic lethality analysis. For each tumor suppressor
the frequency of thresholded copy loss across 10,712
TCGA Pan-Cancer Atlas (Taylor et al. Cancer Cell, 33(4):
676-689, 2018) tumor patient samples is included.

| | | | | TCGA | TCGA |
|---|---|---|---|---|---|
| | | | | samples | samples |
| | | | | with | with |
| | | | | partial or | homo- |
| Human | Entrez | Chromo- | | stronger | zygous |
| Gene | ID | some | Kilobase | loss | loss |
| APC | 324 | 5q22.2 | 112737.86 | 25.9% | 0.80% |
| ATM | 472 | 11q22.3 | 108223.53 | 26.0% | 0.76% |
| BAP1 | 8314 | 3p21.1 | 52401 | 31.7% | 0.72% |
| BCL2L11 | 10018 | 2q13 | 111120.91 | 9.2% | 0.14% |
| BRCA1 | 672 | 17q21.31 | 43044.3 | 17.4% | 0.29% |
| BRCA2 | 675 | 13q13.1 | 32315.48 | 31.0% | 0.71% |
| BRIP1 | 83990 | 17q23.2 | 61679.19 | 11.0% | 0.07% |
| CBL | 867 | 11q23.3 | 119206.28 | 26.2% | 0.59% |
| CDC73 | 79577 | 1q31.2 | 193121.96 | 6.2% | 0.17% |
| CDH1 | 999 | 16q22.1 | 68737.29 | 27.3% | 0.68% |
| CDKN1B | 1027 | 12p13.1 | 12717.27 | 13.2% | 0.58% |
| CDKN2A | 1029 | 9p21.3 | 21967.75 | 38.9% | 13.03% |
| CDKN2B | 1030 | 9p21.3 | 22002.9 | 38.7% | 12.56% |
| CHEK2 | 11200 | 22q12.1 | 28687.74 | 27.9% | 0.38% |
| DICER1 | 23405 | 14q32.13 | 95086.23 | 24.5% | 0.35% |
| DIS3 | 22894 | 13q21.33 | 72755.4 | 29.8% | 0.74% |
| EPCAM | 4072 | 2p21 | 47369.15 | 7.4% | 0.08% |
| ERCC4 | 2072 | 16p13.12 | 13920.16 | 13.8% | 0.02% |
| FAT1 | 2195 | 4q35.2 | 186587.78 | 31.7% | 2.12% |
| FBXW7 | 55294 | 4q31.3 | 152321.26 | 29.4% | 0.60% |
| KEAP1 | 9817 | 19p13.2 | 10486.12 | 18.1% | 0.16% |
| KNSTRN | 90417 | 15q15.1 | 40382.72 | 26.9% | 0.68% |
| LATS2 | 26524 | 13q12.11 | 20973.04 | 30.0% | 0.51% |
| MAP2K4 | 6416 | 17p12 | 12020.82 | 36.7% | 1.05% |
| MDM4 | 4194 | 1q32.1 | 204516.38 | 5.6% | 0.03% |
| MEF2B | 4207 | 19p13.11 | 19145.57 | 15.4% | 0.14% |
| MEN1 | 4221 | 11q13.1 | 64803.51 | 15.3% | 0.10% |
| NF1 | 4763 | 17q11.2 | 31094.93 | 18.5% | 1.05% |
| PBRM1 | 55193 | 3p21.1 | 52545.39 | 31.8% | 0.66% |
| PDGFRB | 5159 | 5q32 | 150113.84 | 22.4% | 0.13% |
| PIK3R1 | 5295 | 5q13.1 | 68288.42 | 27.4% | 0.81% |
| PMS1 | 5378 | 2q32.2 | 189784.09 | 10.4% | 0.11% |
| PMS2 | 5395 | 7p22.1 | 5970.93 | 7.2% | 0.16% |
| PPP6C | 5537 | 9q33.3 | 125146.57 | 23.6% | 0.14% |
| PTEN | 5728 | 10q23.31 | 87863.44 | 31.5% | 4.35% |
| PTPN11 | 5781 | 12q24.13 | 112418.9 | 13.1% | 0.07% |
| PTPRD | 5789 | 9p24.1-p23 | 8314.25 | 34.8% | 3.85% |
| PTPRT | 11122 | 20q12-q13.11 | 42072.75 | 4.4% | 0.15% |
| RBM10 | 8241 | Xp11.3 | 47145.22 | 19.2% | 0.49% |
| RHOA | 387 | 3p21.31 | 49359.14 | 31.6% | 0.62% |
| RUNX1 | 861 | 21q22.12 | 34787.8 | 22.1% | 0.50% |
| SETD2 | 29072 | 3p21.31 | 47016.41 | 31.3% | 0.58% |
| SMAD4 | 4089 | 18q21.2 | 51030.21 | 33.2% | 1.56% |
| SMARCA4 | 6597 | 19p13.2 | 10960.92 | 18.1% | 0.31% |
| SRSF2 | 6427 | 17q25.1 | 76734.12 | 11.2% | 0.09% |
| STK11 | 6794 | 19p13.3 | 1205.8 | 26.3% | 0.77% |
| TP53 | 7157 | 17p13.1 | 7668.4 | 38.1% | 1.17% |
| TSC1 | 7248 | 9q34.13 | 132891.35 | 24.4% | 0.38% |
| TSC2 | 7249 | 16p13.3 | 2048.2 | 16.3% | 0.22% |
| VHL | 7428 | 3p25.3 | 10141.64 | 26.9% | 0.30% |

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for inducing cell death or reducing cell survival of a rhabdomyosarcoma cell characterized by a loss of VPS4B expression, the method comprising contacting the cell with an agent that inhibits the expression or activity of VPS4A, thereby inducing cell death or reducing cell survival of the rhabdomyosarcoma cell; or comprising contacting the cell with an agent that inhibits the expression or activity of VPS4B, thereby inducing cell death or reducing cell survival of the rhabdomyosarcoma cell.

2. The method of claim 1 further comprising contacting the cell with an agent that inhibits the expression or activity of ULK3, CHMP1A, CHMP1B, VTA1, and/or IST1.

3. A method for inducing cell death or reducing cell survival of a neoplastic cell characterized by a loss of VPS4A expression, the method comprising contacting the cell with an agent that inhibits the expression or activity of ULK3, CHMP1A, CHMP1B, VTA1, and/or IST1;
or characterized by a loss of VPS4B expression, the method comprising contacting the cell with an agent that inhibits the expression or activity of ULK3, CHMP1A, CHMP1B, VTA1, and/or IST1, thereby inducing or promoting cell death or reducing cell survival of the neoplastic cell, thereby inducing or promoting cell death or reducing cell survival of the neoplastic cell.

4. The method of claim 3 further comprising contacting the cell with an agent that inhibits the expression or activity of VPS4B or VPS4A.

5. The method of claim 3, wherein the neoplastic cell is a brain, bladder, bile, blood, breast, duct, colon, colorectal, esophageal, gastric, germ cell, liver, ovarian, pancreatic, uterine, or lung cancer cell.

6. The method of claim 5, wherein the neoplastic cell is a pancreatic cancer cell, renal cell carcinoma, pancreatic ductal adrenocarcinoma, sarcoma cell, osteosarcoma cell or a rhabdomyosarcoma cell.

7. The method of claim 1, wherein the rhabdomyosarcoma or neoplastic cell is further characterized by a loss of SMAD4 or CDH1.

8. The method of claim 1 further comprising contacting the cell with an interferon.

9. The method of claim 1, wherein the agent comprises SU6668 and/or MSC1094308.

10. The method of claim 1, wherein the agent is shRNA and comprises a sequence, from 5′ to 3′, selected from the three sequences GCAAGAAGCCAGUCAAAGAGA (SEQ ID NO: 1), CGAGAAGCUGAAGGAUUAUUU (SEQ ID NO: 2), and GCCGAGAAGCUGAAGGAUUAU (SEQ ID NO: 3); any of the three sequences truncated by 1, 2, 3, 4, or 5 nucleotides at the 5′ and/or 3′ end; and variants of any of the three sequences comprising 1, 2, 3, 4, or 5 nucleobase substitutions.

11. The method of claim 1, wherein the agent is a CRISPR-spCas9 system comprising a single-guide RNA (sgRNA) that targets VPS4A and comprises a sequence, from 5′ to 3′, selected from the four sequences ACUCACACUUGAUAGCGUGG (SEQ ID NO: 4), GGGCCGCACGAAGUACCUGG (SEQ ID NO: 5), AUUGUUAUUCCCCACCCCUG (SEQ ID NO: 6), and CCACUUAGAAACAAGAUCAG (SEQ ID NO: 7); any of the four sequences truncated by 1, 2, 3, 4, or 5 nucleotides at the 5′ and/or 3′ end; and variants of any of the four sequences comprising 1, 2, 3, 4, or 5 nucleobase substitutions.

12. A method for treating a subject having a neoplasia characterized by a loss of VPS4A or VPS4B expression, the method comprising administering to the subject an agent that inhibits the expression or activity of ULK3, CHMP1A, CHMP1B, VTA1, or IST1, thereby inducing or promoting cell death or reducing cell survival of the neoplasia.

13. The method of claim 12 further comprising administering an interferon.

14. The method of claim 12, wherein the agent comprises SU6668 and/or MSC1094308.

15. The method of claim 12, wherein the agent is shRNA and comprises a sequence, from 5′ to 3′, selected from the three sequences GCAAGAAGCCAGUCAAAGAGA (SEQ ID NO: 1), CGAGAAGCUGAAGGAUUAUUU (SEQ ID NO: 2), and GCCGAGAAGCUGAAGGAUUAU (SEQ ID NO: 3); any of the three sequences truncated by 1, 2, 3, 4, or 5 nucleotides at the 5′ and/or 3′ end; and variants of any of the three sequences comprising 1, 2, 3, 4, or 5 nucleobase substitutions.

16. The method of claim 12, wherein the agent is a CRISPR-spCas9 system comprising a single-guide RNA (sgRNA) that targets VPS4A and comprises a sequence, from 5′ to 3′, selected from the four sequences ACUCACACUUGAUAGCGUGG (SEQ ID NO: 4), GGGCCGCACGAAGUACCUGG (SEQ ID NO: 5), AUUGUUAUUCCCCACCCCUG (SEQ ID NO: 6), and CCACUUAGAAACAAGAUCAG (SEQ ID NO: 7); any of the four sequences truncated by 1, 2, 3, 4, or 5 nucleotides at the 5′ and/or 3′ end; and variants of any of the four sequences comprising 1, 2, 3, 4, or 5 nucleobase substitutions.

17. A method for treating a selected subject having cancer characterized by a loss of VPS4A expression, the method comprising:
administering an agent that inhibits the expression of VPS4B, ULK3, CHMP1A, CHMP1B, VTA1, and/or IST1, wherein the subject is selected if the cancer is determined to have VPS4A dependency, wherein dependency is determined using a multivariate model, wherein levels of a VPS4B marker and levels of at least one of a CHMP4B, ITCH, and ISG15 marker are used as inputs to the model, thereby treating the subject.

18. The method of claim 17, wherein the cancer is further characterized by a loss of SMAD4 or CDH1.

19. The method of claim 17 further comprising administering an interferon.

20. The method of claim 17, wherein the agent comprises a small molecule compound, polypeptide, or polynucleotide.

21. The method of claim 20, wherein the agent comprises SU6668 and/or MSC1094308.


SEQUENCE LISTING (50 sequences)

SEQ ID NO: 1 - RNA, 21 nt, synthetic construct
  misc_feature 1..21: note = source = /note="Description of Artificial Sequence: Syntheticoligonucleotide"
  source 1..21: mol_type = other RNA
gcaagaagccagtcaaagaga

SEQ ID NO: 2 - RNA, 21 nt, synthetic construct
  misc_feature 1..21: note = source = /note="Description of Artificial Sequence: Syntheticoligonucleotide"
  source 1..21: mol_type = other RNA
cgagaagctgaaggattattt

SEQ ID NO: 3 - RNA, 21 nt, synthetic construct
  misc_feature 1..21: note = source = /note="Description of Artificial Sequence: Syntheticoligonucleotide"
  source 1..21: mol_type = other RNA
gccgagaagctgaaggattat

SEQ ID NO: 4 - RNA, 20 nt, synthetic construct
  misc_feature 1..20: note = source = /note="Description of Artificial Sequence: Syntheticoligonucleotide"
  source 1..20: mol_type = other RNA
actcacacttgatagcgtgg

SEQ ID NO: 5 - RNA, 20 nt, synthetic construct
  misc_feature 1..20: note = source = /note="Description of Artificial Sequence: Syntheticoligonucleotide"
  source 1..20: mol_type = other RNA
gggccgcacgaagtacctgg

SEQ ID NO: 6 - RNA, 20 nt, synthetic construct
  misc_feature 1..20: note = source = /note="Description of Artificial Sequence: Syntheticoligonucleotide"
  source 1..20: mol_type = other RNA
attgttattccccacccctg

SEQ ID NO: 7 - RNA, 20 nt, synthetic construct
  misc_feature 1..20: note = source = /note="Description of Artificial Sequence: Syntheticoligonucleotide"
  source 1..20: mol_type = other RNA
ccacttagaaacaagatcag

SEQ ID NO: 8 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 9 - DNA, 4811 bp, Homo sapiens
  source 1..4811: mol_type = genomic DNA
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaa

SEQ ID NO: 10 - protein, 240 aa, Homo sapiens
MDVHGEAAGEAGQEGGEGLQGGAGQSEEGPSAEKCRVCPCVCRERHPQEERRCELASDGVPRRRSGLQGADSCDYEGGDQEYGPGDQSPGQGPEHHGPAEGLLSDGQVRAAGAEPGRPYIGDGGLHELGHHPDHAAGAGGQPHHADRRGEWPGGAGPAQPAARGRLCRGRELCAQPGGPAVTEVGRLEELAVPRRCAPPLPRDVLEGSCPLPTASCLCADPAGLRPAATLRLSPARPAWP

SEQ ID NO: 11 - DNA, 2322 bp, Homo sapiens
  source 1..2322: mol_type = genomic DNA
ggcgaccccggaagtccccgccgggtgcagcttggtcggttcgatcgccgccgggacctgacaccgcccggagttggcgtcccttctccctctccgagtgctgctcctgtcattgtggccatggacgttcacggcgaagcagctggagaagctggccaagaaggcggagaaggactccaaggcggagcaggccaaagtgaagaaggcccttctgcagaaaaatgtagagtgtgcccgtgtgtatgccgagaacgccatccgcaagaagaacgaaggtgtgaactggcttcggatggcgtcccgcgtagacgcagtggcctccaaggtgcagacagctgtgactatgaagggggtgaccaagaatatggcccaggtgaccaaagccctggacaaggccctgagcaccatggacctgcagaaggtctcctcagtgatggacaggttcgagcagcaggtgcagaacctggacgtccatacatcggtgatggaggactccatgagctcggccaccaccctgaccacgccgcaggagcaggtggacagcctcatcatgcagatcgccgaggagaatggcctggaggtgctggaccagctcagccagctgcccgagggcgcctctgccgtgggcgagagctctgtgcgcagccaggaggaccagctgtcacggaggttggccgccttgaggaactagccgtgccccgccggtgtgcaccgcctctgccccgtgatgtgctggaaggctcctgtcctctccccaccgcgtcttgcctttgtgctgaccccgcggggctgcggccggcagccactctgcgtctctcacctgccaggcctgcgtggccttagggttgttcctgttcttttaggttgggcggtgggtctgtgtcctggtgttgagtttctgcaaatttctgggggtgatttctgtgactctgggcccacagcggggaggccaagaggggccctgtggactttcacccagcactgtgggggccttcagactctggggcagcagacatgctgcttcccatcagccagagggggtcagggctgccctgttgccaaacaactccctgaggcctctccgcaccacctcagcgggcaggaggtcccaccatgtggacagacatagcccaaggaggcaccacaggtctatgtgtgctgggggatgtcaggtgccacccaacgctgtcctggtggtatttacaatgacatcctcctcctccatcactccaggggtggtgtctcggccgcccctaccagctggctgagccccctggcctcctgcgctccctcacttccctcagttcccaaagctgcccagtccatggggacagaaccgtcactcagatccacattcaagtgtgcccaccctgcagtcttcatcctcactcagctgctgcctctggaggtgcctttggccacatgtgctgtgctgtttgtctcctcgacagggagcctgtccaccagcaggctgcggtcccagcgggtgcgtctgcagctcctccccttgggcagcctggttctcccggaggacctttccttggggccctgcttcatgacgatgctgcctgtgtcaccctctaccatctgtaaacaactgggtgccttccccgaccacaccccaatgccttcccagcttggaagccaaggcagctgatgaagggagctcaggagagccgtcttcagctgggctggggttggggctgctgtgaggaaaacctgccattgtggccctggagagtcaccagcagctcttgggaaggacttgctgggaggctgagagaggctttgggcacagcctgctgtcttttccatttcctaaagtttacttcattgtcttgaggcttccaggttttgtttttgtttttgccaaagtagaaaaggcaggtggtgggcggctggcagggagtgcgggtccccgcccctcttcagtcctgccctcccctcctcagtcctgcccaccccgtgcagcccatgctgaggctgcagtggtgtcgtgggtgttacgtgcaggaacgtggagaccctgacgtgggctcactgcgtttggttttcttttcagaacttgggagcccccagggaggggctagtgttggtaggtcctagacgtggttccctccagcctccccaaaatcaaccctggtgttgagagaacgtccttctgtccatcgtgggtaacagccttggggagggtgcagagctctgcagagccatgggccaggtggggctgcctcagtcctgtccccttgggcactgaggagaggggcccattcacctttctcctagaatgctgttgtaaataaacaaatggatccctggaaa

SEQ ID NO: 12 - protein, 199 aa, Homo sapiens
MSNMEKHLFNLKFAAKELSRSAKKCDKEEKAEKAKIKKAIQKGNMEVARIHAENAIRQKNQAVNFLRMSARVDAVAARVQTAVTMGKVTKSMAGVVKSMDATLKTMNLEKISALMDKFEHQFETLDVQTQQMEDTMSSTTTLTTPQNQVDMLLQEMADEAGLDLNMELPQGQTGSVGTSVASAEQDELSQRLARLRDQV

SEQ ID NO: 13 - DNA, 1314 bp, Homo sapiens
  source 1..1314: mol_type = genomic DNA
ctgccttcggcgcgcttctcagcagggccgccgacccaaaggagccgtctgactatgtctaacatggagaaacacctgttcaacctgaagttcgcggccaaagaactgagtaggagtgccaaaaaatgcgataaggaggaaaaggccgaaaaggccaaaattaaaaaggccattcagaagggcaacatggaagttgcgaggatacacgccgaaaatgccatccgccagaagaaccaggcggtgaatttcttgagaatgagtgcgcgagtcgatgcagtggctgccagggtccagacggcggtgacgatgggcaaggtgaccaagtcgatggctggtgtggttaagtcgatggatgcgacattgaagaccatgaatctggagaagatttctgctttgatggacaaattcgagcaccagtttgagactctggacgtccagacgcagcaaatggaagacacgatgagcagcacgacgacgctcaccactccccagaaccaagtggatatgctgctccaggaaatggcagatgaggcgggcctcgacctcaacatggagctgccgcagggccagaccggctccgtgggcacgagcgtggcttcggcggagcaggatgaactgtctcagagactggcccgccttcgggatcaagtgtgacggcagaacccgctctgaggtttcctggccatagccaccctttgaaatgctctctgtgtgttagagagatactataccctagaaactctgaacacgccagaatgctgaaatgcccttctacctttgggtttacagccccctccacataaattaagaaattcagtatttctgcactcttagctggattctaaagttctgtatagctcgtaatgatggtatttttatagcagccttttaacagaactagttaatttcgtgtatatgaatctttctcgaagatctggtcaaaactgtattcagtttcctgcccagaatgatcagattgaaggtggttggtttttattattatttagtgtgattgatagtatctagaatggcaggtggtgcataaaagttaaagagaggggaaagattacttagtttggttatacagttataaacaccatgcagtgtattcggtggactgtgctatttctgtttatcctttgggttttggtttttgttttttttttttgccttcacagtgagactgcaaatgattgttctcataacgtatattattaataaatgtggtcctataatttatactgaaaaaaaaaaaaaaaaaaaaaaaaaaaaaaaaaaaaaaaaaaaaaaaaaaaaaaaaaaaaaaaaaaaaaa

SEQ ID NO: 14 - protein, 224 aa, Homo sapiens
MSVFGKLFGAGGGKAGKGGPTPQEAIQRLRDTEEMLSKKQEFLEKKIEQELTAAKKHGTKNKRAALQALKRKKRYEKQLAQIDGTLSTIEFQREALENANTNTEVLKNMGYAAKAMKAAHDNMDIDKVDELMQDIADQQELAEEISTAISKPVGFGEEFDEDELMAELEELEQEELDKNLLEISGPETVPLPNVPSIALPSKPAKKKEEEDDDMKELENWAGSM

SEQ ID NO: 15 - DNA, 1638 bp, Homo sapiens
  source 1..1638: mol_type = genomic DNA
ggcggcagggagcggcgggactgggagcgggcgcgggagccgacccgagccgagccgagccgagccgagccggagcgggcggcgaaggccggcgcggcgagcagcaaccatgtcggtgttcgggaagctgttcggggctggagggggtaaggccggcaagggcggcccgaccccccaggaggccatccagcggctgcgggacacggaagagatgttaagcaagaaacaggagttcctggagaagaaaatcgagcaggagctgacggccgccaagaagcacggcaccaaaaacaagcgcgcggccctccaggcactgaagcgtaagaagaggtatgagaagcagctggcgcagatcgacggcacattatcaaccatcgagttccagcgggaggccctggagaatgccaacaccaacaccgaggtgctcaagaacatgggctatgccgccaaggccatgaaggcggcccatgacaacatggacatcgataaagttgatgagttaatgcaggacattgctgaccagcaagaacttgcagaggagatttcaacagcaatttcgaaacctgtagggtttggagaagagtttgacgaggatgagctcatggcggaattagaagaactagaacaggaggaactagacaagaatttgctggaaatcagtggacccgaaacagtccctctaccaaatgttccctctatagccctaccatcaaaacccgccaagaagaaagaagaggaggacgacgacatgaaggaattggagaactgggctggatccatgtaatggggtccagcgctggctgggcccagacagactgtggtggcctgcgcagcgagcaggcgtgtgcgtgtgtggggcaggcaggatgtggtgcaggcaggttccatcgctttcgactctcactccaaagcagtagggccgcgttgctgctcactctctgcatagcatggtctgcacctgggagatgggcggggggaggggggcgggcggggtgggaagtgcctgctgtttataatgttgaatttctgtaaaataaactgtatttgcaaatccaacattgagcttctggactacgctgactccactgctgaatcctcaatggaaagggtcgactggttgcagttgaaatgacctgaaatgtagcctctgtccttgtaagtcagttgacttgccgcacatctctttgtgtacttgtacggtactggcagaaaagtcatttttcaaaagccataggcttttccttgcccttagctgtaataatgcatctgattttgatttcctccagagctgtgtttctgtccatcacctgtgtattggccctgtgtttaccactctggcccactcctcacccccttgctcccctggtcttctggagtttgtgacattgatttgaaatggatggtgttctcttgagagcaagtgagattgttagaattaagttccaactatacagttttctaacatagctataaggtccttgttgctgtttgtgataactgatagataactcattggaaacgtgcatacatttatattcagatgaaattatggtttgcactgtctattaaatatctcgattaattttcaaaaaaaaaaaaaaaaaaaaaaaaaaaaaaaaaaaaaaaaaaaaaaaaaaa

SEQ ID NO: 16 - protein, 165 aa, Homo sapiens
MGWDLTVKMLAGNEFQVSLSSSMSVSELKAQITQNIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 17 - DNA, 634 bp, Homo sapiens
  source 1..634: mol_type = genomic DNA
cggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagattggcgtgcacgccttccagcagcgtctggctgtccacccgagcggtgtggcgctgcaggacagggtcccccttgccagccagggcctgggccctggcagcacggtcctgctggtggtggacaaatgcgacgaacctctgagcatcctggtgaggaataacaagggccgcagcagcacctacgaggtccggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaagagaat

SEQ ID NO: 18 - protein, 739 aa, Homo sapiens
GDKEPTETIGDLSICLDGLQLESEVVTNGETTCSESASQNDDGSRSKDETRVSTNGSDDPEDAGAGENRRVSGNNSPSLSNGGFKPSRPPRPSRPPPPTPRRPASVNGSPSATSESDGSSTGSLPPTNTNTNTSEGATSGLIIPLTISGGSGPRPLNPVTQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQSKEFDPLGPLPPGWEKRTDSNGRVYFVNHNTRITQWEDPRSQGQLNEKPLPEGWEMRFTVDGIPYFVDHNRRTTTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECDLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYARTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 19 - DNA, 2377 bp, Homo sapiens
  source 1..2377: mol_type = genomic DNA
ggtgacaaagagccaacagagacaataggagacttgtcaatttgtcttgatgggctacagttagagtctgaagttgttaccaatggtgaaactacatgttcagaaagtgcttctcagaatgatgatggctccagatccaaggatgaaacaagagtgagcacaaatggatcagatgaccctgaagatgcaggagctggtgaaaataggagagtcagtgggaataattctccatcactctcaaatggtggttttaaaccttctagacctccaagaccttcacgaccaccaccacccaccccacgtagaccagcatctgtcaatggttcaccatctgccacttctgaaagtgatgggtctagtacaggctctctgccgccgacaaatacaaatacaaatacatctgaaggagcaacatctggattaataattcctcttactatatctggaggctcaggccctaggccattaaatcctgtaactcaagctcccttgccacctggttgggagcagagagtggaccagcacgggcgagtttactatgtagatcatgttgagaaaagaacaacatgggatagaccagaacctctacctcctggctgggaacggcgggttgacaacatgggacgtatttattatgttgaccatttcacaagaacaacaacgtggcagaggccaacactggaatccgtccggaactatgaacaatggcagctacagcgtagtcagcttcaaggagcaatgcagcagtttaaccagagattcatttatgggaatcaagatttatttgctacatcacaaagtaaagaatttgatcctcttggtccattgccacctggatgggagaagagaacagacagcaatggcagagtatatttcgtcaaccacaacacacgaattacacaatgggaagaccccagaagtcaaggtcaattaaatgaaaagcccttacctgaaggttgggaaatgagattcacagtggatggaattccatattttgtggaccacaatagaagaactaccacctatatagatccccgcacaggaaaatctgccctagacaatggacctcagatagcctatgttcgggacttcaaagcaaaggttcagtatttccggttctggtgtcagcaactggccatgccacagcacataaagattacagtgacaagaaaaacattgtttgaggattcctttcaacagataatgagcttcagtccccaagatctgcgaagacgtttgtgggtgatttttccaggagaagaaggtttagattatggaggtgtagcaagagaatggttctttcttttgtcacatgaagtgttgaacccaatgtattgcctgtttgaatatgcagggaaggataactactgcttgcagataaaccccgcttcttacatcaatccagatcacctgaaatattttcgttttattggcagatttattgccatggctctgttccatgggaaattcatagacacgggtttttctttaccattctataagcgtatcttgaacaaaccagttggactcaaggatttagaatctattgatccagaattttacaattctctcatctgggttaaggaaaacaatattgaggaatgtgatttggaaatgtacttctccgttgacaaagaaattctaggtgaaattaagagtcatgatctgaaacctaatggtggcaatattcttgtaacagaagaaaataaagaggaatacatcagaatggtagctgagtggaggttgtctcgaggtgttgaagaacagacacaagctttctttgaaggctttaatgaaattcttccccagcaatatttgcaatactttgatgcaaaggaattagaggtccttttatgtggaatgcaagagattgatttgaatgactggcaaagacatgccatctaccgtcattatgcaaggaccagcaaacaaatcatgtggttttggcagtttgttaaagaaattgataatgagaagagaatgagacttctgcagtttgttactggaacctgccgattgccagtaggaggatttgctgatctcatggggagcaatggaccacagaaattctgcattgaaaaagttgggaaagaaaattggctacccagaagtcatacctgttttaatcgcctggacctgccaccatacaagagctatgagcaactgaaggaaaagctgttgtttgccatagaagaaacagaaggatttggacaagagtaacttctgagaacttgcaccatgaatgggcaagaacttatttgcaatgtttgtccttctctgcctgttgcacatcttgtaaaattggacaatggctctttagagagttatctgagtgtaagtaaattaatgttctcatttaaaaaaaaaaaaaaaaaaa

SEQ ID NO: 20 - protein, 218 aa, Homo sapiens
MHKLSVEAPPKILVERYLIEIAKNYNVPYEPDSVVMAEAPPGVETDLIDVGFTDDVKKGGPGRGGSGGFTAPVGGPDGTVPMPMPMPMPMPSANTPFSYPLPKGPSDFNGLPMGTYQAFPNIHPPQIPATPPSYESVDDINADKNISSAQIVGPGPKPEASAKLPSRPADNYDNFVLPELPSVPDTLPTASAGASTSASEDIDFDDLSRRFEELKKKT

SEQ ID NO: 21 - DNA, 3583 bp, Homo sapiens
  source 1..3583: mol_type = genomic DNA
ccgcccgcgcttgttgtgctgaggccgagggagtcgccattttggatggtgaaccctgaagtcggtgtctgctgcgttcacggcaggattcggttagctaatgcacaagctgagtgtggaagccccacccaaaatcctggtggagagatacctgattgaaattgcaaagaattacaacgtaccctatgaacctgactctgtggtcatggcagaagctcctcctggggtagagacagatcttattgatgttggattcacagatgatgtgaagaaaggaggccctggaagaggagggagtggtggcttcacagcaccagttggtggacctgatggaacggtgccaatgcccatgcccatgcccatgcctatgccatctgcaaatacgcctttctcatatccactgccaaagggaccatcagatttcaatggactgccaatggggacttatcaggcctttcccaatattcatccacctcagataccagcaactcccccatcgtatgaatctgtagatgacattaatgctgataagaatatctcttctgcacagattgttggtcctggacccaagccagaagcctctgcaaagcttccttccagacctgcagataactatgacaactttgtcctaccagagttgccatctgtgccagacacactaccaactgcatctgctggtgccagcacctcagcatctgaagacattgactttgatgatctttcccggaggtttgaagagctgaaaaagaaaacataggtctcttaaaccaggcaactttcacgttttgggagttgagactgagcaatttctccttgtaacaaagaatctccatgaaattctgtttcatctgttaaccgtcactcagcacaacactccctctgggctctcttcctgctcctccagattctgctgctttccagttctctgttgatcctgagactaacaattggagactgaggccagagcaactggctcctggcagctgtgcttgtccgtttcctgtcagagtgatcccaggtttcctcctggcccgtcccatggtccctccacaggagtgtgagaggatgggggaagcactgtgggaagaccaccaaagatggctggacagtgggagagagcacgttgtgaagcatcccagcctcgtgttgaggttccagacttagaaacagacccctctgtacagggggattgtggtgagtgagaatcaaggccaccttgtgtgttttctcactctcgaatgcaagtgggagagggaaaatgactcgggacgccattgtaacggttcctggaagctgggccctctcattggcatatacagtactcctcgctgcagggcactgtcccaccgggatccagttgcaaagtttgtcttgacagttgaaggcctcgcttagttgtactggattctcagggagccctctgtggccttttgctttgcgtgctgtttcccttgtaccagagggcggcaccgtggaaattctgttttccctgtagcatattgtgttggattgcattactggcagagaaaggacaaggtgccattcaagtcctagggtgggcttccagctgccttaatagaagtactcaagtcttttgggtagtgagctggaaagcctacaggaaaagaggggtacctgttttcatttgaaaactttgattcatggaacctttaaaactaatctcagaaaaatttttggtgcccatgcagctgtagttgttcactgctttcctggatggatgggactcttatgtcataacttctgttactcctttggcccatagctaaggtcatccttccccacaggggtggctttgggattggatgatacagcttttgcttctgtgtagtatacctgtacatacttgtttcaggcagcctttctttaatgttttcagttggtttgtattctgtagctcagtagctgctaataaagttaaagatcctgtgtcctgctttctctactgtttggtcagatgatgaagtataaatctggattttagtggagaatattttaaccaagtgatccatgtaaaccagcccttagtttcaggattcctggagcagtacagagctcattttgatctctgtatttagatagcccagatagcatctgtgaatttccttggtcttcctcagtggcagggctccgcatctgccatgcctcatccttggatgtttatttttagtaagttgcagtgagctcacttctgactgcttcatttcccgcagccatcctgggccatgggtggtgagttctgttacttgctgcttggcagcagagctagtttgtctcgtagtattcttgcattgttaatcctatcttgacagtgccaagatccataagaacttgggaccaaggggattttgattcctaacttacagaattaaaaacaaagtatattataattttaaagctatgccatgcaaagataagtagtaatacgctaataaatactaagccaagtaggagataacaggattaaggtagttcatctaaaacttcagtgtcactgcccactgctgtcgtggctgcttgctcagatgaggttttttggggccaactgattcctaacaaatttgagagcttctgtagcagtgtatctattagtgcagcttctttctgagtattgaagacaaaaagagaaaagtgagaaaattgaaattactgctaatagtggagtaaaaaaagtaccaaagatttttaaaaaattagtaaatgtaaagatactatttctttaataatttgcagtcaggcattggagtgtttccactaatggttgcgagccaactatttataggacaatggctatagaatattatgtagtcttataaattgggtttcctaggaagatagctggcagagcttttgaaactaataaagggaatatgatactgtgcattataaattatgtcagcccgtcatcttaggggcagttacagtggagctttcccagtgatataacagcatgctagtttatcttttagttacctaccttaagcgactttctttcttttccaaaggccatgagaatggccgaaacgaaaagattaattaccacaagtaccatcaagatgacactggtgaggatcagaaaggtgcccaggactgggtctaaagcgaaaacctgggaaaacaataagaacacttgcagtgactgacaatttagtttatactttacaaacataagctatatgctagtgtttgggatcttatcagaagaaaagcttatccgaaggaaacttagggagagagtcaaaagataataagaaggaaaatacttttaaatggacagaagagcaggaatgactcattttaaggaggttaagataattgtgactgcagggctttcacaagaaaacaggtgagttgcagtggaattggaaatgattcaaatgtcacatgagttttggcaaaaaatacatctaatcatggaagctaaaagtgggaggggggtaagcaaacactgaaagattaaatgcataccagaaatgagaatgcacactgtctctgccatatcaagaaactacaaccctaaacacaggagcattccaataaaagttaacaggttggccacagaaaa

SEQ ID NO: 22 - protein, 60 aa, Homo sapiens
PEYWCSIAYFEMDVQVGETFKVPSSCPIVTVDGYVDPSGGDRFCLSQLSNVHRTEAIERA

SEQ ID NO: 23 - DNA, 224 bp, Homo sapiens
  source 1..224: mol_type = genomic DNA
atttatttcctatagctcctgagtattggtgttccattgcttactttgaaatggatgttcaggtaggagagacatttaaggttccttcaagctgccctattgttactgttgatggatacgtggacccttctggaggagatcgcttttgtttgagtcaactctccaatgtccacaggacagaagccattgagagagcaaggtattgattgtatagtcagatagtt

SEQ ID NO: 24 - protein, 483 aa, Homo sapiens
MQRNGSASRGLEKTRLRLCREARIPESAFLTGLTRESWEARCWCAKDTREVVAIKCVAKKSLNKASVENLLTEIEILKGIRHPHIVQLKDFQWDSDNIYLIMEFCAGGDLSRFIHTRRILPEKVARVFMQQLASALQFLHERNISHLDLKPQNILLSSLEKPHLKLADFGFAQHMSPWDEKHVLRGSPLYMAPEMVCQRQYDARVDLWSMGVILYEALFGQPPFASRSFSELEEKIRSNRVIELPLRPLLSRDCRDLLQRLLERDPSRRISFQDFFAHPWVDLEHMPSGESLGRATALVVQAVKKDQEGDSAAALSLYCKALDFFVPALHYEVDAQRKEAIKAKVGQYVSRAEELKAIVSSSNQALLRQGTSARDLLREMARDKPRLLAALEVASAAMAKEEAAGGEQDALDLYQHSLGELLLLLAAEPPGRRRELLHTEVQNLMARAEYLKEQVKMRESRWEADTLDKEGLSESVRSSCTLQ

SEQ ID NO: 25 - DNA, 1631 bp, Homo sapiens
  source 1..1631: mol_type = genomic DNA
agaatgcaaagaaatggatcagctagccgagggttggagaagaccaggctgaggctgtgtcgggaagccaggattccagaatcagcattcctcactggcctcacaagggaaagctgggaagcccggtgctggtgtgcaaaggacactcgtgaagtggtagccataaagtgtgtagccaagaaaagtctgaacaaggcatcggtggagaacctcctcacggagattgagatcctcaagggcattcgacatccccacattgtgcagctgaaagactttcagtgggacagtgacaatatctacctcatcatggagttttgcgcagggggcgacctgtctcgcttcatccatacccgcaggattctgcctgagaaggtggcgcgtgtcttcatgcagcaattagctagcgccctgcaattcctgcatgaacggaatatctctcacctggatctgaagccacagaacattctactgagctccttggagaagccccacctaaaactggcagactttggtttcgcacaacacatgtccccgtgggatgagaagcacgtgctccgtggctcccccctctacatggcccccgagatggtgtgccagcggcagtatgacgcccgcgtggacctctggtccatgggggtcatcctgtatgaagccctcttcgggcagcccccctttgcctccaggtcgttctcggagctggaagagaagatccgtagcaaccgggtcatcgagctccccttgcggcccctgctctcccgagactgccgggacctactgcagcggctcctggagcgggaccccagccgtcgcatctccttccaggacttttttgcgcacccctgggtggacctggagcacatgcccagtggggagagtctggggcgagcaaccgccctggtggtgcaggctgtgaagaaagaccaggagggggattcagcagctgccttatcactctactgcaaggctctggacttctttgtacctgccctgcactatgaagtggatgcccagcggaaggaggcaattaaggcaaaggtggggcagtacgtgtcccgggctgaggagctcaaggccatcgtctcctcttccaatcaggccctgctgaggcaggggacctctgcccgagacctgctcagagagatggcccgggacaagccacgcctcctagctgccctggaagtggcttcagctgccatggccaaggaggaggccgccggcggggagcaggatgccctggacctgtaccagcacagcctgggggagctactgctgttgctggcagcggagcccccgggccggaggcgggagctgcttcacactgaggttcagaacctcatggcccgagctgaatacttgaaggagcaggtcaagatgagggaatctcgctgggaagctgacaccctggacaaagagggactgtcggaatctgttcgtagctcttgcacccttcagtgaccctagaagaatgattggacagtgctgagacccccatatcccagagtccccagcctccctcaggttactctgcaccccacagatggtttgatggctgtgctgtatactggaggggagggcaggactctgggagaacagcacttctttcatgagacctttgttactcggtggttact

SEQ ID NO: 26 - protein, 437 aa, Homo sapiens
MTTSTLQKAIDLVTKATEEDKAKNYEEALRLYQHAVEYFLHAIKYEAHSDKAKESIRAKCVQYLDRAEKLKDYLRSKEKHGKKPVKENQSEGKGSDSDSEGDNPEKKKLQEQLMGAVVMEKPNIRWNDVAGLEGAKEALKEAVILPIKFPHLFTGKRTPWRGILLFGPPGTGKSYLAKAVATEANNSTFFSVSSSDLMSKWLGESEKLVKNLFELARQHKPSIIFIDEVDSLCGSRNENESEAARRIKTEFLVQMQGVGNNNDGTLVLGATNIPWVLDSAIRRRFEKRIYIPLPEEAARAQMFRLHLGSTPHNLTDANIHELARKTEGYSGADISIIVRDSLMQPVRKVQSATHFKKVCGPSRTNPSMMIDDLLTPCSPGDPGAMEMTWMDVPGDKLLEPVVCMSDMLRSLATTRPTVNADDLLKVKKFSEDFGQES

SEQ ID NO: 27 - DNA, 4106 bp, Homo sapiens
  source 1..4106: mol_type = genomic DNA
gccctcggactcggctcccgctgcgagcggccgccctgcccgcgcaccgcgctcagcgcccaccgccgggcttcccgcgccggacccagtacctcggctccccggggccggaccgaggccgcaagcagcgccgcggggtgtggggcggacccaggagatgaaatgacaacgtcaaccctccagaaagccattgatctggtgacgaaagccacagaggaggacaaagccaagaactacgaggaggcgctgcggctgtaccagcatgcggtggagtacttcctccacgctatcaagtatgaggcccacagcgacaaggccaaggagagcattcgagccaagtgcgtgcagtacctagaccgggccgagaagctgaaggattatttacgaagcaaagagaaacacggcaagaagccagtcaaagagaaccagagtgagggcaagggcagtgacagtgacagtgaaggggataatccggagaaaaagaaactgcaagaacagctgatgggtgccgtcgtgatggagaagcccaacatacggtggaacgacgtggccgggctggagggggccaaggaggccctcaaagaagctgtcattttgccaatcaaattcccacacttgttcacaggcaagcgcaccccctggcgggggattctgctgttcggaccccctggcacagggaaatcctacctggccaaagccgtggcaacagaggccaacaactccaccttcttctctgtgtcctcctcagatctgatgtccaagtggctgggggagagtgaaaagctggtcaagaacctgtttgagctggccaggcagcacaagccctccatcatcttcatcgatgaggtggattccctctgcgggtcccgaaatgaaaatgagagtgaggccgcccggaggatcaaaacggagttcttggtccagatgcagggggtggggaataacaatgatgggactctggttcttggagccacaaacatcccatgggtgttggattcggccatcaggaggaggtttgaaaaacgaatttatatccccttgccggaggaagctgcccgcgcccagatgttccggttgcatctcgggagcactccccacaacctcacggatgcaaacatccacgagctggcccggaagacggaaggctactcgggcgcggacatcagcatcatcgtgcgggactctctcatgcagcccgtgaggaaggtgcagtcggccacacacttcaaaaaggtctgtggcccctctcgcaccaaccccagcatgatgattgatgacctcctgactccatgctcaccaggggacccaggagccatggagatgacttggatggatgtccctggggacaaactcttagagcctgtggtttgcatgtcggacatgctgcggtctctggccaccacccggcccacggtgaatgcagacgacctcctgaaagtgaagaaattctcagaggactttgggcaagagagttaaaagctgcttcacttgggcaatggtgaaggtgggaggttgattggggcaaatccaggcactccccatgtcaacagccagacagggctccagggcttgtcccagtcaatacagagttccctctgctgtctggccgtctgccagggagccagaaggaagggccttgcagccacagagacactccactgccctggggcacacagtggacactgctcttcctacttcctcctctcctggatgctcatcagctccttctgcctcccccccttttttttccatcttttgttcccctaaattaatgctgcttggattttcatcttatttataaagataaaatcacctggaagtgtcaaggagtggggcggggtggcgggggagaagcagccgtgctgccaggtcacccagacctccagacagccggctagccccactgcccgttccttttacgcccaagttttgctccttgagagcagattggctgatgcccctgcaaccccagcccaagctctgcctcaaagaccgagtgacataagccattcccaccctcctaggttcacatccagggctgtgtcttccttgggggaggagatggtgtcgtttagatcagggtaaggcagtcaggcgggtgttcaccactgccttttcttcctctgagcgtgagaacactgaacccagccactgcccctgggtccctgtcctggaaatggtctaataaatccttttcccttcttgagctacccaagtgatctcatctttccttgactcttaactgagactctgaagggccacccttgcttccaattaaaagaccctctggttttctgtctcctctcttttctaaagcagagcaggaaacagaacactgcagttcttaggcctgttctccagtcaggtgtgcaaggcctcctccccttaaggcctcagaagtttggctggggatgtttgtgggatccagacagttcttgccgttgttcggcctacagatcagagactgcaaagtgggagccctgcgtaccagacccgtcctgcagctgtgtttgtttggtttggtctgccgctaacatttaaaagtcgagagttgctgggcgcggtggctcacgcctgtaatcccagcactttgggccgctaacatttaaaagtcgagagttgctgggcgcggtggctcacgcctgtaatcccagcactttgggaggccgacgcgggcggatcacaaggtcaggaaatcgagaacatcctggctaacacagtgaaaccccgtctcactaaaaataaaaaaattagccgggcgtggtggtgggcgcctgcagtcccagctacttgggaggctgaggcaggagaatggcgtgaacccgggggacggagcttgcagtgagccgagatcgcgccactgcactccagcctgggcgacagagcaagactctgtctcaaaaaaaaaaaaaaagtcgagagttgacataaaaattcagaattctgacttctagaagattaaattaggtaatgttgggcctgcgtcctcatggggcagcctgtcagcggccactaagtagaggctgctctgtacagaaggggtcccccatccccactcatctgtgttgcctgcccagatagccccatggccatttcagtttgcgacccccaacagaccaaggacccagagctggcagccctggacccacgtccggcaggcagtgaagctgcagaggctgtcccacgtttaccccatgcatataaacagaccaaagtcaaagcacattcacacagaaagacctggcctagtaaatgaagcttatagcctgcaaagggctttgttccaaccttccctctccaaaacccagtactgctcctctacctttcaaacctggttgggccagaactgattacatggccaactcttggctcacactgggcttgcactgggccatttctttgctctggttcagagcctctgcagaagtgagctaactttggatcagtagctccaacacttggctccagtgctggcaggttacccctcaaccatagcagctgggatctttgtggcttgggggtgcttgtgtaagttcttggttatgtgccctcagccaccaggatatgaaccgtgtctgcagagcttcactttagtgagcttacaagtttttaaatttttacttctagttacatttacttgaatcagaacgttgtttcctcattcctactgcttaaacaccttgacaagtcctaggggttaacaaaggttagcatggctatggtccatccctgtgctctagttagagcgtgaagacacctgactttccagttgtctctctccatgaccagcaacaggaaccactgacgctgaactttggacagtggcctcagactctggctgccagcacacaacctgccatcatcgatgttaaacatgctgacatgtgcagaggagtttcctccctgaaatgctctgaaattcacttctctgcctggggattctgttataaaccttctgcctacattggctttcactgtggaagttgatttctaaaactctgatgagctcacaacgttgacatgtatatgcgttttttgtgagtgcttcctgctcaaagtgggagagattttactggaaatgcaataaagtttgcattttattgcta

SEQ ID NO: 28 - protein, 444 aa, Homo sapiens
MSSTSPNLQKAIDLASKAAQEDKAGNYEEALQLYQHAVQYFLHVVKYEAQGDKAKQSIRAKCTEYLDRAEKLKEYLKNKEKKAQKPVKEGQPSPADEKGNDSDGEGESDDPEKKKLQNQLQGAIVIERPNVKWSDVAGLEGAKEALKEAVILPIKFPHLFTGKRTPWRGILLFGPPGTGKSYLAKAVATEANNSTFFSISSSDLVSKWLGESEKLVKNLFQLARENKPSIIFIDEIDSLCGSRSENESEAARRIKTEFLVQMQGVGVDNDGILVLGATNIPWVLDSAIRRRFEKRIYIPLPEPHARAAMFKLHLGTTQNSLTEADFRELGRKTDGYSGADISIIVRDALMQPVRKVQSATHFKKVRGPSRADPNHLVDDLLTPCSPGDPGAIEMTWMDVPGDKLLEPVVSMSDMLRSLSNTKPTVNEHDLLKLKKFTEDFGQEG

SEQ ID NO: 29 - DNA, 3337 bp, Homo sapiens
  source 1..3337: mol_type = genomic DNA
agagggcctcgggattgcggaagtttggtggggagggtcggagctctggtggagagagtgttgtctaaaacaagttccggaagggaggctgcccttcgcggtccgagaaccaccggcctccccagtttgagggctgttaccccgtgcgcgcttcgacgttgctgctgttggctctcctcgcccctcgttcccttgggaaccgcctgggaactccgccatgtcatccacttcgcccaacctccagaaagcgatagatctggctagcaaagcagcgcaagaagacaaggctgggaactacgaagaagcccttcagctctatcagcatgctgtgcagtattttcttcatgtcgttaaatatgaagcacagggtgataaagccaagcaaagtatcagggcaaagtgtacagaatatcttgatagagcagaaaaactaaaggagtacctgaaaaataaagagaaaaaagcacagaagccagtgaaagaaggacagccgagtccagcagatgagaaggggaatgacagtgatggggaaggagaatctgatgatcctgaaaaaaagaaactacagaatcaacttcaaggtgccattgttatagaacgaccaaatgtgaaatggagtgacgttgctggacttgaaggagccaaagaagcactgaaagaggctgtgatactgcctattaaatttcctcatctttttacaggcaagagaacaccttggaggggaatcctattatttgggccgcctggaacaggaaagtcctacttagccaaagctgtagcaacagaagccaacaactcaacatttttttcaatatcttcctctgatcttgtttctaagtggctaggtgaaagtgaaaaactggttaagaatttattccaacttgccagagagaacaagccctccattatcttcattgatgaaattgattctctctgtggttcaagaagtgaaaatgaaagtgaagccgcacgtagaattaagacggagttcctagtgcaaatgcaaggggttggtgtagacaatgatggaattttggttctgggagctacaaatataccctgggttctggattctgccattaggcgaagatttgagaaacgaatttatattcccttgccggaaccccatgcccgagcagcaatgtttaaactgcacctagggaccactcagaacagtctcacggaagcagactttcgggaacttgggaggaaaacagatggttattcaggggcagatataagtatcattgtacgtgatgcccttatgcagcctgttaggaaagtacagtcagctactcattttaaaaaggttcgcggaccttcccgagctgatcctaaccatcttgtagatgatctgctaacaccttgctctccaggtgaccctggtgccattgaaatgacatggatggatgtccctggagataaacttttggagccagttgtttccatgtcggatatgttgcggtcactatctaacacaaaacctacagtcaatgaacatgacttgttgaaattaaagaagtttacagaagattttggtcaagaaggctaagccaaagacaaggaagatgcttaccatatgtattctttctttcatagatatttttgtctatttggatcgcattaattgtttccagtaaaactcttttaccacagggaaatacacatctcacttcagagttccattaggttttatattgtacttttcctccattacttattaaatactcctattaacaaaaggtacaaaataacaggttatgaggaaatgagcgatatatgaacggcataaaaacagaaattacccagtaaaaaggatgtcagaaattgacatacaaatatttacaatttttatgaatggtggtctttgcaaagagcatttatattttcttttttttttactaaaatgatatagggtttattttatattttcaaaaaaattgttaaacatcattcttatcaatgtaaaatttacgttattaaaaaattacaaatgatagaatctttactcagggatgggcaataaaacagcaaagagctttgtgattgggtttgaaagattttgaattatagacagtgctcttaatagtttttaataagttgatatttttttctgtgtagatttaaataatttctttaaaagcgtaagctttggctgggcatggtggcttatgcctgtaatcccagcactttgggaggctacgatgggtggatcacctgaggtcaggagttcaagaccagtctggccaacatggtgaaatgccgtctctactaaaaatacaaaaattagctgggcatgatggcgagtgtctgtaatcccagctactcaggaggctgaggcgggagaatcacttgaacctgggaggcagaggttgcagtgagctgagatcgcgctactgcattccagcctgggcgccgagatagcgccattgcactccagcctgggcgactgaataaaactctgtctcaaaaaaaaacaaaaacggtaagcttttatatttaattaactgatacctttcactgtgcaatctcaagaggaagattttctaaattagacattgtctttaccttaaagaagaaaattgtctatttcagtgtcttttttagcaagattgaccaaaacaggtggtaggtgtgtaatttttaactgtcatgaaatactgtaatgcgcacctttgctaataaggaaatggtctccctttgaattatgggaagtttcttgtctgtgatatggggtgtctgtgtacatatatgtatatttccttttttaatacaggataataatatggggtcttgtccttcaccttttaagttcagggaacagtttgtcctgaccacacatatgttatcagtgtaatgctttatgaagctttagaaatgagctcatgactcattaaaaaataataaacgatgctattttaatattttcaaataaataatctgaggatgtgtttcatactttagcatcaacgctgagttgtgccttgtagaattgcattgtctatacatgcaaaagactattaagttaggatgaactagattccctaattacagtgaaattctatcttgcaaatgctatagaataacttgcattttaaagtgtatttgcacattttacatatgctatgtggttgcctttgggttttctgtacagattgtttttgttattaaatggaaaaggcctgaattatgctcttatgtgaagtaaattgatataacctattgactgtattctgtgtattattctttattttgggaccttctgtgtaaaagaaaatattacctaaattggttagatacaaagaaatcattaaaattattaatgtatatatgaaaa

SEQ ID NO: 30 - protein, 307 aa, Homo sapiens
MAALAPLPPLPAQFKSIQHHLRTAQEHDKRDPVVAYYCRLYAMQTGMKIDSKTPECRKFLSKLMDQLEALKKQLGDNEAITQEIVGCAHLENYALKMFLYADNEDRAGRFHKNMIKSFYTASLLIDVITVFGELTDENVKHRKYARWKATYIHNCLKNGETPQAGPVGIEEDNDIEENEDAGAASLPTQPTQPSSSSTYDPSNMPSGNYTGIQIPPGAHAPANTPAEVPHSTGVASNTIQPTPQTIPAIDPALFNTISQGDVRLTPEDFARAQKYCKYAGSALQYEDVSTAVQNLQKALKLLTTGRE

SEQ ID NO: 31 - DNA, 1936 bp, Homo sapiens
  source 1..1936: mol_type = genomic DNA
aggaagtggtgagttcggagtagagatggccgcgcttgcaccgctgcccccgctccccgcacagttcaagagcatacagcatcatctgaggacggctcaggagcatgacaagcgagaccctgtggtggcttattactgtcgtttatacgcaatgcagactggaatgaagatcgatagtaaaactcctgaatgtcgcaaatttttatcaaagttaatggatcagttagaagctctaaagaagcagttgggtgataatgaagctattactcaagaaatagtgggctgtgcccatttggagaattatgctttgaaaatgtttttgtatgcagacaatgaagatcgtgctggacgatttcacaaaaacatgatcaagtccttctatactgcaagtcttttgatagatgtcataacagtatttggagaactcactgatgaaaatgtgaaacacaggaagtatgccagatggaaggcaacatacatccataattgtttaaagaatggggagactcctcaagcaggccctgttggaattgaagaagataatgatattgaagaaaatgaagatgctggagcagcctctctgcccactcagccaactcagccatcatcatcttcaacttatgacccaagcaacatgccatcaggcaactatactggaatacagattcctccgggtgcacacgctccagctaatacaccagcagaagtgcctcacagcacaggtgtagcaagtaatactatccaacctactccacagactatacctgccattgatcccgcacttttcaatacaatttcccagggggatgttcgtctaaccccagaagactttgctagagctcagaagtactgcaaatatgctggcagtgctttgcagtatgaagatgtaagcactgctgtccagaatctacaaaaggctctcaagttactgacgacaggcagagaatgaagcctttgtatgacagacccatgtatttttggcatgaggaactaacagtccattactctatcttcagcctatcaggatcacagttttaaggaagacttggttttgttgaatatgacaatgaaatctgtgtgtatcagatttttattgaagcattcatcagcagcctcaaccagttttcattgtccatttactagattcaatcgtctctgagtatatagggctgatgttagcaagaccctaaaaatgtccattgaaccctgcttcaaaaaatgaaaacacacctctataaaatgtgtactgggaataagctttgtatttacatacattaggggaattttttaaaatctgtaatgtttggacaaacagatgatattactttgctataaaattataaatgtaacttttaataaagatagccagaatattctaaattagaaattacgtttttgtttccctcaagacataaaacaaatataaacattctaaactgctggatgaatctgaaaagacattaagttcaaattttaatttattctcatattaaatataactccattaaaagtttaaaatttcatgggagaaaatataataaggtaaagaggtagaatcactttcagacttaagaataatgttgatttcccaagtgctttaccttatctgttaaagcgtaagatgaattggtatttgcttcataggcagtttgactgcatgtattagagaatgaaaagaagatatttgtagtaatgcctggaaacttggtgctttaaattaaggtactcctctgctgctgtagaatggattccacacagtggatagctatgggtgattcagaatattatgtttagattcccatttgttaagtttataagttttgtggggaattatgaacttactgtgtactacctgcatttgtgctgtgtgaaaaataaatacaaggattcgtttagctaattcaacaaaaaaaaaaaaaaaaaaaaaaaaaaaaaa

SEQ ID NO: 32 - protein, 6 aa, synthetic construct
  REGION 1..6: note = source = /note="Description of Artificial Sequence: Synthetic6xHis tag"
HHHHHH

SEQ ID NO: 33 - DNA, 20 bp, synthetic construct
  misc_feature 1..20: note = source = /note="Description of Artificial Sequence: Syntheticoligonucleotide"
  source 1..20: mol_type = other DNA
aacggcggattgaccgtaat

SEQ ID NO: 34 - DNA, 20 bp, synthetic construct
  misc_feature 1..20: note = source = /note="Description of Artificial Sequence: Syntheticoligonucleotide"
  source 1..20: mol_type = other DNA
ggtgtgcgtatgaagcagtg

SEQ ID NO: 35 - DNA, 20 bp, synthetic construct
  misc_feature 1..20: note = source = /note="Description of Artificial Sequence: Syntheticoligonucleotide"
  source 1..20: mol_type = other DNA
agggagacatccgtcggaga

SEQ ID NO: 36 - DNA, 20 bp, synthetic construct
  misc_feature 1..20: note = source = /note="Description of Artificial Sequence: Syntheticoligonucleotide"
  source 1..20: mol_type = other DNA
agagactgctgaggagtcca

SEQ ID NO: 37 - DNA, 20 bp, synthetic construct
  misc_feature 1..20: note = source = /note="Description of Artificial Sequence: Syntheticoligonucleotide"
  source 1..20: mol_type = other DNA
aagggtatccgccaacacag

SEQ ID NO: 38 - DNA, 20 bp, synthetic construct
  misc_feature 1..20: note = source = /note="Description of Artificial Sequence: Syntheticoligonucleotide"
  source 1..20: mol_type = other DNA
cagtatagacaccacagttg

SEQ ID NO: 39 - DNA, 20 bp, synthetic construct
  misc_feature 1..20: note = source = /note="Description of Artificial Sequence: Syntheticoligonucleotide"
  source 1..20: mol_type = other DNA
actcacacttgatagcgtgg

SEQ ID NO: 40 - DNA, 20 bp, synthetic construct
  misc_feature 1..20: note = source = /note="Description of Artificial Sequence: Syntheticoligonucleotide"
  source 1..20: mol_type = other DNA
gggccgcacgaagtacctgg

SEQ ID NO: 41 - DNA, 20 bp, synthetic construct
  misc_feature 1..20: note = source = /note="Description of Artificial Sequence: Syntheticoligonucleotide"
  source 1..20: mol_type = other DNA
attgttattccccacccctg

SEQ ID NO: 42 - DNA, 21 bp, synthetic construct
  misc_feature 1..21: note = source = /note="Description of Artificial Sequence: Syntheticoligonucleotide"
  source 1..21: mol_type = other DNA
gcaagaagccagtcaaagaga

SEQ ID NO: 43 - DNA, 21 bp, synthetic construct
  misc_feature 1..21: note = source = /note="Description of Artificial Sequence: Syntheticoligonucleotide"
  source 1..21: mol_type = other DNA
gcaagaagcctcacaaagaga

SEQ ID NO: 44 - DNA, 21 bp, synthetic construct
  misc_feature 1..21: note = source = /note="Description of Artificial Sequence: Syntheticoligonucleotide"
  source 1..21: mol_type = other DNA
cgagaagctgaaggattattt

SEQ ID NO: 45 - DNA, 21 bp, synthetic construct
  misc_feature 1..21: note = source = /note="Description of Artificial Sequence: Syntheticoligonucleotide"
  source 1..21: mol_type = other DNA
cgagaagctgttcgattattt

SEQ ID NO: 46 - DNA, 21 bp, synthetic construct
  misc_feature 1..21: note = source = /note="Description of Artificial Sequence: Syntheticoligonucleotide"
  source 1..21: mol_type = other DNA
gccgagaagctgaaggattat

SEQ ID NO: 47 - DNA, 21 bp, synthetic construct
  misc_feature 1..21: note = source = /note="Description of Artificial Sequence: Syntheticoligonucleotide"
  source 1..21: mol_type = other DNA
gccgagaagcactaggattat

SEQ ID NO: 48 - DNA, 20 bp, synthetic construct
  misc_feature 1..20: note = source = /note="Description of Artificial Sequence: Syntheticoligonucleotide"
  source 1..20: mol_type = other DNA
ccacttagaaacaagatcag

SEQ ID NO: 49 - DNA, 24 bp, synthetic construct
  misc_feature 1..24: note = source = /note="Description of Artificial Sequence: Syntheticprimer"
  source 1..24: mol_type = other DNA
gcctaatcatgtttcaggtacaga

SEQ ID NO: 50 - DNA, 20 bp, synthetic construct
  misc_feature 1..20: note = source = /note="Description of Artificial Sequence: Syntheticprimer"
  source 1..20: mol_type = other DNA
ggcaagagaacaccttggag